(19) United States
(12) Patent Application Publication
(10) Pub. No.: US 2026/0085053 A1
(43) Pub. Date: Mar. 26, 2026

(54) BENZOFURAN DERIVATIVES AS SIK INHIBITORS AND THE USE THEREOF

(71) Applicant: BEONE MEDICINES I GMBH, Basel (CH)

(72) Inventors: Jing Li, Beijing (CN); Fengtao Song, Beijing (CN); Zhiwei Wang, Beijing (CN)

(73) Assignee: BEONE MEDICINES I GMBH, Basel (CH)

(21) Appl. No.: 19/405,502

(22) Filed: Dec. 2, 2025

Related U.S. Application Data
(63) Continuation of application No. PCT/CN24/97684, filed on Jun. 6, 2024.

(30) Foreign Application Priority Data
Jun. 6, 2023 (WO) PCT/CN23/98590

(51) Int. Cl. C07D 307/83; A61K 31/343; A61K 31/351; A61K 31/397; A61K 31/4025; A61K 31/403; A61K 31/4035; A61K 31/404; A61K 31/4155; A61K 31/4178; A61K 31/422; A61K 31/4245; A61K 31/438; A61K 31/443; A61K 31/4525; A61K 31/454; A61K 31/4709; A61K 31/4725; A61K 31/496; A61K 31/501; A61K 31/506; A61K 31/5377; A61K 31/538; A61K 31/553; C07D 405/04; C07D 405/12; C07D 405/14; C07D 407/12; C07D 413/12; C07D 413/14; C07D 471/10; C07D 491/048

(52) U.S. Cl. CPC C07D 307/83; A61K 31/343; A61K 31/351; A61K 31/397; A61K 31/4025; A61K 31/403; A61K 31/4035; A61K 31/404; A61K 31/4155; A61K 31/4178; A61K 31/422; A61K 31/4245; A61K 31/438; A61K 31/443; A61K 31/4525; A61K 31/454; A61K 31/4709; A61K 31/4725; A61K 31/496; A61K 31/501; A61K 31/506; A61K 31/5377; A61K 31/538; A61K 31/553; C07D 405/04; C07D 405/12; C07D 405/14; C07D 407/12; C07D 413/12; C07D 413/14; C07D 471/10; C07D 491/048

(57) ABSTRACT

Provided are compounds of formula (I) as well as their compositions and methods of use, wherein values for variables R¹, R², R⁵, X, X¹, and X² are provided herein. The compounds inhibit salt-inducible kinase activity and are useful in the treatment of various inflammatory diseases.

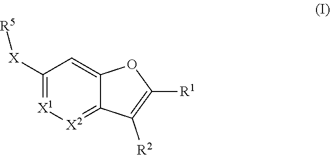

### CROSS REFERENCE TO RELATED APPLICATIONS

This application is a continuation of International Application No. PCT/CN2024/097684, filed on Jun. 6, 2024, which claims priority to International Application No. PCT/CN2023/098590, filed on Jun. 6, 2023, each of which is hereby incorporated by reference in its entirety.

### FIELD OF THE INVENTION

The disclosure herein provides salt-inducible kinase inhibitors as well as their compositions and methods of use.

### BACKGROUND OF THE INVENTION

The salt-inducible kinases (SIK1, SIK2, and SIK3) belong to the AMP-activated protein kinase (AMPK) family.

Small-molecules screening has also identified inhibition of SIKs as a strategy to enhance IL-10 production by macrophages and dendritic cells (Sundberg et al. 2014). Significantly, the IL-10-potentiating effects of the SIK inhibition are also associated with reduced secretion of the pro-inflammatory cytokines IL-1β, IL-6, IL-12, and TNF-α, and these coordinated effects are observed in cells relevant to inflammatory bowel disease (IBD).

It has also been shown that the differentiation from peripheral blood monocytes to macrophages or dendritic cells induced a marked up-regulation of SIKs protein expression (Lombardi et al. 2016). SIKs inhibitors significantly decreased proinflammatory cytokines and increased IL-10 secretion by human myeloid cells upon TLR or IL-1R stimulation.

Thus, there is a need for SIK inhibitors as potential treatments for various diseases and disorders that could benefit from SIK inhibition.

### SUMMARY OF THE INVENTION

In the first embodiment, disclosed herein are benzofuran derivatives of Formula (I). The first embodiment comprises the following aspects:

Aspect 1. A compound of Formula (I):

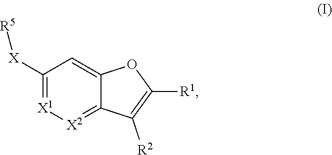

or a pharmaceutically acceptable salt, stereoisomer, or tautomer thereof,

wherein:
X is a divalent heteroaryl ring, a divalent heterocycloalkyl ring, —O—, —CH₂—, —NH— or —O—C₁₋₈alkylene-, wherein each of said divalent heteroaryl ring, heterocycloalkyl ring, —CH₂—, —NH— or —O—C₁₋₈alkylene is independently optionally substituted with at least one R^{a};
   R^{a} is each independently selected from halogen, —C₁₋₈alkyl, —C₁₋₈haloalkyl, —CN, and —C₁₋₈alkoxy;
R⁵ is selected from hydrogen, —C₁₋₈alkyl, —C₁₋₈alkoxy, —C₂₋₈alkynyl, —C₁₋₈haloalkyl, —C₃-C₈cycloalkyl, 4- to 8-membered heterocycloalkyl, aryl, 5- to 12-membered heteroaryl, —NR⁵ⁱR^{5j}, —CONR⁵ⁱR^{5j}, —SR⁵ⁱ, and —NR⁵ⁱCOR^{5j}; wherein said —C₁₋₈alkyl, —C₁₋₈alkoxy, —C₂₋₈alkynyl, —C₁₋₈haloalkyl, —C₃-C₈cycloalkyl, 4- to 8-membered heterocycloalkyl, aryl, or 5- to 12-membered heteroaryl is independently optionally substituted with at least one R^{5a};
   wherein said R^{5a} is each independently selected from halogen, oxo, —OH, —CN, —C₁₋₈alkyl, —C₁₋₈haloalkyl, —C₁₋₈alkoxy, cycloalkyl, heterocycloalkyl, aryl, heteroaryl, —NR^{5b}R^{5c}, and —COR^{5d}; wherein said —C₁₋₈alkyl, —C₁₋₈haloalkyl, —C₁₋₈alkoxy, cycloalkyl, heterocycloalkyl, aryl, or heteroaryl is independently optionally substituted with at least one R^{5g};
      R^{5b}, R^{5c} and R^{5d} are each independently selected from hydrogen, —C₁₋₈alkyl, cycloalkyl, heterocycloalkyl, aryl, and heteroaryl;
      R^{5g} is each independently selected from halogen, oxo, —OH, —CN, —C₁₋₈alkyl, —C₁₋₈haloalkyl, —C₁₋₈alkoxy, C₁₋₈alkyl-CO— and —NO₂,
   wherein said R⁵ⁱ and R^{5j} are each independently selected from hydrogen, —C₁₋₈alkyl, cycloalkyl, heterocycloalkyl, aryl, and heteroaryl; wherein said —C₁₋₈alkyl, cycloalkyl, heterocycloalkyl, aryl, or heteroaryl is independently optionally substituted with at least one R^{5m};
      wherein said R^{5m} is each independently selected from halogen, oxo, —OH, —CN, —C₁₋₈alkyl, —C₁₋₈haloalkyl, —C₁₋₈alkoxy, C₁₋₈alkyl-CO—, and —NO₂;
R¹ is selected from hydrogen, halogen, —C₁₋₈alkyl, —C₁₋₈haloalkyl, and —C₁₋₈alkoxy;
X¹ is N or CR³ and X² is N or CR⁴, with the proviso that X¹ and X² cannot both be N;
   R³ and R⁴ are each independently selected from hydrogen, halogen (F, Cl, Br or I), —C₁₋₈alkyl (preferably —CH₃), —C₁₋₈haloalkyl, —C₁₋₈alkoxy (preferably —OCH₃), —CN, and —NR^{1a}R^{1b}(preferably —NHCH₃);
      R^{1a} and R^{1b} are each independently selected from hydrogen, and —C₁₋₈alkyl (preferably —CH₃);
R² is

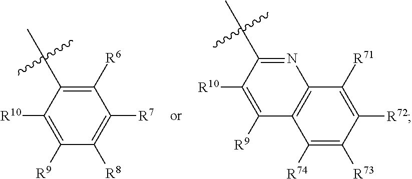

R⁶, and R¹⁰ are each independently selected from hydrogen, halogen, —C₁₋₈alkyl, —C₁₋₈haloalkyl, and —C₁₋₈alkoxy; wherein either
(A) R⁷ and R⁹ are each independently selected from hydrogen, halogen, —C₁₋₈alkyl, cycloalkyl, heterocycloalkyl, —OR^{7a}, —COR^{7a}, —NR^{7d}R^{7e}, —CONR^{7d}R^{7e}, —OH, and —CN;
wherein said —C₁₋₈alkyl, cycloalkyl, or heterocycloalkyl is optionally substituted with at least one R^{7g};
   wherein R^{7g} is each independently selected from hydrogen, halogen, —C₁₋₈alkoxy, —COC₁₋₈alkyl, —NH₂, —OH, and —CN;
   wherein R^{7a} is each independently selected from —C₁₋₈alkyl, C₃₋₈cycloalkyl, and 3- to 10-membered heterocycloalkyl, wherein said —C₁₋₈alkyl, C₃₋₈cycloalkyl, or 3- to 10-membered heterocycloalkyl is optionally substituted with at least one R^{7b};
      wherein R^{7b} is each independently selected from halogen, —OH, —C₁₋₈alkyl, cycloalkyl, heterocycloalkyl, —NH₂, and —C₁₋₈alkoxy, wherein said cycloalkyl, heterocycloalkyl or —C₁₋₈alkoxy is optionally substituted with at least one R^{7c};
      wherein R^{7c} is each independently selected from halogen, —C₁₋₈alkyl, —CN, and —OH;
wherein R^{7d} and R^{7e} are each independently selected from hydrogen, and —C₁₋₈alkyl, wherein said —C₁₋₈alkyl is optionally substituted with at least one R^{7f};
   wherein R^{7f} is each independently halogen, —CN, or —OH;
R⁷², R⁷³, and R⁷⁴ are each independently selected from hydrogen, halogen, —C₁₋₈alkyl, —C₁₋₈haloalkyl, and —C₁₋₈alkoxy;
R⁷¹ is selected from 4- to 15-membered monocyclic, spirocyclic, and bridged polycyclic heterocycloalkyl comprising one, two, or three heteroatoms independently selected from N, O, and S, wherein each of said heterocycloalkyl is independently optionally substituted with at least one R^{71a};
   R^{71a} is each independently selected from hydrogen, halogen, —C₁₋₈alkyl, —C₁₋₈haloalkyl, —C₁₋₈alkoxy and —NH₂;
R⁸ is selected from:
   (i) —CN;
   (ii) 5- to 6-membered monocyclic heteroaryl comprising one, two, or three heteroatoms independently selected from N, O, and S; wherein said heteroaryl is optionally substituted with at least one R^{8a};
      wherein R^{8a} is each independently selected from —C₁₋₈alkyl, and cycloalkyl; and
   (iii) —CONR^{8b}R^{8c}, wherein R^{8b} and R^{8c} are each independently selected from hydrogen, —C₁₋₈alkyl, 5- to 6-membered heterocycloalkyl and C₃-C₈cycloalkyl, wherein said —C₁₋₈alkyl, 5- to 6-membered heterocycloalkyl or C₃-C₈cycloalkyl is optionally substituted with at least one R^{8d}; wherein R^{8d} is each independently selected from halogen, —OH, —NH₂, —CN, —CONH₂, aryl, and heteroaryl; or
(B) R⁸ and R⁹ together with the atoms onto which they are attached form a fused 5-, 6- or 7-membered heterocycloalkyl

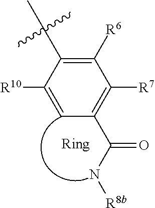

containing —CONR^{8b}— moiety, wherein said heterocycloalkyl is optionally substituted with at least one R^{8e};
   R^{8b} is selected from hydrogen, —C₁₋₈alkyl, 5- to 6-membered heterocycloalkyl and C₃-C₈cycloalkyl, wherein said —C₁₋₈alkyl, 5- to 6-membered heterocycloalkyl or C₃-C₈cycloalkyl is optionally substituted with at least one R^{8d};
      R^{8d} is each independently selected from halogen, —OH, —NH₂, —CN, —CONH₂, aryl, and heteroaryl;
      wherein R^{8e} is selected from —C₁₋₈alkyl, and cycloalkyl;
wherein R⁷ is selected from hydrogen, halogen, —C₁₋₈alkyl, cycloalkyl, heterocycloalkyl, —OR^{7a}, —COR^{7a}, —NR^{7d}R^{7e}, —CONR^{7d}R^{7e}, —OH, and —CN; wherein said —C₁₋₈alkyl, cycloalkyl, or heterocycloalkyl is optionally substituted with at least one R^{7g};
   wherein R^{7g} is each independently selected from hydrogen, halogen, —C₁₋₈alkoxy, —COC₁₋₈alkyl, —NH₂, —OH, and —CN;
   wherein R^{7a} is selected from —C₁₋₈alkyl, C₃₋₈cycloalkyl, and 3- to 10-membered heterocycloalkyl, wherein said —C₁₋₈alkyl, C₃₋₈cycloalkyl, or 3- to 10-membered heterocycloalkyl is optionally substituted with at least one R^{7b};
      wherein R^{7b} is each independently selected from halogen, —OH, —C₁₋₈alkyl, cycloalkyl, heterocycloalkyl, —NH₂, and —C₁₋₈alkoxy, wherein said cycloalkyl, heterocycloalkyl or —C₁₋₈alkoxy is optionally substituted with at least one R^{7c};
      wherein R^{7c} is each independently selected from halogen, —C₁₋₈alkyl, —CN, and —OH;
   wherein R^{7d} and R^{7e} are each independently selected from hydrogen, and —C₁₋₈alkyl, wherein said —C₁₋₈alkyl is optionally substituted with at least one R^{7f};
      wherein R^{7f} is each independently halogen, —CN, or —OH.
         Aspect 2. The compound according to Aspect 1, or a pharmaceutically acceptable salt, stereoisomer, or tautomer thereof, wherein:
R⁵ is selected from:
(i) —C₁₋₈alkyl, aryl and 5- to 12-membered heteroaryl, wherein said —C₁₋₈alkyl, aryl or 5- to 12-membered heteroaryl is optionally substituted with at least one R^{5a};
   wherein R^{5a} is each independently selected from halogen, —OH, —CN, —C₁₋₈alkyl, —C₁₋₈haloalkyl, —C₁₋₈alkoxy, —NR^{5b}R^{5c}, and —COR^{8d};
      R^{5b} and R^{5c} are each independently hydrogen, or —C₁₋₈alkyl;
      R^{5d} is each independently hydrogen, —C₁₋₈alkyl, or cycloalkyl;
(ii) —C₁₋₈alkoxy;
(iii) —C₁₋₈haloalkyl;
(iv) —C₃₋₈cycloalkyl, wherein said —C₃₋₈cycloalkyl is optionally substituted with at least one R^{5a};
   wherein R^{5a} is each independently selected from halogen, —C₁₋₈alkyl, —C₁₋₈haloalkyl, and —C₁₋₈alkoxy;
(v) 3- to 8-membered heterocycloalkyl comprising one, two, or three heteroatoms independently selected from N, O, and S, wherein said 3- to 8-membered heterocycloalkyl is independently optionally substituted with at least one R^{5a};
   wherein R^{5a} is each independently selected from halogen, oxo, —C₁₋₄alkyl, —C₁₋₄alkoxy, C₁₋₄haloalkyl, and —COC₁₋₄alkyl; wherein each of said —C₁₋₄alkyl, —C₁₋₄alkoxy, and —C₁₋₄haloalkyl is independently optionally substituted with at least one R^{5g}; wherein R^{5g} is each independently selected from halogen, —CN, and —C₁₋₈alkyl;
(vi) 5- to 6-membered monocyclic heteroaryl or 8- to 10-membered bicyclic heteroaryl comprising one, two, or three heteroatoms independently selected from N, O, and S; wherein said heteroaryl is each independently optionally substituted with at least one R^{5a},
   wherein R^{5a} is each independently selected from halogen, oxo, —C₁₋₈alkyl, —C₁₋₈alkoxy, and —COC₁₋₈alkyl; and
(vii) —NR⁵ⁱR^{5j}; wherein R⁵ⁱ and R^{5j} are each independently selected from hydrogen, —C₁₋₈alkyl, aryl, and heteroaryl.
   Aspect 3. The compound according to Aspect 1 or 2, or a pharmaceutically acceptable salt, stereoisomer, or tautomer thereof, wherein:
R² is

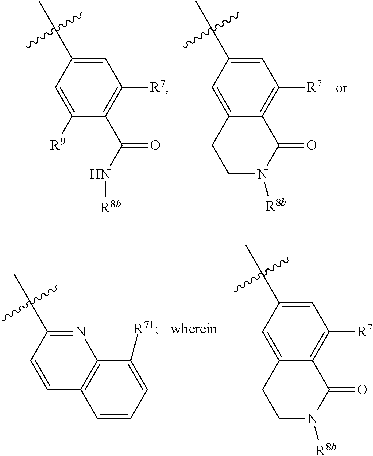

is optionally substituted with at least one R^{8e};
   wherein R^{8b} is each independently selected from hydrogen, —C₁₋₈alkyl, 5- to 6-membered heterocycloalkyl and C₃₋₆cycloalkyl, wherein said —C₁₋₈alkyl, 5- to 6-membered heterocycloalkyl or C₃₋₆cycloalkyl is optionally substituted with at least one R^{8d};
      wherein R^{8d} is each independently selected from halogen (F, Cl, Br and I), —OH, —CN, —NH₂, —CONH₂, aryl, and heteroaryl;
      wherein R^{8e} is each independently selected from —C₁₋₄alkyl, and C₃₋₆cycloalkyl; R⁷¹ is selected from 4- to 15-membered monocyclic, spirocyclic, and bridged polycyclic heterocycloalkyl comprising one, two, or three heteroatoms independently selected from N, O, and S, which each of said heterocycloalkyl is independently optionally substituted with at least one R^{71a};
      R^{71a} is each independently selected from hydrogen, —C₁₋₈alkyl, and —NH₂.
         Aspect 4. The compound according to any one of Aspects 1-3, or a pharmaceutically acceptable salt, stereoisomer, or tautomer thereof, wherein the compound is formula (II-1), (II-2), (II-3), or (II-4),

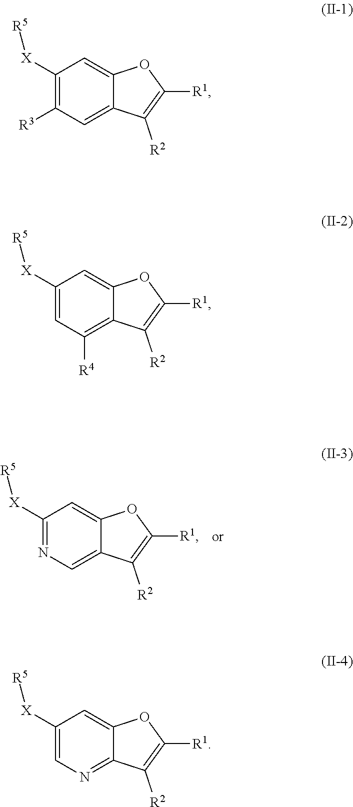

Aspect 5. The compound according to any one of Aspects 1-4, or a pharmaceutically acceptable salt, stereoisomer, or tautomer thereof, wherein
X is selected from:
   a1)

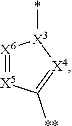

wherein X³, X⁴, X⁵, and X⁶ are each independently selected from N and CR^{A}; R^{A} is each independently selected from hydrogen, halogen, —C₁₋₈alkyl, —C₁₋₈haloalkyl, —CN, and —C₁₋₈alkoxy; and two or three of X³, X⁴, X⁵, and X⁶ are N;
a2)

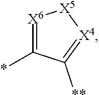

wherein X⁴ and X⁶ are each independently selected from N and CR^{A}; X⁵ is each independently selected from —NR^{B}— or —CR^{C}R^{D}—; two or three of X⁴, X⁵, and X⁶ contain N; and when X⁴ or X⁶ is CR^{A}, R^{A} is each independently selected from hydrogen, halogen, —C₁₋₈alkyl, —C₁₋₈haloalkyl, —CN, and —C₁₋₈alkoxy; when X⁵ is —NR^{B}, R^{B} is each independently selected from hydrogen, —C₁₋₈alkyl, and —C₁₋₈haloalkyl; and when X⁵ is —CR^{C}R^{D}—, R^{C} and R^{D} are each independently selected from hydrogen, halogen, —C₁₋₈alkyl, —C₁₋₈haloalkyl, —CN, and —C₁₋₈alkoxy;
b)

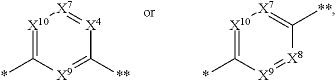

wherein X⁷, X⁸, X⁹, and X¹⁰ are each independently selected from N and CR^{A}, R^{A} is each independently selected from hydrogen, halogen, —C₁₋₈alkyl, —C₁₋₈haloalkyl, —CN, and —C₁₋₈alkoxy; and one, two or three of X⁷, X⁸, X⁹, and X¹⁰ are N;
c)

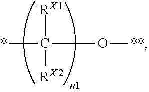

wherein n¹ is 0, 1, 2, 3, or 4; R^{X1} and R^{X2} are each independently selected from hydrogen, halogen, —C₁₋₈alkyl, —C₁₋₈haloalkyl, —CN, and —C₁₋₈alkoxy;
d)

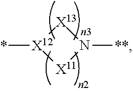

wherein n² and n³ are each independently selected from 0, 1, 2, or 3; wherein X¹² is N or CR^{A}, R^{A} is each independently selected from hydrogen, halogen, —C₁₋₈alkyl, —C₁₋₈haloalkyl, —CN, and —C₁₋₈alkoxy; X¹¹ and X¹³ are each independently selected from —NR^{B}—, and —CR^{C}R^{D}—; when X¹¹ or X¹³ is —NR^{B}—, R^{B} is each independently selected from hydrogen, —C₁₋₈alkyl, and —C₁₋₈haloalkyl; when X¹¹ or X¹³ is —CR^{C}R^{D}—, R^{C} and R^{D} are each independently selected from hydrogen, halogen, —C₁₋₈alkyl, —C₁₋₈haloalkyl, —CN, and —C₁₋₈alkoxy; and
e)

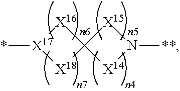

wherein n⁴, n⁵, n⁶ and n⁷ are each independently selected from 0, 1, 2, and 3; wherein X¹⁷ is N or CR^{A}, R^{A} is each independently selected from hydrogen, halogen, —C₁₋₈alkyl, —C₁₋₈haloalkyl, —CN, and —C₁₋₈alkoxy; X¹⁴, X¹⁵, X¹⁶ and X¹⁸ are each independently —NR^{B}—, or —CR^{C}R^{D}—, —wherein R^{B} is each independently selected from hydrogen, —C₁₋₈alkyl, and —C₁₋₈haloalkyl; R^{C} and R^{D} are each independently selected from hydrogen, halogen, —C₁₋₈alkyl, —C₁₋₈haloalkyl, —CN, and —C₁₋₈alkoxy;
wherein * refers to the position attached to R⁵, and ** refers to the position attached to the

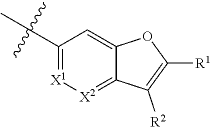

moiety.
   Aspect 6. The compound according to any one of Aspects 1-4, or a pharmaceutically acceptable salt, stereoisomer, or tautomer thereof, wherein
X is selected from

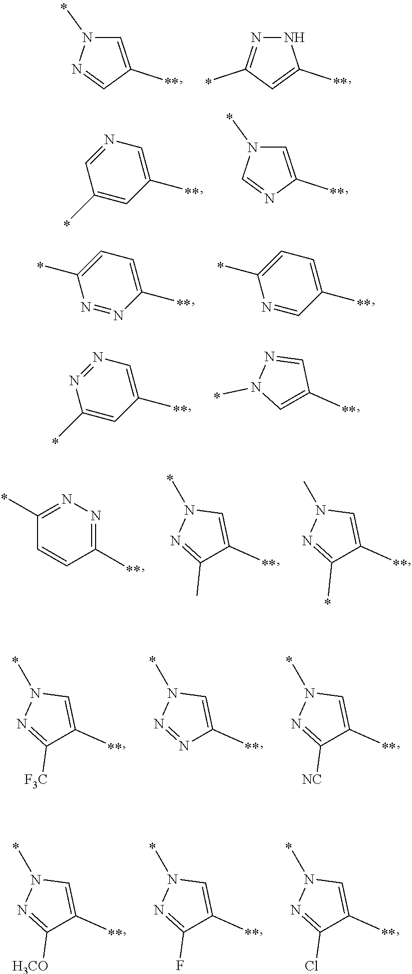

*—O—**, *—CH₂—O—**, *—CH₂—**, *—NH—**, *—CH₂—CH₂—O—**, *—CH₂—CH₂—CH₂—O—**, *—CH₂—CH₂ (CH₃)—O—**

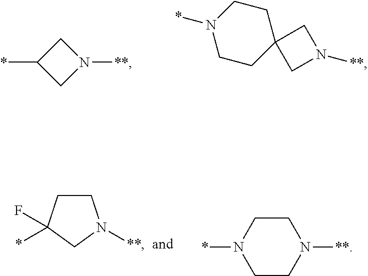

Aspect 7. The compound according to any one of Aspects 1-6, or a pharmaceutically acceptable salt, stereoisomer, or tautomer thereof, wherein
R⁵ is selected from:
   (i) —C₁₋₄alkyl, aryl and 5- to 12-membered heteroaryl, wherein said —C₁₋₄alkyl, aryl or 5- to 12-membered heteroaryl is optionally substituted with at least one R^{5a};
      wherein R^{5a} is each independently selected from halogen, —OH, —CN, —C₁₋₈alkyl, —C₁₋₈haloalkyl, —C₁₋₈alkoxy, —NR^{5b}R^{5c}, or —COR^{5d};
         R^{5b} and R^{5c} are each independently hydrogen, or —C₁₋₈alkyl;
         R^{5d} is selected from hydrogen, —C₁₋₈alkyl, or —C₃₋₆cycloalkyl;
   (ii) —C₁₋₄haloalkyl;
   (iii) —C₁₋₄alkoxy;
   (iv) —C₃₋₆ monocyclic cycloalkyl, wherein each of said —C₃₋₆ monocyclic cycloalkyl is independently optionally substituted with at least one R^{5a},
      wherein R^{5a} is each independently selected from halogen, —C₁₋₈alkyl, —C₁₋₈-haloalkyl, and —C₁₋₈alkoxy;
   (v) 3- to 6-membered monocyclic heterocycloalkyl comprising one, two, or three heteroatoms independently selected from N, O, and S, which each of said 3- to 6-membered monocyclic heterocycloalkyl is independently optionally substituted with at least one R^{5a},
      wherein R^{5a} is each independently selected from selected halogen, oxo, —C₁₋₄alkyl, —C₁₋₄alkoxy, and —COC₁₋₄alkyl; wherein each of said —C₁₋₄alkyl, —C₁₋₄alkoxy, or —COC₁₋₄alkyl is independently optionally substituted with at least one R^{5g};
         wherein R^{5g} is each independently selected from halogen, —CN, and —C₁₋₈alkyl,
   (vi) 5- to 6-membered monocyclic, and 8- to 10-membered fused bicyclic heteroaryl comprising one, two, or three heteroatoms independently selected from N, O, and S; wherein said monocyclic, or 8- to 10-membered fused bicyclic heteroaryl is each independently optionally substituted with at least one R^{5a};
      wherein R^{5a} is each independently selected from halogen, oxo, —C₁₋₈alkyl, —C₁₋₈alkoxy, or —COC₁₋₈alkyl, and
   (vii) —NR⁵ⁱR^{5j}; wherein R⁵ⁱ and R^{5j} are each independently selected from hydrogen, —C₁₋₈alkyl, 5- to 6-membered monocyclic aryl, or 5- to 6-membered monocyclic heteroaryl.
      Aspect 8. The compound according to any one of Aspects 1-7, or a pharmaceutically acceptable salt, stereoisomer, or tautomer thereof, wherein
R⁵ is selected from —CH₃, —CH₂CH₃, —CH(CH₃)₂,

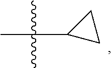 [SMILES: *C1CC1]

-OCH₂CH₃, —OCH₃, —CF₃,

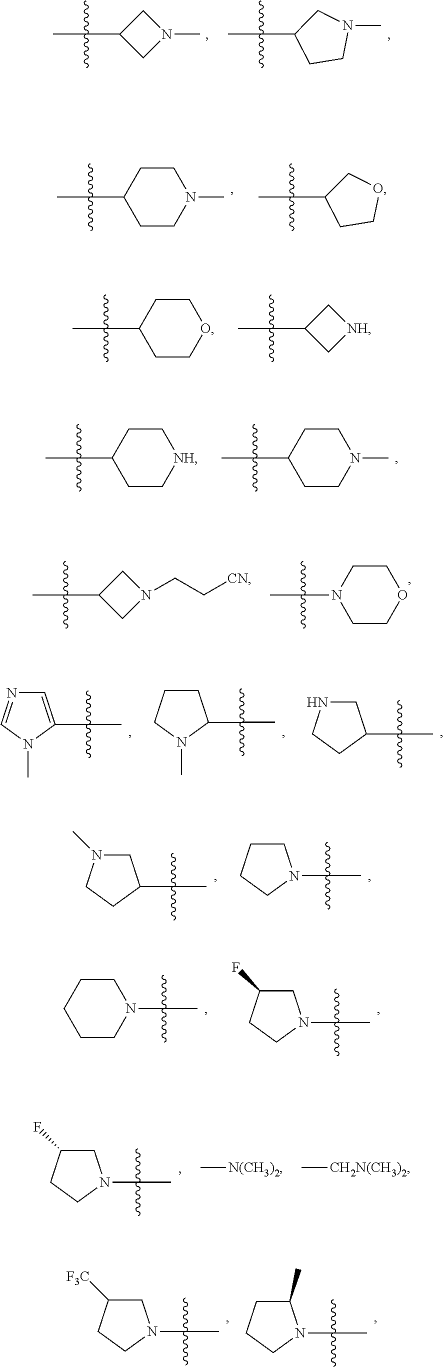

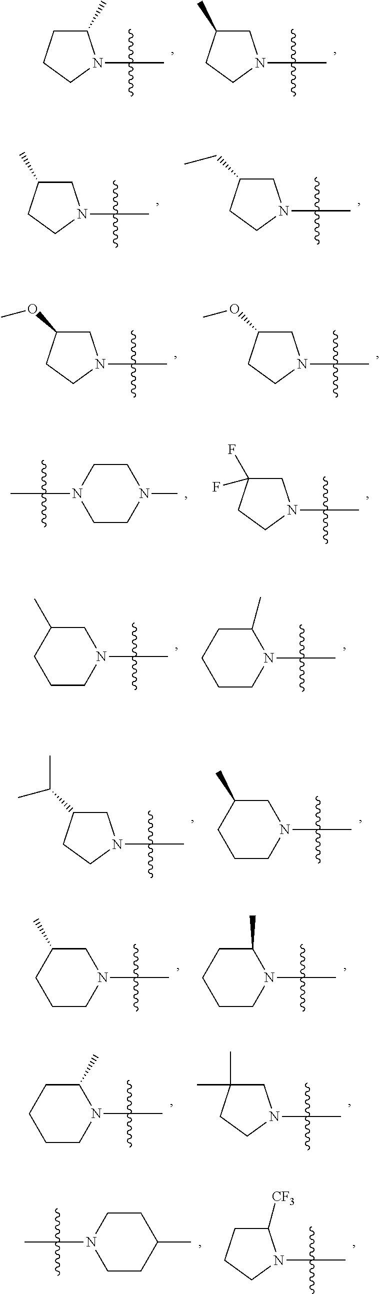

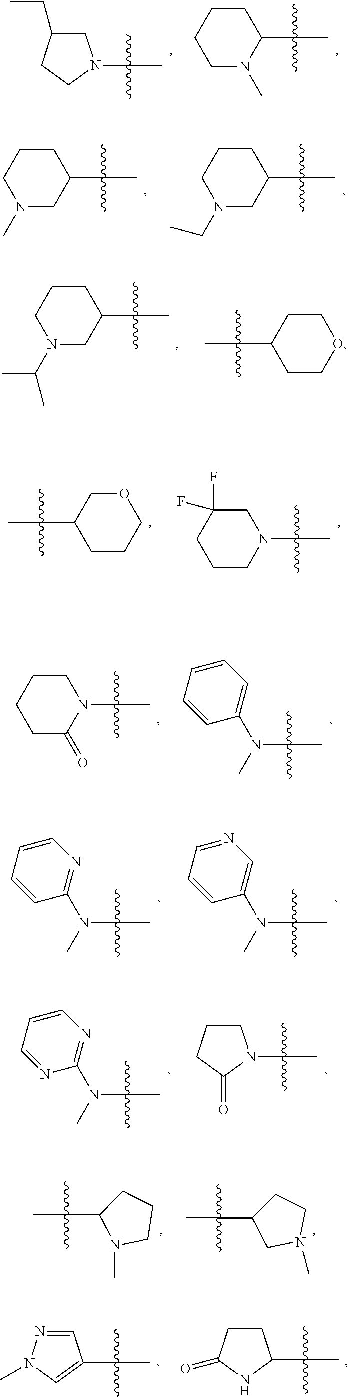

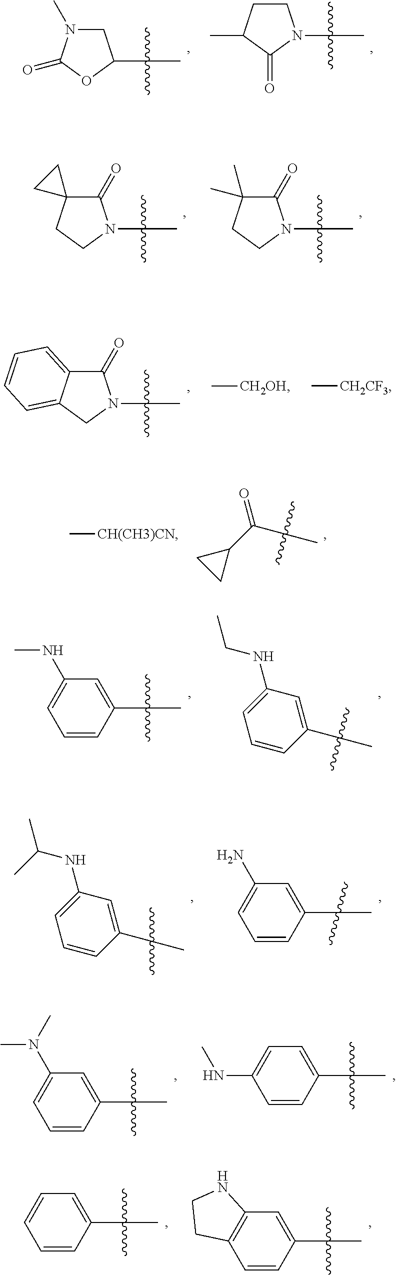

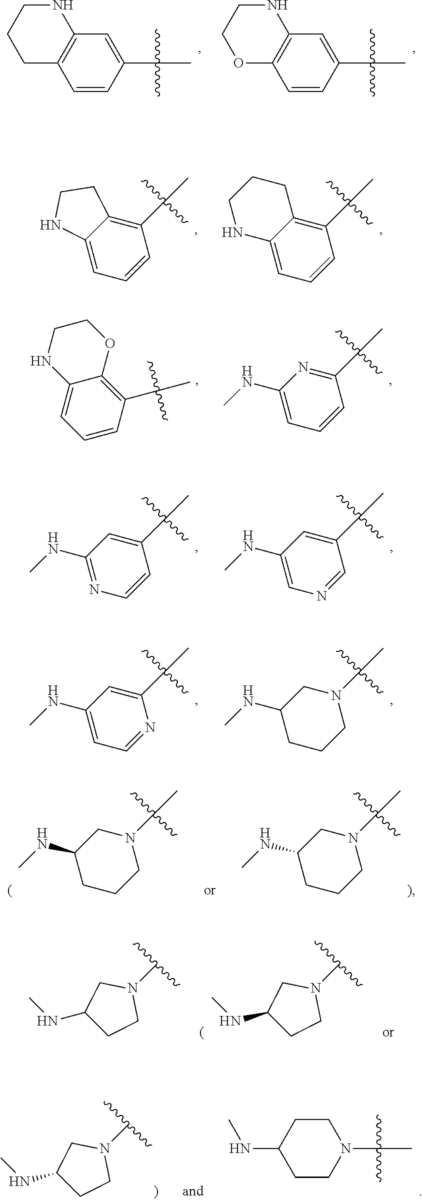

Aspect 9. The compound according to any one of Aspects 1-2, or a pharmaceutically acceptable salt, stereoisomer, or tautomer thereof, wherein
R² is selected from:
a)

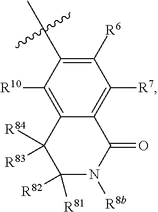

wherein R⁶ is H; R¹⁰ is H;
   R⁷ is hydrogen or —OR^{7a};
   wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b};
   wherein each independently R^{7b} is selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c};
      wherein R^{7c} is each independently-C₁₋₈alkyl, or OH;
   R⁸¹, R⁸², R⁸³, R⁸⁴ are each independently selected from hydrogen, and —C₁₋₈alkyl; and
   R^{8b} is selected from hydrogen, —C₁₋₈alkyl, and C₃-C₈cycloalkyl, wherein said —C₁₋₈alkyl, or C₃-C₈cycloalkyl is optionally substituted with at least one R^{8d};
      R^{8d} is each independently selected from halogen, —OH, —NH₂, —CN, —CONH₂, aryl, and heteroaryl;
b)

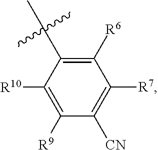

wherein R⁶, R⁹, and R¹⁰ are each independently selected from hydrogen, —C₁₋₈alkyl, and —C₁₋₈alkoxy;
   R⁷ is selected from hydrogen, —C₁₋₈alkyl, C₃-C₈cycloalkyl, and —OR^{7a}, wherein said —C₁₋₈alkyl, or C₃-C₈cycloalkyl is optionally substituted with at least one substituent selected from halogen, —C₁₋₈alkoxy, —COC₁₋₈alkyl, —NH₂, —OH, and —CN;
      R^{7a} is selected from —C₁₋₈alkyl, C₃₋₈cycloalkyl, and 3- to 10-membered heterocycloalkyl; wherein said —C₁₋₈alkyl, C₃₋₈cycloalkyl, or 3- to 10-membered heterocycloalkyl is optionally substituted with at least one R^{7b}, wherein R^{7b} is halogen;
c)

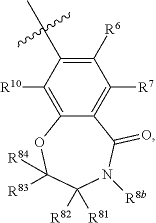

wherein R⁶ is H; R¹⁰ is H;
R⁷ is hydrogen or —OR^{7a};
   wherein R^{7a} is selected from —C₁₋₈alkyl, cycloalkyl, or heterocycloalkyl, wherein said —C₁₋₈alkyl, cycloalkyl, or heterocycloalkyl is optionally substituted with at least one R^{7b};
      wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c};
         wherein R^{7c} is selected from —C₁₋₈alkyl, and OH;
   R⁸¹, R⁸², R⁸³, R⁸⁴ are each independently selected from hydrogen, or —C₁₋₈alkyl; and
   R^{8b} is selected from hydrogen, —C₁₋₈alkyl, and C₃-C₈cycloalkyl, wherein said —C₁₋₈alkyl, or C₃-C₈cycloalkyl is optionally substituted with at least one R^{8d};
      R^{8d} is each independently selected from halogen, —OH, —NH₂, —CN, —CONH₂, aryl, and heteroaryl;
d)

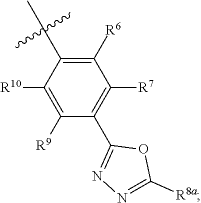

wherein R⁷ and R⁹ are each independently hydrogen or —OR^{7a},
   wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b};
      wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c};
         wherein R^{7c} is each independently selected from —C₁₋₈alkyl, and OH,
      R⁶ and R¹⁰ are both H; R^{8a} is selected from —C₁₋₈alkyl, and cycloalkyl;
e)

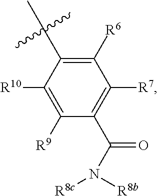

wherein R⁷ and R⁹ are each independently hydrogen or —OR^{7a};
   wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b};
      wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c};
         wherein R^{7c} is each independently selected from —C₁₋₈alkyl, and OH;
   wherein R^{8b} and R^{8c} are each independently selected from hydrogen, —C₁₋₈alkyl, and C₃-C₈cycloalkyl, wherein said —C₁₋₈alkyl, or C₃-C₈cycloalkyl is optionally substituted with at least one R^{8d}; wherein R^{8d} is each independently selected from halogen, —OH, —NH₂, —CN, —CONH₂, aryl, and heteroaryl;
      R⁶ and R¹⁰ are both H; and
f)

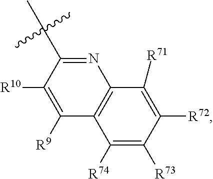

wherein R⁹ and R¹⁰ are each independently selected from hydrogen, halogen, —C₁₋₈alkyl, and —C₁₋₈alkoxy;
   R⁷², R⁷³, and R⁷⁴ are each independently selected from hydrogen, halogen, —C₁₋₈alkyl, —C₁₋₈haloalkyl, and —C₁₋₈alkoxy;
   R⁷¹ is selected from 4- to 15-membered monocyclic, spirocyclic, and bridged polycyclic heterocycloalkyl comprising one, two, or three heteroatoms independently selected from N, O, and S, which each of said heterocycloalkyl is independently optionally substituted with at least one R^{71a};
      R^{71a} is each independently selected from hydrogen, —C₁₋₈alkyl, or —NH₂.
         Aspect 10. The compound according to Aspect 9, a pharmaceutically acceptable salt, stereoisomer, or tautomer thereof, wherein
R² is e)

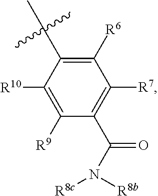

wherein R⁶ and R¹⁰ are both H; R⁷ and R⁹ are each independently-OR^{7a}, wherein R^{7a} is —C₁₋₈alkyl, wherein said —C₁₋₈alkyl is optionally substituted with at least one halogen;
R^{8c} is H; R^{8b} is selected from —(CH₂)ₘ₁CHₘ₂F₃₋ₘ₂, —(CH₂)ₘ₁CN, and

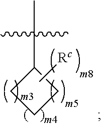

wherein m1 is 1, 2 or 3; m2 is 0, 1, or 2;
m3, m4, and m5 are each independently selected from 0, 1, 2 or 3; and m3+m4+m5>2;
R^{C} is each independently selected from —F, —OH, —CN, and —CONH₂.
   Aspect 11. The compound according to Aspect 9, or a pharmaceutically acceptable salt, stereoisomer, or tautomer thereof, wherein
R² is e)

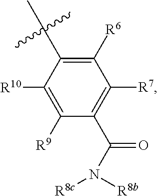

wherein R^{8c} is H; R^{8b} is selected from —(CH₂)ₘ₁CHₘ₂F₃₋ₘ₂, —(CH₂)ₘ₁CN,

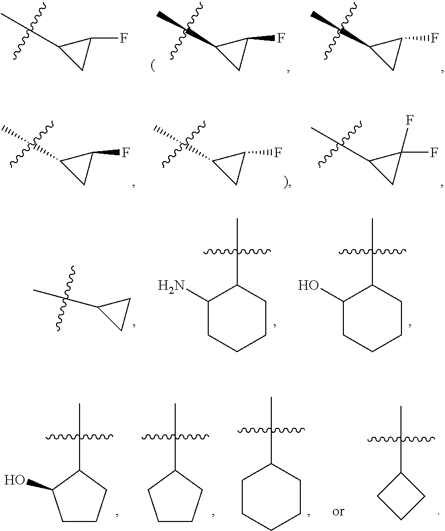

Aspect 12. The compound according to Aspect 9, or a pharmaceutically acceptable salt, stereoisomer, or tautomer thereof, wherein R² is e)

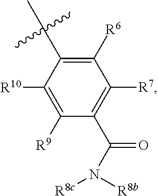

R⁶ and R¹⁰ are both H; R⁷ and R⁹ are each independently selected from hydrogen, —OCH₃, —OCHF₂, —F,

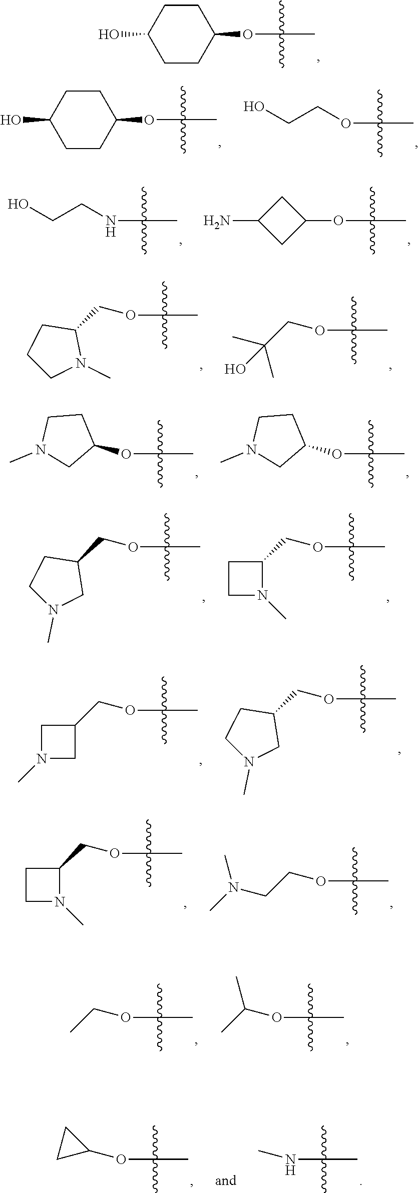

Aspect 13. The compound according to Aspect 9, or a pharmaceutically acceptable salt, stereoisomer, or tautomer thereof, wherein R² is selected from
a)

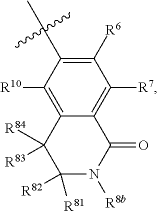

wherein R^{8b} is selected from

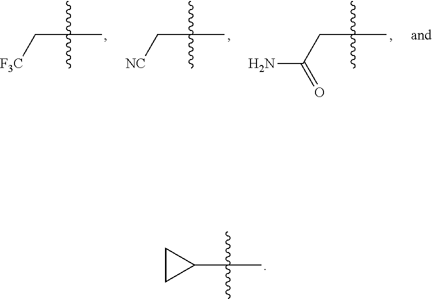

Aspect 14. The compound according to Aspect 9, or a pharmaceutically acceptable salt, stereoisomer, or tautomer thereof, wherein R² is d)

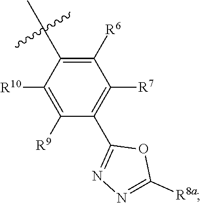

wherein R^{8b} is selected from —CH₃, —CH₂CH₃, and

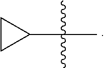 [SMILES: *C1CC1]

Aspect 15. The compound according to Aspect 9, or a pharmaceutically acceptable salt, stereoisomer, or tautomer thereof, wherein R² is c)

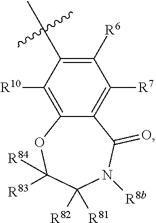

wherein R^{8b} is

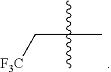 [SMILES: *CC(F)(F)F]

Aspect 16. The compound according to any one of Aspect 9, or a pharmaceutically acceptable salt, stereoisomer, or tautomer thereof, wherein the compound is formula (III-1) or (III-2),

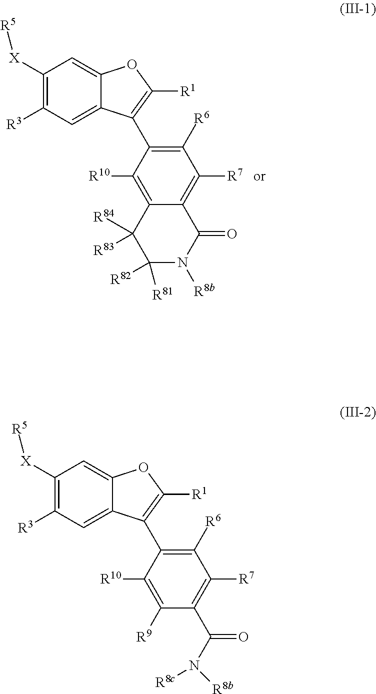

wherein X, R¹, R³, R⁵, R⁶, R⁷, R⁹, R¹⁰, R^{8b}, R^{8c}, R⁸¹, R⁸², R⁸³, R⁸⁴, R^{8b}, are R^{8c} are defined as Aspect 9.
   Aspect 17. The compound according to Aspect 16, or a pharmaceutically acceptable salt, stereoisomer, or tautomer thereof, X is *—CH₂—O—**, *—CH₂—CH₂—O—**, *—CH₂—CH₂—CH₂—O—**, or *—CH₂—CH₂ (CH₃)—O—**; wherein * refers to the position attached to R⁵, ** refers to the position attached to the

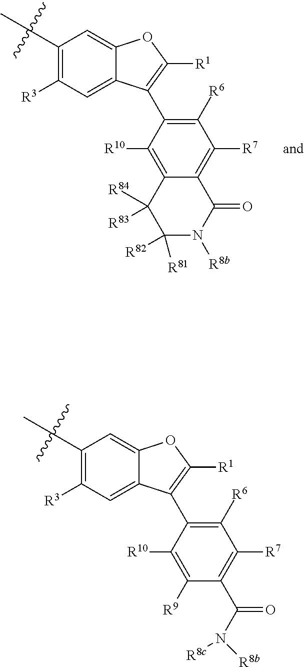

moiety.
Aspect 18. The compound according to Aspect 17, or a pharmaceutically acceptable salt, stereoisomer, or tautomer thereof, the compound is formula (IV-1), (IV-2), (IV-3), (IV-4) or (IV-5)

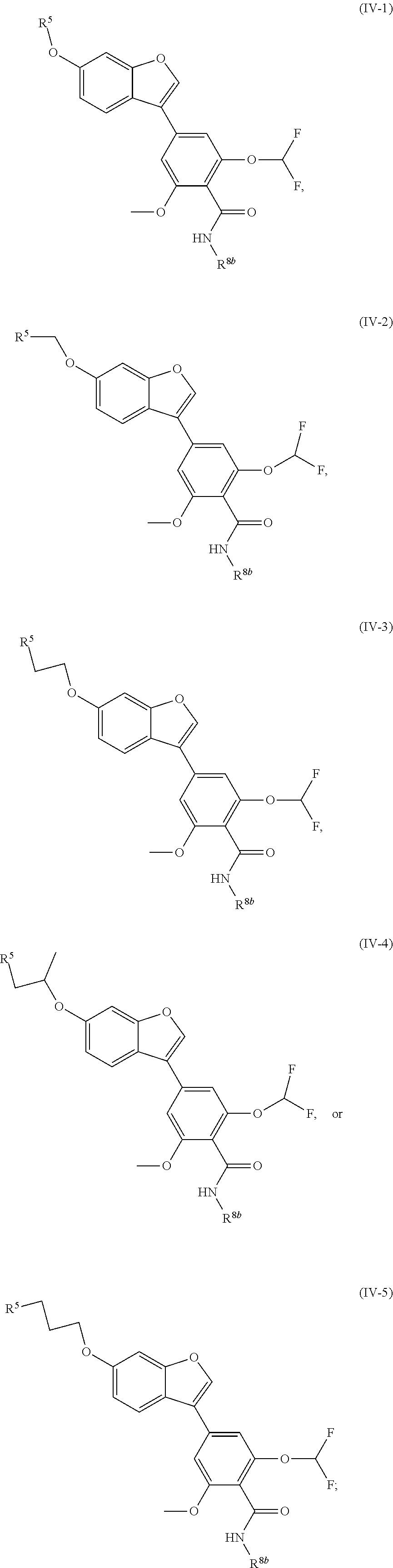

wherein R⁵, and R^{8b} are defined as Aspect 17.
   Aspect 19. The compound according to Aspect 18, or a pharmaceutically acceptable salt, stereoisomer, or tautomer thereof, wherein R⁵ is selected from:
(i) —C₁₋₈alkyl, aryl and 5- to 12-membered heteroaryl, wherein said —C₁₋₈alkyl, aryl or 5- to 12-membered heteroaryl is optionally substituted with at least one R^{5a};
   wherein R^{5a} is each independently selected from halogen, —OH, —CN, —C₁₋₈alkyl, —C₁₋₈haloalkyl, —C₁₋₈alkoxy, —NR^{5b}R^{5c}, and —COR^{5d};
      R^{5b} and R^{5c} are each independently hydrogen, or —C₁₋₈alkyl;
      R^{5d} is hydrogen, —C₁₋₈alkyl, or cycloalkyl;
(ii) —C₁₋₈alkoxy;
(iii) —C₁₋₈haloalkyl;
(iv) —C₃₋₈cycloalkyl, wherein each of said —C₃₋₈cycloalkyl is independently optionally substituted with at least one R^{5a}; wherein R^{5a} is each independently selected from halogen, —C₁₋₈alkyl, —C₁₋₈haloalkyl, and —C₁₋₈alkoxy;
(v) 3- to 8-membered heterocycloalkyl comprising one, two, or three heteroatoms independently selected from N, O, and S, which each of said 3- to 8-membered heterocycloalkyl is independently optionally substituted with at least one R^{5a}, wherein R^{5a} is each independently selected from halogen, oxo, —C₁₋₈alkyl, —C₁₋₄alkoxy, C₁₋₄haloalkyl, and —COC₁₋₄alkyl; wherein each of said —C₁₋₄alkyl, —C₁₋₄alkoxy, and C₁₋₄haloalkyl is independently optionally substituted with at least one R^{5g}; wherein R^{5g} is each independently selected from halogen, —CN, and —C₁₋₈alkyl;
(vi) 5- to 6-membered monocyclic heteroaryl or 8- to 10-membered bicyclic heteroaryl comprising one, two, or three heteroatoms independently selected from N, O, and S; wherein said heteroaryl is each independently optionally substituted with at least one R^{5a}; wherein R^{5a} is each independently selected from selected halogen, oxo, —C₁₋₈alkyl, —C₁₋₈alkoxy, and —COC₁₋₈alkyl; and
(vii) —NR⁵ⁱR^{5j}; wherein R⁵ⁱ and R^{5j} are each independently selected from hydrogen, —C₁₋₈alkyl, aryl, and heteroaryl.
   Aspect 20. The compound according to Aspects 18 or 19, or a pharmaceutically acceptable salt, stereoisomer, or tautomer thereof, wherein R^{8b} is selected from

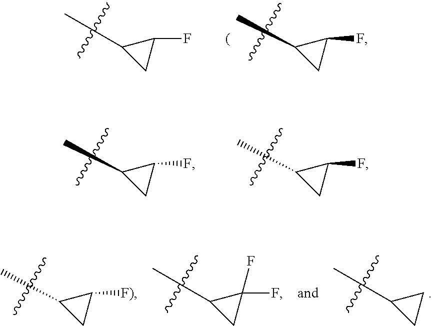

Aspect 21. The compound according to any one of Aspects 1-20, or a pharmaceutically acceptable salt, stereoisomer, or tautomer thereof, wherein R¹ is selected from hydrogen, halogen, and —C₁₋₈alkyl.
Aspect 22. The compound according to any one of Aspects 1-20, or a pharmaceutically acceptable salt, stereoisomer, or tautomer thereof, R¹ is selected from hydrogen, F, or —CH₃.
Aspect 23. A compound or a pharmaceutically acceptable salt, stereoisomer, or tautomer thereof, wherein the compound is selected from

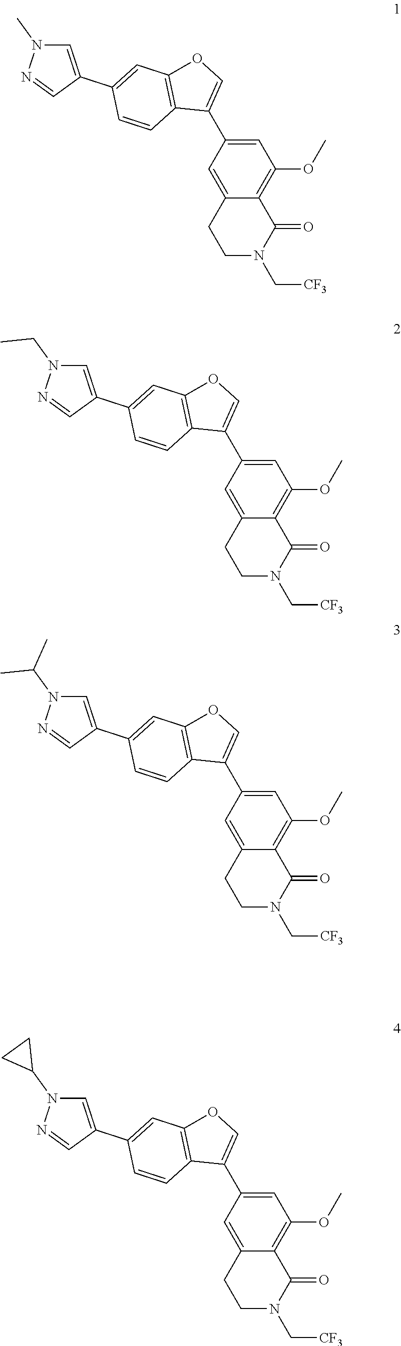

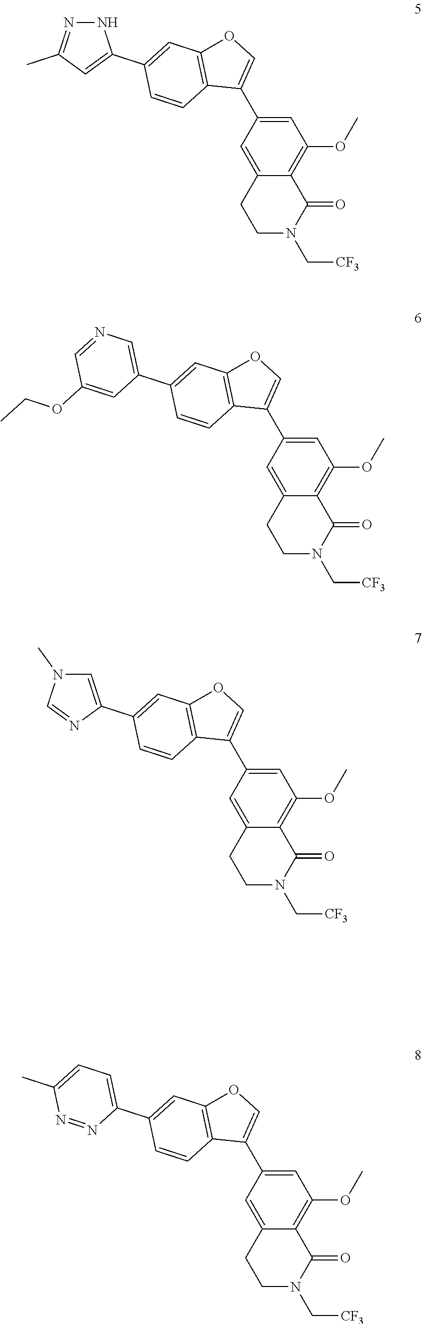

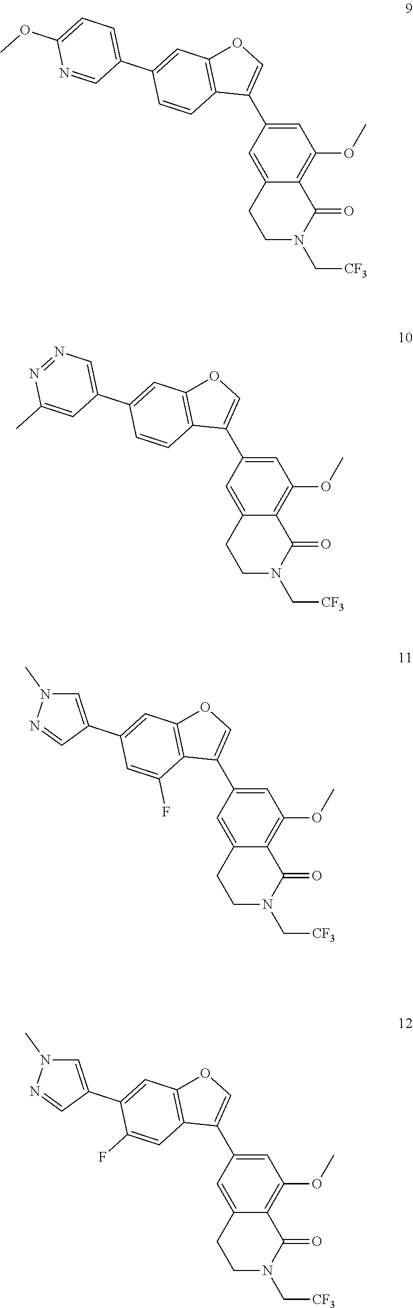

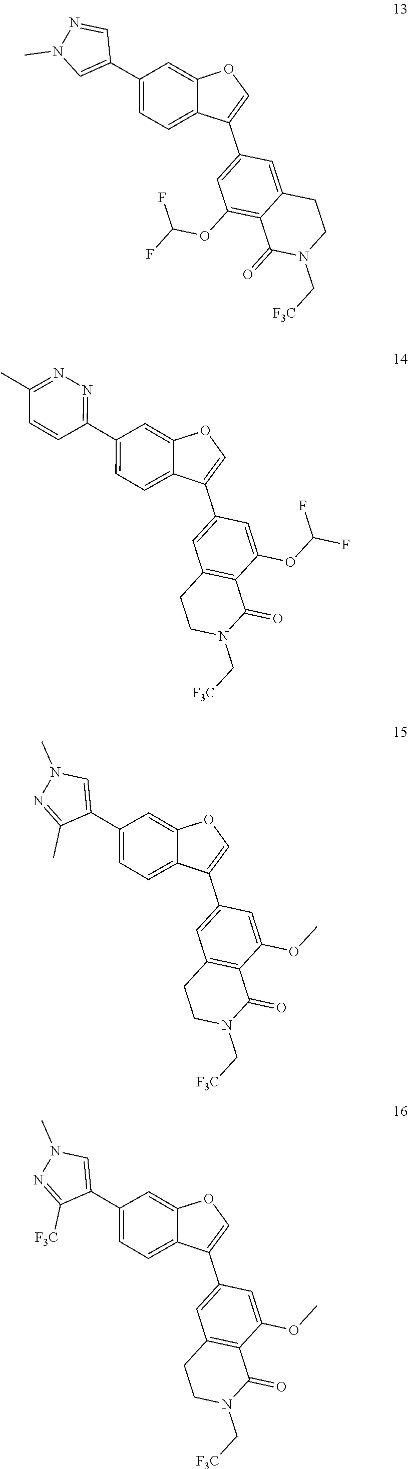 [SMILES: Cn1cc(-c2ccc3c(-c4cc5c(c(OC(F)F)c4)C(=O)N(CC(F)(F)F)CC5)coc3c2)cn1 ; Cc1ccc(-c2ccc3c(-c4cc5c(c(OC(F)F)c4)C(=O)N(CC(F)(F)F)CC5)coc3c2)nn1 ; COc1cc(-c2coc3cc(-c4cn(C)nc4C)ccc23)cc2c1C(=O)N(CC(F)(F)F)CC2 ; COc1cc(-c2coc3cc(-c4cn(C)nc4C(F)(F)F)ccc23)cc2c1C(=O)N(CC(F)(F)F)CC2]

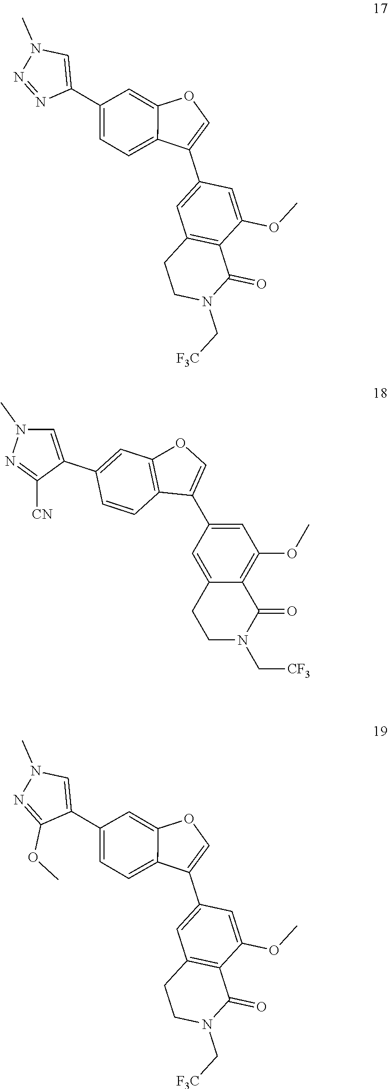

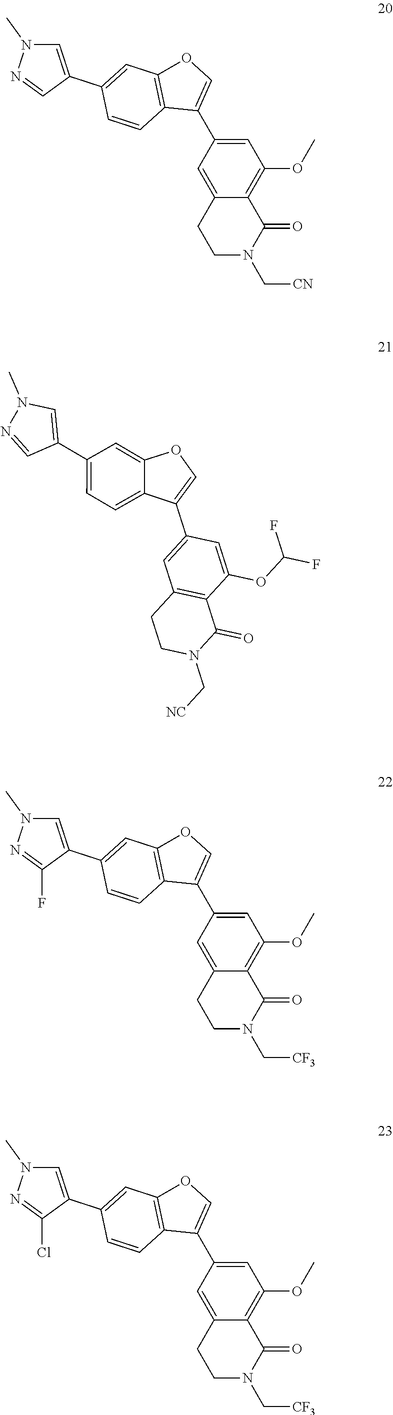

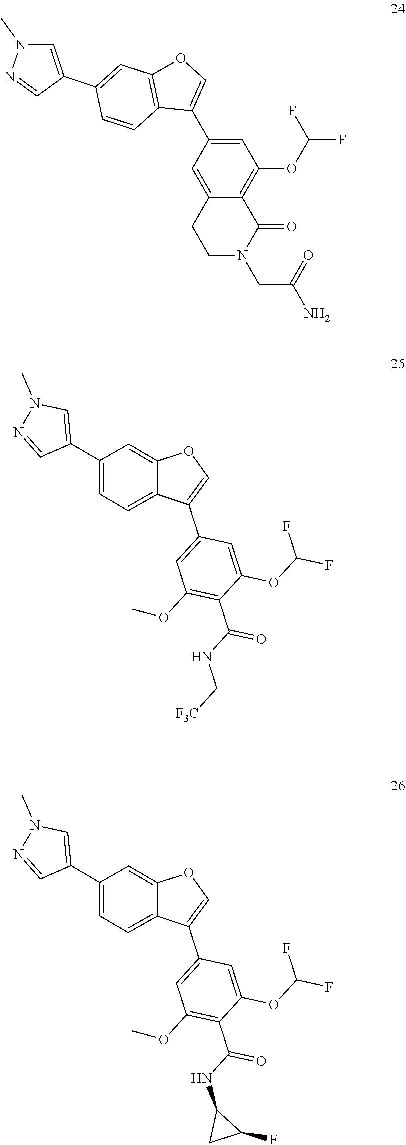 [SMILES: Cn1cc(-c2ccc3c(-c4cc5c(c(OC(F)F)c4)C(=O)N(CC(N)=O)CC5)coc3c2)cn1 ; COc1cc(-c2coc3cc(-c4cnn(C)c4)ccc23)cc(OC(F)F)c1C(=O)NCC(F)(F)F ; COc1cc(-c2coc3cc(-c4cnn(C)c4)ccc23)cc(OC(F)F)c1C(=O)N[C@@H]1C[C@@H]1F]

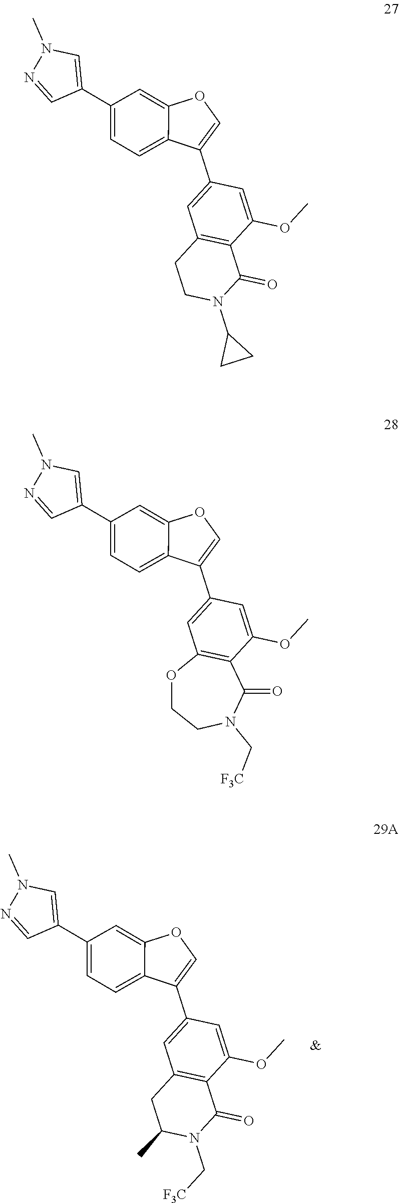 [SMILES: COc1cc(-c2coc3cc(-c4cnn(C)c4)ccc23)cc2c1C(=O)N(C1CC1)CC2 ; COc1cc(-c2coc3cc(-c4cnn(C)c4)ccc23)cc2c1C(=O)N(CC(F)(F)F)CCO2 ; COc1cc(-c2coc3cc(-c4cnn(C)c4)ccc23)cc2c1C(=O)N(CC(F)(F)F)[C@@H](C)C2]

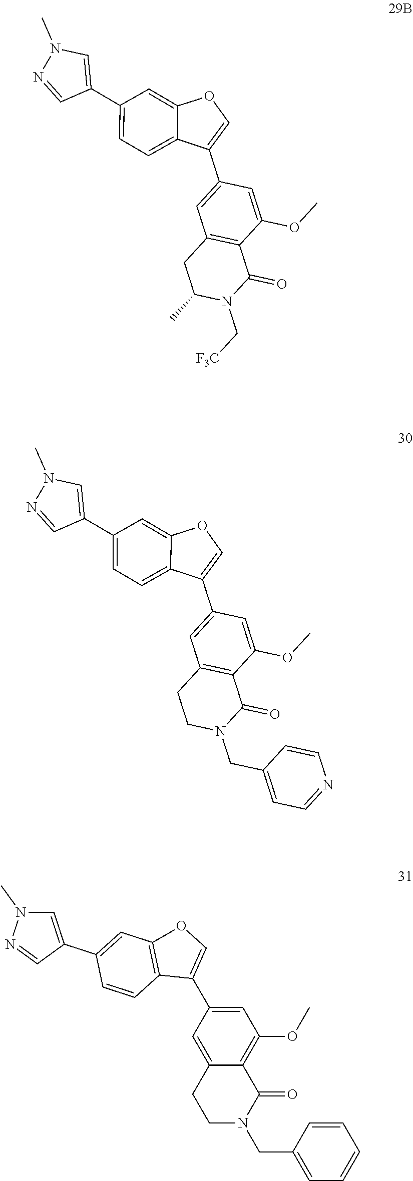

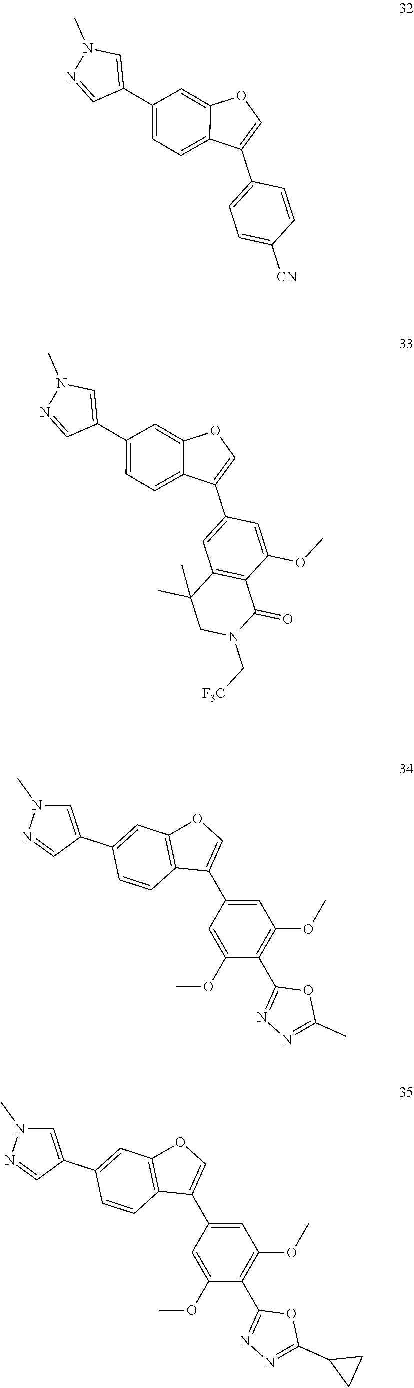

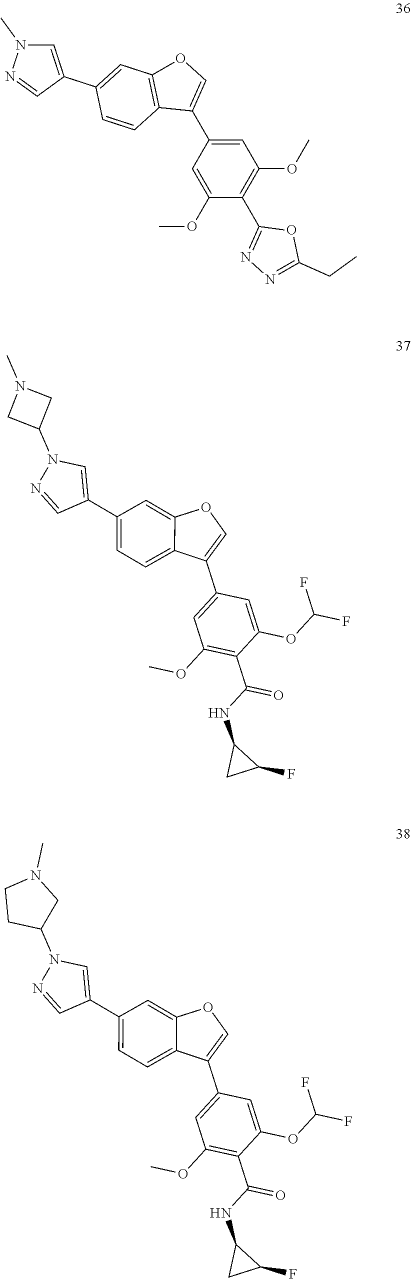

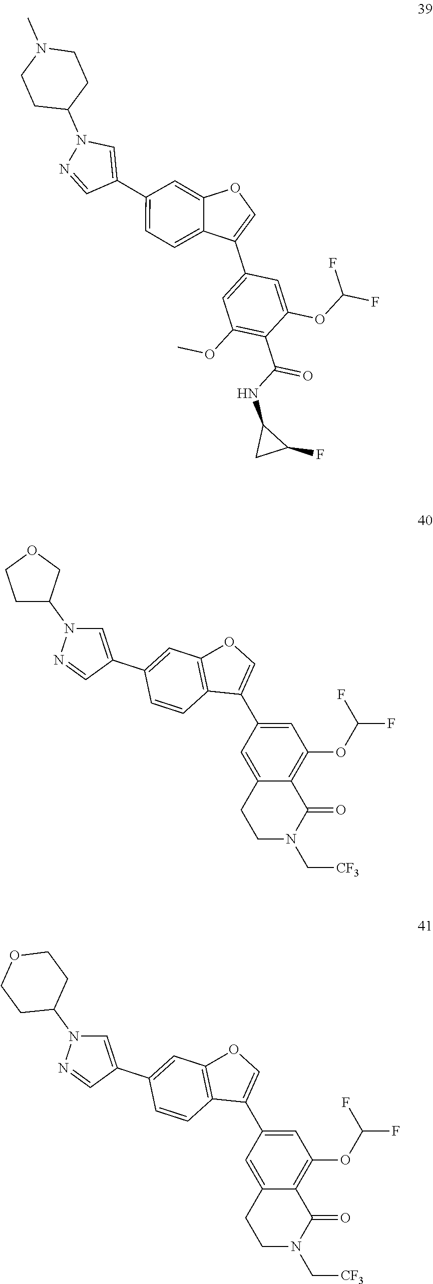 [SMILES: COc1cc(-c2coc3cc(-c4cnn(C5CCN(C)CC5)c4)ccc23)cc(OC(F)F)c1C(=O)N[C@@H]1C[C@@H]1F ; O=C1c2c(cc(-c3coc4cc(-c5cnn(C6CCOC6)c5)ccc34)cc2OC(F)F)CCN1CC(F)(F)F ; O=C1c2c(cc(-c3coc4cc(-c5cnn(C6CCOCC6)c5)ccc34)cc2OC(F)F)CCN1CC(F)(F)F]

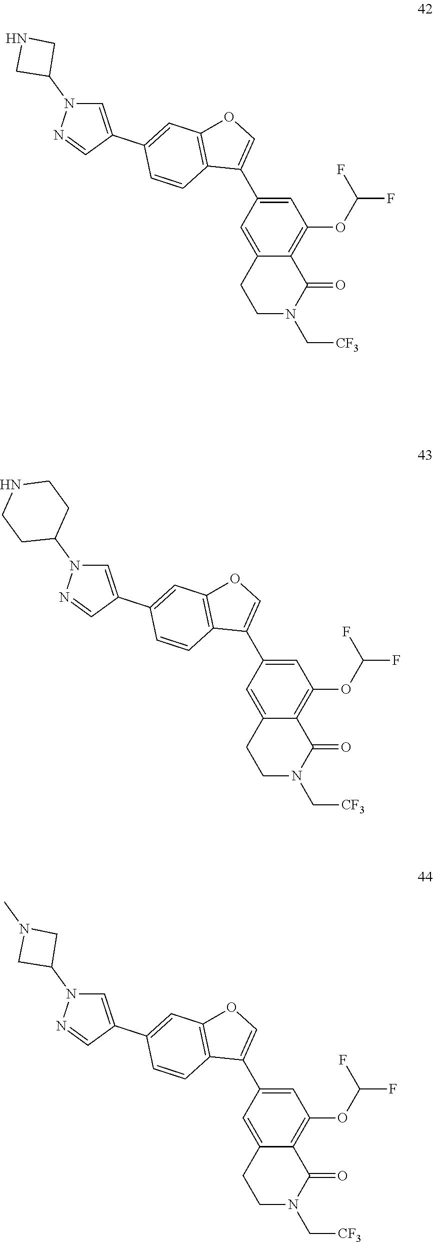 [SMILES: O=C1c2c(cc(-c3coc4cc(-c5cnn(C6CNC6)c5)ccc34)cc2OC(F)F)CCN1CC(F)(F)F ; O=C1c2c(cc(-c3coc4cc(-c5cnn(C6CCNCC6)c5)ccc34)cc2OC(F)F)CCN1CC(F)(F)F ; CN1CC(n2cc(-c3ccc4c(-c5cc6c(c(OC(F)F)c5)C(=O)N(CC(F)(F)F)CC6)coc4c3)cn2)C1]

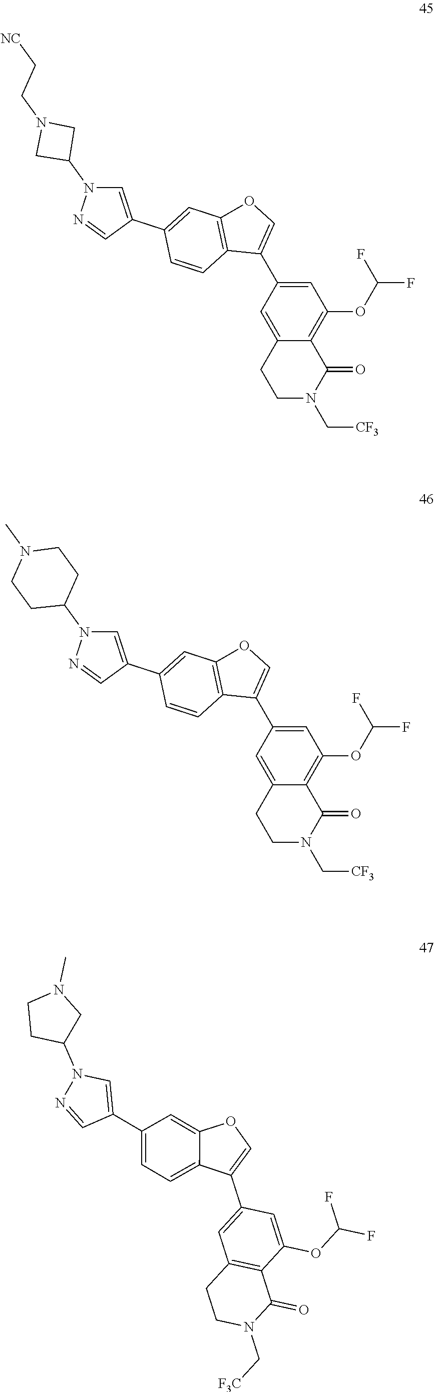 [SMILES: N#CCCN1CC(n2cc(-c3ccc4c(-c5cc6c(c(OC(F)F)c5)C(=O)N(CC(F)(F)F)CC6)coc4c3)cn2)C1 ; CN1CCC(n2cc(-c3ccc4c(-c5cc6c(c(OC(F)F)c5)C(=O)N(CC(F)(F)F)CC6)coc4c3)cn2)CC1 ; CN1CCC(n2cc(-c3ccc4c(-c5cc6c(c(OC(F)F)c5)C(=O)N(CC(F)(F)F)CC6)coc4c3)cn2)C1]

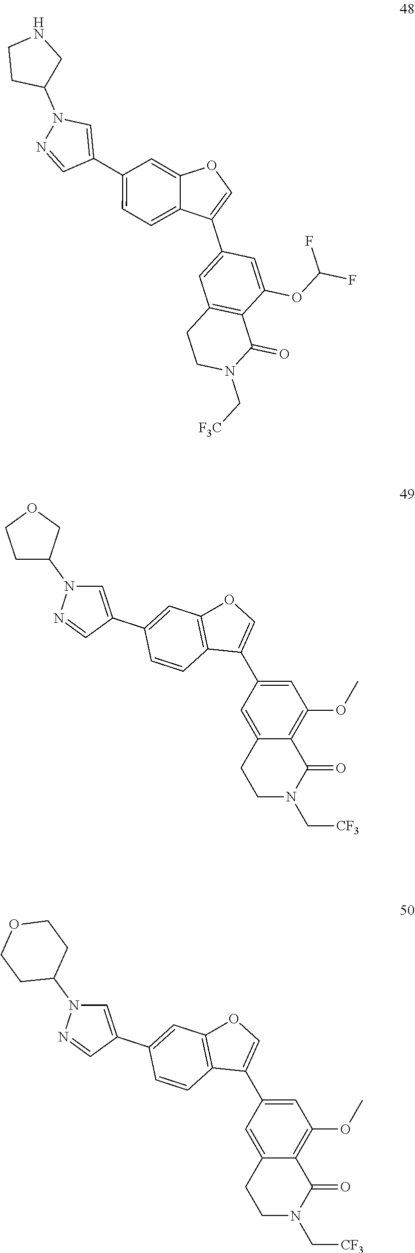 [SMILES: O=C1c2c(cc(-c3coc4cc(-c5cnn(C6CCNC6)c5)ccc34)cc2OC(F)F)CCN1CC(F)(F)F ; COc1cc(-c2coc3cc(-c4cnn(C5CCOC5)c4)ccc23)cc2c1C(=O)N(CC(F)(F)F)CC2 ; COc1cc(-c2coc3cc(-c4cnn(C5CCOCC5)c4)ccc23)cc2c1C(=O)N(CC(F)(F)F)CC2]

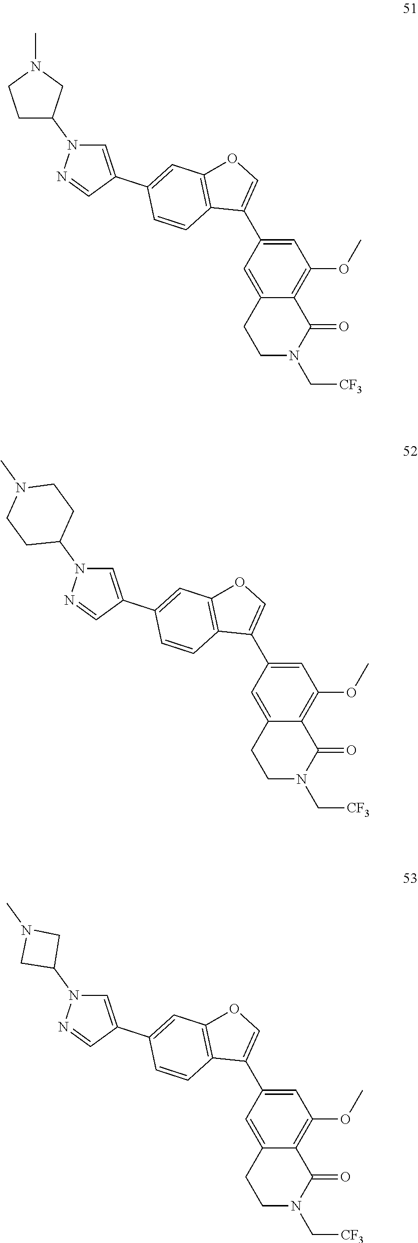

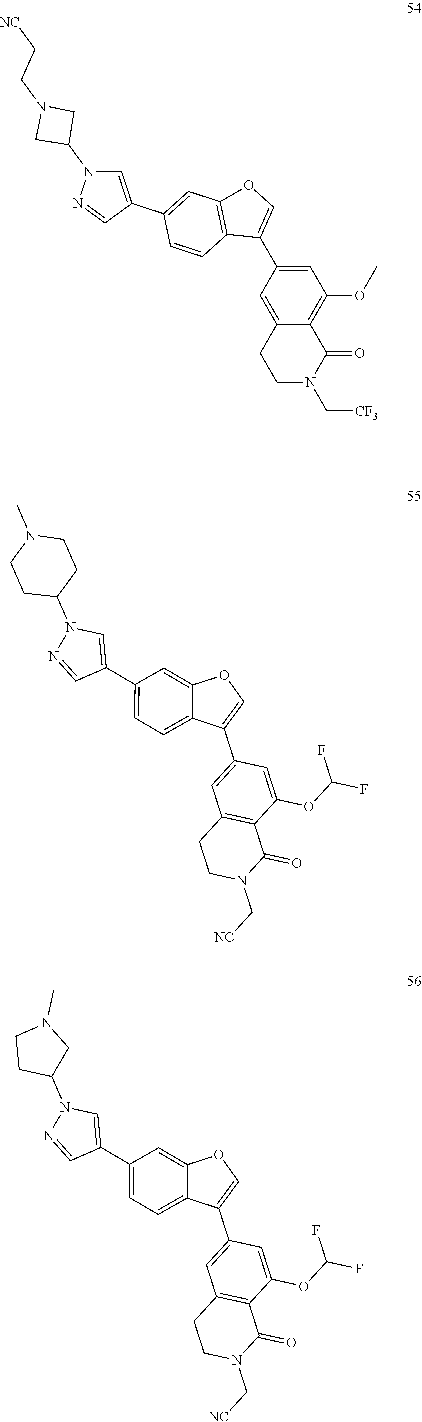 [SMILES: COc1cc(-c2coc3cc(-c4cnn(C5CN(CCC#N)C5)c4)ccc23)cc2c1C(=O)N(CC(F)(F)F)CC2 ; CN1CCC(n2cc(-c3ccc4c(-c5cc6c(c(OC(F)F)c5)C(=O)N(CC#N)CC6)coc4c3)cn2)CC1 ; CN1CCC(n2cc(-c3ccc4c(-c5cc6c(c(OC(F)F)c5)C(=O)N(CC#N)CC6)coc4c3)cn2)C1]

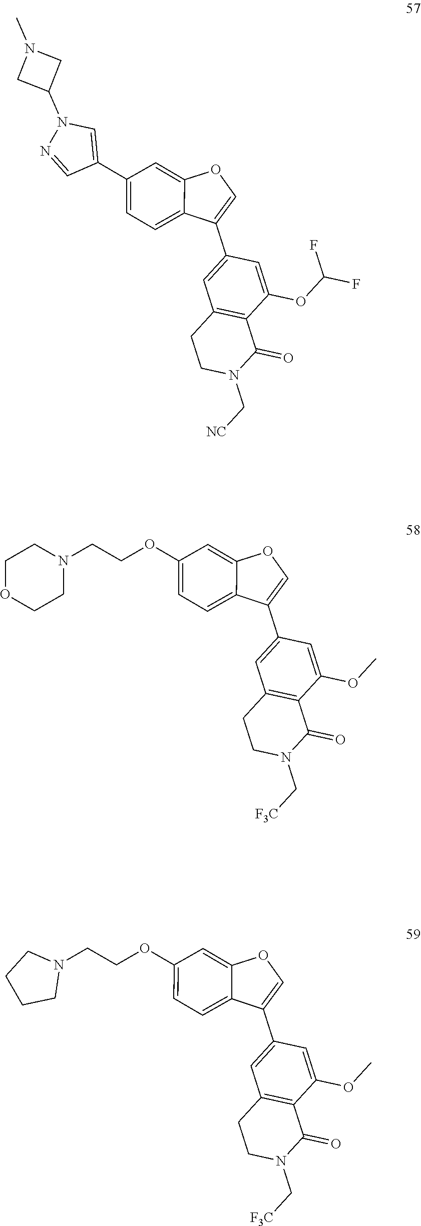 [SMILES: CN1CC(n2cc(-c3ccc4c(-c5cc6c(c(OC(F)F)c5)C(=O)N(CC#N)CC6)coc4c3)cn2)C1 ; COc1cc(-c2coc3cc(OCCN4CCOCC4)ccc23)cc2c1C(=O)N(CC(F)(F)F)CC2 ; COc1cc(-c2coc3cc(OCCN4CCCC4)ccc23)cc2c1C(=O)N(CC(F)(F)F)CC2]

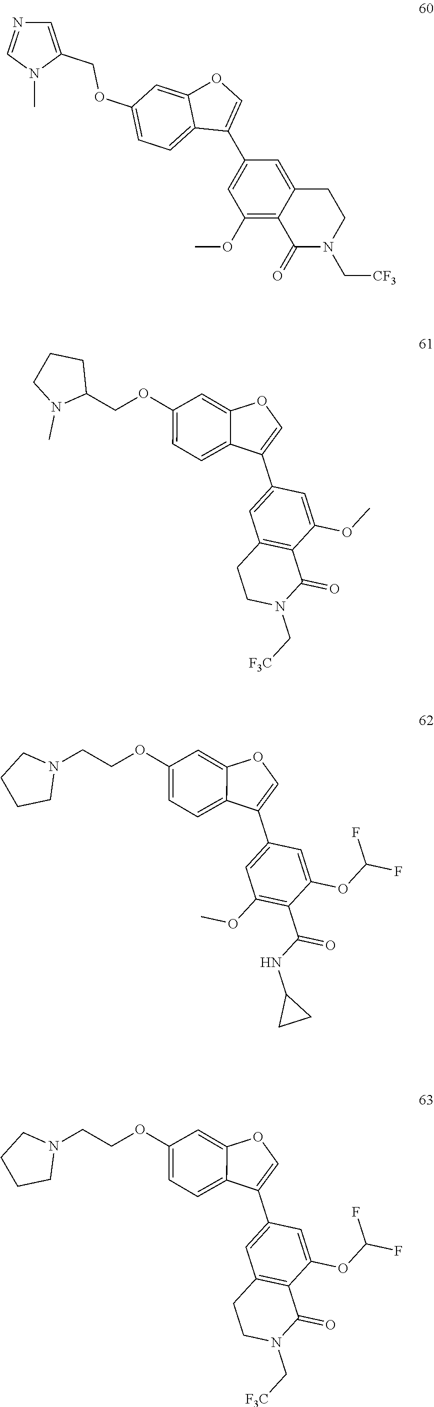 [SMILES: COc1cc(-c2coc3cc(OCc4cncn4C)ccc23)cc2c1C(=O)N(CC(F)(F)F)CC2 ; COc1cc(-c2coc3cc(OCC4CCCN4C)ccc23)cc2c1C(=O)N(CC(F)(F)F)CC2 ; COc1cc(-c2coc3cc(OCCN4CCCC4)ccc23)cc(OC(F)F)c1C(=O)NC1CC1 ; O=C1c2c(cc(-c3coc4cc(OCCN5CCCC5)ccc34)cc2OC(F)F)CCN1CC(F)(F)F]

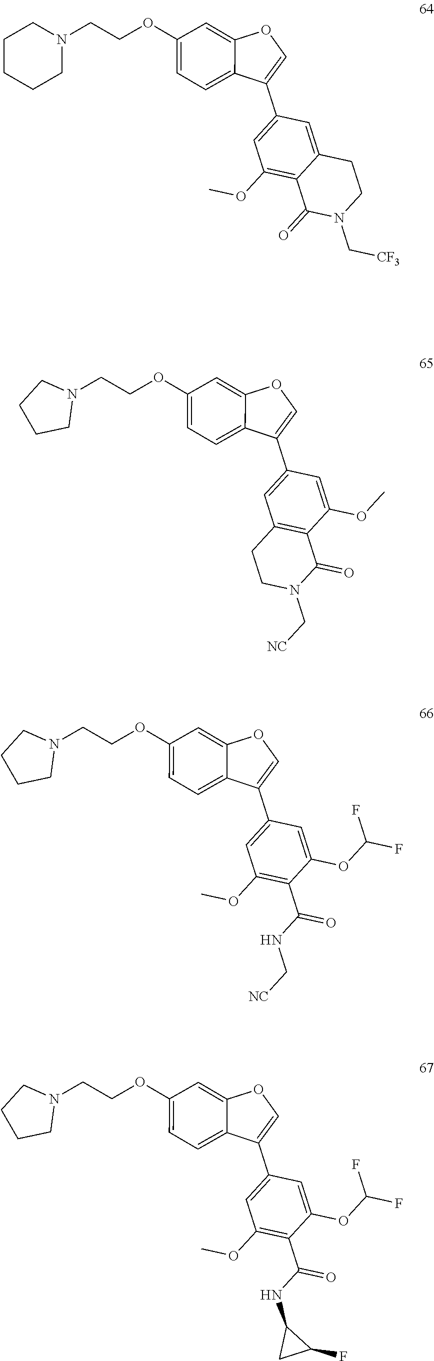 [SMILES: COc1cc(-c2coc3cc(OCCN4CCCCC4)ccc23)cc2c1C(=O)N(CC(F)(F)F)CC2 ; COc1cc(-c2coc3cc(OCCN4CCCC4)ccc23)cc2c1C(=O)N(CC#N)CC2 ; COc1cc(-c2coc3cc(OCCN4CCCC4)ccc23)cc(OC(F)F)c1C(=O)NCC#N ; COc1cc(-c2coc3cc(OCCN4CCCC4)ccc23)cc(OC(F)F)c1C(=O)N[C@@H]1C[C@@H]1F]

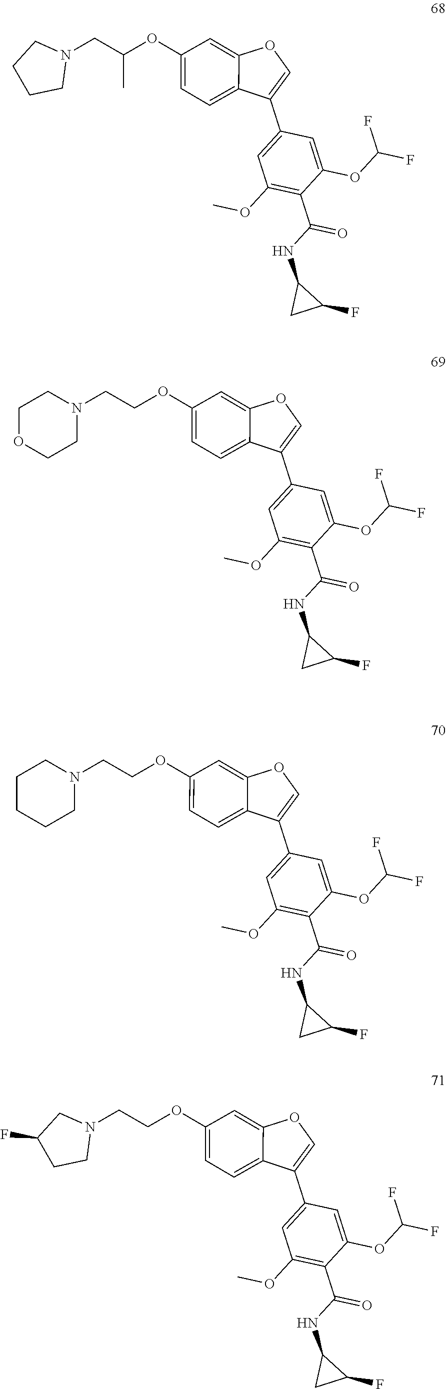 [SMILES: COc1cc(-c2coc3cc(OC(C)CN4CCCC4)ccc23)cc(OC(F)F)c1C(=O)N[C@@H]1C[C@@H]1F ; COc1cc(-c2coc3cc(OCCN4CCOCC4)ccc23)cc(OC(F)F)c1C(=O)N[C@@H]1C[C@@H]1F ; COc1cc(-c2coc3cc(OCCN4CCCCC4)ccc23)cc(OC(F)F)c1C(=O)N[C@@H]1C[C@@H]1F ; COc1cc(-c2coc3cc(OCCN4CC[C@@H](F)C4)ccc23)cc(OC(F)F)c1C(=O)N[C@@H]1C[C@@H]1F]

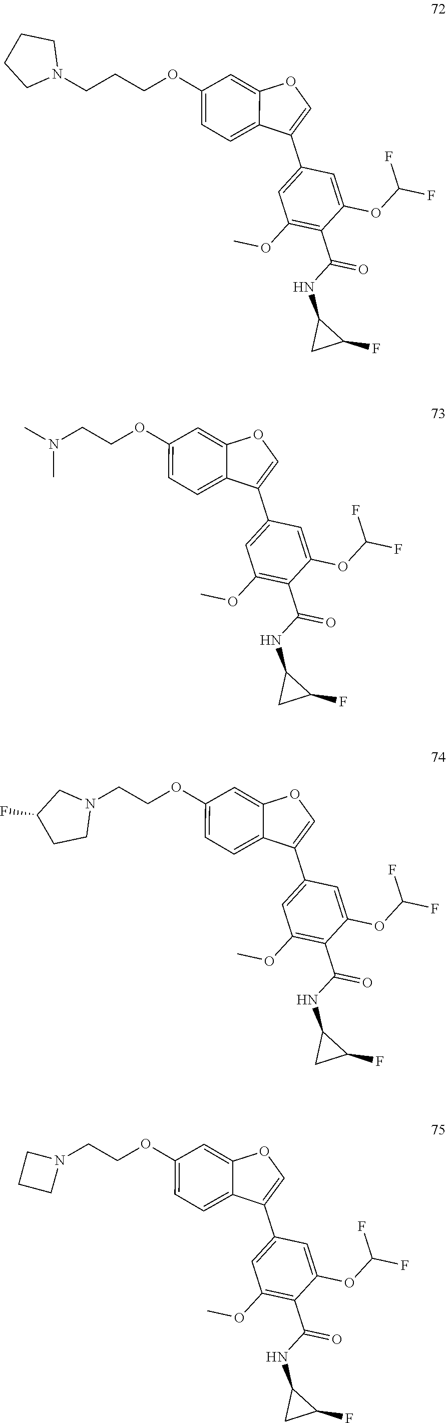 [SMILES: COc1cc(-c2coc3cc(OCCCN4CCCC4)ccc23)cc(OC(F)F)c1C(=O)N[C@@H]1C[C@@H]1F ; COc1cc(-c2coc3cc(OCCN(C)C)ccc23)cc(OC(F)F)c1C(=O)N[C@@H]1C[C@@H]1F ; COc1cc(-c2coc3cc(OCCN4CC[C@H](F)C4)ccc23)cc(OC(F)F)c1C(=O)N[C@@H]1C[C@@H]1F ; COc1cc(-c2coc3cc(OCCN4CCC4)ccc23)cc(OC(F)F)c1C(=O)N[C@@H]1C[C@@H]1F]

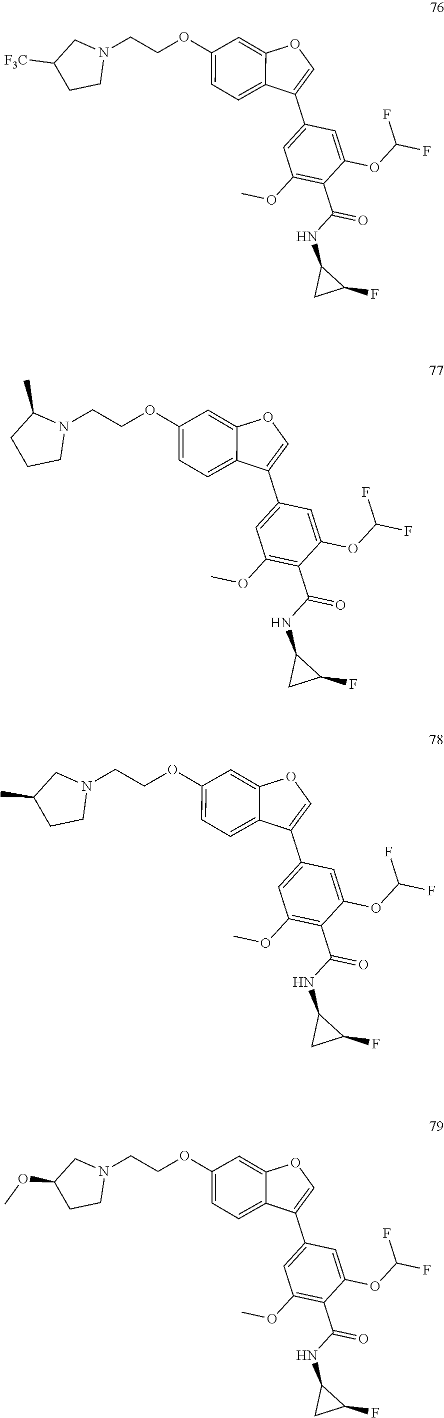 [SMILES: COc1cc(-c2coc3cc(OCCN4CCC(C(F)(F)F)C4)ccc23)cc(OC(F)F)c1C(=O)N[C@@H]1C[C@@H]1F ; COc1cc(-c2coc3cc(OCCN4CCC[C@H]4C)ccc23)cc(OC(F)F)c1C(=O)N[C@@H]1C[C@@H]1F ; COc1cc(-c2coc3cc(OCCN4CC[C@@H](C)C4)ccc23)cc(OC(F)F)c1C(=O)N[C@@H]1C[C@@H]1F ; COc1cc(-c2coc3cc(OCCN4CC[C@@H](OC)C4)ccc23)cc(OC(F)F)c1C(=O)N[C@@H]1C[C@@H]1F]

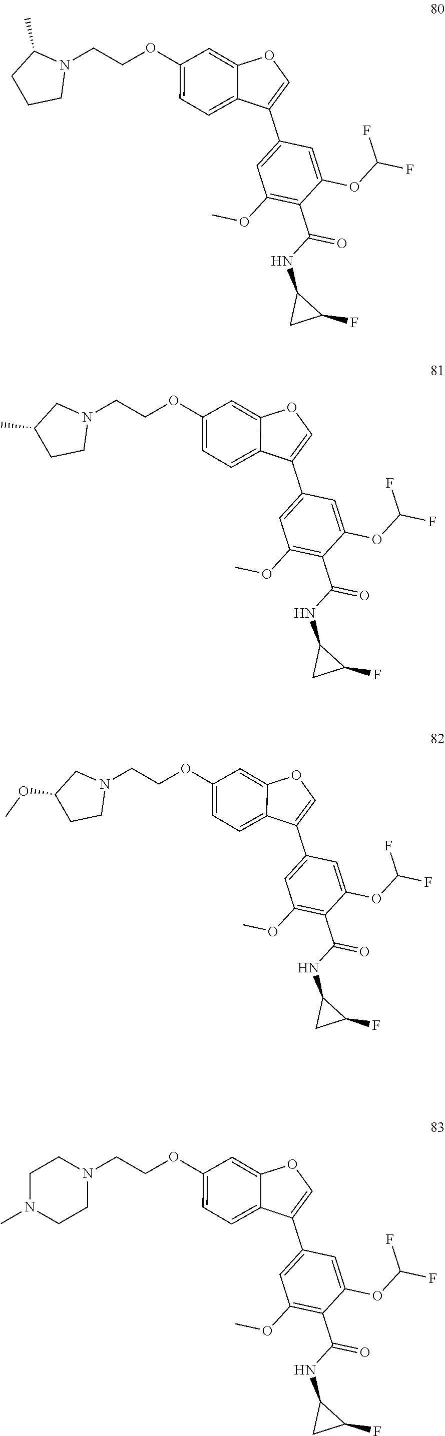 [SMILES: COc1cc(-c2coc3cc(OCCN4CCC[C@@H]4C)ccc23)cc(OC(F)F)c1C(=O)N[C@@H]1C[C@@H]1F ; COc1cc(-c2coc3cc(OCCN4CC[C@H](C)C4)ccc23)cc(OC(F)F)c1C(=O)N[C@@H]1C[C@@H]1F ; COc1cc(-c2coc3cc(OCCN4CC[C@H](OC)C4)ccc23)cc(OC(F)F)c1C(=O)N[C@@H]1C[C@@H]1F ; COc1cc(-c2coc3cc(OCCN4CCN(C)CC4)ccc23)cc(OC(F)F)c1C(=O)N[C@@H]1C[C@@H]1F]

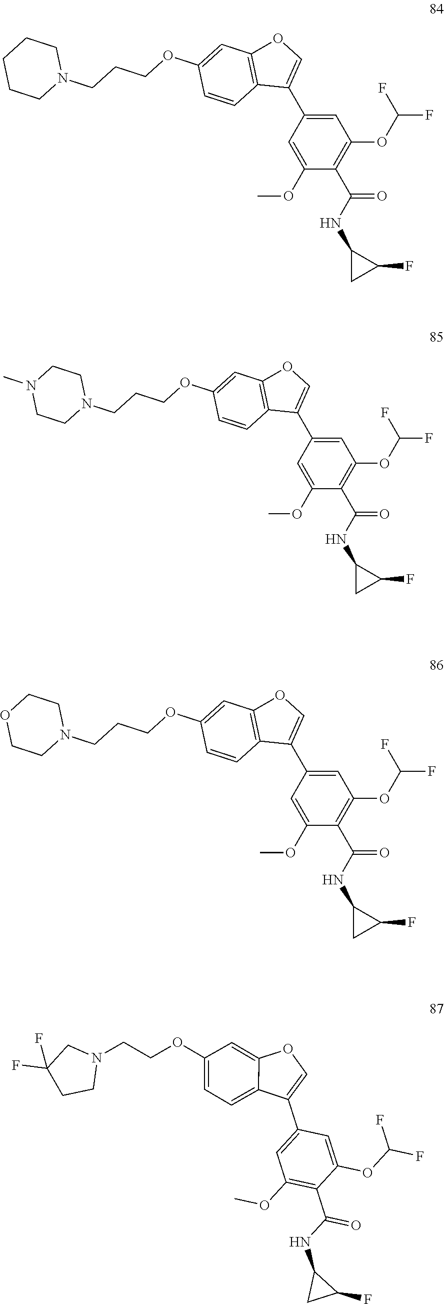 [SMILES: COc1cc(-c2coc3cc(OCCCN4CCCCC4)ccc23)cc(OC(F)F)c1C(=O)N[C@@H]1C[C@@H]1F ; COc1cc(-c2coc3cc(OCCCN4CCN(C)CC4)ccc23)cc(OC(F)F)c1C(=O)N[C@@H]1C[C@@H]1F ; COc1cc(-c2coc3cc(OCCCN4CCOCC4)ccc23)cc(OC(F)F)c1C(=O)N[C@@H]1C[C@@H]1F ; COc1cc(-c2coc3cc(OCCN4CCC(F)(F)C4)ccc23)cc(OC(F)F)c1C(=O)N[C@@H]1C[C@@H]1F]

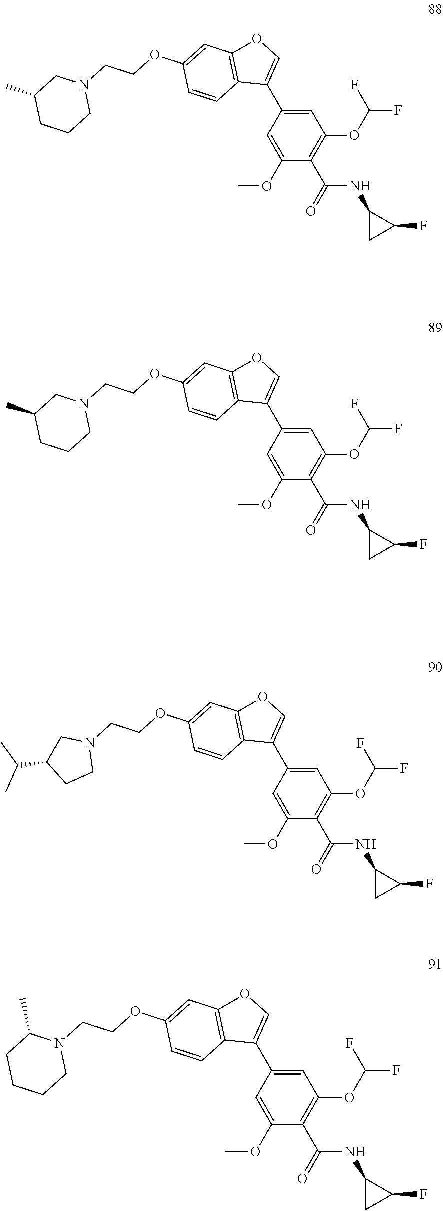 [SMILES: COc1cc(-c2coc3cc(OCCN4CCC[C@H](C)C4)ccc23)cc(OC(F)F)c1C(=O)N[C@@H]1C[C@@H]1F ; COc1cc(-c2coc3cc(OCCN4CCC[C@@H](C)C4)ccc23)cc(OC(F)F)c1C(=O)N[C@@H]1C[C@@H]1F ; COc1cc(-c2coc3cc(OCCN4CC[C@H](C(C)C)C4)ccc23)cc(OC(F)F)c1C(=O)N[C@@H]1C[C@@H]1F ; COc1cc(-c2coc3cc(OCCN4CCCC[C@@H]4C)ccc23)cc(OC(F)F)c1C(=O)N[C@@H]1C[C@@H]1F]

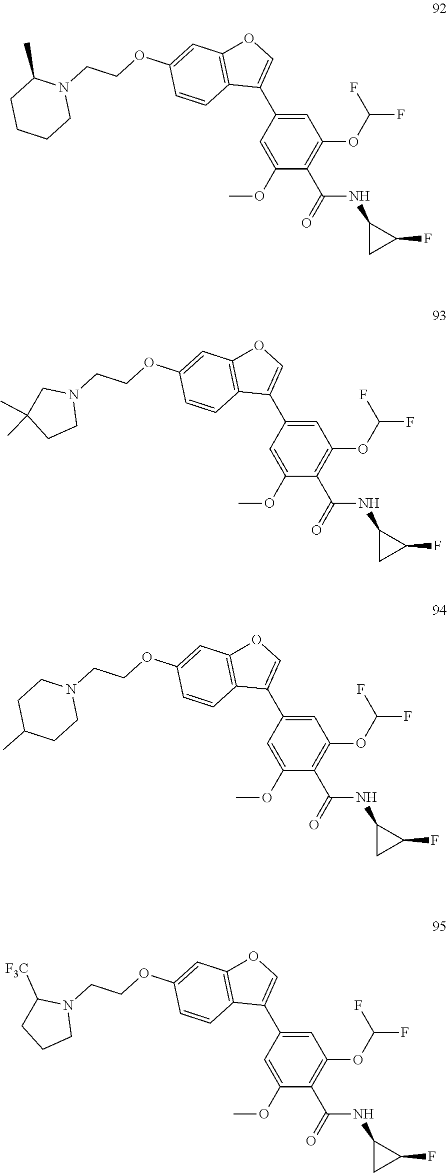 [SMILES: COc1cc(-c2coc3cc(OCCN4CCCC[C@H]4C)ccc23)cc(OC(F)F)c1C(=O)N[C@@H]1C[C@@H]1F ; COc1cc(-c2coc3cc(OCCN4CCC(C)(C)C4)ccc23)cc(OC(F)F)c1C(=O)N[C@@H]1C[C@@H]1F ; COc1cc(-c2coc3cc(OCCN4CCC(C)CC4)ccc23)cc(OC(F)F)c1C(=O)N[C@@H]1C[C@@H]1F ; COc1cc(-c2coc3cc(OCCN4CCCC4C(F)(F)F)ccc23)cc(OC(F)F)c1C(=O)N[C@@H]1C[C@@H]1F]

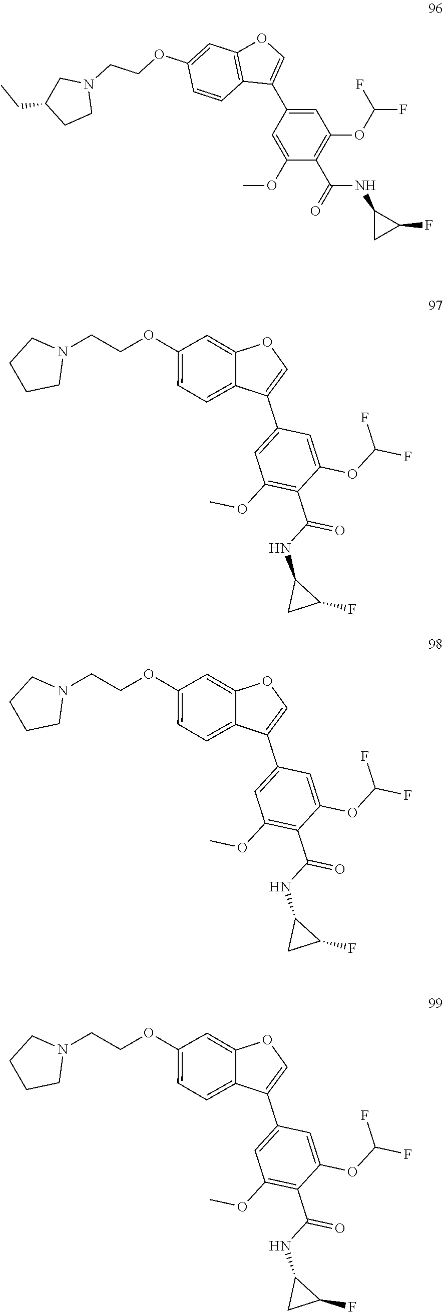 [SMILES: CC[C@H]1CCN(CCOc2ccc3c(-c4cc(OC)c(C(=O)N[C@@H]5C[C@@H]5F)c(OC(F)F)c4)coc3c2)C1 ; COc1cc(-c2coc3cc(OCCN4CCCC4)ccc23)cc(OC(F)F)c1C(=O)N[C@@H]1C[C@H]1F ; COc1cc(-c2coc3cc(OCCN4CCCC4)ccc23)cc(OC(F)F)c1C(=O)N[C@H]1C[C@H]1F ; COc1cc(-c2coc3cc(OCCN4CCCC4)ccc23)cc(OC(F)F)c1C(=O)N[C@H]1C[C@@H]1F]

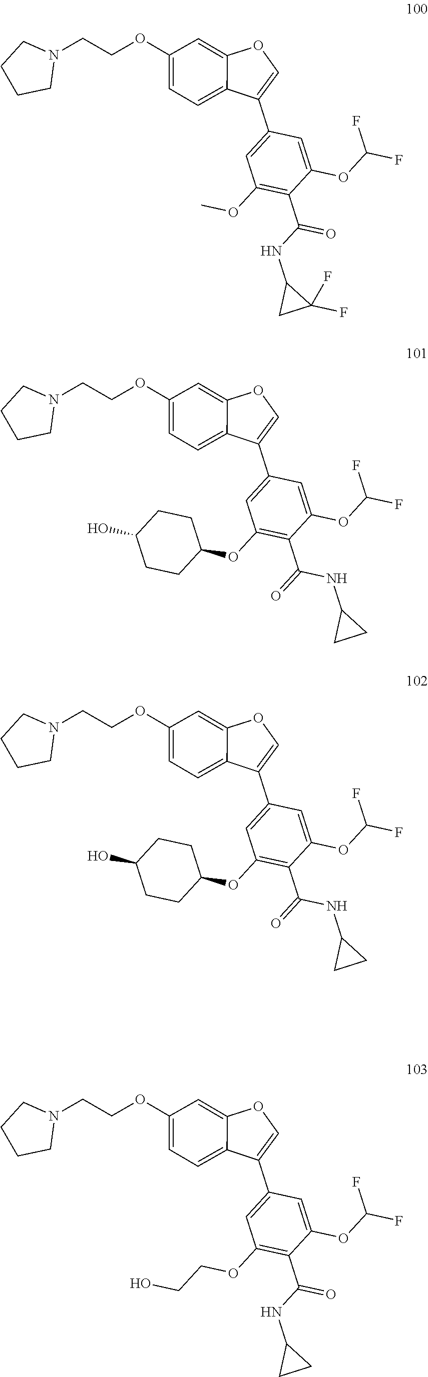 [SMILES: COc1cc(-c2coc3cc(OCCN4CCCC4)ccc23)cc(OC(F)F)c1C(=O)NC1CC1(F)F ; O=C(NC1CC1)c1c(OC(F)F)cc(-c2coc3cc(OCCN4CCCC4)ccc23)cc1O[C@H]1CC[C@H](O)CC1 ; O=C(NC1CC1)c1c(OC(F)F)cc(-c2coc3cc(OCCN4CCCC4)ccc23)cc1O[C@H]1CC[C@@H](O)CC1 ; O=C(NC1CC1)c1c(OCCO)cc(-c2coc3cc(OCCN4CCCC4)ccc23)cc1OC(F)F]

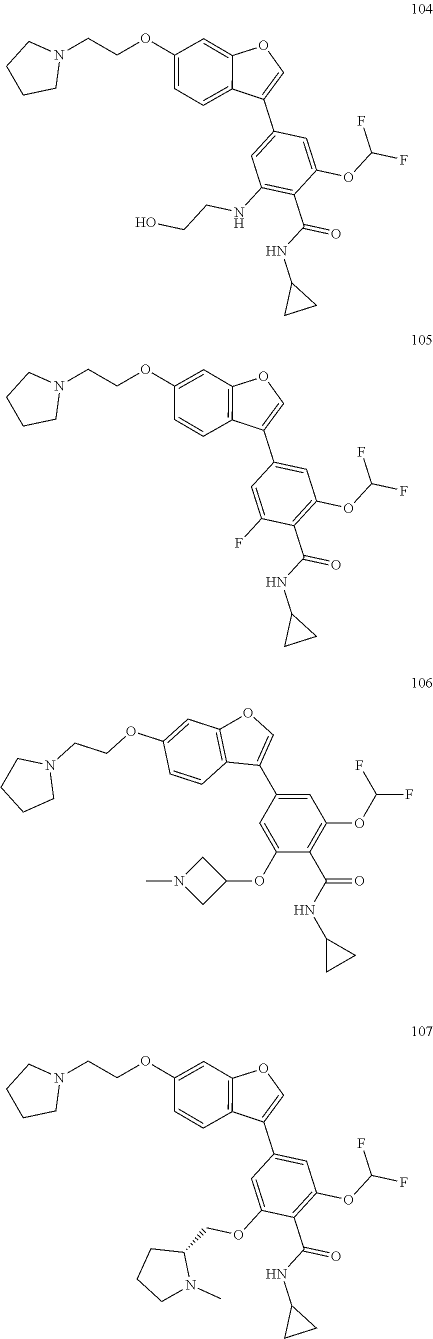 [SMILES: O=C(NC1CC1)c1c(NCCO)cc(-c2coc3cc(OCCN4CCCC4)ccc23)cc1OC(F)F ; O=C(NC1CC1)c1c(F)cc(-c2coc3cc(OCCN4CCCC4)ccc23)cc1OC(F)F ; CN1CC(Oc2cc(-c3coc4cc(OCCN5CCCC5)ccc34)cc(OC(F)F)c2C(=O)NC2CC2)C1 ; CN1CCC[C@@H]1COc1cc(-c2coc3cc(OCCN4CCCC4)ccc23)cc(OC(F)F)c1C(=O)NC1CC1]

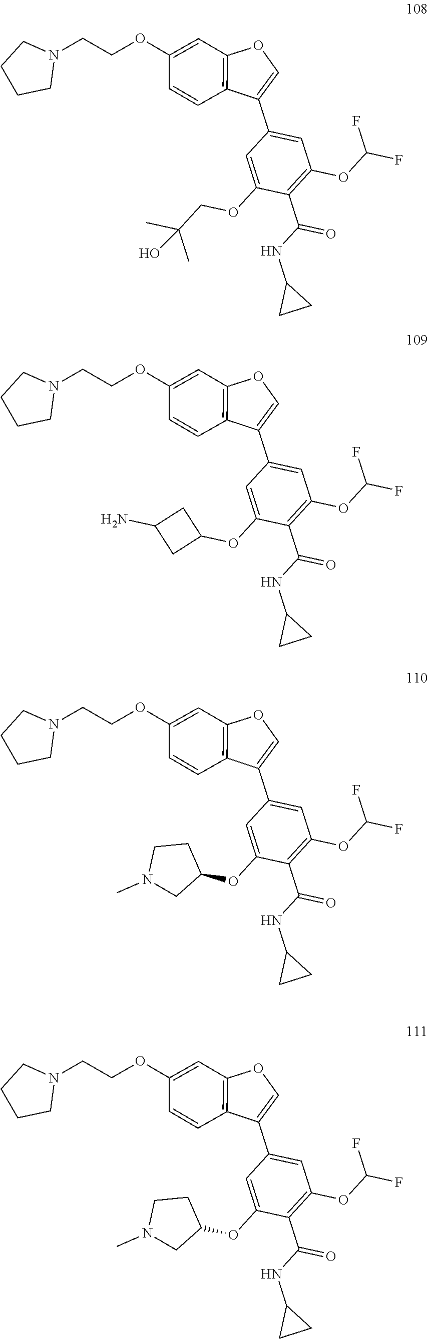

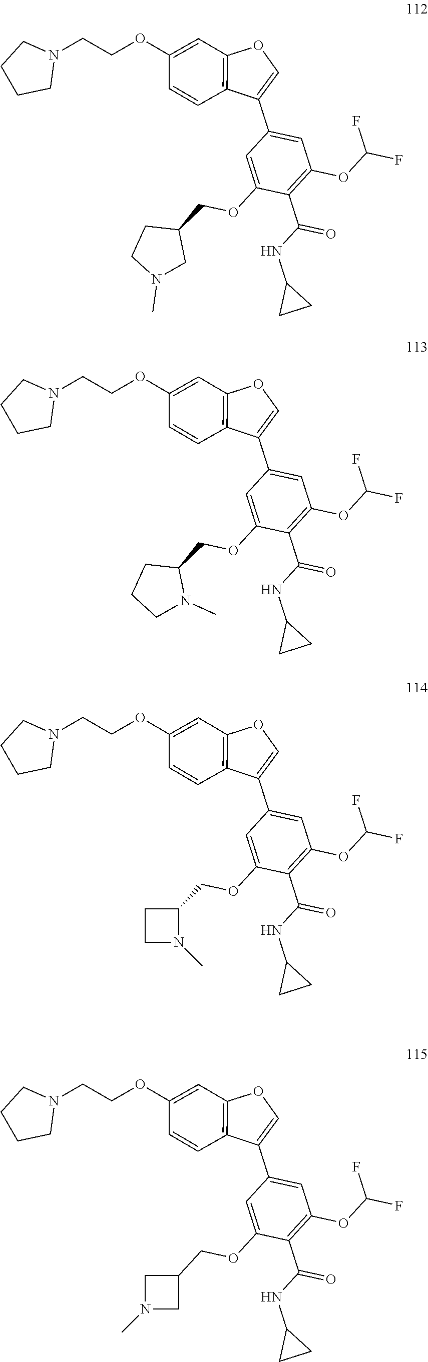 [SMILES: CN1CC[C@@H](COc2cc(-c3coc4cc(OCCN5CCCC5)ccc34)cc(OC(F)F)c2C(=O)NC2CC2)C1 ; CN1CCC[C@H]1COc1cc(-c2coc3cc(OCCN4CCCC4)ccc23)cc(OC(F)F)c1C(=O)NC1CC1 ; CN1CC[C@@H]1COc1cc(-c2coc3cc(OCCN4CCCC4)ccc23)cc(OC(F)F)c1C(=O)NC1CC1 ; CN1CC(COc2cc(-c3coc4cc(OCCN5CCCC5)ccc34)cc(OC(F)F)c2C(=O)NC2CC2)C1]

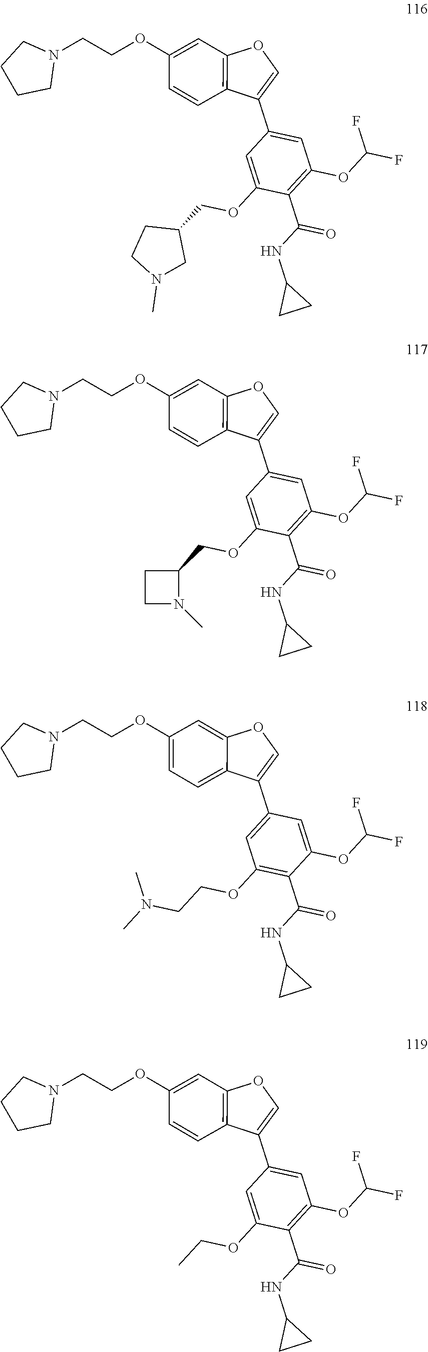 [SMILES: CN1CC[C@H](COc2cc(-c3coc4cc(OCCN5CCCC5)ccc34)cc(OC(F)F)c2C(=O)NC2CC2)C1 ; CN1CC[C@H]1COc1cc(-c2coc3cc(OCCN4CCCC4)ccc23)cc(OC(F)F)c1C(=O)NC1CC1 ; CN(C)CCOc1cc(-c2coc3cc(OCCN4CCCC4)ccc23)cc(OC(F)F)c1C(=O)NC1CC1 ; CCOc1cc(-c2coc3cc(OCCN4CCCC4)ccc23)cc(OC(F)F)c1C(=O)NC1CC1]

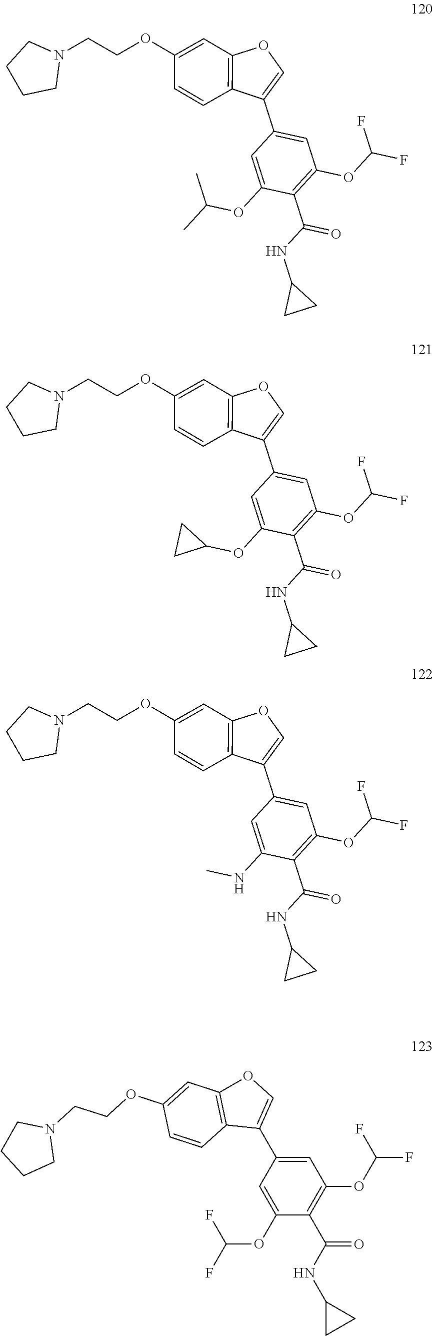 [SMILES: CC(C)Oc1cc(-c2coc3cc(OCCN4CCCC4)ccc23)cc(OC(F)F)c1C(=O)NC1CC1 ; O=C(NC1CC1)c1c(OC(F)F)cc(-c2coc3cc(OCCN4CCCC4)ccc23)cc1OC1CC1 ; CNc1cc(-c2coc3cc(OCCN4CCCC4)ccc23)cc(OC(F)F)c1C(=O)NC1CC1 ; O=C(NC1CC1)c1c(OC(F)F)cc(-c2coc3cc(OCCN4CCCC4)ccc23)cc1OC(F)F]

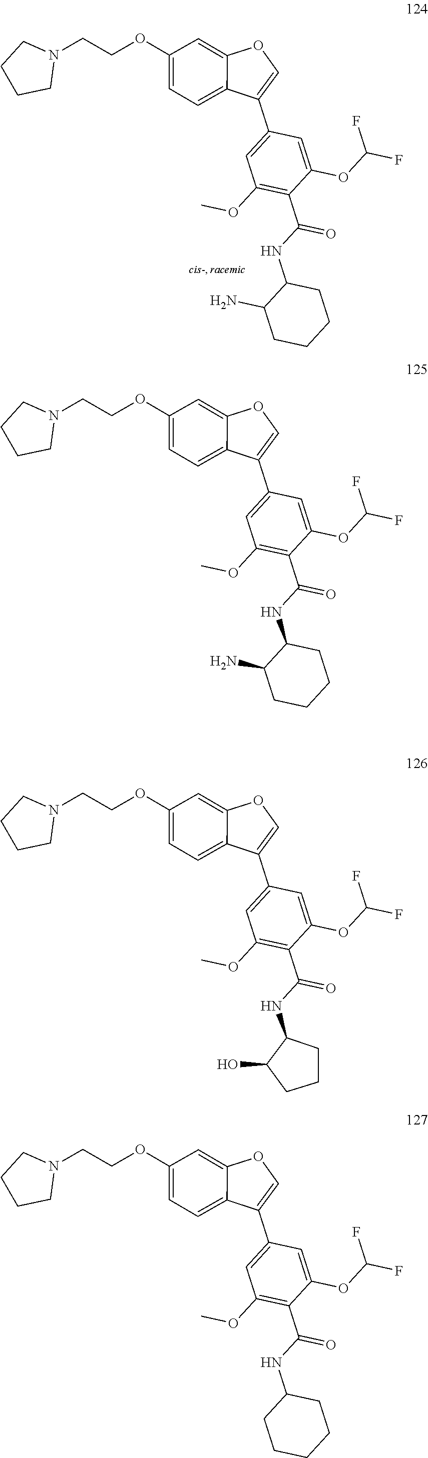 [SMILES: COc1cc(-c2coc3cc(OCCN4CCCC4)ccc23)cc(OC(F)F)c1C(=O)NC1CCCCC1N ; COc1cc(-c2coc3cc(OCCN4CCCC4)ccc23)cc(OC(F)F)c1C(=O)N[C@H]1CCCC[C@H]1N ; COc1cc(-c2coc3cc(OCCN4CCCC4)ccc23)cc(OC(F)F)c1C(=O)N[C@H]1CCC[C@H]1O ; COc1cc(-c2coc3cc(OCCN4CCCC4)ccc23)cc(OC(F)F)c1C(=O)NC1CCCCC1]

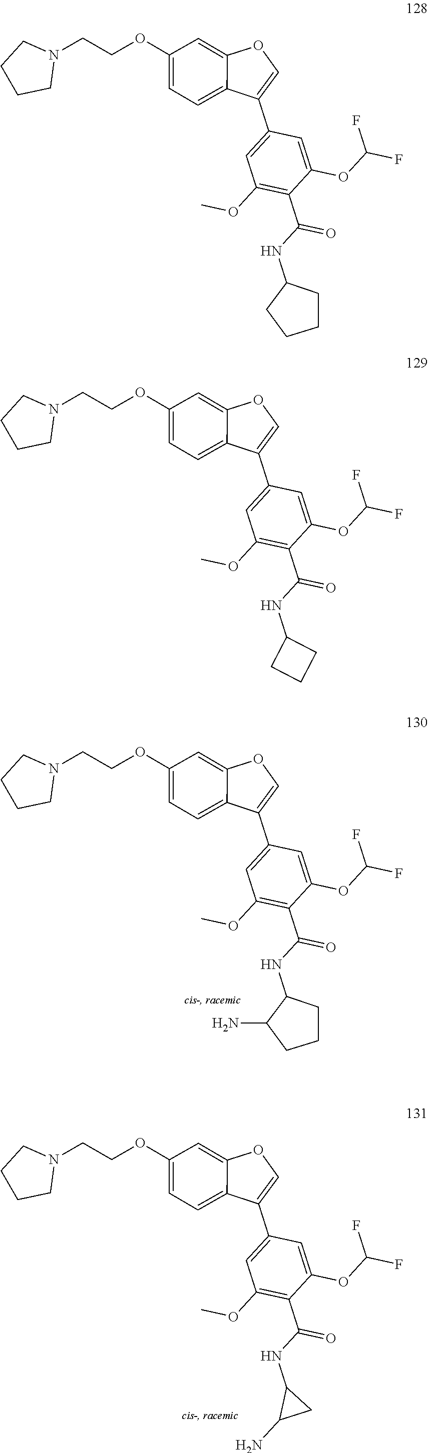 [SMILES: COc1cc(-c2coc3cc(OCCN4CCCC4)ccc23)cc(OC(F)F)c1C(=O)NC1CCCC1 ; COc1cc(-c2coc3cc(OCCN4CCCC4)ccc23)cc(OC(F)F)c1C(=O)NC1CCC1 ; COc1cc(-c2coc3cc(OCCN4CCCC4)ccc23)cc(OC(F)F)c1C(=O)NC1CCCC1N ; COc1cc(-c2coc3cc(OCCN4CCCC4)ccc23)cc(OC(F)F)c1C(=O)NC1CC1N]

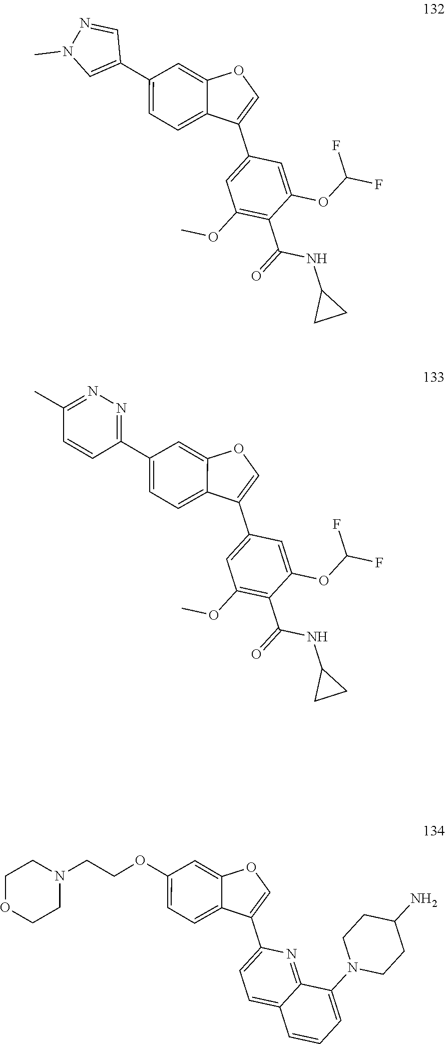 [SMILES: COc1cc(-c2coc3cc(-c4cnn(C)c4)ccc23)cc(OC(F)F)c1C(=O)NC1CC1 ; COc1cc(-c2coc3cc(-c4ccc(C)nn4)ccc23)cc(OC(F)F)c1C(=O)NC1CC1 ; NC1CCN(c2cccc3ccc(-c4coc5cc(OCCN6CCOCC6)ccc45)nc23)CC1]

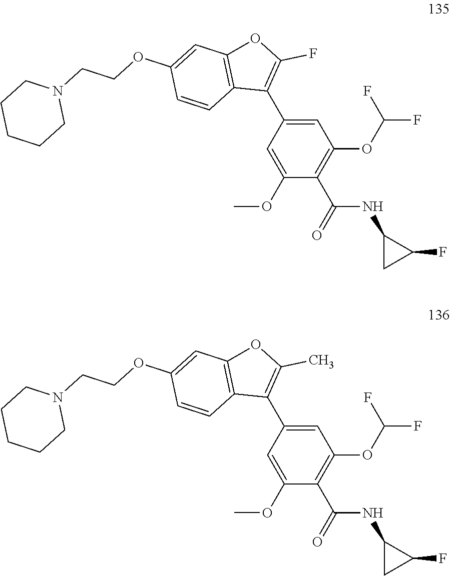 [SMILES: COc1cc(-c2c(F)oc3cc(OCCN4CCCCC4)ccc23)cc(OC(F)F)c1C(=O)N[C@@H]1C[C@@H]1F ; COc1cc(-c2c(C)oc3cc(OCCN4CCCCC4)ccc23)cc(OC(F)F)c1C(=O)N[C@@H]1C[C@@H]1F]

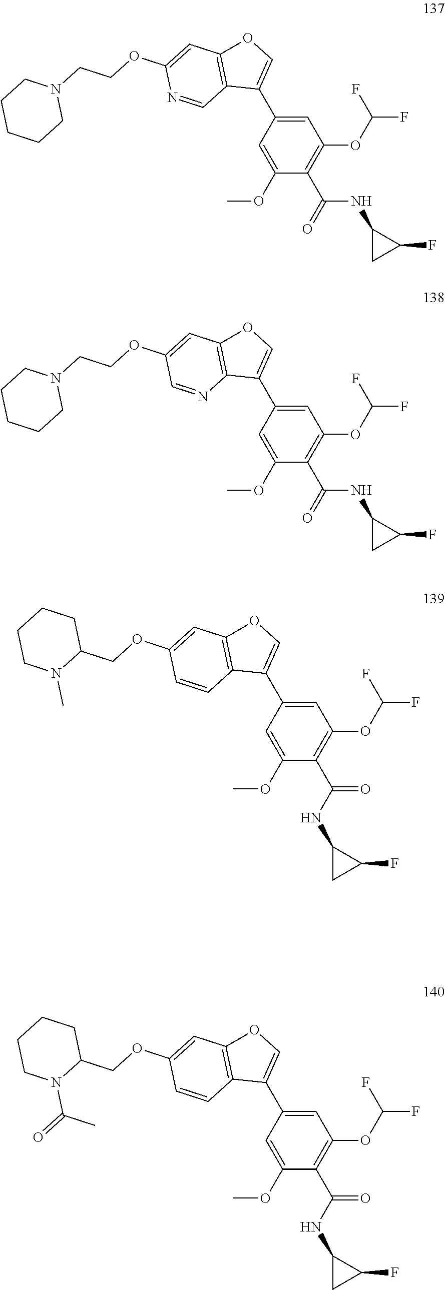 [SMILES: COc1cc(-c2coc3cc(OCCN4CCCCC4)ncc23)cc(OC(F)F)c1C(=O)N[C@@H]1C[C@@H]1F ; COc1cc(-c2coc3cc(OCCN4CCCCC4)cnc23)cc(OC(F)F)c1C(=O)N[C@@H]1C[C@@H]1F ; COc1cc(-c2coc3cc(OCC4CCCCN4C)ccc23)cc(OC(F)F)c1C(=O)N[C@@H]1C[C@@H]1F ; COc1cc(-c2coc3cc(OCC4CCCCN4C(C)=O)ccc23)cc(OC(F)F)c1C(=O)N[C@@H]1C[C@@H]1F]

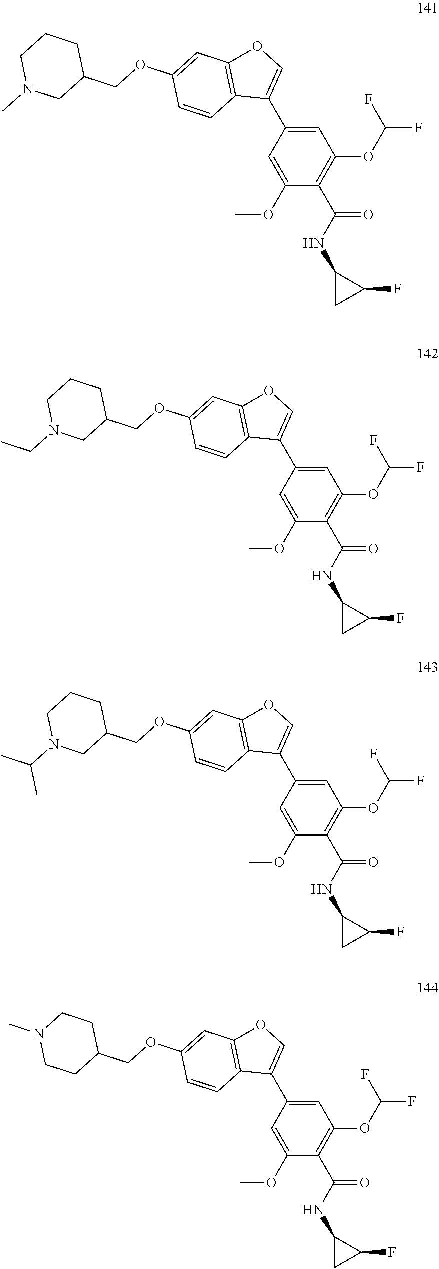 [SMILES: COc1cc(-c2coc3cc(OCC4CCCN(C)C4)ccc23)cc(OC(F)F)c1C(=O)N[C@@H]1C[C@@H]1F ; CCN1CCCC(COc2ccc3c(-c4cc(OC)c(C(=O)N[C@@H]5C[C@@H]5F)c(OC(F)F)c4)coc3c2)C1 ; COc1cc(-c2coc3cc(OCC4CCCN(C(C)C)C4)ccc23)cc(OC(F)F)c1C(=O)N[C@@H]1C[C@@H]1F ; COc1cc(-c2coc3cc(OCC4CCN(C)CC4)ccc23)cc(OC(F)F)c1C(=O)N[C@@H]1C[C@@H]1F]

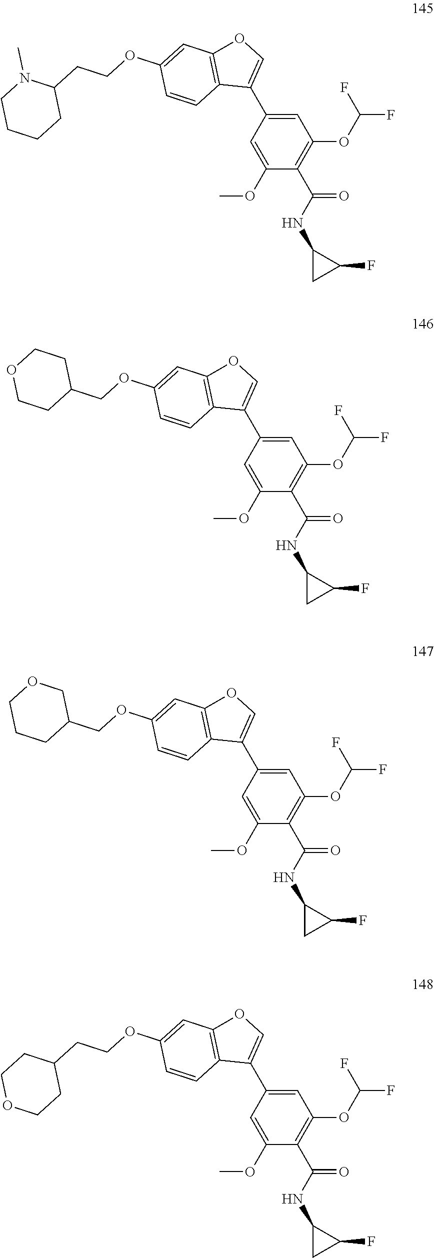 [SMILES: COc1cc(-c2coc3cc(OCCC4CCCCN4C)ccc23)cc(OC(F)F)c1C(=O)N[C@@H]1C[C@@H]1F ; COc1cc(-c2coc3cc(OCC4CCOCC4)ccc23)cc(OC(F)F)c1C(=O)N[C@@H]1C[C@@H]1F ; COc1cc(-c2coc3cc(OCC4CCCOC4)ccc23)cc(OC(F)F)c1C(=O)N[C@@H]1C[C@@H]1F ; COc1cc(-c2coc3cc(OCCC4CCOCC4)ccc23)cc(OC(F)F)c1C(=O)N[C@@H]1C[C@@H]1F]

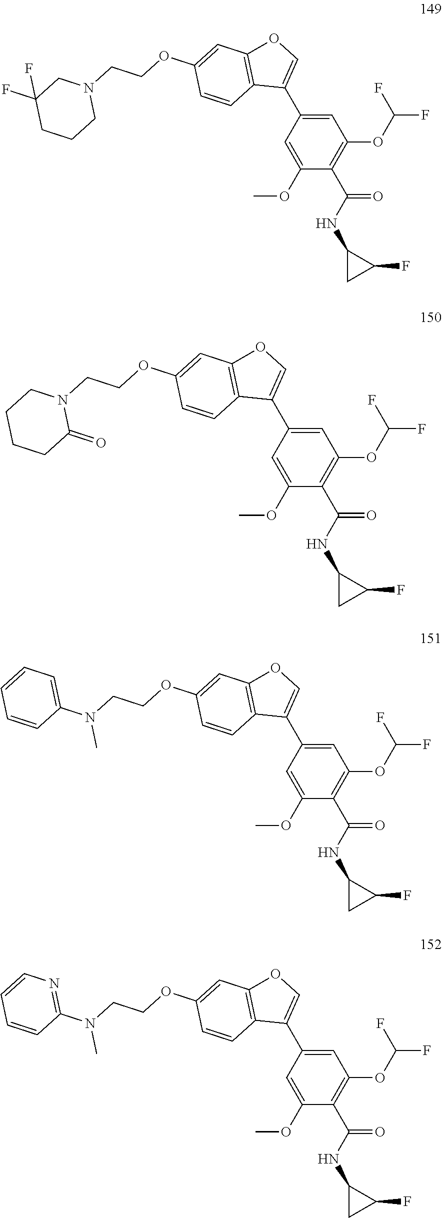 [SMILES: COc1cc(-c2coc3cc(OCCN4CCCC(F)(F)C4)ccc23)cc(OC(F)F)c1C(=O)N[C@@H]1C[C@@H]1F ; COc1cc(-c2coc3cc(OCCN4CCCCC4=O)ccc23)cc(OC(F)F)c1C(=O)N[C@@H]1C[C@@H]1F ; COc1cc(-c2coc3cc(OCCN(C)c4ccccc4)ccc23)cc(OC(F)F)c1C(=O)N[C@@H]1C[C@@H]1F ; COc1cc(-c2coc3cc(OCCN(C)c4ccccn4)ccc23)cc(OC(F)F)c1C(=O)N[C@@H]1C[C@@H]1F]

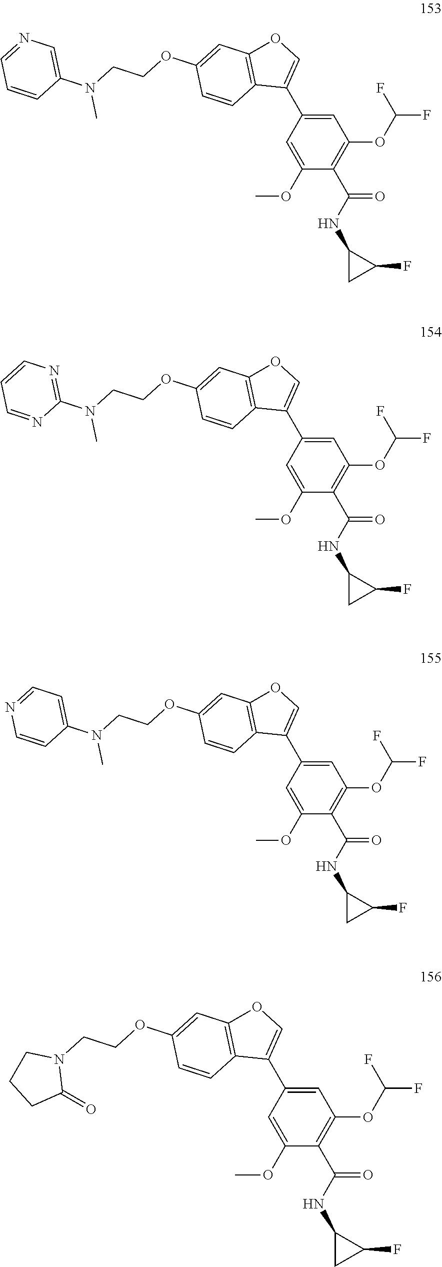 [SMILES: COc1cc(-c2coc3cc(OCCN(C)c4cccnc4)ccc23)cc(OC(F)F)c1C(=O)N[C@@H]1C[C@@H]1F ; COc1cc(-c2coc3cc(OCCN(C)c4ncccn4)ccc23)cc(OC(F)F)c1C(=O)N[C@@H]1C[C@@H]1F ; COc1cc(-c2coc3cc(OCCN(C)c4ccncc4)ccc23)cc(OC(F)F)c1C(=O)N[C@@H]1C[C@@H]1F ; COc1cc(-c2coc3cc(OCCN4CCCC4=O)ccc23)cc(OC(F)F)c1C(=O)N[C@@H]1C[C@@H]1F]

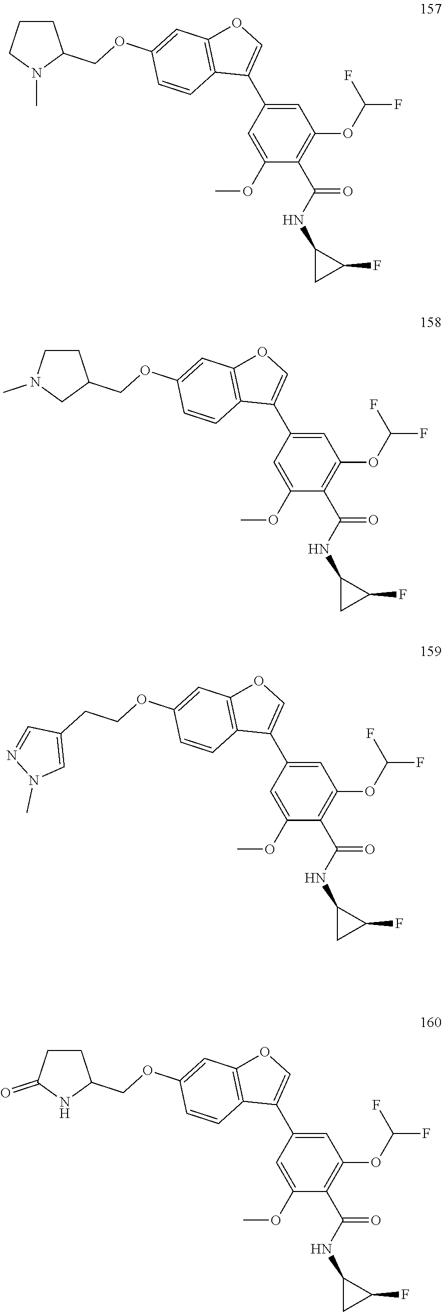 [SMILES: COc1cc(-c2coc3cc(OCC4CCCN4C)ccc23)cc(OC(F)F)c1C(=O)N[C@@H]1C[C@@H]1F ; COc1cc(-c2coc3cc(OCC4CCN(C)C4)ccc23)cc(OC(F)F)c1C(=O)N[C@@H]1C[C@@H]1F ; COc1cc(-c2coc3cc(OCCc4cnn(C)c4)ccc23)cc(OC(F)F)c1C(=O)N[C@@H]1C[C@@H]1F ; COc1cc(-c2coc3cc(OCC4CCC(=O)N4)ccc23)cc(OC(F)F)c1C(=O)N[C@@H]1C[C@@H]1F]

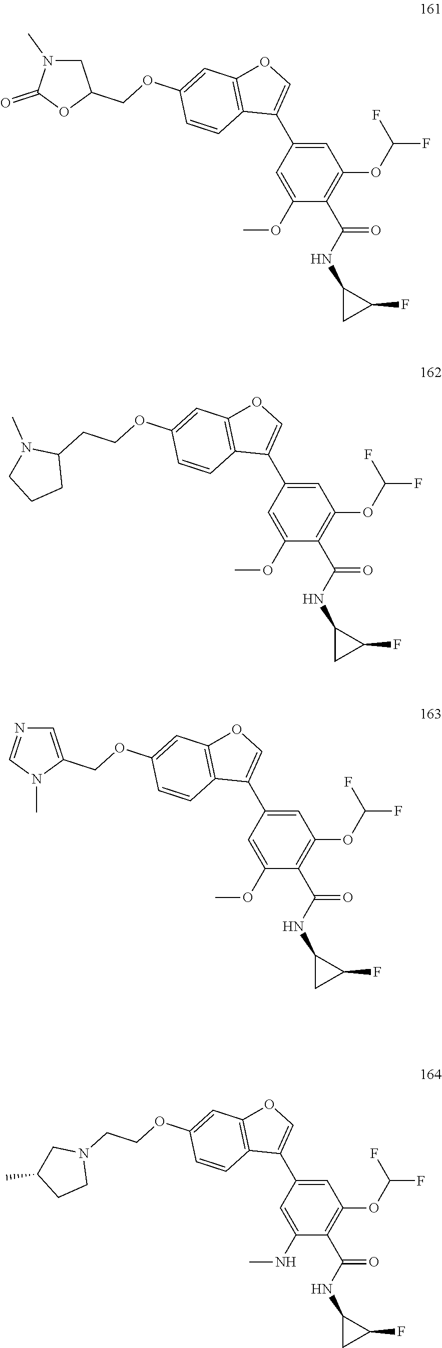 [SMILES: COc1cc(-c2coc3cc(OCC4CN(C)C(=O)O4)ccc23)cc(OC(F)F)c1C(=O)N[C@@H]1C[C@@H]1F ; COc1cc(-c2coc3cc(OCCC4CCCN4C)ccc23)cc(OC(F)F)c1C(=O)N[C@@H]1C[C@@H]1F ; COc1cc(-c2coc3cc(OCc4cncn4C)ccc23)cc(OC(F)F)c1C(=O)N[C@@H]1C[C@@H]1F ; CNc1cc(-c2coc3cc(OCCN4CC[C@H](C)C4)ccc23)cc(OC(F)F)c1C(=O)N[C@@H]1C[C@@H]1F]

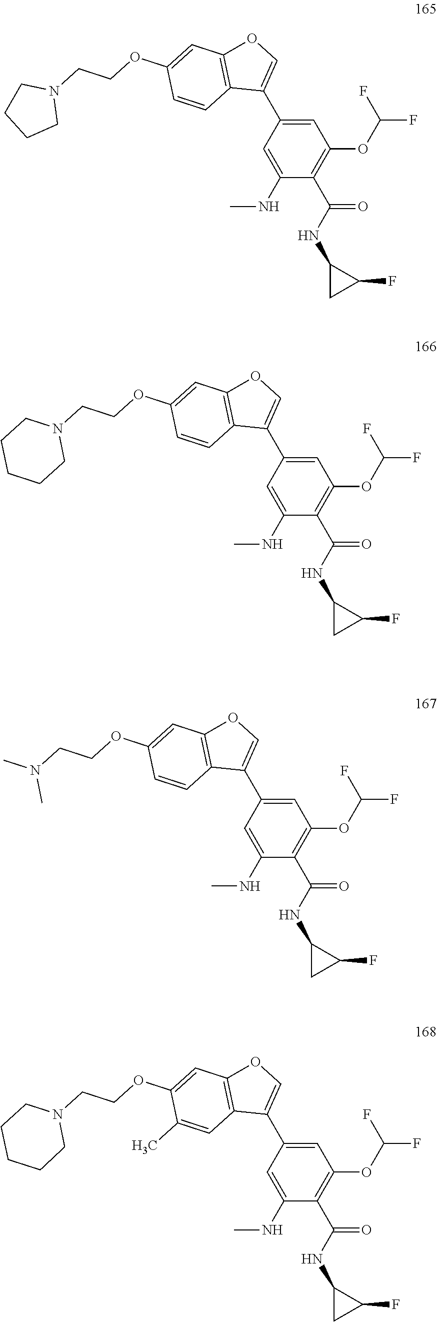 [SMILES: CNc1cc(-c2coc3cc(OCCN4CCCC4)ccc23)cc(OC(F)F)c1C(=O)N[C@@H]1C[C@@H]1F ; CNc1cc(-c2coc3cc(OCCN4CCCCC4)ccc23)cc(OC(F)F)c1C(=O)N[C@@H]1C[C@@H]1F ; CNc1cc(-c2coc3cc(OCCN(C)C)ccc23)cc(OC(F)F)c1C(=O)N[C@@H]1C[C@@H]1F ; CNc1cc(-c2coc3cc(OCCN4CCCCC4)c(C)cc23)cc(OC(F)F)c1C(=O)N[C@@H]1C[C@@H]1F]

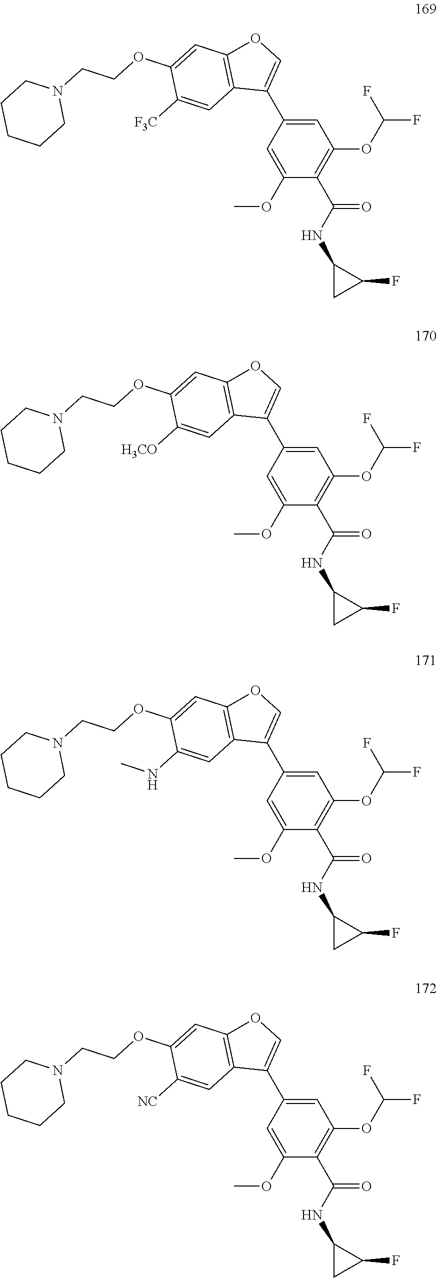 [SMILES: COc1cc(-c2coc3cc(OCCN4CCCCC4)c(C(F)(F)F)cc23)cc(OC(F)F)c1C(=O)N[C@@H]1C[C@@H]1F ; COc1cc2c(-c3cc(OC)c(C(=O)N[C@@H]4C[C@@H]4F)c(OC(F)F)c3)coc2cc1OCCN1CCCCC1 ; CNc1cc2c(-c3cc(OC)c(C(=O)N[C@@H]4C[C@@H]4F)c(OC(F)F)c3)coc2cc1OCCN1CCCCC1 ; COc1cc(-c2coc3cc(OCCN4CCCCC4)c(C#N)cc23)cc(OC(F)F)c1C(=O)N[C@@H]1C[C@@H]1F]

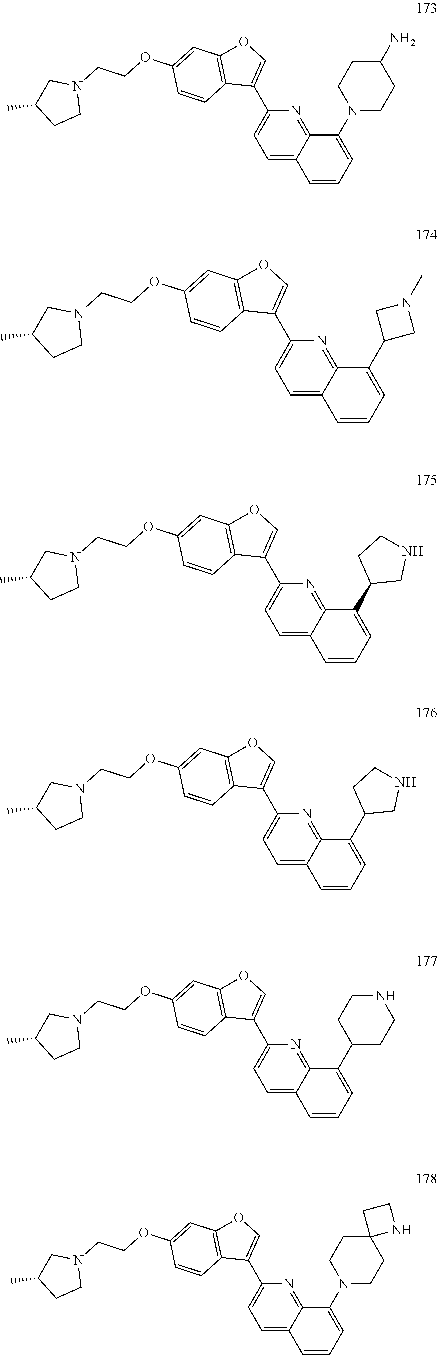

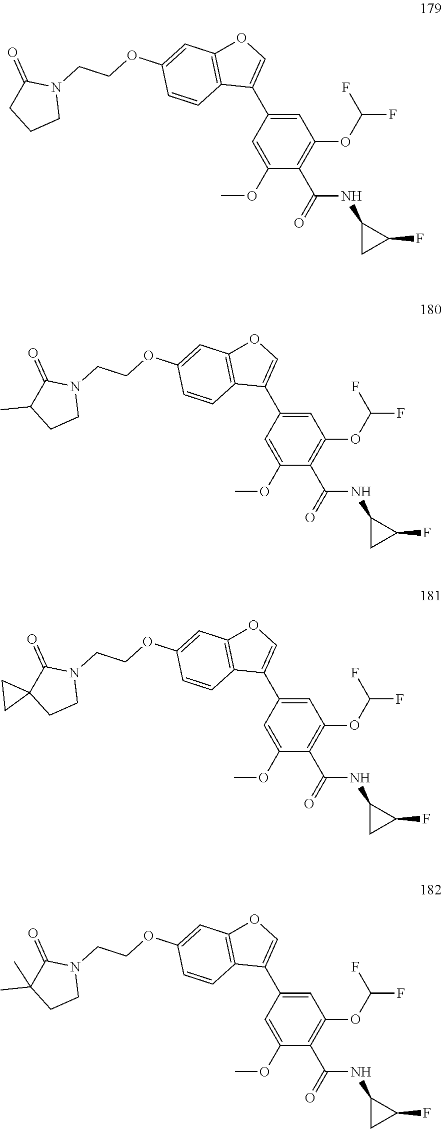 [SMILES: COc1cc(-c2coc3cc(OCCN4CCCC4=O)ccc23)cc(OC(F)F)c1C(=O)N[C@@H]1C[C@@H]1F ; COc1cc(-c2coc3cc(OCCN4CCC(C)C4=O)ccc23)cc(OC(F)F)c1C(=O)N[C@@H]1C[C@@H]1F ; COc1cc(-c2coc3cc(OCCN4CCC5(CC5)C4=O)ccc23)cc(OC(F)F)c1C(=O)N[C@@H]1C[C@@H]1F ; COc1cc(-c2coc3cc(OCCN4CCC(C)(C)C4=O)ccc23)cc(OC(F)F)c1C(=O)N[C@@H]1C[C@@H]1F]

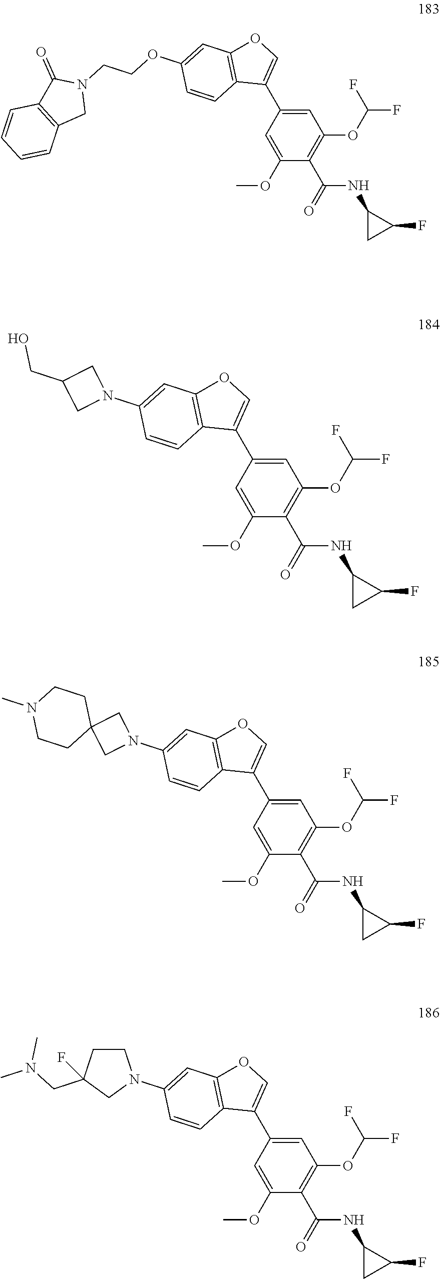 [SMILES: COc1cc(-c2coc3cc(OCCN4Cc5ccccc5C4=O)ccc23)cc(OC(F)F)c1C(=O)N[C@@H]1C[C@@H]1F ; COc1cc(-c2coc3cc(N4CC(CO)C4)ccc23)cc(OC(F)F)c1C(=O)N[C@@H]1C[C@@H]1F ; COc1cc(-c2coc3cc(N4CC5(CCN(C)CC5)C4)ccc23)cc(OC(F)F)c1C(=O)N[C@@H]1C[C@@H]1F ; COc1cc(-c2coc3cc(N4CCC(F)(CN(C)C)C4)ccc23)cc(OC(F)F)c1C(=O)N[C@@H]1C[C@@H]1F]

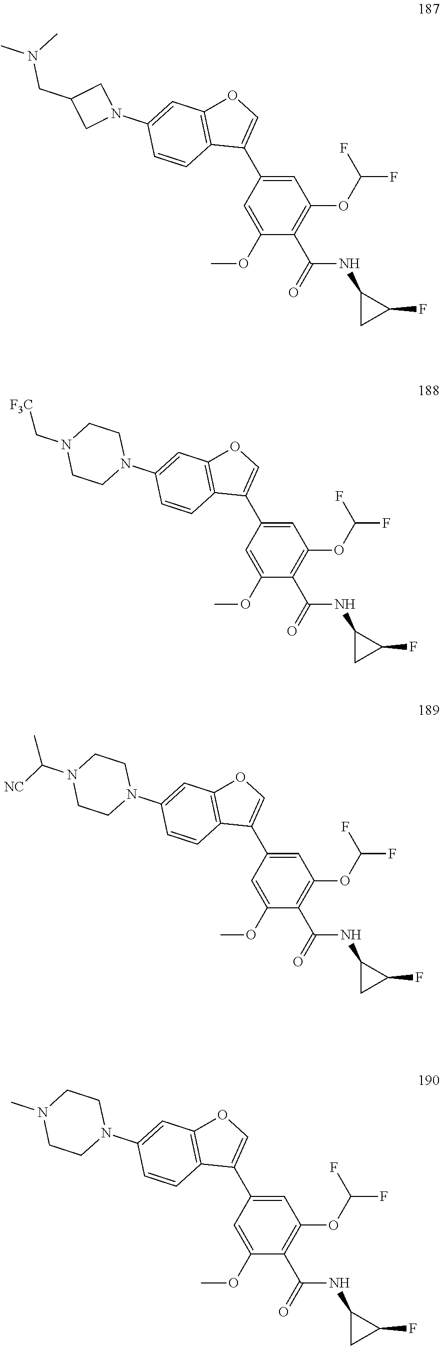 [SMILES: COc1cc(-c2coc3cc(N4CC(CN(C)C)C4)ccc23)cc(OC(F)F)c1C(=O)N[C@@H]1C[C@@H]1F ; COc1cc(-c2coc3cc(N4CCN(CC(F)(F)F)CC4)ccc23)cc(OC(F)F)c1C(=O)N[C@@H]1C[C@@H]1F ; COc1cc(-c2coc3cc(N4CCN(C(C)C#N)CC4)ccc23)cc(OC(F)F)c1C(=O)N[C@@H]1C[C@@H]1F ; COc1cc(-c2coc3cc(N4CCN(C)CC4)ccc23)cc(OC(F)F)c1C(=O)N[C@@H]1C[C@@H]1F]

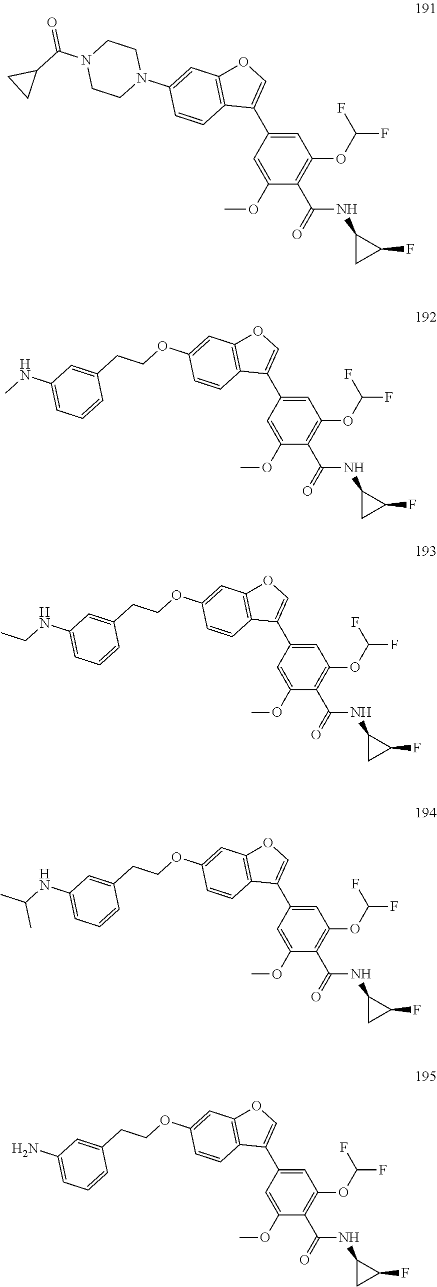 [SMILES: COc1cc(-c2coc3cc(N4CCN(C(=O)C5CC5)CC4)ccc23)cc(OC(F)F)c1C(=O)N[C@@H]1C[C@@H]1F ; CNc1cccc(CCOc2ccc3c(-c4cc(OC)c(C(=O)N[C@@H]5C[C@@H]5F)c(OC(F)F)c4)coc3c2)c1 ; CCNc1cccc(CCOc2ccc3c(-c4cc(OC)c(C(=O)N[C@@H]5C[C@@H]5F)c(OC(F)F)c4)coc3c2)c1 ; COc1cc(-c2coc3cc(OCCc4cccc(NC(C)C)c4)ccc23)cc(OC(F)F)c1C(=O)N[C@@H]1C[C@@H]1F ; COc1cc(-c2coc3cc(OCCc4cccc(N)c4)ccc23)cc(OC(F)F)c1C(=O)N[C@@H]1C[C@@H]1F]

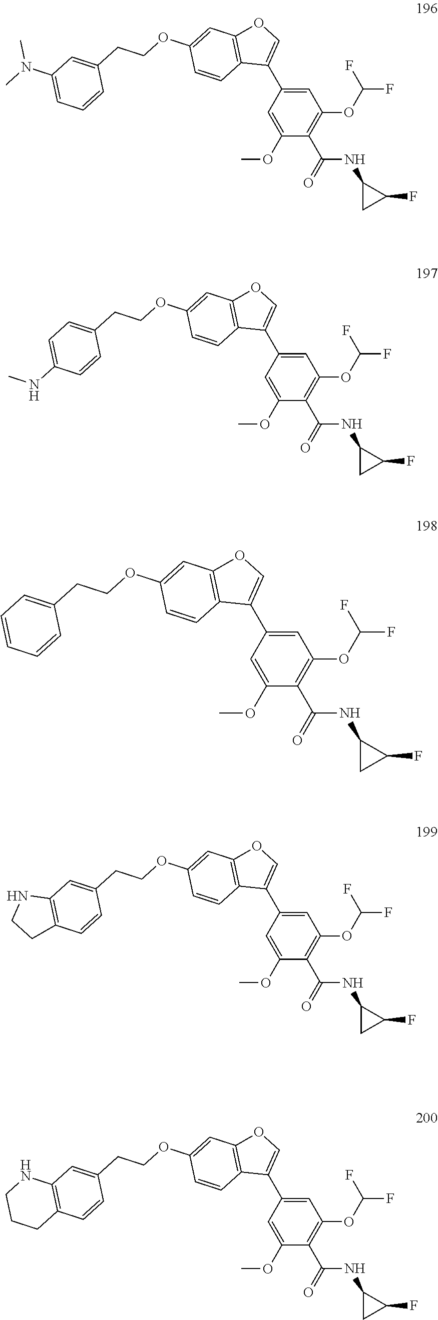 [SMILES: COc1cc(-c2coc3cc(OCCc4cccc(N(C)C)c4)ccc23)cc(OC(F)F)c1C(=O)N[C@@H]1C[C@@H]1F ; CNc1ccc(CCOc2ccc3c(-c4cc(OC)c(C(=O)N[C@@H]5C[C@@H]5F)c(OC(F)F)c4)coc3c2)cc1 ; COc1cc(-c2coc3cc(OCCc4ccccc4)ccc23)cc(OC(F)F)c1C(=O)N[C@@H]1C[C@@H]1F ; COc1cc(-c2coc3cc(OCCc4ccc5c(c4)NCC5)ccc23)cc(OC(F)F)c1C(=O)N[C@@H]1C[C@@H]1F ; COc1cc(-c2coc3cc(OCCc4ccc5c(c4)NCCC5)ccc23)cc(OC(F)F)c1C(=O)N[C@@H]1C[C@@H]1F]

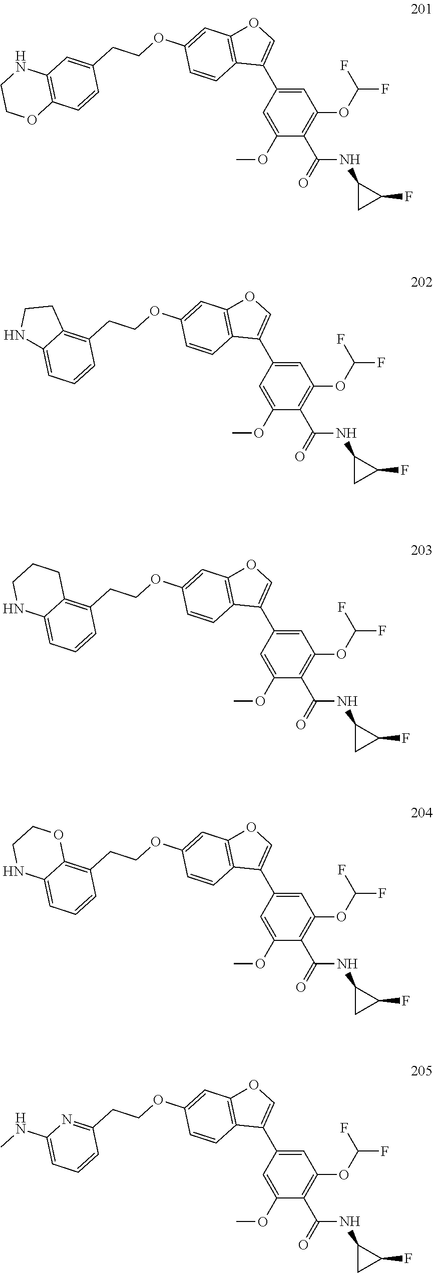 [SMILES: COc1cc(-c2coc3cc(OCCc4ccc5c(c4)NCCO5)ccc23)cc(OC(F)F)c1C(=O)N[C@@H]1C[C@@H]1F ; COc1cc(-c2coc3cc(OCCc4cccc5c4CCN5)ccc23)cc(OC(F)F)c1C(=O)N[C@@H]1C[C@@H]1F ; COc1cc(-c2coc3cc(OCCc4cccc5c4CCCN5)ccc23)cc(OC(F)F)c1C(=O)N[C@@H]1C[C@@H]1F ; COc1cc(-c2coc3cc(OCCc4cccc5c4OCCN5)ccc23)cc(OC(F)F)c1C(=O)N[C@@H]1C[C@@H]1F ; CNc1cccc(CCOc2ccc3c(-c4cc(OC)c(C(=O)N[C@@H]5C[C@@H]5F)c(OC(F)F)c4)coc3c2)n1]

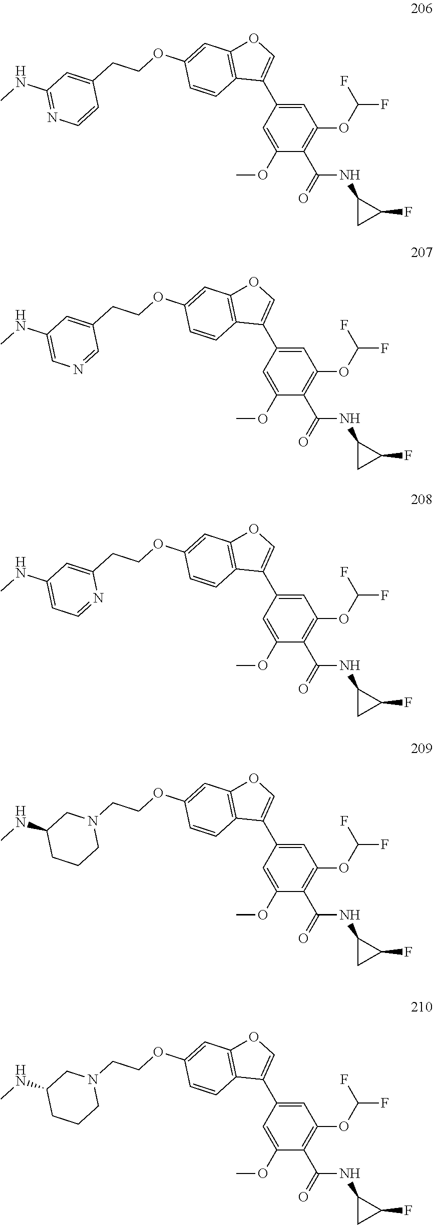 [SMILES: CNc1cc(CCOc2ccc3c(-c4cc(OC)c(C(=O)N[C@@H]5C[C@@H]5F)c(OC(F)F)c4)coc3c2)ccn1 ; CNc1cncc(CCOc2ccc3c(-c4cc(OC)c(C(=O)N[C@@H]5C[C@@H]5F)c(OC(F)F)c4)coc3c2)c1 ; CNc1ccnc(CCOc2ccc3c(-c4cc(OC)c(C(=O)N[C@@H]5C[C@@H]5F)c(OC(F)F)c4)coc3c2)c1 ; CN[C@@H]1CCCN(CCOc2ccc3c(-c4cc(OC)c(C(=O)N[C@@H]5C[C@@H]5F)c(OC(F)F)c4)coc3c2)C1 ; CN[C@H]1CCCN(CCOc2ccc3c(-c4cc(OC)c(C(=O)N[C@@H]5C[C@@H]5F)c(OC(F)F)c4)coc3c2)C1]

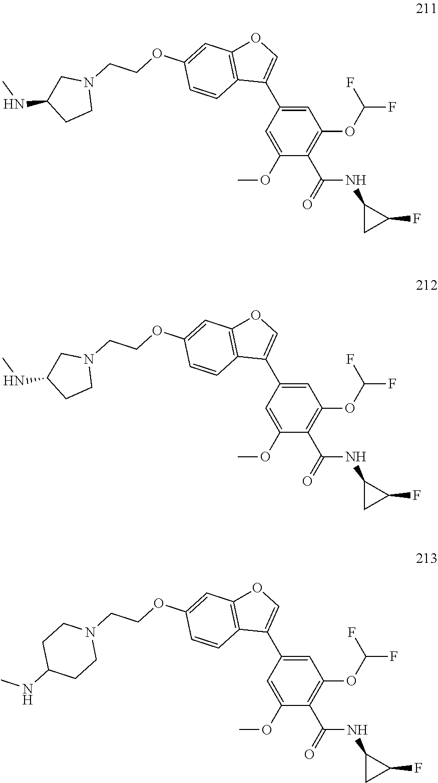 [SMILES: CN[C@@H]1CCN(CCOc2ccc3c(-c4cc(OC)c(C(=O)N[C@@H]5C[C@@H]5F)c(OC(F)F)c4)coc3c2)C1 ; CN[C@H]1CCN(CCOc2ccc3c(-c4cc(OC)c(C(=O)N[C@@H]5C[C@@H]5F)c(OC(F)F)c4)coc3c2)C1 ; CNC1CCN(CCOc2ccc3c(-c4cc(OC)c(C(=O)N[C@@H]5C[C@@H]5F)c(OC(F)F)c4)coc3c2)CC1]

Aspect 24. A pharmaceutical composition comprising the compound of any of Aspects 1-23, or a pharmaceutically acceptable salt, stereoisomer, or tautomer thereof and a pharmaceutically acceptable excipient.
Aspect 25. A method of treating a disease or disorder, comprising administering to a subject in need thereof t a therapeutically effective amount of the compound of any of Aspects 1-23 or a pharmaceutically acceptable salt, stereoisomer, or tautomer thereof.
Aspect 26. The method of Aspect 25, wherein the disease or disorder is selected from inflammatory diseases, autoinflammatory diseases, and autoimmune diseases.

### DETAILED DESCRIPTION OF THE INVENTION

The following terms have the indicated meanings throughout the specification:

As used herein, including the appended Aspects, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CHCl₂, CF₃, and the like.

The term “alkenyl” refers to a hydrocarbon group selected from linear and branched hydrocarbon groups comprising at least one C≡C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy includes, but not limited to, methoxy, ethoxy, isopropoxy, propoxy, n-butoxy, tert-butoxy, pentoxy and hexoxy and the like.

The term “alkoxy-alkyl-” refers to an alkyl group as defined above further substituted with an alkoxy as defined above. Examples of an alkoxy-alkyl-, e.g., C₁₋₈alkoxy-C₁₋₈alkyl- includes, but not limited to, methoxymethyl, ethoxymethyl, isopropoxymethyl, or propoxymethyl and the like.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems, such as

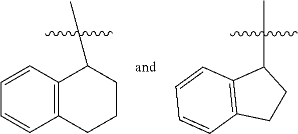 [SMILES: *C1CCCc2ccccc21 ; *C1CCc2ccccc21]

wherein the wavy lines indicate the points of attachment. The ring may be saturated or have at least one double bond (i.e. partially unsaturated), but is not fully conjugated, and is not aromatic, as aromatic is defined herein.

The term “spiro cycloalkyl” refers to a cyclic structure which contains carbon atoms and is formed by at least two rings sharing one atom. The term “7- to 12-membered spiro cycloalkyl” refers to a cyclic structure which contains 7 to 12 carbon atoms and is formed by at least two rings sharing one atom.

The term “fused cycloalkyl” refers to a fused ring which contains carbon atoms and is formed by two or more rings sharing two adjacent atoms. The term “4- to 10-membered fused cycloalkyl” refers to a fused ring which contains 4 to 10 ring carbon atoms and is formed by two or more rings sharing two adjacent atoms.

Examples include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3- to 8-membered cycloalkyl, benzo C₄₋₆ cycloalkenyl, 2,3-dihydro-1H-indenyl, 1H-indenyl, 1,2,3,4-tetrazolyl, 1,4-dihydronaphthyl, etc. Preferred embodiments are 8- to 9-membered fused ring, which refer to cyclic structures containing 8 to 9 ring atoms within the above examples.

The term “bridged cycloalkyl” refers to a cyclic structure which contains carbon atoms and is formed by two rings sharing two atoms which are not adjacent to each other. The term “7- to 10-membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl(1-cyclopent-1-enyl, 1-cyclopent-2-enyl, 1-cyclopent-3-enyl) or cyclohexenyl(1-cyclohex-1-enyl, 1-cyclohex-2-enyl, 1-cyclohex-3-enyl), preferably cyclohexenyl.

The term “cycloalkynyl” refers to non-aromatic cycloalkyl groups of from 5 to 10 carbon atoms having single or multiple rings and having at least one triple bond.

The term “aryl” used alone or in combination with other terms refers to a group selected from:
5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur(S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur(S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur(S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur(S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

“heterocycloalkyl”, “Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refer to S, SO or SO₂.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include, but not limited to the following groups: 2,3-dihydrospiro[indene-1,2′-pyrrolidine] (e.g., 2,3-dihydrospiro[indene-1,2′-pyrrolidine]-1′-yl), 1,3-dihydrospiro[indene-2,2′-pyrrolidine] (e.g., 1,3-dihydrospiro[indene-2,2′-pyrrolidine]-1′-yl), azaspiro[2.4]heptane (e.g., 5-azaspiro[2.4]heptane-5-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1,7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, preferably 7 to 12-membered and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindolin-2-yl or isoindolin-5-yl), octahydro-benzo[b][1,4]dioxin, dihydropyridooxazinyl (e.g., 2,3-dihydro-1H-pyrido[2,3-b][1,4]oxazinyl) or dihydrobenzooxazepinyl (e.g., 5-oxo-3,4-dihydrobenzo[f][1,4]oxazepiny), benzazepinyl (e.g., 2,3,4,5-tetrahydro-1-oxo-2-benzazepin-6-yl), benzoxazepinyl (e.g., 5-oxo-2,3,4,5-tetrahydro-1,4-benzoxazepin-8-yl), dihydroisoquinolinyl (e.g., 1-oxo-2-methyl-3,4-dihydroisoquinolin-6-yl), tetrahydroisoquinolinyl (e.g., 2-methyl-1-oxo-1,2,3,4-tetrahydroisoquinolin-6-yl), dihydrobenzoxazine (e . . . g, 3,4-dihydro-2H-1,4-benzoxazin-6-yl), hexahydrofuro[3,4-c]pyrrolyl.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7- to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

The term “alkylene” refers to a divalent alkyl radical as defined above. The term “alkenylene” refers to a divalent alkenyl radical as defined above. The term “alkynylene” refers to a divalent alkynyl radical as defined above. The term “cycloalkylene” refers to a divalent cycloalkyl radical as defined above. The term “heterocyclylene” refers to a divalent heterocyclyl radical as defined above. The term “arylene” refers to a divalent aryl radical as defined above. The term “heteroarylene” refers to a divalent heteroarylene radical as defined above.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Tautomer” refers to alternate forms of a compound which differ only electronic bonding of atoms and/or in the position of electrons, such as enol-keto and imine-enamine tautomers, or the tautomeric forms of heteroaryl groups containing a —N—C(H)—NH— ring atom arrangement, such as pyrazoles, imidazoles, benzimidazoles, triazoles, and tetrazoles.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of an appropriate protecting group can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC is used for the retention time analysis of different chiral examples, the conditions are divided into the methods as below according to the column, mobile phase, the solvent ratio used.

The general synthetic schemes were described as below:

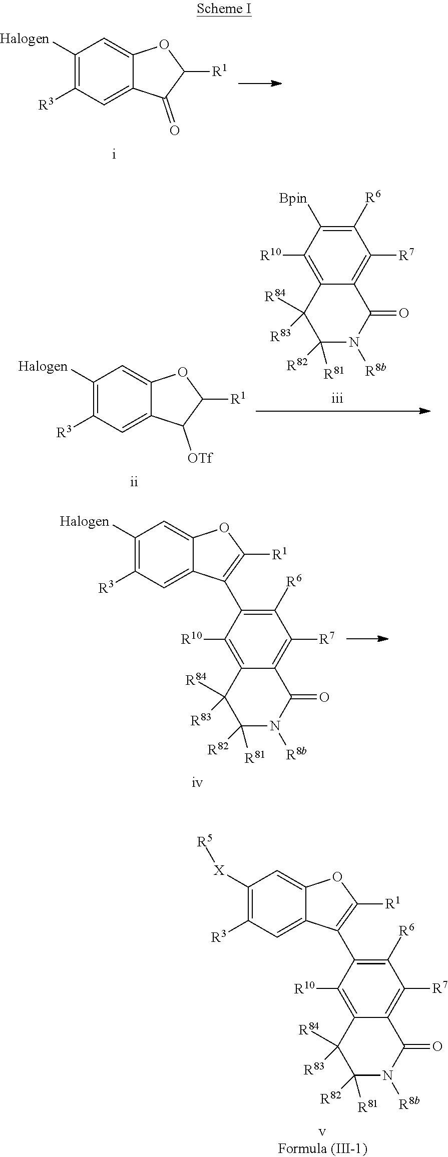

Compounds of Formula (III-1) can be generally prepared as shown in scheme I. reaction of halide-functionalized furanone (compound i) with triflic anhydride afforded compound (ii), followed by Suzuki coupling with compound iii to give compound iv. Compound iv reacted with various functionalized heterocycles via Suzuki coupling to afford compound (v) described in Formula (III-1).

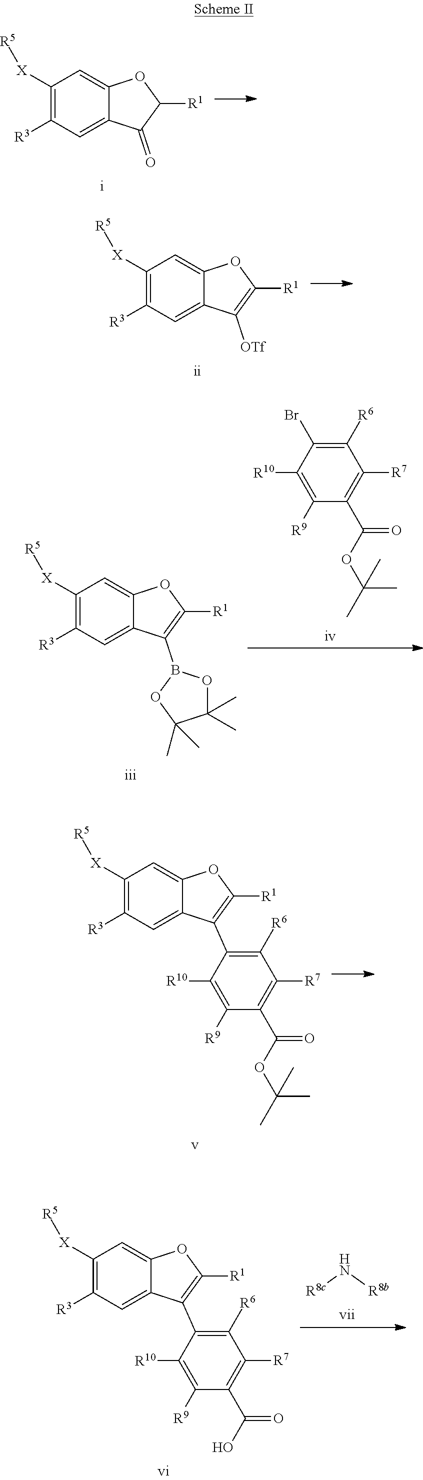

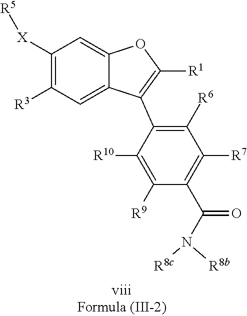

Compounds of Formula (III-2) can be generally prepared as shown in scheme II. Reaction of furanone (compound i) with triflic anhydride afforded compound (ii), compound (ii) was converted to compound (iii) via palladium-catalyzed borylation. Suzuki coupling between compound (iii) and compound (iv) afforded compound (v), followed by acidic hydrolysis to give the acid (compound vi), compound vi condensed with various amine (compound vii) to afford compound (viii) described in Formula (III-2).

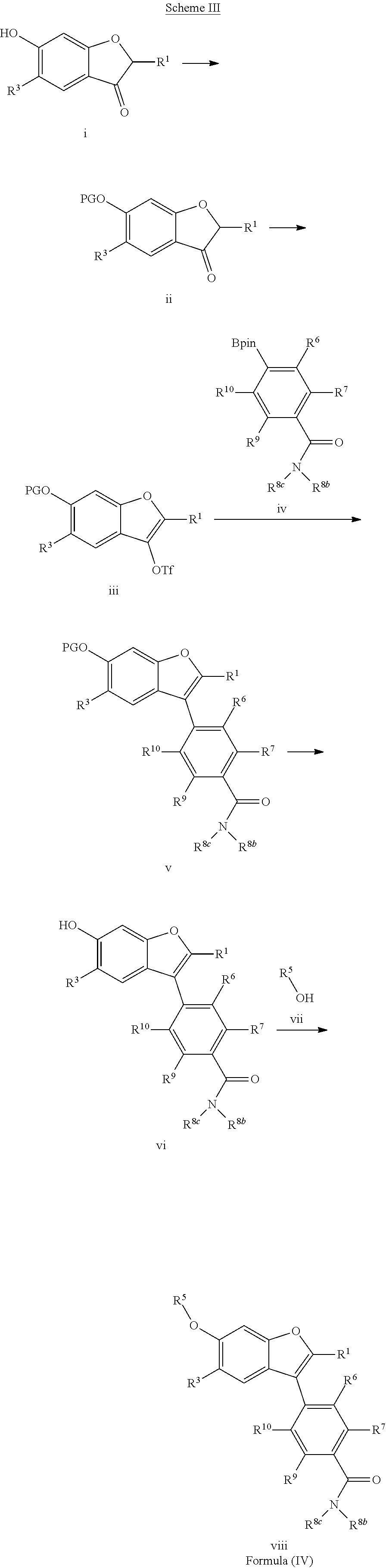

Compounds of Formula (IV) can be generally prepared as shown in scheme (III). Protection of phenol in compound (i) afforded compound (ii), followed by reaction with triflic anhydride to give compound (iii), compound (iii) reacted with compound (iv) via Suzuki coupling to afford compound (v), followed by acidic hydrolysis to afford deprotection compound (vi). Finally, compound (vi) coupled with various alcohol (compound vii) via Mitsunobu reaction to give compound (viii) described in Formula (IV).

### Abbreviations

PE Petroleum ether
EtOAc Ethyl acetate
THF Tetrahydrofuran
DCM Dichloromethane
MeOH Methanol
DMSO Dimethyl sulfoxide
DMF Dimethyl formamide
DIEA N, N-diisopropylethylamine
TEA Triethylamine
eq Equivalent
Tf₂O Triflic anhydride
TLC Thin layer chromatography
LiHMDS Lithium bis(trimethylsilyl)amide
KOAc Potassium acetate
K₂CO₃ Potassium carbonate
K₃PO₄ Potassium phosphate
Na₂SO₄ Sodium sulphate
MeONa Sodium methoxide
Pd(dppf)Cl₂ [1,1′-Bis(diphenylphosphino) ferrocene]dichloropalladium (II)
Xphos 2-Dicyclohexylphosphino-2′,4′,6′-triisopropylbiphenyl
Pd₂(dba)₃ Tris(dibenzylideneacetone)dipalladium(0)
NaH Sodium hydride
NH₄C₁ Ammonium chloride
BBr₃ Boron tribromide
KOH Potassium hydroxide
LiOH Lithium hydroxide
Prep-HPLC Preparative high performance liquid chromatography

### Example 1: 8-methoxy-6-(6-(1-methyl-1H-pyrazol-4-yl)benzofuran-3-yl)-2-(2,2,2-trifluoroethyl)-3,4-dihydroisoquinolin-1 (2H)-one (compound 1)

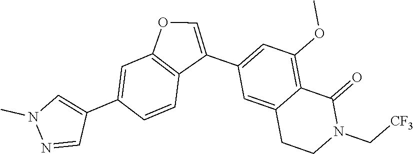 [SMILES: COc1cc(-c2coc3cc(-c4cnn(C)c4)ccc23)cc2c1C(=O)N(CC(F)(F)F)CC2]

### Step 1: 6-chlorobenzofuran-3-yl trifluoromethanesulfonate

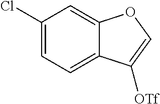 [SMILES: O=S(=O)(Oc1coc2cc(Cl)ccc12)C(F)(F)F]

To a solution of 6-chlorobenzofuran-3 (2H)-one (8 g, 47.46 mmol) in DCM (80 mL) was added DIEA (7.97 g, 61.69 mmol, 10.75 mL), The mixture was stirred at 0° C. for 10 min, and Tf₂O (20.08 g, 71.18 mmol, 11.74 mL) was added. The mixture was stirred at 0˜25° C. for 2 hr. TLC (PE/EtOAc=5/1, Rf=0.8) indicated the starting material was consumed completely. The reaction mixture was added H₂O (100 mL), extracted with DCM (50 mL*2, twice, 50 mL each time). The combined organic layers were washed with brine, dried over Na₂SO₄, filtered and concentrated under reduced pressure. The residue was purified by silica gel column chromatography, eluting with PE/EtOAc (v/v=20/1 to 0/1), to give the title product (13.7 g, 96% yield). ¹H NMR (400 MHz, CDCl₃) δ 7.83 (s, 1H), 7.56-7.53 (m, 2H), 7.89 (s, 1H), 7.37 (dd, J=1.6, 8.4 Hz, 1H).

### Step 2: 6-bromo-8-methoxy-3,4-dihydroisoquinolin-1 (2H)-one

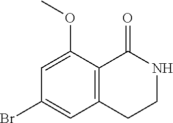

To a solution of 6-bromo-8-fluoro-3,4-dihydroisoquinolin-1 (2H)-one (7 g, 28.68 mmol) in THF (100 mL) was added NaOMe (5.63 g, 34.42 mmol, 33% purity in MeOH). The mixture was stirred at 20° C. for 16 hrs. LCMS showed the reaction was completed. The mixture was quenched by NH₄Cl aq (50 mL) and extracted with EtOAc (100 mL*3, three times, 100 mL each time). The combined organic phase was washed with brine, dried with anhydrous Na₂SO₄, filtered and concentrated under reduced pressure to give the title product (7 g, 95% yield). ¹H NMR (400 MHz, DMSO-d₆) δ 7.78 (s, 1H), 7.13 (d, J=18.4 Hz, 2H), 3.78 (s, 3H), 3.29-3.07 (m, 2H), 2.79 (t, J=6.4 Hz, 2H).

### Step 3: 6-bromo-8-methoxy-2-(2,2,2-trifluoroethyl)-3,4-dihydroisoquinolin-1 (2H)-one

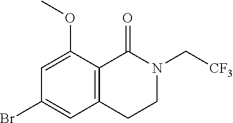 [SMILES: COc1cc(Br)cc2c1C(=O)N(CC(F)(F)F)CC2]

To a solution of 6-bromo-8-methoxy-3,4-dihydroisoquinolin-1 (2H)-one (7 g, 27.33 mmol) in THF (200 mL) was added Lithium bis(trimethylsilyl) amide (LiHMDS) (1 M, 41.00 mL) under 0° C. The mixture was stirred at 0° C. for 1 h and 2,2,2-trifluoroethyl trifluoromethanesulfonate (9.52 g, 41.00 mmol) was added to the mixture. The reaction was stirred at 50° C. for 6 hrs. The mixture was poured into saturated NH₄Cl (50 mL). The aqueous phase was extracted with EtOAc (100 mL*3). The combined organic phase was washed with brine, dried with anhydrous Na₂SO₄, filtered and concentrated under reduced pressure. The residue was purified by silica gel column chromatography, eluting with PE/EtOAc (v/v=100/1 to 0/1), to give the title product (5 g, 54% yield). ¹H NMR (400 MHz, DMSO-d₆) δ 7.20 (s, 1H), 7.13 (s, 1H), 4.30 (q, J=9.6 Hz, 2H), 3.80 (s, 3H), 3.56 (t, J=6.0 Hz, 2H), 2.90 (t, J=6.0 Hz, 2H).

### Step 4: 8-methoxy-6-(4,4,5,5-tetramethyl-1,3,2-dioxaborolan-2-yl)-2-(2,2,2-trifluoroethyl)-3,4-dihydroisoquinolin-1 (2H)-one

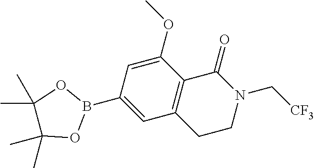 [SMILES: COc1cc(B2OC(C)(C)C(C)(C)O2)cc2c1C(=O)N(CC(F)(F)F)CC2]

To a solution of 6-bromo-8-methoxy-2-(2,2,2-trifluoroethyl)-3,4-dihydroisoquinolin-1 (2H)-one (5 g, 14.79 mmol) in dioxane (50 mL) was added 4,4,4′,4′,5,5,5′,5′-octamethyl-2,2′-bi (1,3,2-dioxaborolane) (5.63 g, 22.18 mmol), KOAc (7.26 g, 73.94 mmol) and Pd(dppf)Cl₂ (1.08 g, 1.48 mmol). The mixture was stirred at 100° C. for 5 hrs. LCMS showed the reaction was completed. The mixture was filtered and the filter cake was washed by EtOAc (50 mL*3). The filtrated was concentrated under reduced pressure. The residue was purified by silica gel column chromatography, eluting with PE/EtOAc (v/v=100/1 to 0/1), to give the title product (5 g, 87% yield). ¹H NMR (400 MHz, CDCl₃) δ 7.30 (s, 1H), 7.22 (s, 1H), 4.21 (q, J=8.8 Hz, 2H), 3.97 (s, 3H), 3.62 (t, J=6.4 Hz, 2H), 2.97 (t, J=6.4 Hz, 2H), 1.37 (s, 12H).

### Step 5: 6-(6-chlorobenzofuran-3-yl)-8-methoxy-2-(2,2,2-trifluoroethyl)-3,4-dihydroisoquinolin-1(2H)-one

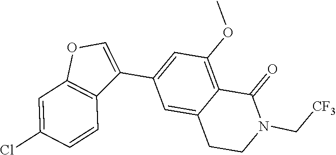 [SMILES: COc1cc(-c2coc3cc(Cl)ccc23)cc2c1C(=O)N(CC(F)(F)F)CC2]

To a solution of 8-methoxy-6-(4,4,5,5-tetramethyl-1,3,2-dioxaborolan-2-yl)-2-(2,2,2-trifluoroethyl)-3,4-dihydroisoquinolin-1 (2H)-one (930.17 mg, 2.41 mmol) in dioxane (10 mL) and H₂O (1 mL) was added 6-chlorobenzofuran-3-yl trifluoromethanesulfonate (0.66 g, 2.20 mmol) and K₃PO₄ (931.99 mg, 4.39 mmol) and Pd(dppf)Cl₂ (160.63 mg, 219.53 μmol) under N₂. The mixture was stirred at 100° C. for 3 hrs under N₂. LCMS showed the reactant was consumed completely. The mixture was diluted with EtOAc (10 mL), filtered through a celite pad and washed with EtOAc (10 mL). The filtrate was concentrated under reduced pressure. The residue was purified by silica gel column chromatography, eluting with PE/EtOAc (v/v=10/1 to 0/1), to give the title product (0.85 g, 94% yield). LC-MS: [M+H]⁺=410.1.

### Step 6: 8-methoxy-6-(6-(1-methyl-1H-pyrazol-4-yl)benzofuran-3-yl)-2-(2,2,2-trifluoroethyl)-3,4-dihydroisoquinolin-1 (2H)-one (compound 1)

To a solution of 6-(6-chlorobenzofuran-3-yl)-8-methoxy-2-(2,2,2-trifluoroethyl)-3,4-dihydroisoquinolin-1 (2H)-one (0.05 g, 122.02 μmol, 1 eq) in dioxane (2 mL) was added 1-methyl-4-(4,4,5,5-tetramethyl-1,3,2-dioxaborolan-2-yl)-1H-pyrazole (30.46 mg, 146.42 μmol), AcOK (29.94 mg, 305.04 μmol), xPhos (5.82 mg, 12.20 μmol) and Pd₂(dba)₃ (11.17 mg, 12.20 μmol) under N₂. The mixture was stirred at 100° C. for 3 hrs under N₂. LC-MS showed the reaction was complete. The mixture was diluted with EtOAc (10 mL), filtered through a celite pad and washed with EtOAc (10 mL). The filtrate was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 35%-55% ACN over 12.0 min) to give compound 1 (86 mg, 15% yield). ¹H NMR (400 MHz, DMSO-d₆) δ 8.50 (s, 1H), 8.24 (s, 1H), 8.01-7.95 (m, 2H), 7.89 (s, 1H), 7.60 (d. J=8.0 Hz, 1H), 7.29 (d. J=9.2 Hz, 2H), 4.39-4.31 (m, 2H), 3.89 (d, J=7.6 Hz, 6H), 3.63 (t, J=6.0 Hz, 2H), 3.01 (t, J=6.0 Hz, 2H). LC-MS: [M+H]⁺=456.1.

### Example 2: 6-(6-(1-ethyl-1H-pyrazol-4-yl)benzofuran-3-yl)-8-methoxy-2-(2,2,2-trifluoroethyl)-3,4-dihydroisoquinolin-1 (2H)-one (compound 2)

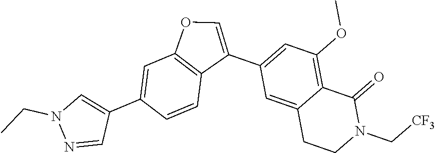 [SMILES: CCn1cc(-c2ccc3c(-c4cc5c(c(OC)c4)C(=O)N(CC(F)(F)F)CC5)coc3c2)cn1]

### Step 1: 6-(6-(1-ethyl-1H-pyrazol-4-yl)benzofuran-3-yl)-8-methoxy-2-(2,2,2-trifluoroethyl)-3,4-dihydroisoquinolin-1 (2H)-one (compound 2)

Compound 2 (6.7 mg, 11% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 1-ethyl-4-(4,4,5,5-tetramethyl-1,3,2-dioxaborolan-2-yl)-1H-pyrazole. ¹H NMR (400 MHz, DMSO-d₆) δ 8.50 (s, 1H), 8.30 (s, 1H), 7.99-7.94 (m, 2H), 7.92-7.88 (m, 1H), 7.63-7.59 (m, 1H), 7.29 (d, J=8.8 Hz, 2H), 4.38-4.31 (m, 2H), 4.17 (q, J=7.6 Hz, 2H), 3.90 (s, 3H), 3.63 (t, J=6.0 Hz, 2H), 3.01 (t. J=6.0 Hz, 2H), 1.43 (t, J=7.6 Hz, 3H). LC-MS: [M+H]⁺=470.1.

### Example 3: 6-(6-(1-isopropyl-1H-pyrazol-4-yl)benzofuran-3-yl)-8-methoxy-2-(2,2,2-trifluoroethyl)-3,4-dihydroisoquinolin-1 (2H)-one (compound 3)

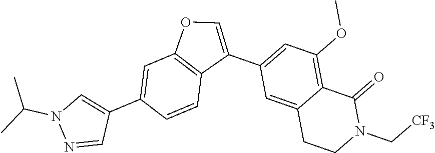 [SMILES: COc1cc(-c2coc3cc(-c4cnn(C(C)C)c4)ccc23)cc2c1C(=O)N(CC(F)(F)F)CC2]

### Step 1: 6-(6-(1-isopropyl-1H-pyrazol-4-yl)benzofuran-3-yl)-8-methoxy-2-(2,2,2-trifluoroethyl)-3,4-dihydroisoquinolin-1 (2H)-one (compound 3)

Compound 3 (6.6 mg, 11% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 1-isopropyl-4-(4,4,5,5-tetramethyl-1,3,2-dioxaborolan-2-yl)-1H-pyrazole. ¹H NMR (400 MHz, DMSO-d₆) § 8.50 (s, 1H), 8.34 (s, 1H), 7.99-7.90 (m, 3H), 7.62 (d, J=8.0 Hz, 1H), 7.29 (d, J=9.2 Hz, 2H), 4.57-4.47 (m, 1H), 4.38-4.31 (m, 2H), 3.90 (s, 3H), 3.63 (d, J=5.2 Hz, 2H), 3.01 (s, 2H), 1.47 (d, J=6.4 Hz, 6H). LC-MS: [M+H]⁺=484.1.

### Example 4: 6-(6-(1-cyclopropyl-1H-pyrazol-4-yl)benzofuran-3-yl)-8-methoxy-2-(2,2,2-trifluoroethyl)-3,4-dihydroisoquinolin-1 (2H)-one (compound 4)

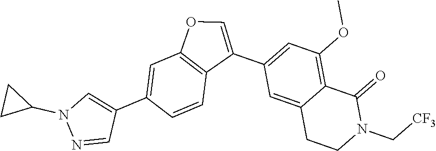 [SMILES: COc1cc(-c2coc3cc(-c4cnn(C5CC5)c4)ccc23)cc2c1C(=O)N(CC(F)(F)F)CC2]

### Step 1: 6-(6-(1-cyclopropyl-1H-pyrazol-4-yl)benzofuran-3-yl)-8-methoxy-2-(2,2,2-trifluoroethyl)-3,4-dihydroisoquinolin-1 (2H)-one (compound 4)

Compound 4 (6.7 mg, 11% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 1-cyclopropyl-4-(4,4,5,5-tetramethyl-1,3,2-dioxaborolan-2-yl)-1H-pyrazole. ¹H NMR (400 MHz, DMSO-d₆) δ 8.50 (s, 1H), 8.35 (s, 1H), 7.98-7.90 (m, 3H), 7.62 (d, J=8.4 Hz, 1H), 7.29 (d, J=9.2 Hz, 2H), 4.39-4.31 (m, 2H), 3.90 (s, 3H), 3.80-3.71 (m, 1H), 3.63 (t, J=6.0 Hz, 2H), 3.01 (t, J=6.0 Hz, 2H), 1.12-1.06 (m, 2H), 1.03-0.97 (m, 2H). LC-MS: [M+H]⁺=482.1.

### Example 5: 8-methoxy-6-(6-(3-methyl-1H-pyrazol-5-yl)benzofuran-3-yl)-2-(2,2,2-trifluoroethyl)-3,4-dihydroisoquinolin-1 (2H)-one (compound 5)

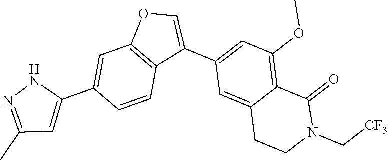 [SMILES: COc1cc(-c2coc3cc(-c4cc(C)n[nH]4)ccc23)cc2c1C(=O)N(CC(F)(F)F)CC2]

### Step 1: 8-methoxy-6-(6-(3-methyl-1H-pyrazol-5-yl)benzofuran-3-yl)-2-(2,2,2-trifluoroethyl)-3,4-dihydroisoquinolin-1 (2H)-one (compound 5)

Compound 5 (14.3 mg, 25% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and (3-methyl-1H-pyrazol-5-yl) boronic acid. ¹H NMR (400 MHz, DMSO-d₆) δ 8.54 (s, 1H), 8.09-7.96 (m, 2H), 7.81 (d, J=8.4 Hz, 1H), 7.30 (d, J=8.4 Hz, 2H), 6.56 (s, 1H), 4.43-4.27 (m, 2H), 3.91 (s, 3H), 3.63 (t, J=6.0 Hz, 2H), 3.01 (t, J=6.0 Hz, 2H), 2.27 (s, 3H). LC-MS: [M+H]⁺=456.1.

### Example 6: 6-(6-(5-ethoxypyridin-3-yl)benzofuran-3-yl)-8-methoxy-2-(2,2,2-trifluoroethyl)-3,4-dihydroisoquinolin-1 (2H)-one (compound 6)

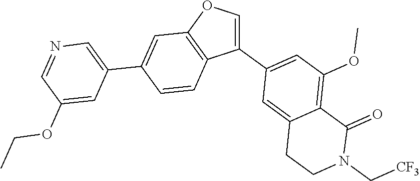 [SMILES: CCOc1cncc(-c2ccc3c(-c4cc5c(c(OC)c4)C(=O)N(CC(F)(F)F)CC5)coc3c2)c1]

Compound 6 (8.1 mg, 13% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 3-ethoxy-5-(4,4,5,5-tetramethyl-1,3,2-dioxaborolan-2-yl)pyridine. ¹H NMR (400 MHz, DMSO-d₆) δ 8.63 (s, 1H), 8.59 (d, J=1.6 Hz, 1H), 8.30 (d, J=2.8 Hz, 1H), 8.17-8.09 (m, 2H), 7.78 (d, J=8.4 Hz, 1H), 7.73 (t, J=2.0 Hz, 1H), 7.32 (d, J=8.4 Hz, 2H), 4.40-4.28 (m, 2H), 4.24 (q, J=6.8 Hz, 2H), 3.92 (s, 3H), 3.64 (t, J=6.0 Hz, 2H), 3.02 (t, J=6.0 Hz, 2H), 1.39 (t, J=7.2 Hz, 3H). LC-MS: [M+H]⁺=497.1.

### Example 7: 8-methoxy-6-(6-(1-methyl-1H-imidazol-4-yl)benzofuran-3-yl)-2-(2,2,2-trifluoroethyl)-3,4-dihydroisoquinolin-1 (2H)-one (compound 7)

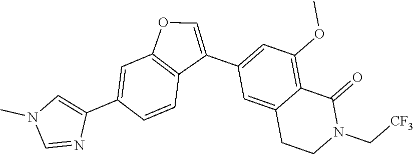 [SMILES: COc1cc(-c2coc3cc(-c4cn(C)cn4)ccc23)cc2c1C(=O)N(CC(F)(F)F)CC2]

### Step 1: 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one

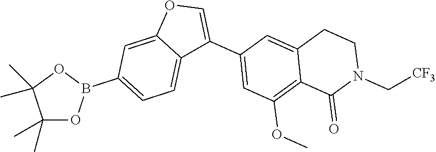 [SMILES: COc1cc(-c2coc3cc(B4OC(C)(C)C(C)(C)O4)ccc23)cc2c1C(=O)N(CC(F)(F)F)CC2]

The title compound (330 mg, 85% yield) was prepared in a manner similar to that described in Example 1 step 4 from 6-(6-chlorobenzofuran-3-yl)-8-methoxy-2-(2,2,2-trifluoroethyl)-3,4-dihydroisoquinolin-1 (2H)-one and 4,4,4′,4′,5,5,5′,5′-octamethyl-2,2′-bi (1,3,2-dioxaborolane). LC-MS: [M+H]⁺=502.3.

### Step 2: 8-methoxy-6-(6-(1-methyl-1H-imidazol-4-yl)benzofuran-3-yl)-2-(2,2,2-trifluoroethyl)-3,4-dihydroisoquinolin-1 (2H)-one (compound 7)

Compound 7 (4.8 mg, 5% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-iodo-1-methyl-1H-imidazole. ¹H NMR (400 MHz, DMSO-d₆) δ 8.50 (s, 1H), 8.01-7.94 (m, 2H), 7.79 (dd, J₁=8.0 Hz, J₂=1.2 Hz, 1H), 7.72 (s, 1H), 7.67 (s, 1H), 7.29 (d, J=7.6 Hz, 2H), 4.35 (q, J=9.6 Hz, 2H), 3.91 (s, 3H), 3.71 (s, 3H), 3.63 (t, J=6.0 Hz, 2H), 3.01 (t, J=6.0 Hz, 2H). LC-MS: [M+H]⁺=456.1.

### Example 8: 8-methoxy-6-(6-(6-methylpyridazin-3-yl)benzofuran-3-yl)-2-(2,2,2-trifluoroethyl)-3,4-dihydroisoquinolin-1 (2H)-one (compound 8)

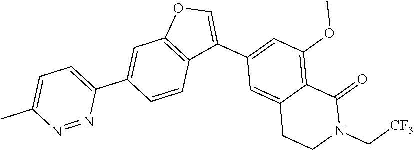 [SMILES: COc1cc(-c2coc3cc(-c4ccc(C)nn4)ccc23)cc2c1C(=O)N(CC(F)(F)F)CC2]

### Step 1: 8-methoxy-6-(6-(6-methylpyridazin-3-yl)benzofuran-3-yl)-2-(2,2,2-trifluoroethyl)-3,4-dihydroisoquinolin-1 (2H)-one (compound 8)

Compound 8 (12.7 mg, 27% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 3-bromo-6-methylpyridazine. ¹H NMR (400 MHz, DMSO-d₆) δ 8.68 (s, 1H), 8.47 (s, 1H), 8.27 (d, J=8.8 Hz, 1H), 8.24-8.17 (m, 2H), 7.70 (d, J=8.8 Hz, 1H), 7.34 (d, J=6.0 Hz, 2H), 4.36 (q, J=9.6 Hz, 2H), 3.93 (s, 3H), 3.65 (t, J=5.6 Hz, 2H), 3.03 (t, J=6.4 Hz, 2H), 2.69 (s, 3H). LC-MS: [M+H]⁺=468.1.

### Example 9: 8-methoxy-6-(6-(6-methoxypyridin-3-yl)benzofuran-3-yl)-2-(2,2,2-trifluoroethyl)-3,4-dihydroisoquinolin-1 (2H)-one (compound 9)

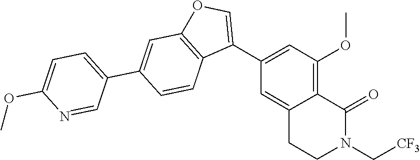 [SMILES: COc1ccc(-c2ccc3c(-c4cc5c(c(OC)c4)C(=O)N(CC(F)(F)F)CC5)coc3c2)cn1]

Compound 9 (6.5 mg, 11% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 2-methoxy-5-(4,4,5,5-tetramethyl-1,3,2-dioxaborolan-2-yl)pyridine. ¹H NMR (400 MHz, DMSO-d₆) δ 8.62-8.58 (m, 2H), 8.16-8.08 (m, 2H), 8.03 (d, J=1.2 Hz, 1H), 7.70 (dd, J₁=8.4 Hz, J₂=1.6 Hz, 1H), 7.32 (d, J=8.0 Hz, 2H), 6.95 (d, J=8.4 Hz, 1H), 4.35 (q, J=9.6 Hz, 2H), 3.92 (s, 6H), 3.64 (t, J=6.0 Hz, 2H), 3.02 (t, J=6.1 Hz, 2H). LC-MS: [M+H]⁺=483.1.

### Example 10: 8-methoxy-6-(6-(6-methylpyridazin-4-yl)benzofuran-3-yl)-2-(2,2,2-trifluoroethyl)-3,4-dihydroisoquinolin-1 (2H)-one (compound 10)

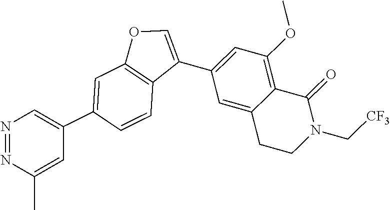 [SMILES: COc1cc(-c2coc3cc(-c4cnnc(C)c4)ccc23)cc2c1C(=O)N(CC(F)(F)F)CC2]

### Step 1: 8-methoxy-6-(6-(6-methylpyridazin-4-yl)benzofuran-3-yl)-2-(2,2,2-trifluoroethyl)-3,4-dihydroisoquinolin-1 (2H)-one (compound 10)

Compound 10 (6.8 mg, 11% yield) was prepared in a manner similar to that described in Example 1 step 6 from 6-(6-chlorobenzofuran-3-yl)-8-methoxy-2-(2,2,2-trifluoroethyl)-3,4-dihydroisoquinolin-1 (2H)-one and 3-methyl-5-(4,4,5,5-tetramethyl-1,3,2-dioxaborolan-2-yl)pyridazine. ¹H NMR (400 MHz, DMSO-d₆) δ 9.60 (d, J=2.0 Hz, 1H), 8.69 (s, 1H), 8.35 (s, 1H), 8.19 (d, J=8.0 Hz, 1H), 8.03 (d. J=2.0 Hz, 1H), 7.95 (dd, J₁=8.4 Hz, J₂=1.6 Hz, 1H), 7.33 (d, J=6.0 Hz, 2H), 4.35 (q, J=9.6 Hz, 2H), 3.92 (s, 3H), 3.64 (t, J=6.0 Hz, 2H), 3.03 (t, J=6.0 Hz, 2H), 2.71 (s, 3H). LC-MS: [M+H]⁺=468.1.

### Example 11: 6-(4-fluoro-6-(1-methyl-1H-pyrazol-4-yl)benzofuran-3-yl)-8-methoxy-2-(2,2,2-trifluoroethyl)-3,4-dihydroisoquinolin-1 (2H)-one (compound 11)

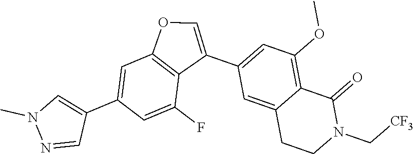 [SMILES: COc1cc(-c2coc3cc(-c4cnn(C)c4)cc(F)c23)cc2c1C(=O)N(CC(F)(F)F)CC2]

### Step 1: ethyl 6-bromo-4-fluoro-3-oxo-2,3-dihydrobenzofuran-2-carboxylate

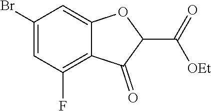 [SMILES: CCOC(=O)C1Oc2cc(Br)cc(F)c2C1=O]

To a solution of ethyl 2-hydroxyacetate (1.87 g, 17.93 mmol) in THF (30 mL) was added NaH (1.43 g, 35.85 mmol, 60% purity) under 0° C. The mixture was stirred at 0° C. for 1 h and then methyl 4-bromo-2, 6-difluorobenzoate (3 g, 11.95 mmol) was added dropwise. The mixture was stirred at 20° C. for 16 hrs. LCMS showed the reaction was completed. The residue was poured into saturated NH₄Cl (30 mL). The aqueous phase was extracted with ethyl acetate (15 mL*2). The combined organic phase was washed with brine, dried with anhydrous Na₂SO₄, filtered and concentrated in vacuum. The residue was purified by silica gel column chromatography, eluting with PE/EtOAc (v/v=100/1 to 0/1), to give the title product (0.5 g, 13% yield). ¹H NMR (400 MHz, CDCl₃) δ 8.19 (s, 1H), 7.46 (s, 1H), 7.15 (dd, J=1.2, 8.8 Hz, 1H), 4.50 (q, J=7.2 Hz, 2H), 1.47 (t, J=7.2 Hz, 3H).

### Step 2: 6-bromo-4-fluorobenzofuran-3 (2H)-one

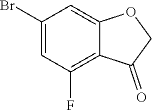 [SMILES: O=C1COc2cc(Br)cc(F)c21]

The solution of ethyl 6-bromo-4-fluoro-3-oxo-2,3-dihydrobenzofuran-2-carboxylate (0.05 g, 164.97 μmol) in MeOH (0.4 mL) and HCl (2 mL) was stirred at 80° C. for 2 hrs. LCMS showed the reaction was completed. The residue was poured into ice-water (10 mL). The aqueous phase was extracted with ethyl acetate (10 mL*2). The combined organic phase was washed with brine, dried with anhydrous Na₂SO₄, filtered and concentrated in vacuum. The residue was purified by prep-TLC, eluting with PE/EtOAc (v/v=5/1), to give the title product (0.02 g, 52% yield). ¹H NMR (400 MHz, CDCl₃) & 7.16 (s, 1H), 6.93 (d, J=8.0 Hz, 1H), 4.67 (s, 2H).

### Step 3: 4-fluoro-6-(1-methyl-1H-pyrazol-4-yl)benzofuran-3 (2H)-one

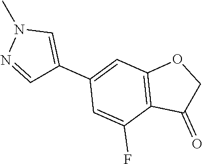 [SMILES: Cn1cc(-c2cc(F)c3c(c2)OCC3=O)cn1]

The title compound (50 mg, 26% yield) was prepared in a manner similar to that described in Example 1 step 6 from 6-bromo-4-fluorobenzofuran-3 (2H)-one and 1-methyl-4-(4,4,5,5-tetramethyl-1,3,2-dioxaborolan-2-yl)-1H-pyrazole. ¹H NMR (400 MHz, CDCl₃) δ 7.82 (s, 1H), 7.72 (s, 1H), 6.99 (d, J=1.2 Hz, 1H), 6.82 (dd, J=0.8, 10.0 Hz, 1H), 4.68 (s, 2H), 3.98 (s, 3H).

### Step 4: 4-fluoro-6-(1-methyl-1H-pyrazol-4-yl)benzofuran-3-yl trifluoromethanesulfonate

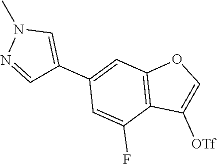 [SMILES: Cn1cc(-c2cc(F)c3c(OS(=O)(=O)C(F)(F)F)coc3c2)cn1]

The title compound (10 mg, 31% yield) was prepared in a manner similar to that described in Example 1 step 1 from 4-fluoro-6-(1-methyl-1H-pyrazol-4-yl)benzofuran-3 (2H)-one and Tf₂O. ¹H NMR (400 MHz, CDCl₃) δ 7.79 (s, 1H), 7.75 (s, 1H), 7.67 (s, 1H), 7.40 (d, J=0.8 Hz, 1H), 7.16 (d, J=10.4 Hz, 1H), 3.99 (s, 3H).

### Step 5: 6-(4-fluoro-6-(1-methyl-1H-pyrazol-4-yl)benzofuran-3-yl)-8-methoxy-2-(2,2,2-trifluoroethyl)-3,4-dihydroisoquinolin-1 (2H)-one (compound 11)

Compound 11 (5.2 mg, 5% yield) was prepared in a manner similar to that described in Example 1 step 5 from 4-fluoro-6-(1-methyl-1H-pyrazol-4-yl)benzofuran-3-yl trifluoromethanesulfonate and 8-methoxy-6-(4,4,5,5-tetramethyl-1,3,2-dioxaborolan-2-yl)-2-(2,2,2-trifluoroethyl)-3,4-dihydroisoquinolin-1 (2H)-one. ¹H NMR (400 MHz, DMSO-d₆) δ 8.40 (s, 1H), 8.30 (s, 1H), 8.03 (s, 1H), 7.79 (d, J=0.8 Hz, 1H), 7.46 (d, J=12.4 Hz, 1H), 7.30 (s, 1H), 7.17 (s, 1H), 4-39-4.30 (m, 2H), 3.88 (s, 3H), 3.86 (s, 3H), 3.63 (t. J=6.0 Hz, 2H), 2.97 (t, J=6.0 Hz, 2H). LC-MS: [M+H]⁺=474.2.

### Example 12: 6-(5-fluoro-6-(1-methyl-1H-pyrazol-4-yl)benzofuran-3-yl)-8-methoxy-2-(2,2,2-trifluoroethyl)-3,4-dihydroisoquinolin-1 (2H)-one (compound 12)

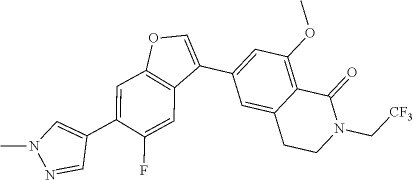 [SMILES: COc1cc(-c2coc3cc(-c4cnn(C)c4)c(F)cc23)cc2c1C(=O)N(CC(F)(F)F)CC2]

### Step 1: ethyl 6-bromo-5-fluoro-3-oxo-2,3-dihydrobenzofuran-2-carboxylate

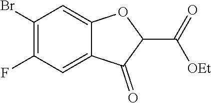 [SMILES: CCOC(=O)C1Oc2cc(Br)c(F)cc2C1=O]

The title compound (3 g, 44% yield) was prepared in a manner similar to that described in Example 11 step 1 from ethyl 2-hydroxyacetate and methyl 4-bromo-2,5-difluorobenzoate.

### Step 2: 6-bromo-5-fluorobenzofuran-3 (2H)-one

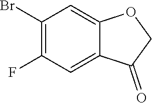 [SMILES: O=C1COc2cc(Br)c(F)cc21]

The title compound (65 mg, 14% yield) was prepared in a manner similar to that described in Example 11 step 2 from ethyl 6-bromo-5-fluoro-3-oxo-2,3-dihydrobenzofuran-2-carboxylate. LC-MS: [M−H]⁻=229.0.

### Step 3: 5-fluoro-6-(1-methyl-1H-pyrazol-4-yl)benzofuran-3 (2H)-one

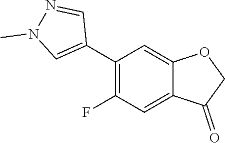 [SMILES: Cn1cc(-c2cc3c(cc2F)C(=O)CO3)cn1]

The title compound (40 mg, 61% yield) was prepared in a manner similar to that described in Example 1 step 6 from 6-bromo-5-fluorobenzofuran-3 (2H)-one and 1-methyl-4-(4,4,5,5-tetramethyl-1,3,2-dioxaborolan-2-yl)-1H-pyrazole. ¹H NMR (400 MHz, CDCl₃) δ 7.95-7.87 (m, 2H), 7.43-7.36 (m, 1H), 7.29 (s, 1H), 4.74-4.65 (m, 2H), 4.06-3.95 (m, 3H).

### Step 4: 5-fluoro-6-(1-methyl-1H-pyrazol-4-yl)benzofuran-3-yl trifluoromethanesulfonate

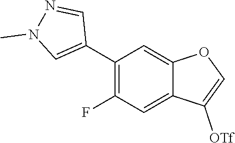 [SMILES: Cn1cc(-c2cc3occ(OS(=O)(=O)C(F)(F)F)c3cc2F)cn1]

The title compound (20 mg, 31% yield) was prepared in a manner similar to that described in Example 1 step 1 from 5-fluoro-6-(1-methyl-1H-pyrazol-4-yl)benzofuran-3 (2H)-one and Tf₂O. ¹H NMR (400 MHz, CDCl₃) δ 7.90-7.87 (m, 1H), 7.86-7.82 (m, 2H), 7.66 (d, J=6.0 Hz, 1H), 7.34 (d, J=10.0 Hz, 1H), 4.00 (s, 3H).

### Step 5: 6-(5-fluoro-6-(1-methyl-1H-pyrazol-4-yl)benzofuran-3-yl)-8-methoxy-2-(2,2,2-trifluoroethyl)-3,4-dihydroisoquinolin-1 (2H)-one (compound 12)

Compound 12 (4.8 mg, 18% yield) was prepared in a manner similar to that described in Example 1 step 5 from 5-fluoro-6-(1-methyl-1H-pyrazol-4-yl)benzofuran-3-yl trifluoromethanesulfonate and 8-methoxy-6-(4,4,5,5-tetramethyl-1,3,2-dioxaborolan-2-yl)-2-(2,2,2-trifluoroethyl)-3,4-dihydroisoquinolin-1 (2H)-one. ¹H NMR (400 MHz, DMSO-d₆) δ 8.60 (s, 1H), 8.21 (d. J=2.4 Hz, 1H), 8.06 (d. J=6.0 Hz, 1H), 8.01 (s, 1H), 7.89 (d, J=11.6 Hz, 1H), 7.28 (s, 2H), 4.34 (q, J=9.6 Hz, 2H), 3.92 (s 3H), 3.91 (s, 3H), 3.63 (t, J=6.4 Hz, 2H), 3.02 (t, J=6.4 Hz, 2H). LC-MS: [M+H]⁺=474.2.

### Example 13: 8-(difluoromethoxy)-6-(6-(1-methyl-1H-pyrazol-4-yl)benzofuran-3-yl)-2-(2,2,2-trifluoroethyl)-3,4-dihydroisoquinolin-1 (2H)-one (compound 13)

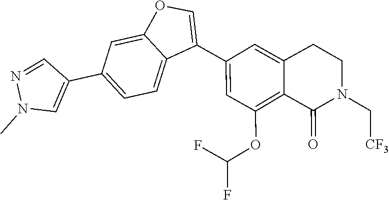 [SMILES: Cn1cc(-c2ccc3c(-c4cc5c(c(OC(F)F)c4)C(=O)N(CC(F)(F)F)CC5)coc3c2)cn1]

### Step 1: 6-bromo-8-hydroxy-2-(2,2,2-trifluoroethyl)-3,4-dihydroisoquinolin-1 (2H)-one

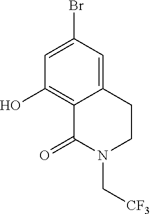 [SMILES: O=C1c2c(O)cc(Br)cc2CCN1CC(F)(F)F]

To a solution of 6-bromo-8-methoxy-2-(2,2,2-trifluoroethyl)-3,4-dihydroisoquinolin-1 (2H)-one (0.3 g, 0.89 mmol) in DCM (10 mL) was added 2 M BBr₃ (1.34 mL, 2.67 mmol) at 0° C. The mixture was stirred for 2 hours at room temperature, and 50 mL of saturated aq. NaHCO₃ solution was added slowly to quench the reaction. The aqueous phase was extracted by DCM (2×50 mL) and the combined organic phases were washed with brine, dried over anhydrous Na₂SO₄ and evaporated in vacuum. The crude product was purified by Prep-TLC, eluting with PE/EtOAc (v/v=1/1), to give the title product (0.28 g, 98% yield). LC-MS: [M+H]⁺=324.3.

### Step 2: 6-bromo-8-(difluoromethoxy)-2-(2,2,2-trifluoroethyl)-3,4-dihydroisoquinolin-1 (2H)-one

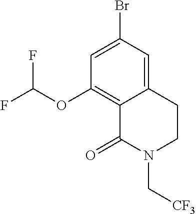 [SMILES: O=C1c2c(cc(Br)cc2OC(F)F)CCN1CC(F)(F)F]

A solution of 6-bromo-8-hydroxy-2-(2,2,2-trifluoroethyl)-3,4-dihydroisoquinolin-1 (2H)-one (0.28 g, 0.87 mmol) in CH₃CN (10 mL) was cooled to 0° C. KOH (0.5 g, 8.7 mmol) in H₂O (10 mL) was added, the mixture was stirred for 30 min in an ice batch. Diethyl (bromodifluoromethyl)phosphonate (0.33 g, 1.21 mmol) was then added dropwise and stirred for 1 hour in an ice batch. 50 mL of brine was added and extracted by EA (2×50 mL) and the combined organic phases were washed with brine, dried over anhydrous Na₂SO₄ and evaporated in vacuum. The crude product was purified by Prep-TLC, eluting with PE/EtOAc (v/v=3/1), to give the title (0.21 g, 64% yield). LC-MS: [M+H]⁺=374.4.

### Step 3: 8-(difluoromethoxy)-6-(4,4,5,5-tetramethyl-1,3,2-dioxaborolan-2-yl)-2-(2,2,2-trifluoroethyl)-3,4-dihydroisoquinolin-1 (2H)-one

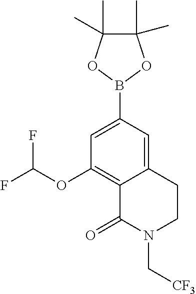 [SMILES: CC1(C)OB(c2cc3c(c(OC(F)F)c2)C(=O)N(CC(F)(F)F)CC3)OC1(C)C]

The title compound of step 3 (1.4 g, 91% yield) was prepared in a manner similar to that described in Example 1 step 4 from 6-bromo-8-(difluoromethoxy)-2-(2,2,2-trifluoroethyl)-3,4-dihydroisoquinolin-1 (2H)-one. LC-MS: [M+H]⁺=422.1.

### Step 4: 3-oxo-2,3-dihydrobenzofuran-6-yl acetate

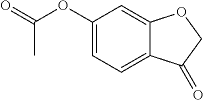

6-hydroxybenzofuran-3 (2H)-one (5 g, 33.33 mmol) and pyridine (3.2 mL) were dissolved in DCM (50 mL). Ac₂O (3.42 g, 33.33 mmol) was added dropwise at 0° C. and the mixture was stirred at room temperature for overnight. The mixture was concentrated and the residue was triturated with diethyl ether twice to give the title product (5.4 g, 84% yield). LC-MS: [M+H]⁺=193.0.

### Step 5: 3-(((trifluoromethyl) sulfonyl)oxy)benzofuran-6-yl acetate

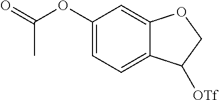 [SMILES: CC(=O)Oc1ccc2c(c1)OCC2OS(=O)(=O)C(F)(F)F]

The title compound of step 5 (7.6 g, 83% yield) was prepared in a manner similar to that described in Example 1 step 1 from 3-oxo-2,3-dihydrobenzofuran-6-yl acetate and triflic anhydride. LC-MS: [M+H]⁺=325.0.

### Step 6: 3-(8-(difluoromethoxy)-1-oxo-2-(2,2,2-trifluoroethyl)-1,2,3,4-tetrahydroisoquinolin-6-yl)benzofuran-6-yl acetate

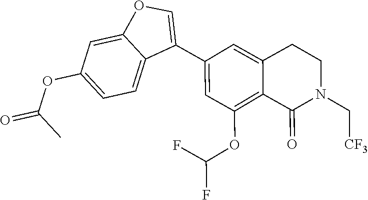 [SMILES: CC(=O)Oc1ccc2c(-c3cc4c(c(OC(F)F)c3)C(=O)N(CC(F)(F)F)CC4)coc2c1]

The title compound of step 6 (80 mg, 89% yield) was prepared in a manner similar to that described in Example 1 step 5 from 8-(difluoromethoxy)-6-(4,4,5,5-tetramethyl-1,3,2-dioxaborolan-2-yl)-2-(2,2,2-trifluoroethyl)-3,4-dihydroisoquinolin-1 (2H)-one and 3-(((trifluoromethyl) sulfonyl)oxy)benzofuran-6-yl acetate. LC-MS: [M+H]⁺=470.3.

### Step 7: 8-(difluoromethoxy)-6-(6-hydroxybenzofuran-3-yl)-2-(2,2,2-trifluoroethyl)-3,4-dihydroisoquinolin-1 (2H)-one

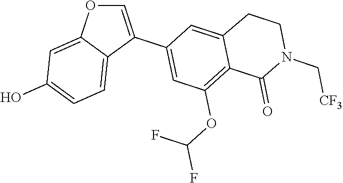 [SMILES: O=C1c2c(cc(-c3coc4cc(O)ccc34)cc2OC(F)F)CCN1CC(F)(F)F]

To a solution of 3-(8-(difluoromethoxy)-1-oxo-2-(2,2,2-trifluoroethyl)-1,2,3,4-tetrahydroisoquinolin-6-yl)benzofuran-6-yl acetate (80 mg, 0.17 mmol) in MeOH (5 mL) was added K₂CO₃ (47 mg, 0.34 mmol). The mixture was stirred for 1 hour at room temperature, and 50 mL of saturated NaHCO₃ solution was added slowly to quench the reaction. The aqueous phase was extracted by DCM (2×50 mL), the combined organic phases were washed with brine, dried over anhydrous Na₂SO₄ and evaporated in vacuum. The crude product was purified by Prep-TLC, eluting with PE/EtOAc (v/v=2/1), to give the title product (70 mg, 96% yield). LC-MS: [M+H]⁺=428.3.

### Step 8: 3-(8-(difluoromethoxy)-1-oxo-2-(2,2,2-trifluoroethyl)-1,2,3,4-tetrahydroisoquinolin-6-yl)benzofuran-6-yl trifluoromethanesulfonate

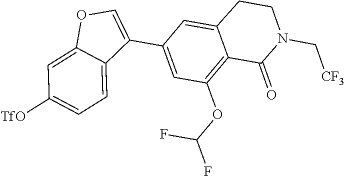 [SMILES: O=C1c2c(cc(-c3coc4cc(OS(=O)(=O)C(F)(F)F)ccc34)cc2OC(F)F)CCN1CC(F)(F)F]

To a solution of 8-(difluoromethoxy)-6-(6-hydroxybenzofuran-3-yl)-2-(2,2,2-trifluoroethyl)-3,4-dihydroisoquinolin-1 (2H)-one (70 mg, 0.164 mmol) and Et₃N (33 mg, 0.33 mmol) in DCM (8 mL) was added N-Phenylbis(trifluoromethanesulfonimide) (58.5 mg, 0.164 mmol). The suspension was stirred for 16 hours at room temperature, then 50 mL of water was added and extracted by EA (2×50 mL). The combined organic phases were washed with brine, dried over anhydrous Na₂SO₄ and evaporated in vacuum. The crude product was purified by Prep-TLC, eluting with PE/EtOAc (v/v=1/1), to give the title product (40 mg, 44% yield). LC-MS: [M+H]⁺=560.2.

### Step 9: 8-(difluoromethoxy)-6-(6-(1-methyl-1H-pyrazol-4-yl)benzofuran-3-yl)-2-(2,2,2-trifluoroethyl)-3,4-dihydroisoquinolin-1 (2H)-one (compound 13)

Compound 13 (3 mg, 8% yield) was prepared in a manner similar to that described in Example 1 step 5 from 3-(8-(difluoromethoxy)-1-oxo-2-(2,2,2-trifluoroethyl)-1,2,3,4-tetrahydroisoquinolin-6-yl)benzofuran-6-yl trifluoromethanesulfonate and 1-methyl-4-(4,4,5,5-tetramethyl-1,3,2-dioxaborolan-2-yl)-1H-pyrazole. ¹H NMR (500 MHz, DMSO-d₆) δ 8.55 (s, 1H), 8.25 (s, 1H), 7.98-7.91 (m, 3H), 7.69 (s, 1H), 7.62 (d, J=8.2 Hz, 1H), 7.50 (s, 1H), 7.17 (t, J=75.4 Hz, 1H), 4.39 (q, J=9.5 Hz, 2H), 3.88 (s, 3H), 3.70 (t, J=6.0 Hz, 2H), 3.11 (t, J=6.0 Hz, 2H). LC-MS: [M+H]⁺=492.3.

### Example 14: 8-(difluoromethoxy)-6-(6-(6-methylpyridazin-3-yl)benzofuran-3-yl)-2-(2,2,2-trifluoroethyl)-3,4-dihydroisoquinolin-1 (2H)-one (compound 14)

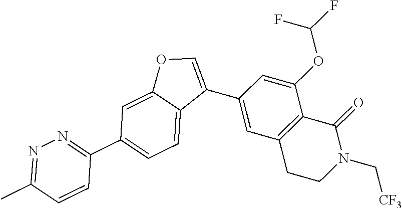 [SMILES: Cc1ccc(-c2ccc3c(-c4cc5c(c(OC(F)F)c4)C(=O)N(CC(F)(F)F)CC5)coc3c2)nn1]

### Step 1: 8-(difluoromethoxy)-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one

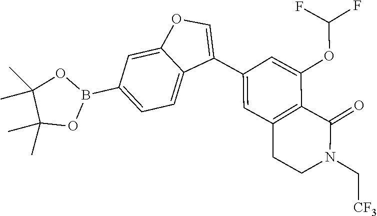 [SMILES: CC1(C)OB(c2ccc3c(-c4cc5c(c(OC(F)F)c4)C(=O)N(CC(F)(F)F)CC5)coc3c2)OC1(C)C]

The title compound of step 1 (375 mg, 90% yield) was prepared in a manner similar to that described in Example 1 step 4 from 3-(8-(difluoromethoxy)-1-oxo-2-(2,2,2-trifluoroethyl)-1,2,3,4-tetrahydroisoquinolin-6-yl)benzofuran-6-yl trifluoromethanesulfonate and 4,4,4′,4′,5,5,5′,5′-octamethyl-2,2′-bi (1,3,2-dioxaborolane). LC-MS: [M+H]⁺=538.3.

### Step 2: 8-(difluoromethoxy)-6-(6-(6-methylpyridazin-3-yl)benzofuran-3-yl)-2-(2,2,2-trifluoroethyl)-3,4-dihydroisoquinolin-1 (2H)-one (compound 14)

Compound 14 (20 mg, 43% yield) was prepared in a manner similar to that described in Example 1 step 6 from 8-(difluoromethoxy)-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 3-bromo-6-methylpyridazine. ¹H NMR (500 MHz, DMSO-d₆) δ 8.73 (s, 1H), 8.48 (s, 1H), 8.30-8.20 (m, 2H), 8.18-8.13 (m, 1H), 7.75 (s, 1H), 7.69 (d, J=8.8 Hz, 1H), 7.55 (s, 1H), 7.18 (t, J=75.0 Hz, 1H), 4.40 (q, J=9.5 Hz, 2H), 3.72 (t, J=6.0 Hz, 2H), 3.13 (t, J=5.9 Hz, 2H), 2.69 (s, 3H). LC-MS: [M+H]⁺=504.3.

### Example 15: 6-(6-(1,3-dimethyl-1H-pyrazol-4-yl)benzofuran-3-yl)-8-methoxy-2-(2,2,2-trifluoroethyl)-3,4-dihydroisoquinolin-1 (2H)-one (compound 15)

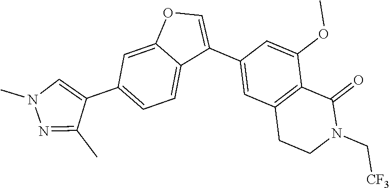 [SMILES: COc1cc(-c2coc3cc(-c4cn(C)nc4C)ccc23)cc2c1C(=O)N(CC(F)(F)F)CC2]

### Step 1: 3-(8-methoxy-1-oxo-2-(2,2,2-trifluoroethyl)-1,2,3,4-tetrahydroisoquinolin-6-yl)benzofuran-6-yl acetate

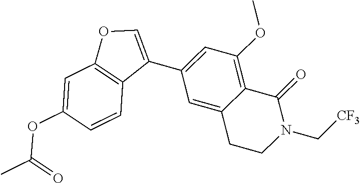 [SMILES: COc1cc(-c2coc3cc(OC(C)=O)ccc23)cc2c1C(=O)N(CC(F)(F)F)CC2]

The title compound of step 1 (600 mg, 80% yield) was prepared in a manner similar to that described in Example 1 step 5 from 8-methoxy-6-(4,4,5,5-tetramethyl-1,3,2-dioxaborolan-2-yl)-2-(2,2,2-trifluoroethyl)-3,4-dihydroisoquinolin-1 (2H)-one and 3-(((trifluoromethyl) sulfonyl)oxy)benzofuran-6-yl acetate. LC-MS: [M+H]⁺=434.3. Step 2:6-(6-hydroxybenzofuran-3-yl)-8-methoxy-2-(2,2,2-trifluoroethyl)-3,4-dihydroisoquinolin-1 (2H)-one

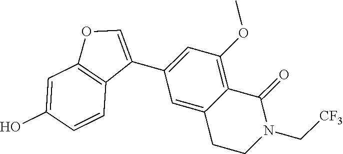 [SMILES: COc1cc(-c2coc3cc(O)ccc23)cc2c1C(=O)N(CC(F)(F)F)CC2]

The title compound of step 2 (480 mg, 90% yield) was prepared in a manner similar to that described in Example 13 step 7 from 3-(8-methoxy-1-oxo-2-(2,2,2-trifluoroethyl)-1,2,3,4-tetrahydroisoquinolin-6-yl)benzofuran-6-yl acetate. LC-MS: [M+H]⁺=392.3.

### Step 3: 3-(8-methoxy-1-oxo-2-(2,2,2-trifluoroethyl)-1,2,3,4-tetrahydroisoquinolin-6-yl)benzofuran-6-yl trifluoromethanesulfonate

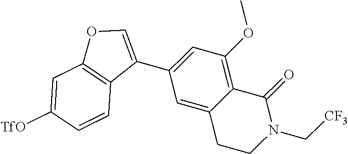 [SMILES: COc1cc(-c2coc3cc(OS(=O)(=O)C(F)(F)F)ccc23)cc2c1C(=O)N(CC(F)(F)F)CC2]

The title compound of step 3 (300 mg, 40% yield) was prepared in a manner similar to that described in Example 13 step 8 from 6-(6-hydroxybenzofuran-3-yl)-8-methoxy-2-(2,2,2-trifluoroethyl)-3,4-dihydroisoquinolin-1 (2H)-one and trifilic anhydride. LC-MS: [M+H]⁺=523.4.

### Step 4: 6-(6-(1,3-dimethyl-1H-pyrazol-4-yl)benzofuran-3-yl)-8-methoxy-2-(2,2,2-trifluoroethyl)-3,4-dihydroisoquinolin-1 (2H)-one (compound 15)

Compound 15 (10 mg, 28% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and (1,3-dimethyl-1H-pyrazol-4-yl) boronic acid. ¹H NMR (500 MHz, DMSO-d₆) δ 8.53 (s, 1H), 8.00 (d, J=7.6 Hz, 1H), 7.98 (s, 1H), 7.70 (s, 1H), 7.45 (d, J=8.2 Hz, 1H), 7.30 (d, J=11.5 Hz, 2H), 4.35 (q, J=9.6 Hz, 2H), 3.91 (s, 3H), 3.81 (s, 3H), 3.63 (t, J=6.1 Hz, 2H), 3.01 (t, J=5.9 Hz, 2H), 2.36 (s, 3H). LC-MS: [M+H]⁺=470.3.

### Example 16: 8-methoxy-6-(6-(1-methyl-3-(trifluoromethyl)-1H-pyrazol-4-yl)benzofuran-3-yl)-2-(2,2,2-trifluoroethyl)-3,4-dihydroisoquinolin-1 (2H)-one (compound 16)

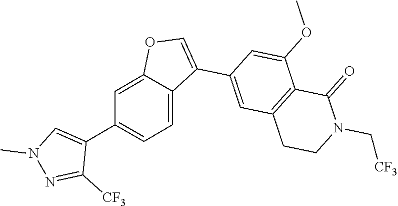 [SMILES: COc1cc(-c2coc3cc(-c4cn(C)nc4C(F)(F)F)ccc23)cc2c1C(=O)N(CC(F)(F)F)CC2]

### Step 1: 8-methoxy-6-(6-(1-methyl-3-(trifluoromethyl)-1H-pyrazol-4-yl)benzofuran-3-yl)-2-(2,2,2-trifluoroethyl)-3,4-dihydroisoquinolin-1 (2H)-one (compound 16)

Compound 16 (2 mg, 6% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and (1-methyl-3-(trifluoromethyl)-1H-pyrazol-4-yl) boronic acid. ¹H NMR (500 MHz, DMSO-d₆) δ 8.60 (s, 1H), 8.25 (s, 1H), 8.09 (d, J=8.2 Hz, 1H), 7.70 (s, 1H), 7.42 (d, J=8.2 Hz, 1H), 7.31 (d. J=9.2 Hz, 2H), 4.35 (q, J=9.6 Hz, 1H), 3.99 (s, 3H), 3.91 (s, 3H), 3.64 (t, J=5.9 Hz, 2H), 3.01 (t, J=5.8 Hz, 2H). LC-MS: [M+H]⁺=524.3.

### Example 17: 8-methoxy-6-(6-(1-methyl-1H-1,2,3-triazol-4-yl)benzofuran-3-yl)-2-(2,2,2-trifluoroethyl)-3,4-dihydroisoquinolin-1 (2H)-one (compound 17)

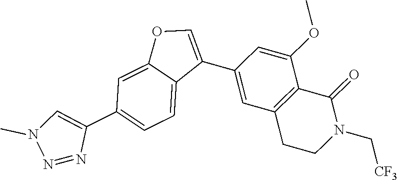 [SMILES: COc1cc(-c2coc3cc(-c4cn(C)nn4)ccc23)cc2c1C(=O)N(CC(F)(F)F)CC2]

### Step 1: 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one

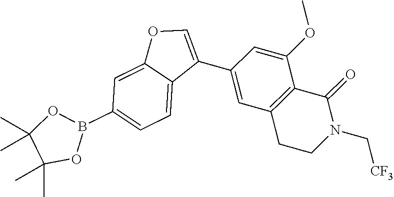 [SMILES: COc1cc(-c2coc3cc(B4OC(C)(C)C(C)(C)O4)ccc23)cc2c1C(=O)N(CC(F)(F)F)CC2]

The title compound of step 1 (150 mg, 79% yield) was prepared in a manner similar to that described in Example 1 step 4 from 3-(8-methoxy-1-oxo-2-(2,2,2-trifluoroethyl)-1,2,3,4-tetrahydroisoquinolin-6-yl)benzofuran-6-yl trifluoromethanesulfonate and 4,4,4′,4′,5,5,5′,5′-octamethyl-2,2′-bi (1,3,2-dioxaborolane). LC-MS: [M+H]⁺=502.3.

### Step 2: 8-methoxy-6-(6-(1-methyl-1H-1,2,3-triazol-4-yl)benzofuran-3-yl)-2-(2,2,2-trifluoroethyl)-3,4-dihydroisoquinolin-1 (2H)-one (compound 17)

Compound 17 (0.79 mg, 4% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-bromo-1-methyl-1H-1,2,3-triazole. ¹H NMR (500 MHz, DMSO-d₆) δ 8.63 (s, 1H), 8.58 (s, 1H), 8.13 (s, 1H), 8.09 (d, J=8.2 Hz, 1H), 7.89 (dd, J₁=8.2 Hz, J₂=1.3 Hz, 1H), 7.31 (d, J=8.7 Hz, 2H), 4.35 (q, J=9.4 Hz, 2H), 4.12 (s, 3H), 3.91 (s, 3H), 3.64 (t, J=6.1 Hz, 2H), 3.01 (t, J=6.1 Hz, 2H). LC-MS: [M+H]⁺=457.3.

### Example 18: 4-(3-(8-methoxy-1-oxo-2-(2,2,2-trifluoroethyl)-1,2,3,4-tetrahydroisoquinolin-6-yl)benzofuran-6-yl)-1-methyl-1H-pyrazole-3-carbonitrile (compound 18)

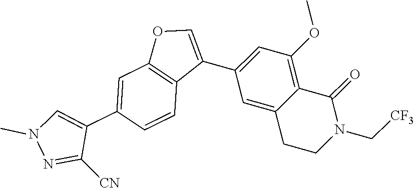 [SMILES: COc1cc(-c2coc3cc(-c4cn(C)nc4C#N)ccc23)cc2c1C(=O)N(CC(F)(F)F)CC2]

### Step 1: 4-(3-(8-methoxy-1-oxo-2-(2,2,2-trifluoroethyl)-1,2,3,4-tetrahydroisoquinolin-6-yl)benzofuran-6-yl)-1-methyl-1H-pyrazole-3-carbonitrile (compound 18)

Compound 18 (5 mg, 26% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-bromo-1-methyl-1H-pyrazole-3-carbonitrile. ¹H NMR (500 MHz, DMSO-d₆) δ 8.63 (s, 1H), 8.52 (s, 1H), 8.16 (d, J=8.2 Hz, 1H), 7.96 (d, J=1.1 Hz, 1H), 7.68 (dd, J₁=8.2 Hz, J₂=1.4 Hz, 1H), 7.32 (d, J=6.6 Hz, 2H), 4.35 (q, J=9.6 Hz, 2H), 4.01 (s, 3H), 3.91 (s, 3H), 3.64 (t, J=6.1 Hz, 2H), 3.02 (t, J=6.1 Hz, 2H). LC-MS: [M+H]⁺=481.3.

### Example 19: 8-methoxy-6-(6-(3-methoxy-1-methyl-1H-pyrazol-4-yl)benzofuran-3-yl)-2-(2,2,2-trifluoroethyl)-3,4-dihydroisoquinolin-1 (2H)-one (compound 19)

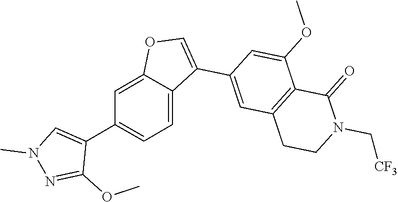 [SMILES: COc1cc(-c2coc3cc(-c4cn(C)nc4OC)ccc23)cc2c1C(=O)N(CC(F)(F)F)CC2]

Compound 19 (8.5 mg, 18% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-bromo-3-methoxy-1-methyl-1H-pyrazole. ¹H NMR (400 MHz, DMSO-d₆) δ ¹H NMR (500 MHz, DMSO) δ 8.48 (s, 1H), 8.11 (s, 1H), 7.96 (d, J=8.3 Hz, 1H), 7.87 (s, 1H), 7.64 (d, J=8.4 Hz, 1H), 7.28 (d, J=13.4 Hz, 2H), 4.34 (q, J=9.8 Hz, 2H), 3.95 (s, 3H), 3.90 (s, 3H), 3.75 (s, 3H), 3.63 (t, J=6.1 Hz, 2H), 3.00 (t, J=5.9 Hz, 2H). LC-MS: [M+H]⁺=486.3.

### Example 20: 2-(8-methoxy-6-(6-(1-methyl-1H-pyrazol-4-yl)benzofuran-3-yl)-1-oxo-3,4-dihydroisoquinolin-2 (1H)-yl) acetonitrile (compound 20)

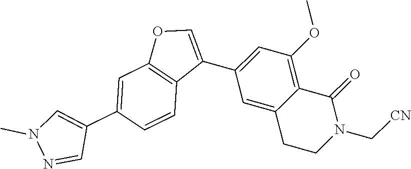 [SMILES: COc1cc(-c2coc3cc(-c4cnn(C)c4)ccc23)cc2c1C(=O)N(CC#N)CC2]

### Step 1: 2-(6-bromo-8-methoxy-1-oxo-3,4-dihydroisoquinolin-2 (1H)-yl) acetonitrile

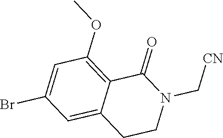 [SMILES: COc1cc(Br)cc2c1C(=O)N(CC#N)CC2]

The title compound of step 1 (460 mg, 66% yield) was prepared in a manner similar to that described in Example 1 step 3 from 6-bromo-8-methoxy-3,4-dihydroisoquinolin-1 (2H)-one and 2-bromoacetonitrile. LC-MS: [M+H]⁺=295.0.

### Step 2: 2-(8-methoxy-1-oxo-6-(4,4,5,5-tetramethyl-1,3,2-dioxaborolan-2-yl)-3,4-dihydroisoquinolin-2 (1H)-yl) acetonitrile

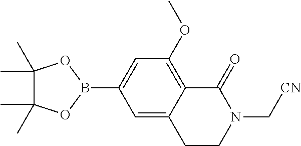 [SMILES: COc1cc(B2OC(C)(C)C(C)(C)O2)cc2c1C(=O)N(CC#N)CC2]

The title compound of step 2 (200 mg, 58% yield) was prepared in a manner similar to that described in Example 1 step 4 from 2-(6-bromo-8-methoxy-1-oxo-3,4-dihydroisoquinolin-2 (1H)-yl) acetonitrile and 4,4,4′,4′,5,5,5′,5′-octamethyl-2,2′-bi (1,3,2-dioxaborolane). LC-MS: [M+H]⁺=343.2.

### Step 3: 2-(6-(6-chlorobenzofuran-3-yl)-8-methoxy-1-oxo-3,4-dihydroisoquinolin-2 (1H)-yl)acetonitrile

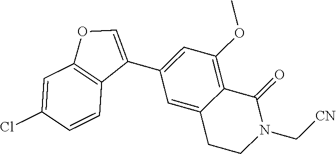 [SMILES: COc1cc(-c2coc3cc(Cl)ccc23)cc2c1C(=O)N(CC#N)CC2]

The title compound of step 3 (150 mg, 70% yield) was prepared in a manner similar to that described in Example 1 step 5 from 2-(8-methoxy-1-oxo-6-(4,4,5,5-tetramethyl-1,3,2-dioxaborolan-2-yl)-3,4-dihydroisoquinolin-2 (1H)-yl) acetonitrile and 6-chlorobenzofuran-3-yl trifluoromethanesulfonate. LC-MS: [M+H]⁺=367.8.

### Step 4: 2-(8-methoxy-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile

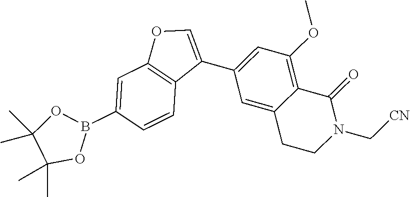 [SMILES: COc1cc(-c2coc3cc(B4OC(C)(C)C(C)(C)O4)ccc23)cc2c1C(=O)N(CC#N)CC2]

The title compound of step 4 (70 mg, 56% yield) was prepared in a manner similar to that described in Example 1 step 4 from 2-(6-(6-chlorobenzofuran-3-yl)-8-methoxy-1-oxo-3,4-dihydroisoquinolin-2 (1H)-yl) acetonitrile and 4,4,4′,4′,5,5,5′,5′-octamethyl-2,2′-bi (1,3,2-dioxaborolane). LC-MS: [M+H]⁺=459.3.

### Step 5: 2-(8-methoxy-6-(6-(1-methyl-1H-pyrazol-4-yl)benzofuran-3-yl)-1-oxo-3,4-dihydroisoquinolin-2 (1H)-yl) acetonitrile (compound 20)

Compound 20 (10 mg, 22% yield) was prepared in a manner similar to that described in Example 1 step 6 from 2-(8-methoxy-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile and 4-iodo-1-methyl-1H-pyrazole. ¹H NMR (500 MHz, DMSO-d₆) δ 8.51 (s, 1H), 8.24 (s, 1H), 7.97 (d, J=5.4 Hz, 2H), 7.89 (s, 1H), 7.60 (d, J=9.3 Hz, 1H), 7.29 (d, J=12.6 Hz, 2H), 4.56 (s, 2H), 3.92 (s, 3H), 3.90 (s, 3H), 3.63 (t, J=6.2 Hz, 2H), 3.05 (t, J=6.2 Hz, 2H). LC-MS: [M+H]⁺=413.3.

### Example 21: 2-(8-(difluoromethoxy)-6-(6-(1-methyl-1H-pyrazol-4-yl)benzofuran-3-yl)-1-oxo-3,4-dihydroisoquinolin-2 (1H)-yl) acetonitrile (compound 21)

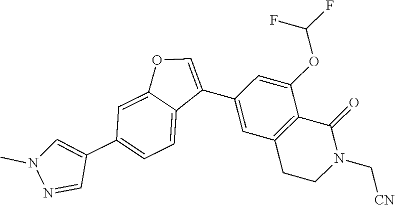 [SMILES: Cn1cc(-c2ccc3c(-c4cc5c(c(OC(F)F)c4)C(=O)N(CC#N)CC5)coc3c2)cn1]

### Step 1: 2-(6-bromo-8-hydroxy-1-oxo-3,4-dihydroisoquinolin-2 (1H)-yl) acetonitrile

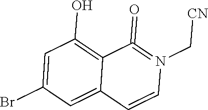

The title compound of step 1 (150 mg, 79% yield) was prepared in a manner similar to that described in Example 13 step 1 from 2-(6-bromo-8-methoxy-1-oxo-3,4-dihydroisoquinolin-2 (1H)-yl) acetonitrile. LC-MS: [M+H]⁺=282.1.

### Step 2: 2-(6-bromo-8-(difluoromethoxy)-1-oxo-3,4-dihydroisoquinolin-2 (1H)-yl) acetonitrile

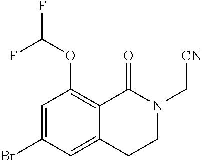 [SMILES: N#CCN1CCc2cc(Br)cc(OC(F)F)c2C1=O]

The title compound of step 2 (460 mg, 57% yield) was prepared in a manner similar to that described in Example 13 step 2 from 2-(6-bromo-8-hydroxy-1-oxo-3,4-dihydroisoquinolin-2 (1H)-yl) acetonitrile and diethyl(bromodifluoromethyl)phosphonate. LC-MS: [M+H]⁺=332.1.

### Step 3: 2-(8-(difluoromethoxy)-1-oxo-6-(4,4,5,5-tetramethyl-1,3,2-dioxaborolan-2-yl)-3,4-dihydroisoquinolin-2 (1H)-yl) acetonitrile

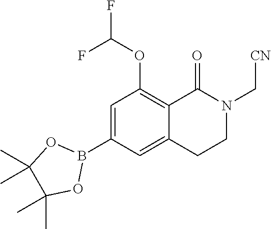 [SMILES: CC1(C)OB(c2cc3c(c(OC(F)F)c2)C(=O)N(CC#N)CC3)OC1(C)C]

The title compound of step 3 (100 mg, 87% yield) was prepared in a manner similar to that described in Example 1 step 4 from 2-(6-bromo-8-(difluoromethoxy)-1-oxo-3,4-dihydroisoquinolin-2 (1H)-yl) acetonitrile and 4,4,4′,4′,5,5,5′,5′-octamethyl-2,2′-bi (1,3,2-dioxaborolane). LC-MS: [M+H]⁺=379.2.

### Step 4: 2-(6-(6-chlorobenzofuran-3-yl)-8-(difluoromethoxy)-1-oxo-3,4-dihydroisoquinolin-2 (1H)-yl) acetonitrile

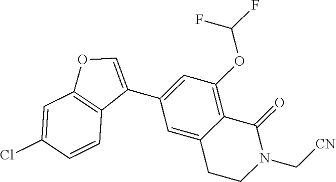 [SMILES: N#CCN1CCc2cc(-c3coc4cc(Cl)ccc34)cc(OC(F)F)c2C1=O]

The title compound of step 4 (100 mg, 94% yield) was prepared in a manner similar to that described in Example 1 step 5 from 2-(8-(difluoromethoxy)-1-oxo-6-(4,4,5,5-tetramethyl-1,3,2-dioxaborolan-2-yl)-3,4-dihydroisoquinolin-2 (1H)-yl) acetonitrile and 6-chlorobenzofuran-3-yl trifluoromethanesulfonate. LC-MS: [M+H]⁺=403.8.

### Step 5: 2-(8-(difluoromethoxy)-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile

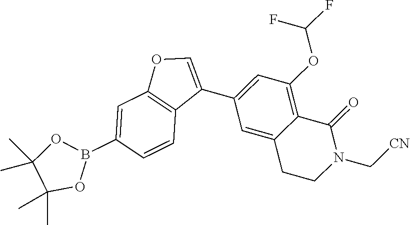 [SMILES: CC1(C)OB(c2ccc3c(-c4cc5c(c(OC(F)F)c4)C(=O)N(CC#N)CC5)coc3c2)OC1(C)C]

The title compound of step 5 (96 mg, 82% yield) was prepared in a manner similar to that described in Example 1 step 4 from 2-(6-(6-chlorobenzofuran-3-yl)-8-(difluoromethoxy)-1-oxo-3,4-dihydroisoquinolin-2 (1H)-yl) acetonitrile and 4,4,4′,4′,5,5,5′,5′-octamethyl-2,2′-bi (1,3,2-dioxaborolane). LC-MS: [M+H]⁺=495.3.

### Step 6: 2-(8-(difluoromethoxy)-6-(6-(1-methyl-1H-pyrazol-4-yl)benzofuran-3-yl)-1-oxo-3,4-dihydroisoquinolin-2 (1H)-yl) acetonitrile (compound 21)

Compound 21 (3 mg, 7% yield) was prepared in a manner similar to that described in Example 1 step 6 from 2-(8-(difluoromethoxy)-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile and 4-iodo-1-methyl-1H-pyrazole. ¹H NMR (500 MHz, DMSO-d₆) § 8.57 (s, 1H), 8.25 (s, 1H), 8.00-7.92 (m, 3H), 7.70 (s, 1H), 7.62 (d, J=8.2 Hz, 1H), 7.50 (s, 1H), 7.18 (t, J=75.5 Hz, 1H), 4.62 (s, 2H), 3.89 (s, 3H), 3.70 (t, J=6.3 Hz, 2H), 3.15 (t. J=6.3 Hz, 2H). LC-MS: [M+H]⁺=449.3.

### Example 22: 6-(6-(3-fluoro-1-methyl-1H-pyrazol-4-yl)benzofuran-3-yl)-8-methoxy-2-(2,2,2-trifluoroethyl)-3,4-dihydroisoquinolin-1 (2H)-one (compound 22)

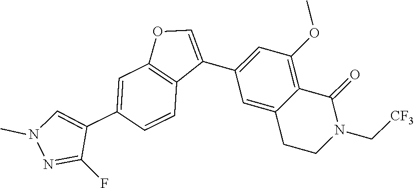 [SMILES: COc1cc(-c2coc3cc(-c4cn(C)nc4F)ccc23)cc2c1C(=O)N(CC(F)(F)F)CC2]

### Step 1: 6-(6-(3-fluoro-1-methyl-1H-pyrazol-4-yl)benzofuran-3-yl)-8-methoxy-2-(2,2,2-trifluoroethyl)-3,4-dihydroisoquinolin-1 (2H)-one (compound 22)

Compound 22 (5 mg, 18% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-bromo-3-fluoro-1-methyl-1H-pyrazole. ¹H NMR (500 MHz, DMSO-d₆) δ 8.55 (s, 1H), 8.24 (d, J=2.1 Hz, 1H), 8.04 (d, J=8.2 Hz, 1H), 7.80 (s, 1H), 7.59-7.53 (m, 1H), 7.29 (d, J=11.1 Hz, 2H), 4.35 (q, J=9.5 Hz, 2H), 3.91 (s, 3H), 3.79 (s, 3H), 3.63 (t, J=6.1 Hz, 2H), 3.01 (t, J=6.0 Hz, 2H). LC-MS: [M+H]⁺=474.2.

### Example 23: 6-(6-(3-chloro-1-methyl-1H-pyrazol-4-yl)benzofuran-3-yl)-8-methoxy-2-(2,2,2-trifluoroethyl)-3,4-dihydroisoquinolin-1 (2H)-one (compound 23)

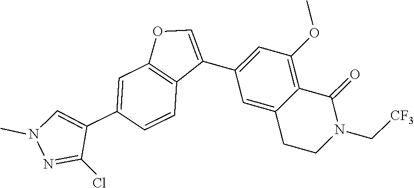 [SMILES: COc1cc(-c2coc3cc(-c4cn(C)nc4Cl)ccc23)cc2c1C(=O)N(CC(F)(F)F)CC2]

### Step 1: 6-(6-(3-chloro-1-methyl-1H-pyrazol-4-yl)benzofuran-3-yl)-8-methoxy-2-(2,2,2-trifluoroethyl)-3,4-dihydroisoquinolin-1 (2H)-one (compound 23)

Compound 23 (0.9 mg, 1% yield) was prepared in a manner similar to that described in Example 1 step 6 from 8-methoxy-6-(6-(4,4,5,5-tetramethyl-1,3,2-dioxaborolan-2-yl)benzofuran-3-yl)-2-(2,2,2-trifluoroethyl)-3,4-dihydroisoquinolin-1 (2H)-one and 4-bromo-3-chloro-1-methyl-1H-pyrazole. ¹H NMR (500 MHz, DMSO-d₆) δ 8.57 (s, 1H), 8.25 (s, 1H), 8.07 (d, J=8.2 Hz, 1H), 7.87 (d, J=0.8 Hz, 1H), 7.61 (dd, J₁=8.2, J₂=1.3 Hz, 1H), 7.31 (d, J=10.4 Hz, 2H), 4.35 (q, J=9.6 Hz, 2H), 3.91 (s, 3H), 3.85 (s, 3H), 3.64 (t, J=6.0 Hz, 2H), 3.01 (t, J=6.0 Hz, 2H). LC-MS: [M+H]⁺=490.2.

### Example 24: 2-(8-(difluoromethoxy)-6-(6-(1-methyl-1H-pyrazol-4-yl)benzofuran-3-yl)-1-oxo-3,4-dihydroisoquinolin-2 (1H)-yl) acetamide (compound 24)

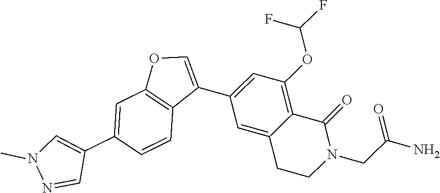 [SMILES: Cn1cc(-c2ccc3c(-c4cc5c(c(OC(F)F)c4)C(=O)N(CC(N)=O)CC5)coc3c2)cn1]

### Step 1: 2-(8-(difluoromethoxy)-6-(6-(1-methyl-1H-pyrazol-4-yl)benzofuran-3-yl)-1-oxo-3,4-dihydroisoquinolin-2 (1H)-yl) acetamide (compound 24)

Compound 24 (3.5 mg, 6% yield) was prepared in a manner similar to that described in Example 1 step 6 from 2-(6-(6-chlorobenzofuran-3-yl)-8-(difluoromethoxy)-1-oxo-3,4-dihydroisoquinolin-2 (1H)-yl) acetonitrile and (1-methyl-1H-pyrazol-4-yl) boronic acid. ¹H NMR (500 MHz, DMSO-d₆) δ 8.55 (s, 1H), 8.25 (s, 1H), 7.98 (s, 1H), 7.94 (d, J=8.2 Hz, 1H), 7.90 (d, J=10.6 Hz, 1H), 7.68 (s, 1H), 7.62 (d, J=8.3 Hz, 1H), 7.47 (s, 2H), 7.32-6.95 (m, 2H), 4.13 (s, 2H), 3.88 (s, 3H), 3.60 (t, J=6.1 Hz, 2H), 3.12 (t, J=6.1 Hz, 2H). LC-MS: [M+H]⁺=467.2.

### Example 25:2-(difluoromethoxy)-6-methoxy-4-(6-(1-methyl-1H-pyrazol-4-yl)benzofuran-3-yl)-N-(2,2,2-trifluoroethyl)benzamide (compound 25)

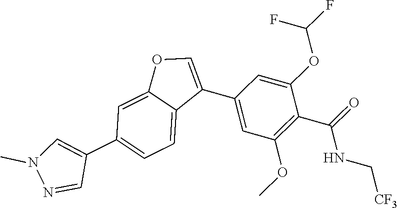 [SMILES: COc1cc(-c2coc3cc(-c4cnn(C)c4)ccc23)cc(OC(F)F)c1C(=O)NCC(F)(F)F]

### Step 1: tert-butyl 4-bromo-2,6-difluorobenzoate

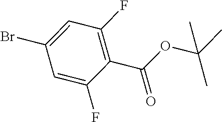 [SMILES: CC(C)(C)OC(=O)c1c(F)cc(Br)cc1F]

To a solution of 4-bromo-2,6-difluorobenzoic acid (45.0 g, 189.9 mmol) and Boc₂O (82.9 g, 379.8 mmol) in t-BuOH (450 mL) was added DMAP (7.0 g, 57.0 mmol). Then the reaction mixture was stirred at 50° C. for 2 h. The mixture was concentrated, added with 10% citric acid (100 mL) and extracted with EtOAc (500 mL×3). The combined organic phase was washed with brine (500 mL×3), dried over Na₂SO₄ and concentrated to give a residue. The residue was purified by silica gel column chromatography, eluting with PE, to give the title product (47.0 g, 84.5% yield). ¹H NMR (300 MHz, CDCl₃) δ 7.12 (d, J=6.9 Hz, 2H), 1.58 (s, 9H).

### Step 2: tert-butyl 4-bromo-2-fluoro-6-methoxybenzoate

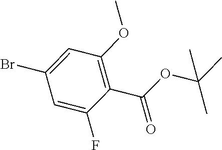 [SMILES: COc1cc(Br)cc(F)c1C(=O)OC(C)(C)C]

The title compound of step 2 (9 g, 43% yield) was prepared in a manner similar to that described in Example 1 step 2 from tert-butyl 4-bromo-2,6-difluorobenzoate and sodium methoxide. ¹H NMR (300 MHz, CDCl₃) δ 6-93-6.82 (m, 2H), 3.85 (s, 3H), 1.57 (s, 9H).

### Step 3: tert-butyl 4-bromo-2-hydroxy-6-methoxybenzoate

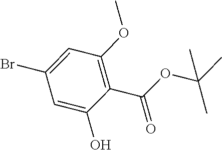 [SMILES: COc1cc(Br)cc(O)c1C(=O)OC(C)(C)C]

To a solution of 2-(methylsulfonyl) ethanol (3.0 g, 24.58 mmol) in DMF (20 mL) was added NaH (60% dispersion in mineral oil, 1.8 g, 44.50 mmol) in portions at 0° C. Then the reaction mixture was stirred at 0° C. for 1 h. After the addition of tert-butyl 4-bromo-2-fluoro-6-methoxybenzoate (5.0 g, 16.39 mmol) in DMF (30 mL) at 0° C., the reaction mixture was allowed to warm to room temperature for 2 h. The mixture was quenched with hydrochloric acid (2.0 mol/L, 65.54 mmol, 33 mL) at 0° C. The mixture was extracted with EtOAc (100 mL×3). The combined organic phase was washed with brine (200 mL×2), dried over Na₂SO₄ and concentrated to give a residue. The residue was purified by silica gel column chromatography, eluting with PE, to give the title product (2.5 g, 50.3% yield). ¹H NMR (300 MHz, CDCl₃) § 11.81 (s, 1H), 6.76 (d, J=1.8 Hz, 1H), 6.53 (d. J=1.8 Hz, 1H), 3.81 (s, 3H), 1.59 (s, 9H).

### Step 4: tert-butyl 4-bromo-2-(difluoromethoxy)-6-methoxybenzoate

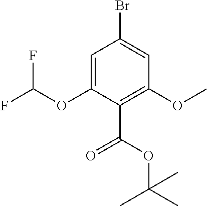 [SMILES: COc1cc(Br)cc(OC(F)F)c1C(=O)OC(C)(C)C]

The title compound of step 4 (1.8 g, 69% yield) was prepared in a manner similar to that described in Example 13 step 2 from tert-butyl 4-bromo-2-hydroxy-6-methoxybenzoate and diethyl (bromodifluoromethyl)phosphonate. ¹H NMR (300 MHz, CDCl₃) δ 6.96 (s, 1H), 6.94 (s, 1H), 6.46 (t, J=74.0 Hz, 1H), 3.85 (s, 3H), 1.56 (s, 9H).

### Step 5: tert-butyl 2-(difluoromethoxy)-6-methoxy-4-(4,4,5,5-tetramethyl-1,3,2-dioxaborolan-2-yl)benzoate

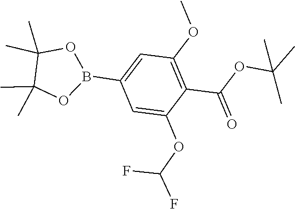 [SMILES: COc1cc(B2OC(C)(C)C(C)(C)O2)cc(OC(F)F)c1C(=O)OC(C)(C)C]

The title compound of step 5 (1.1 g, 90% yield) was prepared in a manner similar to that described in Example 1 step 4 from tert-butyl 4-bromo-2-(difluoromethoxy)-6-methoxybenzoate and 4,4,4′,4′,5,5,5′,5′-octamethyl-2,2′-bi (1,3,2-dioxaborolane). LC-MS: [M+H]⁺=401.0.

### Step 6: tert-butyl 4-(6-chlorobenzofuran-3-yl)-2-(difluoromethoxy)-6-methoxybenzoate

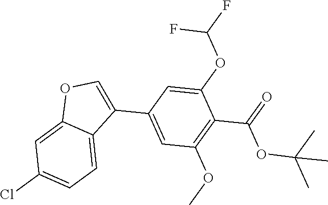 [SMILES: COc1cc(-c2coc3cc(Cl)ccc23)cc(OC(F)F)c1C(=O)OC(C)(C)C]

The title compound of step 6 (1.1 g, 95% yield) was prepared in a manner similar to that described in Example 1 step 5 from tert-butyl 2-(difluoromethoxy)-6-methoxy-4-(4,4,5,5-tetramethyl-1,3,2-dioxaborolan-2-yl)benzoate and 6-chlorobenzofuran-3-yl trifluoromethanesulfonate. LC-MS: [M+H]⁺=425.0.

### Step 7: tert-butyl 2-(difluoromethoxy)-6-methoxy-4-(6-(1-methyl-1H-pyrazol-4-yl)benzofuran-3-yl)benzoate

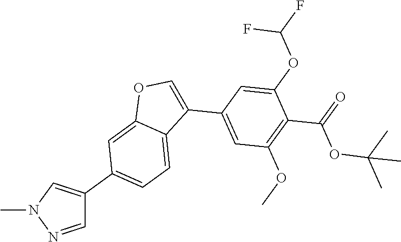 [SMILES: COc1cc(-c2coc3cc(-c4cnn(C)c4)ccc23)cc(OC(F)F)c1C(=O)OC(C)(C)C]

The title compound of step 7 (443 mg, 90% yield) was prepared in a manner similar to that described in Example 1 step 6 from tert-butyl 4-(6-chlorobenzofuran-3-yl)-2-(difluoromethoxy)-6-methoxybenzoate and 1-methyl-4-(4,4,5,5-tetramethyl-1,3,2-dioxaborolan-2-yl)-1H-pyrazole. LC-MS: [M+H]⁺=471.0.

### Step 8:2-(difluoromethoxy)-6-methoxy-4-(6-(1-methyl-1H-pyrazol-4-yl)benzofuran-3-yl)benzoic acid

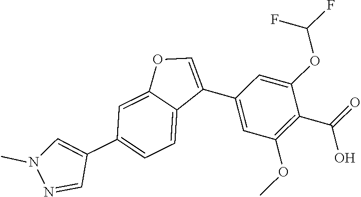 [SMILES: COc1cc(-c2coc3cc(-c4cnn(C)c4)ccc23)cc(OC(F)F)c1C(=O)O]

A solution of tert-butyl 2-(difluoromethoxy)-6-methoxy-4-(6-(1-methyl-1H-pyrazol-4-yl)benzofuran-3-yl)benzoate (443 mg, 0.94 mmol) in DCM (20 mL) was added TFA (4 mL) dropwise at 0° C. the mixture was stirred at room temperature for 2 h. The solvent was concentrated and the crude (390 mg) was directly used in next step without further purification. LC-MS: [M+H]⁺=415.0.

### Step 9:2-(difluoromethoxy)-6-methoxy-4-(6-(1-methyl-1H-pyrazol-4-yl)benzofuran-3-yl)-N-(2,2,2-trifluoroethyl)benzamide (compound 25)

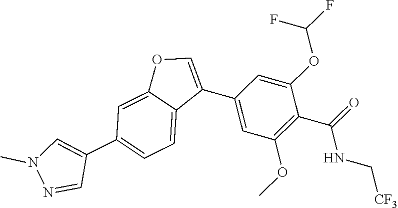 [SMILES: COc1cc(-c2coc3cc(-c4cnn(C)c4)ccc23)cc(OC(F)F)c1C(=O)NCC(F)(F)F]

2-(difluoromethoxy)-6-methoxy-4-(6-(1-methyl-1H-pyrazol-4-yl)benzofuran-3-yl)benzoic acid (150 mg, 0.363 mmol), DIPEA (141 mg, 1.089 mmol), 2,2,2-trifluoroethan-1-amine (72 mg, 0.725 mmol, 2 eq) and HATU (207 mg, 0.5445 mmol) were dissolved in DMF (2 mL) and the mixture was stirred at room temperature for overnight. Water (20 mL) was added and extracted with EA. The combine organic layers were washed with brine, dried over Na₂SO₄ and concentrated. The residue was purified by pre-HPLC to give compound 25 (63 mg, 35% yield). ¹H NMR (500 MHz, DMSO-d₆) δ 9.01 (t, J=6.3 Hz, 1H), 8.48 (s, 1H), 8.24 (s, 1H), 7.98 (s, 1H), 7.89 (d, J=10.3 Hz, 2H), 7.66-7.58 (m, 1H), 7.45-7.10 (m, 3H), 4.14-4.00 (m, 2H), 3.90 (s, 3H), 3.89 (s, 3H). LC-MS: [M+H]⁺=496.0.

### Example 26: 2-(difluoromethoxy)-N-((1R,2S)-2-fluorocyclopropyl)-6-methoxy-4-(6-(1-methyl-1H-pyrazol-4-yl)benzofuran-3-yl)benzamide (compound 26)

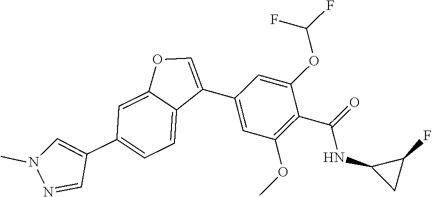 [SMILES: COc1cc(-c2coc3cc(-c4cnn(C)c4)ccc23)cc(OC(F)F)c1C(=O)N[C@@H]1C[C@@H]1F]

Compound 26 (45 mg, 87% yield) was prepared in a manner similar to that described in Example 25 step 9 from 2-(difluoromethoxy)-6-methoxy-4-(6-(1-methyl-1H-pyrazol-4-yl)benzofuran-3-yl)benzoic acid and (1R,2S)-2-fluorocyclopropan-1-amine 4-methylbenzenesulfonate. ¹H NMR (500 MHz, DMSO-d₆) δ 8.50 (t, J=7.5 Hz, 1H), 8.47 (d, J=8.8 Hz, 1H), 8.24 (s, 1H), 7.98 (s, 1H), 7.93-7.82 (m, 2H), 7.62 (d, J=8.1 Hz, 1H), 7.43-7.04 (m, 3H), 4.91-4.52 (m, 1H), 3.90 (s, 3H), 3.88 (s, 3H), 2.86-2.80 (m, 1H), 1.14-1.06 (m, 1H), 1.02-0.83 (m, 1H). LC-MS: [M+H]⁺=472.0.

### Example 27:2-cyclopropyl-8-methoxy-6-(6-(1-methyl-1H-pyrazol-4-yl)benzofuran-3-yl)-3,4-dihydroisoquinolin-1 (2H)-one (compound 27)

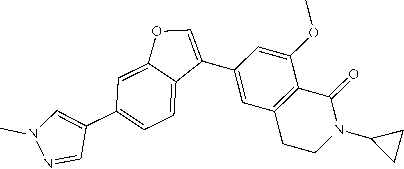 [SMILES: COc1cc(-c2coc3cc(-c4cnn(C)c4)ccc23)cc2c1C(=O)N(C1CC1)CC2]

### Step 1:6-bromo-2-cyclopropyl-8-methoxy-3,4-dihydroisoquinolin-1 (2H)-one

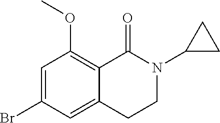 [SMILES: COc1cc(Br)cc2c1C(=O)N(C1CC1)CC2]

The reaction mixture of 6-bromo-8-methoxy-3,4-dihydroisoquinolin-1 (2H)-one (256 mg, 1.0 mmol cyclopropylboronic acid (129 mg, 1.5 mmol), cu (OAc) 2 (18 mg, 0.1 mmol) and Et₃N (101 mg, 1 mmol) in pyridine (2 mL) and THF (10 mL) was stirred at 70° C. for overnight under nitrogen. The mixture was filtered and the filtrate was evaporated. The residue was purified by silica gel column chromatography, eluting with PE/EtOAc (v/v=1/1), to give the title product (148 mg, 50% yield). LC-MS: [M+H]⁺=296.3.

### Step 2:2-cyclopropyl-8-methoxy-6-(4,4,5,5-tetramethyl-1,3,2-dioxaborolan-2-yl)-3,4-dihydroisoquinolin-1 (2H)-one

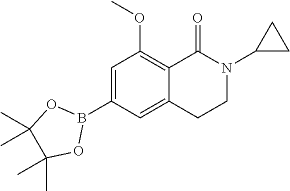 [SMILES: COc1cc(B2OC(C)(C)C(C)(C)O2)cc2c1C(=O)N(C1CC1)CC2]

The title compound of step 2 (171 mg, 90% yield) was prepared in a manner similar to that described in Example 1 step 4 from 6-bromo-2-cyclopropyl-8-methoxy-3,4-dihydroisoquinolin-1 (2H)-one and 4,4,4′,4′,5,5,5′,5′-octamethyl-2,2′-bi (1,3,2-dioxaborolane). LC-MS: [M+H]⁺=344.3.

### Step 3:3-(2-cyclopropyl-8-methoxy-1-oxo-1,2,3,4-tetrahydroisoquinolin-6-yl)benzofuran-6-yl acetate

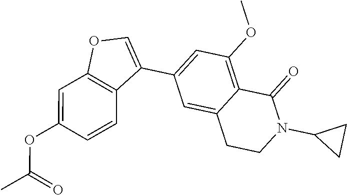 [SMILES: COc1cc(-c2coc3cc(OC(C)=O)ccc23)cc2c1C(=O)N(C1CC1)CC2]

The title compound of step 3 (196 mg, 100% yield) was prepared in a manner similar to that described in Example 1 step 5 from 2-cyclopropyl-8-methoxy-6-(4,4,5,5-tetramethyl-1,3,2-dioxaborolan-2-yl)-3,4-dihydroisoquinolin-1 (2H)-one and 3-(((trifluoromethyl) sulfonyl)oxy)benzofuran-6-yl acetate. LC-MS: [M+H]⁺=392.1.

### Step 4: 2-cyclopropyl-6-(6-hydroxybenzofuran-3-yl)-8-methoxy-3,4-dihydroisoquinolin-1 (2H)-one

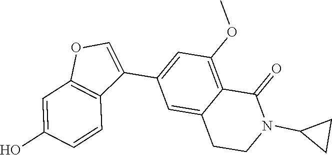 [SMILES: COc1cc(-c2coc3cc(O)ccc23)cc2c1C(=O)N(C1CC1)CC2]

The title compound of step 4 (174 mg, 100% yield) was prepared in a manner similar to that described in Example 13 step 7 from 3-(2-cyclopropyl-8-methoxy-1-oxo-1,2,3,4-tetrahydroisoquinolin-6-yl)benzofuran-6-yl acetate. LC-MS: [M+H]⁺=350.3.

### Step 5: 3-(2-cyclopropyl-8-methoxy-1-oxo-1,2,3,4-tetrahydroisoquinolin-6-yl)benzofuran-6-yl trifluoromethanesulfonate

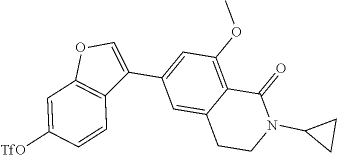 [SMILES: COc1cc(-c2coc3cc(OS(=O)(=O)C(F)(F)F)ccc23)cc2c1C(=O)N(C1CC1)CC2]

The title compound of step 5 (250 mg, 91% yield) was prepared in a manner similar to that described in Example 13 step 8 from 2-cyclopropyl-6-(6-hydroxybenzofuran-3-yl)-8-methoxy-3,4-dihydroisoquinolin-1 (2H)-one and N-Phenylbis(trifluoromethanesulfonimide). LC-MS: [M+H]⁺=482.3.

### Step 6: 2-cyclopropyl-8-methoxy-6-(6-(1-methyl-1H-pyrazol-4-yl)benzofuran-3-yl)-3,4-dihydroisoquinolin-1 (2H)-one (compound 27)

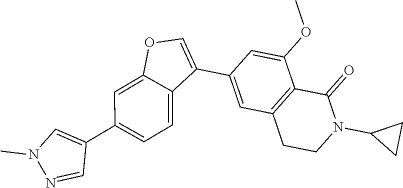 [SMILES: COc1cc(-c2coc3cc(-c4cnn(C)c4)ccc23)cc2c1C(=O)N(C1CC1)CC2]

Compound 27 (9 mg, 4% yield) was prepared in a manner similar to that described in Example 1 step 5 from 3-(2-cyclopropyl-8-methoxy-1-oxo-1,2,3,4-tetrahydroisoquinolin-6-yl)benzofuran-6-yl trifluoromethanesulfonate and (1-methyl-1H-pyrazol-4-yl) boronic acid. ¹H NMR (500 MHz, DMSO-d₆) δ 8.46 (s, 1H), 8.24 (s, 1H), 7.97 (s, 1H), 7.95 (d, J=8.2 Hz, 1H), 7.88 (s, 1H), 7.59 (d, J=8.2 Hz, 1H), 7.24 (d, J=9.6 Hz, 2H), 3.88 (s, 3H), 3.87 (s, 3H), 3.39 (t, J=6.1 Hz, 2H), 2.93 (t, J=6.1 Hz, 2H), 2.90-2.83 (m, 1H), 0.78-0.74 (m 2H), 0.68-0.59 (m, 2H). LC-MS: [M+H]⁺=414.3.

### Example 28: 6-methoxy-8-(6-(1-methyl-1H-pyrazol-4-yl)benzofuran-3-yl)-4-(2,2,2-trifluoroethyl)-3,4-dihydrobenzo[f][1,4]oxazepin-5 (2H)-one (compound 28)

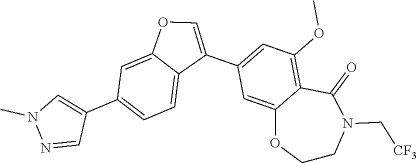 [SMILES: COc1cc(-c2coc3cc(-c4cnn(C)c4)ccc23)cc2c1C(=O)N(CC(F)(F)F)CCO2]

### Step 1: tert-butyl 4-bromo-2-(2-((tert-butoxycarbonyl)amino) ethoxy)-6-fluorobenzoate

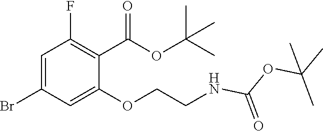 [SMILES: CC(C)(C)OC(=O)NCCOc1cc(Br)cc(F)c1C(=O)OC(C)(C)C]

A solution of tert-butyl 4-bromo-2,6-difluorobenzoate (11.7 g, 40 mmol) and tert-butyl N-(2-hydroxyethyl) carbamate (7.08 g, 44 mmol) in anhydrous THF (60 mL) was cooled to −10° C. NaH (60% suspension in mineral oil, 2.4 g, 133 mmol) was added portion-wise over 10 min. The reaction mixture was stirred for 1 h and quenched using saturated NH₄Cl solution (100 mL). Extraction was carried out using EtOAc (100 mL*2); the combined organic layers were washed with brine (200 mL), dried over anhydrous Na₂SO₄, filtered and concentrated under reduced pressure to provide the crude title product (12 g), which was directly used in next step without further purification. LC-MS [M+H]⁺=434.3.

### Step 2: 2-(2-aminoethoxy)-4-bromo-6-fluorobenzoic acid

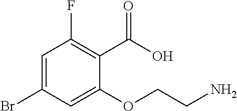 [SMILES: NCCOc1cc(Br)cc(F)c1C(=O)O]

To a solution of tert-butyl 4-bromo-2-(2-((tert-butoxycarbonyl)amino) ethoxy)-6-fluorobenzoate (crude, 6 g) in anhydrous CH₂Cl₂ (50 mL) was added a solution of 4M HCl in dioxane (30 mL); and the reaction mixture was stirred at room temperature for 16 h. Finally, solvents were removed under reduced pressure; the residue was washed with diethyl ether (50 mL*2) and dried to provide desired compound (3.5 g, 31% yield). LC-MS [M+H]⁺=277.9, 279.9.

### Step 3: 8-bromo-6-fluoro-3,4-dihydrobenzo[f][1,4]oxazepin-5 (2H)-one

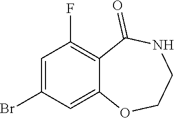 [SMILES: O=C1NCCOc2cc(Br)cc(F)c21]

A solution of 2-(2-aminoethoxy)-4-bromo-6-fluorobenzoic acid (1 g, 3.6 mmol) and EDCI (0.824 g, 4.3 mmol) in anhydrous pyridine (30 mL) was stirred at 80° C. for 1 h. The mixture was quenched using saturated NH₄Cl solution (20 mL). Extraction was carried out using EtOAc (20 mL*2); the combined organic layers were washed with brine (30 mL), dried over anhydrous Na₂SO₄, filtered and concentrated under reduced pressure. The residue was purified by silica gel column chromatography, eluting with PE/EtOAc (v/v=1/1), to give the title product (480 mg, 51% yield). LC-MS [M+H]⁺=260.1.

### Step 4: 8-bromo-6-methoxy-3,4-dihydrobenzo[f][1,4]oxazepin-5 (2H)-one

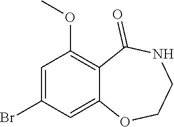

The title compound of step 4 (270 mg, 53% yield) was prepared in a manner similar to that described in Example 1 step 2 from 8-bromo-6-fluoro-3,4-dihydrobenzo[f][1,4]oxazepin-5 (2H)-one and sodium methoxide. LC-MS [M+H]⁺=272.1.

### Step 5: 8-bromo-6-methoxy-4-(2,2,2-trifluoroethyl)-3,4-dihydrobenzo[f][1,4]oxazepin-5 (2H)-one

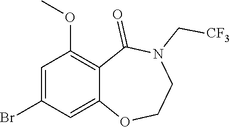 [SMILES: COc1cc(Br)cc2c1C(=O)N(CC(F)(F)F)CCO2]

The title compound of step 5 (130 mg, 37% yield) was prepared in a manner similar to that described in Example 1 step 3 from 8-bromo-6-methoxy-3,4-dihydrobenzo[f][1,4]oxazepin-5 (2H)-one and 2,2,2-trifluoroethyl trifluoromethanesulfonate. LC-MS [M+H]⁺=354.1.

### Step 6: 6-methoxy-8-(4,4,5,5-tetramethyl-1,3,2-dioxaborolan-2-yl)-4-(2,2,2-trifluoroethyl)-3,4-dihydrobenzo[f][1,4]oxazepin-5 (2H)-one

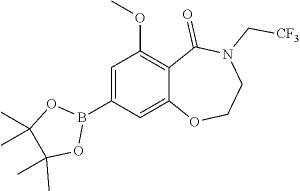 [SMILES: COc1cc(B2OC(C)(C)C(C)(C)O2)cc2c1C(=O)N(CC(F)(F)F)CCO2]

The title compound of step 6 (149 mg, 100% yield) was prepared in a manner similar to that described in Example 1 step 4 from 8-bromo-6-methoxy-4-(2,2,2-trifluoroethyl)-3,4-dihydrobenzo[f][1.4]oxazepin-5 (2H)-one and 4,4,4′,4′,5,5,5′,5′-octamethyl-2,2′-bi (1,3,2-dioxaborolane). LC-MS [M+H]⁺=402.1.

### Step 7: 8-(6-chlorobenzofuran-3-yl)-6-methoxy-4-(2,2,2-trifluoroethyl)-3,4-dihydrobenzo[f][1,4]oxazepin-5 (2H)-one

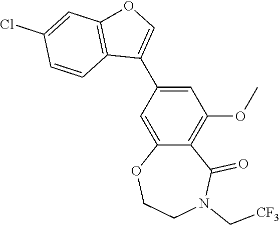 [SMILES: COc1cc(-c2coc3cc(Cl)ccc23)cc2c1C(=O)N(CC(F)(F)F)CCO2]

The title compound of step 7 (100 mg, 64% yield) was prepared in a manner similar to that described in Example 1 step 5 from 6-methoxy-8-(4,4,5,5-tetramethyl-1,3,2-dioxaborolan-2-yl)-4-(2,2,2-trifluoroethyl)-3,4-dihydrobenzo[f][1,4]oxazepin-5 (2H)-one and 6-chlorobenzofuran-3-yl trifluoromethanesulfonate. LC-MS [M+H]⁺=426.1.

### Step 8: 6-methoxy-8-(6-(1-methyl-1H-pyrazol-4-yl)benzofuran-3-yl)-4-(2,2,2-trifluoroethyl)-3,4-dihydrobenzo[f][1,4]oxazepin-5 (2H)-one (compound 28)

Compound 28 (2.5 mg, 2% yield) was prepared in a manner similar to that descried in Example 1 step 6 from 8-(6-chlorobenzofuran-3-yl)-6-methoxy-4-(2,2,2-trifluoroethyl)-3,4-dihydrobenzo[f][1.4]oxazepin-5 (2H)-one and (1-methyl-1H-pyrazol-4-yl) boronic acid. ¹H NMR (500 MHz, DMSO-d₆) δ 8.49 (s, 1H), 8.24 (s, 1H), 7.97 (s, 1H), 7.93 (d, J=8.2 Hz, 1H), 7.89 (s, 1H), 7.60 (dd, J₁=8.2 Hz, J₂=1.1 Hz, 1H), 7.24 (s, 1H), 7.07 (d, J=1.0 Hz, 1H), 4-42-4.39 (m, 2H), 4.26 (t, J=5.4 Hz, 2H), 3.90 (s, 3H), 3.88 (s, 3H), 3.64 (t, J=5.3 Hz, 2H). LC-MS [M+H]⁺=472.3.

### Example 29A&B: (S)-8-methoxy-3-methyl-6-(6-(1-methyl-1H-pyrazol-4-yl)benzofuran-3-yl)-2-(2,2,2-trifluoroethyl)-3,4-dihydroisoquinolin-1 (2H)-one (29A) and (R)-8-methoxy-3-methyl-6-(6-(1-methyl-1H-pyrazol-4-yl)benzofuran-3-yl)-2-(2,2,2-trifluoroethyl)-3,4-dihydroisoquinolin-1 (2H)-one (29B)

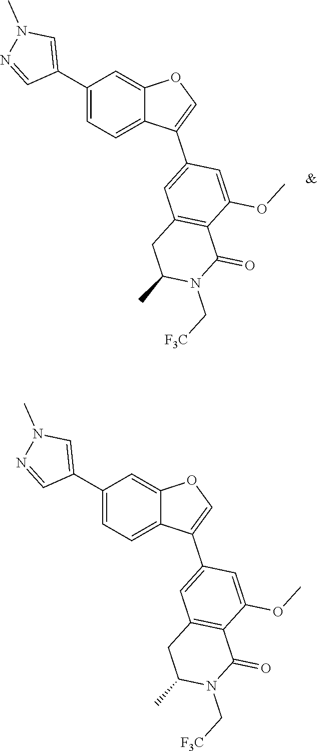 [SMILES: COc1cc(-c2coc3cc(-c4cnn(C)c4)ccc23)cc2c1C(=O)N(CC(F)(F)F)[C@@H](C)C2 ; COc1cc(-c2coc3cc(-c4cnn(C)c4)ccc23)cc2c1C(=O)N(CC(F)(F)F)[C@H](C)C2]

### Step 1: 5-bromo-7-fluoro-2-methyl-2,3-dihydro-1H-inden-1-one

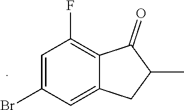

5-bromo-7-fluoro-2,3-dihydro-1H-inden-1-one (4.5 g, 19.65 mmol) was dissolved in dry THF (50 mL). lithium diisopropylamide (2N, 11.8 mL, 23.58 mmol) was added at −65° C. under N₂ and the mixture was stirred at −65° C. for 30 min. CH₃I (3.346 g, 23.58 mmol) was added dropwise at −65° C. and the mixture was stirred at room temperature for overnight. NH₄Cl (sat. 20 mL) was added at 0° C. and extracted with EA. The combine organic layers were washed with brine and dried over Na₂SO₄. The residue was purified by silica gel column chromatography, eluting with PE/EtOAc (v/v=1/1), to give the title product as a white solid (755 mg, 15% yield). LC-MS [M+H]⁺=243.0.

### Step 2: 5-bromo-7-fluoro-2-methyl-2,3-dihydro-1H-inden-1-one oxime

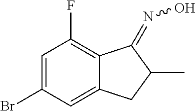

5-bromo-7-fluoro-2-methyl-2,3-dihydro-1H-inden-1-one (555 mg, 2.283 mmol), NaOAc (280.94 mg, 3.425 mmol) and hydroxylamine hydrochloride (238 mg, 3.425 mmol) were dissolved in EtOH (20 mL) and water (10 mL) and the mixture was refluxed for overnight. Solvent was removed in vacuum. Water (20 mL) was added and extracted with EA. The combine organic layers were washed with brine, dried over Na₂SO₄ and concentrated to afford the title product (589.3 mg, 100% yield). LC-MS [M+H]⁺=258.0.

### Step 3: 6-bromo-8-fluoro-3-methyl-3,4-dihydroisoquinolin-1 (2H)-one

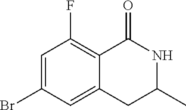

5-bromo-7-fluoro-2-methyl-2,3-dihydro-1H-inden-1-one oxime (549.3 mg, 2.13 mmol) was dissolved in SOCl₂ (10 mL) and the mixture was refluxed for 7 hrs. SOCl₂ was removed by reduced pressure. The residue was purified by silica gel column chromatography, eluting with PE/EtOAc (v/v=1/1), to give the title product (230 mg, 41% yield). LC-MS [M+H]⁺=258.0.

### Step 4: 6-bromo-8-methoxy-3-methyl-3,4-dihydroisoquinolin-1 (2H)-one

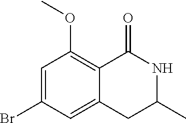

The title compound of step 4 (247 mg, 100% yield) was prepared in a manner similar to that described in Example 1 step 2 from 6-bromo-8-fluoro-3-methyl-3,4-dihydroisoquinolin-1 (2H)-one and sodium methoxide. LC-MS [M+H]⁺=270.0.

### Step 5: 6-bromo-8-methoxy-3-methyl-2-(2,2,2-trifluoroethyl)-3,4-dihydroisoquinolin-1 (2H)-one

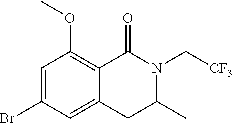 [SMILES: COc1cc(Br)cc2c1C(=O)N(CC(F)(F)F)C(C)C2]

The title compound of step 5 (196 mg, 60% yield) was prepared in a manner similar to that described in Example 1 step 3 from 6-bromo-8-methoxy-3-methyl-3,4-dihydroisoquinolin-1 (2H)-one and 2,2,2-trifluoroethyl trifluoromethanesulfonate. LC-MS [M+H]⁺=352.0.

### Step 6: 8-methoxy-3-methyl-6-(4,4,5,5-tetramethyl-1,3,2-dioxaborolan-2-yl)-2-(2,2,2-trifluoroethyl)-3,4-dihydroisoquinolin-1 (2H)-one

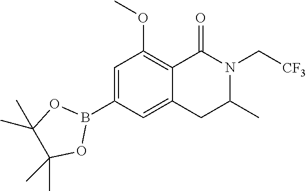 [SMILES: COc1cc(B2OC(C)(C)C(C)(C)O2)cc2c1C(=O)N(CC(F)(F)F)C(C)C2]

The title compound of step 6 (222 mg, 100% yield) was prepared in a manner similar to that described in Example 1 step 4 from 6-bromo-8-methoxy-3-methyl-2-(2,2,2-trifluoroethyl)-3,4-dihydroisoquinolin-1 (2H)-one and 4,4,4′,4′,5,5,5′,5′-octamethyl-2,2′-bi (1,3,2-dioxaborolane). LC-MS [M+H]⁺=400.0.

### Step 7: 6-(6-chlorobenzofuran-3-yl)-8-methoxy-3-methyl-2-(2,2,2-trifluoroethyl)-3,4-dihydroisoquinolin-1 (2H)-one

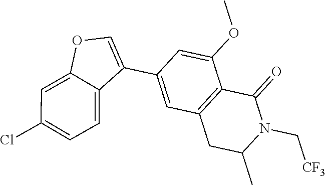 [SMILES: COc1cc(-c2coc3cc(Cl)ccc23)cc2c1C(=O)N(CC(F)(F)F)C(C)C2]

The title compound of step 7 (190 mg, 80% yield) was prepared in a manner similar to that described in Example 1 step 5 from 8-methoxy-3-methyl-6-(4,4,5,5-tetramethyl-1,3,2-dioxaborolan-2-yl)-2-(2,2,2-trifluoroethyl)-3,4-dihydroisoquinolin-1 (2H)-one and 6-chlorobenzofuran-3-yl trifluoromethanesulfonate. LC-MS [M+H]⁺=424.0.

### Step 8: 8-methoxy-3-methyl-6-(6-(1-methyl-1H-pyrazol-4-yl)benzofuran-3-yl)-2-(2,2,2-trifluoroethyl)-3,4-dihydroisoquinolin-1 (2H)-one

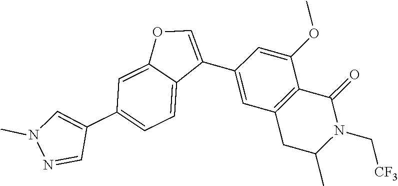 [SMILES: COc1cc(-c2coc3cc(-c4cnn(C)c4)ccc23)cc2c1C(=O)N(CC(F)(F)F)C(C)C2]

The title compound of step 8 (65 mg, 31% yield) was prepared in a manner similar to that described in Example 1 step 6 from 6-(6-chlorobenzofuran-3-yl)-8-methoxy-3-methyl-2-(2,2,2-trifluoroethyl)-3,4-dihydroisoquinolin-1 (2H)-one and (1-methyl-1H-pyrazol-4-yl) boronic acid. LC-MS [M+H]⁺=470.0.

### Step 9: (S)-8-methoxy-3-methyl-6-(6-(1-methyl-1H-pyrazol-4-yl)benzofuran-3-yl)-2-(2,2,2-trifluoroethyl)-3,4-dihydroisoquinolin-1 (2H)-one (29A) and (R)-8-methoxy-3-methyl-6-(6-(1-methyl-1H-pyrazol-4-yl)benzofuran-3-yl)-2-(2,2,2-trifluoroethyl)-3,4-dihydroisoquinolin-1 (2H)-one (29B)

The compound obtained in step 8 was purified by chiral-HPLC (column: CHIRALPAK IA-3 4.6*100 mm 3 um; gradient: hexane/EtOH containing 0.1% diethylamine (v/v=7/3); flow rate: 0.8 mL/min) to give compound 29A (30 mg) and compound 29B (26 mg).

Compound 29A: ¹H NMR (500 MHz, DMSO-d₆) δ 8.52 (s, 1H), 8.24 (s, 1H), 7.98 (t, J=4.1 Hz, 2H), 7.90 (s, 1H), 7.60 (d, J=8.2 Hz, 1H), 7.31 (s, 1H), 7.28 (s, 1H), 4.56-4.47 (m, 1H), 4.13-4.05 (m, 1H), 3.94-3.88 (m, 7H), 3.31-3.27 (m, 1H), 2.88 (dd, J₁=15.8 Hz, J₂=1.9 Hz, 1H), 1.11 (d. J=6.5 Hz, 3H). LC-MS [M+H]⁺=470.0. retention time: 5.96 min.

Compound 29B: ¹H NMR (500 MHz, DMSO-d₆) δ 8.52 (s, 1H), 8.24 (s, 1H), 7.98 (t, J=4.1 Hz, 2H), 7.89 (d, J=0.9 Hz, 1H), 7.60 (dd, J=8.2, 1.3 Hz, 1H), 7.31 (s, 1H), 7.28 (s, 1H), 4.56-4.47 (m, 1H), 4.13-4.04 (m, 1H), 3.94-3.88 (m, 7H), 3.30-3.27 (m, 1H), 2.87 (dd, J₁=15.8, J₂=1.9 Hz, 1H), 1.11 (d, J=6.6 Hz, 3H). LC-MS [M+H]⁺=470.0. retention time: 7.84 min.

### Example 30: 8-methoxy-6-(6-(1-methyl-1H-pyrazol-4-yl)benzofuran-3-yl)-2-(pyridin-4-ylmethyl)-3,4-dihydroisoquinolin-1 (2H)-one (compound 30)

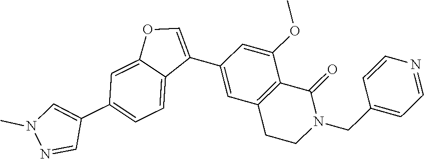 [SMILES: COc1cc(-c2coc3cc(-c4cnn(C)c4)ccc23)cc2c1C(=O)N(Cc1ccncc1)CC2]

### Step 1: 6-bromo-8-methoxy-2-(pyridin-4-ylmethyl)-3,4-dihydroisoquinolin-1 (2H)-one

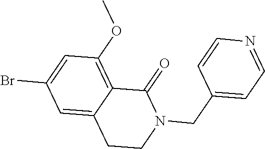 [SMILES: COc1cc(Br)cc2c1C(=O)N(Cc1ccncc1)CC2]

To a solution of 6-bromo-8-methoxy-3,4-dihydroisoquinolin-1 (2H)-one (200 mg, 0.78 mmol) in THF (5 mL) was added NaH (60 mg, 1.56 mmol). The mixture was stirred at 0° C. for 30 minutes. 4-(bromomethyl)pyridine hydrobromide (300 mg, 1.18 mmol.) was added into the above mixture. The mixture was stirred at RT for 3 hours. Water (10 mL) was added, extracted with ethyl acetate (20 mL). the combined organic layers were washed with brine (10 mL), dried with over sodium sulfate, and concentrated under reduced pressure. The residue was purified by silica gel column chromatography, eluting with PE/EtOAc (v/v=7/3), to give the title product (150 mg, 55% yield). LC-MS [M+H]⁺=347.0.

### Step 2: 8-methoxy-2-(pyridin-4-ylmethyl)-6-(4,4,5,5-tetramethyl-1,3,2-dioxaborolan-2-yl)-3,4-dihydroisoquinolin-1 (2H)-one

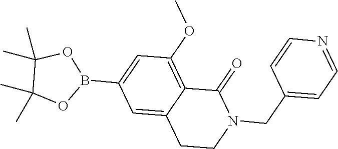 [SMILES: COc1cc(B2OC(C)(C)C(C)(C)O2)cc2c1C(=O)N(Cc1ccncc1)CC2]

The title compound of step 2 (140 mg, 82% yield) was prepared in a manner similar to that described in Example 1 step 4 from 6-bromo-8-methoxy-2-(pyridin-4-ylmethyl)-3,4-dihydroisoquinolin-1 (2H)-one and 4,4,4′,4′,5,5,5′,5′-octamethyl-2,2′-bi (1,3,2-dioxaborolane). LC-MS [M+H]⁺=395.2.

### Step 3: 6-(6-chlorobenzofuran-3-yl)-8-methoxy-2-(pyridin-4-ylmethyl)-3,4-dihydroisoquinolin-1 (2H)-one

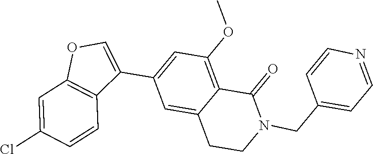 [SMILES: COc1cc(-c2coc3cc(Cl)ccc23)cc2c1C(=O)N(Cc1ccncc1)CC2]

The title compound of step 3 (100 mg, 68% yield) was prepared in a manner similar to that described in Example 1 step 5 from 8-methoxy-2-(pyridin-4-ylmethyl)-6-(4,4,5,5-tetramethyl-1,3,2-dioxaborolan-2-yl)-3,4-dihydroisoquinolin-1 (2H)-one and 6-chlorobenzofuran-3-yl trifluoromethanesulfonate. LC-MS [M+H]⁺=419.9.

### Step 4: 8-methoxy-6-(6-(1-methyl-1H-pyrazol-4-yl)benzofuran-3-yl)-2-(pyridin-4-ylmethyl)-3,4-dihydroisoquinolin-1 (2H)-one (compound 30)

Compound 30 (5 mg, 9% yield) was prepared in a manner similar to that described in Example 1 step 6 from 6-(6-chlorobenzofuran-3-yl)-8-methoxy-2-(pyridin-4-ylmethyl)-3,4-dihydroisoquinolin-1 (2H)-one and (1-methyl-1H-pyrazol-4-yl) boronic acid. ¹H NMR (500 MHz, DMSO-d₆) δ 8.53 (dd, J₁=4.5 Hz, J₂=1.4 Hz, 2H), 8.49 (s, 1H), 8.24 (s, 1H), 7.97 (d, J=8.1 Hz, 2H), 7.89 (s, 1H), 7.60 (dd, J₁=8.2 Hz, J 2=1.3 Hz, 1H), 7.31 (d, J=6.0 Hz, 3H), 7.27 (s, 1H), 4.72 (s, 2H), 3.90 (s, 3H), 3.89 (s, 3H), 3.52 (t, J=6.2 Hz, 2H), 3.01 (t, J=6.2 Hz, 2H). LC-MS [M+H]⁺=465.3.

### Example 31: 2-benzyl-8-methoxy-6-(6-(1-methyl-1H-pyrazol-4-yl)benzofuran-3-yl)-3,4-dihydroisoquinolin-1 (2H)-one (compound 31)

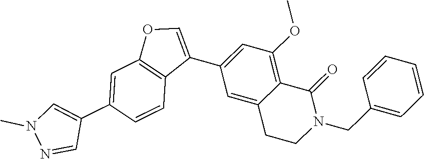 [SMILES: COc1cc(-c2coc3cc(-c4cnn(C)c4)ccc23)cc2c1C(=O)N(Cc1ccccc1)CC2]

### Step 1: 2-benzyl-6-bromo-8-methoxy-3,4-dihydroisoquinolin-1 (2H)-one

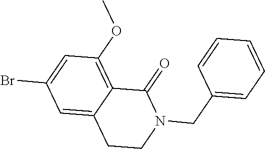 [SMILES: COc1cc(Br)cc2c1C(=O)N(Cc1ccccc1)CC2]

The title compound of step 1 (160 mg, 74% yield) was prepared in a manner similar to that described in Example 30 step 1 from 6-bromo-8-methoxy-3,4-dihydroisoquinolin-1 (2H)-one and benzyl chloride. LC-MS [M+H]⁺=347.2.

### Step 2: 2-benzyl-8-methoxy-6-(4,4,5,5-tetramethyl-1,3,2-dioxaborolan-2-yl)-3,4-dihydroisoquinolin-1 (2H)-one

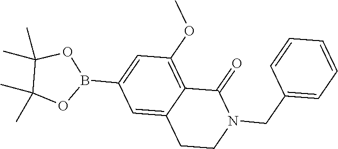 [SMILES: COc1cc(B2OC(C)(C)C(C)(C)O2)cc2c1C(=O)N(Cc1ccccc1)CC2]

The title compound of step 2 (150 mg, 95% yield) was prepared in a manner similar to that described in Example 1 step 4 from 2-benzyl-6-bromo-8-methoxy-3,4-dihydroisoquinolin-1 (2H)-one and 4,4,4′,4′,5,5,5′,5′-octamethyl-2,2′-bi (1,3,2-dioxaborolane). LC-MS [M+H]⁺=394.3.

### Step 3: 2-benzyl-6-(6-chlorobenzofuran-3-yl)-8-methoxy-3,4-dihydroisoquinolin-1 (2H)-one

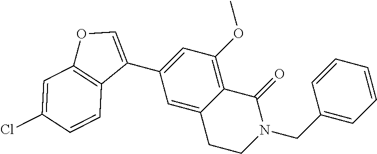 [SMILES: COc1cc(-c2coc3cc(Cl)ccc23)cc2c1C(=O)N(Cc1ccccc1)CC2]

The title compound of step 3 (100 mg, 53% yield) was prepared in a manner similar to that described in Example 1 step 5 from 2-benzyl-8-methoxy-6-(4,4,5,5-tetramethyl-1,3,2-dioxaborolan-2-yl)-3,4-dihydroisoquinolin-1 (2H)-one and 6-chlorobenzofuran-3-yl trifluoromethanesulfonate. LC-MS [M+H]⁺=418.9.

### Step 4: 2-benzyl-8-methoxy-6-(6-(1-methyl-1H-pyrazol-4-yl)benzofuran-3-yl)-3,4-dihydroisoquinolin-1 (2H)-one (compound 31)

Compound 31 (4 mg, 9% yield) was prepared in a manner similar to that described in Example 1 step 6 from 2-benzyl-6-(6-chlorobenzofuran-3-yl)-8-methoxy-3,4-dihydroisoquinolin-1 (2H)-one and (1-methyl-1H-pyrazol-4-yl) boronic acid. ¹H NMR (500 MHz, DMSO-d₆) δ 8.48 (s, 1H), 8.24 (s, 1H), 7.98-7.85 (m, 3H), 7.59 (dd, J₁=8.2 Hz, J₂=1.3 Hz, 1H), 7.44-7.22 (m, 7H), 4.69 (s, 2H), 3.90 (s, 3H), 3.88 (s, 3H), 3.45 (t, J=6.3 Hz, 2H), 2.94 (t, J=6.2 Hz, 2H). LC-MS [M+H]⁺=464.2.

### Example 32: 4-(6-(1-methyl-1H-pyrazol-4-yl)benzofuran-3-yl)benzonitrile (compound 32)

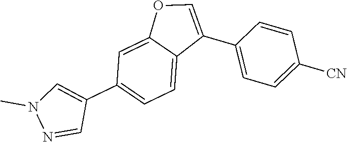 [SMILES: Cn1cc(-c2ccc3c(-c4ccc(C#N)cc4)coc3c2)cn1]

### Step 1: 4-(6-chlorobenzofuran-3-yl)benzonitrile

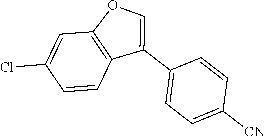 [SMILES: N#Cc1ccc(-c2coc3cc(Cl)ccc23)cc1]

The title compound of step 1 (150 mg, 87% yield) was prepared in a manner similar to that described in Example 1 step 5 from (4-cyanophenyl) boronic acid and 6-chlorobenzofuran-3-yl trifluoromethanesulfonate. LC-MS [M+H]⁺=254.0.

### Step 2: 4-(6-(4,4,5,5-tetramethyl-1,3,2-dioxaborolan-2-yl)benzofuran-3-yl)benzonitrile

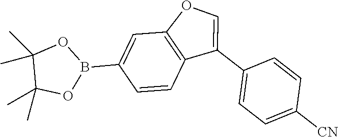 [SMILES: CC1(C)OB(c2ccc3c(-c4ccc(C#N)cc4)coc3c2)OC1(C)C]

The title compound of step 2 (130 mg, 77% yield) was prepared in a manner similar to that described in Example 1 step 4 from 4-(6-chlorobenzofuran-3-yl)benzonitrile and 4,4,4′,4′,5,5,5′,5′-octamethyl-2,2′-bi (1,3,2-dioxaborolane). LC-MS [M+H]⁺=346.2.

### Step 3: 4-(6-(1-methyl-1H-pyrazol-4-yl)benzofuran-3-yl)benzonitrile (compound 32)

Compound 32 (19 mg, 17% yield) was prepared in a manner similar to that described in Example 1 step 6 from 4-(6-(4,4,5,5-tetramethyl-1,3,2-dioxaborolan-2-yl)benzofuran-3-yl)benzonitrile and 4-iodo-1-methyl-1H-pyrazole. ¹H NMR (500 MHz, DMSO-d₆) δ 8.56 (s, 1H), 8.26 (s, 1H), 8.04-7.75 (m, 7H), 7.61 (dd, J₁=8.2 Hz, J₂=1.5 Hz, 1H), 3.95 (s, 3H). LC-MS [M+H]⁺=300.2.

### Example 33: 8-methoxy-4,4-dimethyl-6-(6-(1-methyl-1H-pyrazol-4-yl)benzofuran-3-yl)-2-(2,2,2-trifluoroethyl)-3,4-dihydroisoquinolin-1 (2H)-one (compound 33)

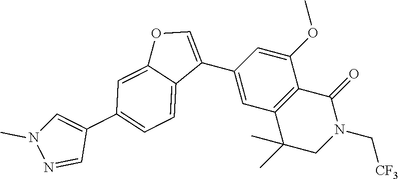 [SMILES: COc1cc(-c2coc3cc(-c4cnn(C)c4)ccc23)cc2c1C(=O)N(CC(F)(F)F)CC2(C)C]

### Step 1: 6-bromo-4-fluoro-1,1-dimethyl-2,3-dihydro-1H-indene

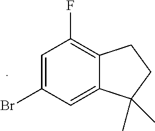

TiCl₄ (3.9 g, 20.52 mmol) was added to dry DCM (90 mL) under N₂ at −60° C. and the mixture was stirred at −60° C. for 30 min. Zn(CH₃)₂ (29.4 mL, 1N solution in THF, 29.344 mmol) was added at −60° C. and stirred at −60° C. for 30 min. 6-bromo-4-fluoro-2,3-dihydro-1H-inden-1-one (4.7 g, 20.52 mmol) in dry DCM (20 mL) was added at −65° C. and the mixture was stirred at room temperature overnight. NH₄Cl (sat. 20 mL) was added at 0° C. and extracted with DCM. The combined organic layers were washed with brine and dried over Na₂SO₄. The residue was purified by silica gel column chromatography, eluting with PE/EtOAc (v/v=1/1), to give the title product (4.2 g, 84% yield). LC-MS [M+H]⁺=243.0.

### Step 2: 5-bromo-7-fluoro-3,3-dimethyl-2,3-dihydro-1H-inden-1-one

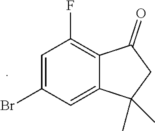

6-bromo-4-fluoro-1,1-dimethyl-2,3-dihydro-1H-indene (1 g, 4.12 mmol) was dissolved in AcOH (15 mL). CrO₃ (2.47 g, 24.68 mmol) was added and heated to 60° C. for 3 hrs. Solvent was removed by reduced pressure. Water (20 mL) was added and extracted with EA. The combined organic layers were washed with NaHCO₃ (sat.), brine and dried over Na₂SO₄. The solvent was concentrated to give the title product (690 mg, 65% yield). LC-MS [M+H]⁺=257.0.

### Step 3: 6-bromo-8-fluoro-4,4-dimethyl-3,4-dihydroisoquinolin-1 (2H)-one

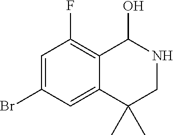

5-bromo-7-fluoro-3,3-dimethyl-2,3-dihydro-1H-inden-1-one (200 mg, 0.778 mmol) and MeSO₃H (314.3 mg, 3.27 mmol) were dissolved in DCM (10 mL) at 0° C. NaN₃ (76.86 mg, 1.167 mmol) was added at 0° C. and the mixture was stirred at room temperature for overnight. The pH was adjusted to 8-9 with 2N NaOH aqueous solution and extracted with EA. The combined organic layers were washed with NaHCO₃ (sat.), brine and dried over Na₂SO₄. The residue was purified by silica gel column chromatography to give the title product (162 mg, 76% yield). LC-MS [M+H]⁺=272.0.

### Step 4: 6-bromo-8-methoxy-4,4-dimethyl-3,4-dihydroisoquinolin-1 (2H)-one

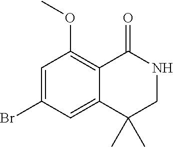 [SMILES: COc1cc(Br)cc2c1C(=O)NCC2(C)C]

The title compound of step 4 (101 mg, 59% yield) was prepared in a manner similar to that described in Example 1 step 2 from 6-bromo-8-fluoro-4,4-dimethyl-3,4-dihydroisoquinolin-1 (2H)-one and sodium methoxide. LC-MS [M+H]⁺=284.0.

### Step 5: 6-bromo-8-methoxy-4,4-dimethyl-2-(2,2,2-trifluoroethyl)-3,4-dihydroisoquinolin-1 (2H)-one

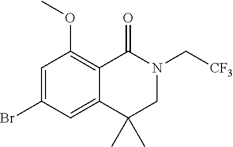 [SMILES: COc1cc(Br)cc2c1C(=O)N(CC(F)(F)F)CC2(C)C]

The title compound of step 5 (90 mg, 69% yield) was prepared in a manner similar to that described in Example 1 step 3 from 6-bromo-8-methoxy-4,4-dimethyl-3,4-dihydroisoquinolin-1 (2H)-one and 2,2,2-trifluoroethyl trifluoromethanesulfonate. LC-MS [M+H]⁺=366.0.

### Step 6: 8-methoxy-4,4-dimethyl-6-(4,4,5,5-tetramethyl-1,3,2-dioxaborolan-2-yl)-2-(2,2,2-trifluoroethyl)-3,4-dihydroisoquinolin-1 (2H)-one

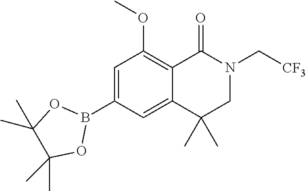 [SMILES: COc1cc(B2OC(C)(C)C(C)(C)O2)cc2c1C(=O)N(CC(F)(F)F)CC2(C)C]

The title compound of step 6 (101 mg, 100% yield) was prepared in a manner similar to that described in Example 1 step 4 from 6-bromo-8-methoxy-4,4-dimethyl-2-(2,2,2-trifluoroethyl)-3,4-dihydroisoquinolin-1 (2H)-one and 4,4,4′,4′,5,5,5′,5′-octamethyl-2,2′-bi (1,3,2-dioxaborolane). LC-MS [M+H]⁺=414.0.

### Step 7: 6-(6-chlorobenzofuran-3-yl)-8-methoxy-4,4-dimethyl-2-(2,2,2-trifluoroethyl)-3,4-dihydroisoquinolin-1 (2H)-one

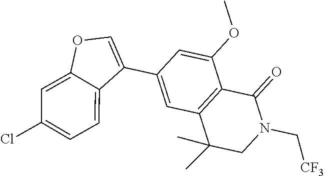 [SMILES: COc1cc(-c2coc3cc(Cl)ccc23)cc2c1C(=O)N(CC(F)(F)F)CC2(C)C]

The title compound of step 7 (99 mg, 100% yield) was prepared in a manner similar to that described in Example 1 step 5 from 8-methoxy-4,4-dimethyl-6-(4,4,5,5-tetramethyl-1,3,2-dioxaborolan-2-yl)-2-(2,2,2-trifluoroethyl)-3,4-dihydroisoquinolin-1 (2H)-one and 6-chlorobenzofuran-3-yl trifluoromethanesulfonate. LC-MS [M+H]⁺=438.0.

### Step 8: 8-methoxy-4,4-dimethyl-6-(6-(1-methyl-1H-pyrazol-4-yl)benzofuran-3-yl)-2-(2,2,2-trifluoroethyl)-3,4-dihydroisoquinolin-1 (2H)-one (compound 33)

Compound 33 (23 mg, 10% yield) was prepared in a manner similar to that described in Example 1 step 6 from 6-(6-chlorobenzofuran-3-yl)-8-methoxy-4,4-dimethyl-2-(2,2,2-trifluoroethyl)-3,4-dihydroisoquinolin-1 (2H)-one and (1-methyl-1H-pyrazol-4-yl) boronic acid. ¹H NMR (500 MHz, DMSO-d₆) δ 8.52 (s, 1H), 8.24 (s, 1H), 7.98 (s, 1H), 7.91 (d, J=8.4 Hz, 2H), 7.61 (dd, J₁=8.1 Hz, J₂=1.4 Hz, 1H), 7.30 (s, 2H), 4.36 (q. J=9.6 Hz, 2H), 3.90 (s, 3H), 3.89 (s, 3H), 3.45 (s, 2H), 1.33 (s, 6H). LC-MS [M+H]⁺=484.5.

### Example 34: 2-(2,6-dimethoxy-4-(6-(1-methyl-1H-pyrazol-4-yl)benzofuran-3-yl)phenyl)-5-methyl-1,3,4-oxadiazole (compound 34)

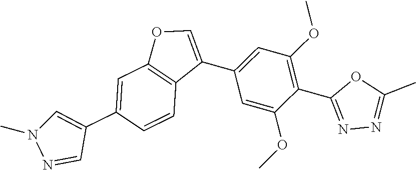

### Step 1: methyl 4-bromo-2,6-dimethoxybenzoate

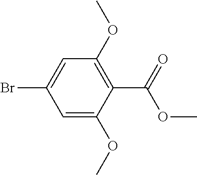

To a solution of methyl 4-bromo-2,6-difluorobenzoate (1.0 g, 4 mmol) in MeOH (20 mL) was added NaOMe in MeOH (30% solution, 3.6 g, 16 mmol). The mixture was stirred at 80° C. for 3 hours. Water (100 mL) was added, extracted with ethyl acetate (300 mL). The combined organic layers were washed with brine (100 mL), dried with over sodium sulfate, and concentrated under reduced pressure. The residue was purified by silica gel column chromatography, eluting with PE/EtOAc (v/v=7/3), to give the title product (800 mg, 73% yield). LC-MS [M+H]⁺=276.0.

### Step 2: methyl 2,6-dimethoxy-4-(4,4,5,5-tetramethyl-1,3,2-dioxaborolan-2-yl)benzoate

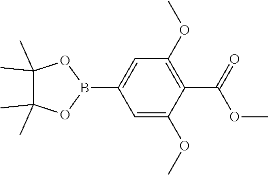

The title compound of step 2 (850 mg, 90% yield) was prepared in a manner similar to that described in Example 1 step 4 from methyl 4-bromo-2,6-dimethoxybenzoate and 4,4,4′,4′,5,5,5′,5′-octamethyl-2,2′-bi (1,3,2-dioxaborolane). LC-MS [M+H]⁺=323.2.

### Step 3: methyl 4-(6-chlorobenzofuran-3-yl)-2,6-dimethoxybenzoate

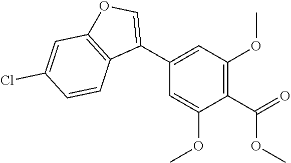 [SMILES: COC(=O)c1c(OC)cc(-c2coc3cc(Cl)ccc23)cc1OC]

The title compound of step 3 (600 mg, 66% yield) was prepared in a manner similar to that described in Example 1 step 5 from methyl 2,6-dimethoxy-4-(4,4,5,5-tetramethyl-1,3,2-dioxaborolan-2-yl)benzoate and 6-chlorobenzofuran-3-yl trifluoromethanesulfonate. LC-MS [M+H]⁺=347.8.

### Step 4: methyl 2,6-dimethoxy-4-(6-(1-methyl-1H-pyrazol-4-yl)benzofuran-3-yl)benzoate

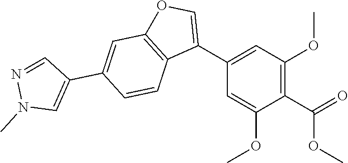 [SMILES: COC(=O)c1c(OC)cc(-c2coc3cc(-c4cnn(C)c4)ccc23)cc1OC]

The title compound of step 4 (400 mg, 59% yield) was prepared in a manner similar to that described in Example 1 step 6 from methyl 4-(6-chlorobenzofuran-3-yl)-2,6-dimethoxybenzoate and (1-methyl-1H-pyrazol-4-yl) boronic acid. LC-MS [M+H]⁺=393.4.

### Step 5: 2,6-dimethoxy-4-(6-(1-methyl-1H-pyrazol-4-yl)benzofuran-3-yl)benzoic acid

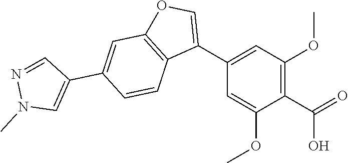 [SMILES: COc1cc(-c2coc3cc(-c4cnn(C)c4)ccc23)cc(OC)c1C(=O)O]

To a solution of methyl 2,6-dimethoxy-4-(6-(1-methyl-1H-pyrazol-4-yl)benzofuran-3-yl)benzoate (400 mg, 1.02 mmol) in MeOH (10 mL) and water (2 mL) was added NaOH (123 mg, 3.06 mmol). The mixture was stirred at 60° C. for 3 hours. Water (20 mL) was added, the pH of the mixture was adjusted to 2˜3 with 2 N HCl. The solid was filtered and dried to afford the title product as a white solid (300 mg, 98% yield), which was directly used in next step without further purification. LC-MS [M+H]⁺=379.3.

### Step 6: N′-acetyl-2,6-dimethoxy-4-(6-(1-methyl-1H-pyrazol-4-yl)benzofuran-3-yl)benzohydrazide

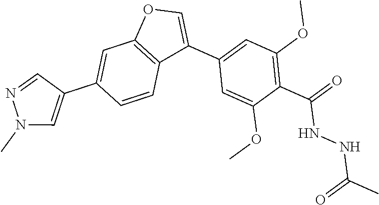 [SMILES: COc1cc(-c2coc3cc(-c4cnn(C)c4)ccc23)cc(OC)c1C(=O)NNC(C)=O]

To a solution of 2,6-dimethoxy-4-(6-(1-methyl-1H-pyrazol-4-yl)benzofuran-3-yl)benzoic acid (100 mg, 0.26 mmol.) in ACN (10 mL) was added EDCI (77.5 mg, 0.40 mmol, 1.5 eq.), HOBT (54.6 mg, 0.40 mmol, 1.5 eq.), acetohydrazide (24 mg, 0.32 mmol) and DIEA (69.4 mg, 0.54 mmol). The mixture was stirred at 80° C. for 3 hours. Water (20 mL) was added, extracted with ethyl acetate (30 mL); the combined organic layers were washed with brine (10 mL), dried with over sodium sulfate, and concentrated under reduced pressure. The solid was filtered and dried to afford the title product (70 mg, 63% yield).

### Step 7: 2-(2,6-dimethoxy-4-(6-(1-methyl-1H-pyrazol-4-yl)benzofuran-3-yl)phenyl)-5-methyl-1,3,4-oxadiazole (compound 34)

To a solution of N′-acetyl-2,6-dimethoxy-4-(6-(1-methyl-1H-pyrazol-4-yl)benzofuran-3-yl)benzohydrazide (50 mg, 0.11 mmol) in toluene (2 mL) was added N,N-Diethyl-N-[[(methoxycarbonyl)amino]sulfonyl]ethanaminium inner salt (55 mg, 0.22 mmol). The mixture was stirred at 110° C. for 3 hours. Water (10 mL) was added, extracted with ethyl acetate (20 mL); the combined organic layers were washed with brine (10 mL), dried with over sodium sulfate, and concentrated under reduced pressure. The residue was purified by prep-HPLC to afford compound 34 (5 mg, 10% yield). ¹H NMR (500 MHz, DMSO-d₆) δ 8.53 (s, 1H), 8.25 (s, 1H), 7.99 (d, J=8.5 Hz, 2H), 7.91 (s, 1H), 7.61 (dd, J₁=8.2 Hz, J₂=1.3 Hz, 1H), 7.11 (s, 2H), 3.89 (s, 3H), 3.87 (s, 6H), 2.57 (s, 3H). LC-MS [M+H]⁺=417.3.

### Example 35: 2-cyclopropyl-5-(2,6-dimethoxy-4-(6-(1-methyl-1H-pyrazol-4-yl)benzofuran-3-yl)phenyl)-1,3,4-oxadiazole (compound 35)

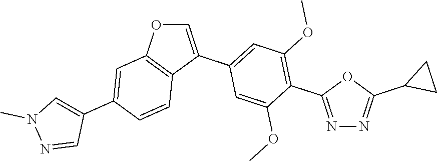 [SMILES: COc1cc(-c2coc3cc(-c4cnn(C)c4)ccc23)cc(OC)c1-c1nnc(C2CC2)o1]

### Step 1: N′-(cyclopropanecarbonyl)-2,6-dimethoxy-4-(6-(1-methyl-1H-pyrazol-4-yl)benzofuran-3-yl)benzohydrazide

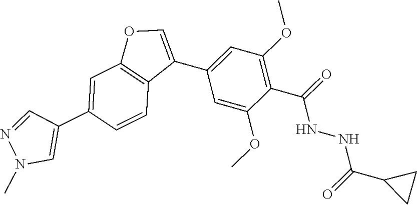 [SMILES: COc1cc(-c2coc3cc(-c4cnn(C)c4)ccc23)cc(OC)c1C(=O)NNC(=O)C1CC1]

The title compound of step 1 (80 mg, 66% yield) was prepared in a manner similar to that described in Example 34 step 6 from 2,6-dimethoxy-4-(6-(1-methyl-1H-pyrazol-4-yl)benzofuran-3-yl)benzoic acid and cyclopropanecarbohydrazide. LC-MS [M+H]⁺=461.5.

### Step 2: 2-cyclopropyl-5-(2,6-dimethoxy-4-(6-(1-methyl-1H-pyrazol-4-yl)benzofuran-3-yl)phenyl)-1,3,4-oxadiazole (compound 35)

Compound 35 (4 mg, 8% yield) was prepared in a manner similar to that described in Example 34 step 7 from N′-(cyclopropanecarbonyl)-2,6-dimethoxy-4-(6-(1-methyl-1H-pyrazol-4-yl)benzofuran-3-yl)benzohydrazide. ¹H NMR (500 MHz, DMSO-d₆) δ 8.52 (t. J=3.6 Hz, 1H), 8.34-8.13 (m, 1H), 7.99 (t, J=3.8 Hz, 2H), 7.91 (s, 1H), 7.67-7.59 (m, 1H), 7.10 (d, J=4.0 Hz, 2H), 3.89 (s, 3H), 3.88 (s, 3H), 3.87 (s, 3H), 2.29 (dt, J₁=12.6 Hz, J₂=4.2 Hz, 1H), 1.19-1.12 (m, 2H), 1.06-1.01 (m, 2H). LC-MS [M+H]⁺=443.4.

### Example 36: 2-(2,6-dimethoxy-4-(6-(1-methyl-1H-pyrazol-4-yl)benzofuran-3-yl)phenyl)-5-ethyl-1,3,4-oxadiazole (compound 36)

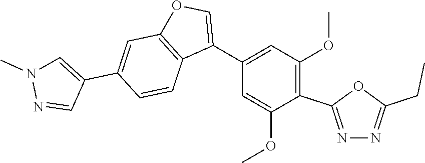 [SMILES: CCc1nnc(-c2c(OC)cc(-c3coc4cc(-c5cnn(C)c5)ccc34)cc2OC)o1]

### Step 1: 2,6-dimethoxy-4-(6-(1-methyl-1H-pyrazol-4-yl)benzofuran-3-yl)-N′-propionylbenzohydrazide

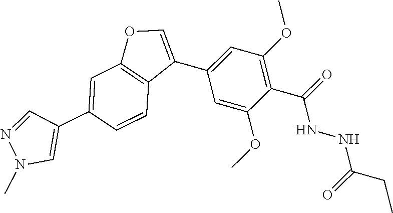 [SMILES: CCC(=O)NNC(=O)c1c(OC)cc(-c2coc3cc(-c4cnn(C)c4)ccc23)cc1OC]

The title compound of step 1 (90 mg, 94% yield) was prepared in a manner similar to that described in Example 34 step 6 from 2,6-dimethoxy-4-(6-(1-methyl-1H-pyrazol-4-yl)benzofuran-3-yl)benzoic acid and propionohydrazide. LC-MS [M+H]⁺=449.2.

### Step 2: 2-(2,6-dimethoxy-4-(6-(1-methyl-1H-pyrazol-4-yl)benzofuran-3-yl)phenyl)-5-ethyl-1,3,4-oxadiazole (compound 36)

The title compound of step 2 (4 mg, 8% yield) was prepared in a manner similar to that described in Example 34 step 7 from 2,6-dimethoxy-4-(6-(1-methyl-1H-pyrazol-4-yl)benzofuran-3-yl)-N′-propionylbenzohydrazide. ¹H NMR (500 MHz, DMSO-d₆) δ 8.53 (s, 1H), 8.24 (s, 1H), 7.91 (d, J=10.0 Hz, 2H), 7.84 (s, 1H), 7.54 (dd, J₁=8.2 Hz, J₂=1.2 Hz, 1H), 7.11 (s, 2H), 3.88 (s, 3H), 3.87 (s, 6H), 2.92 (q, J=7.5 Hz, 2H), 1.31 (t, J=7.5 Hz, 3H). LC-MS [M+H]⁺=431.4.

### Example 37: 2-(difluoromethoxy)-N-((1R,2S)-2-fluorocyclopropyl)-6-methoxy-4-(6-(1-(1-methylazetidin-3-yl)-1H-pyrazol-4-yl)benzofuran-3-yl)benzamide (compound 37)

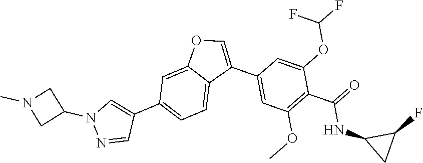 [SMILES: COc1cc(-c2coc3cc(-c4cnn(C5CN(C)C5)c4)ccc23)cc(OC(F)F)c1C(=O)N[C@@H]1C[C@@H]1F]

### Step 1:4-bromo-2-(difluoromethoxy)-6-methoxybenzoic acid

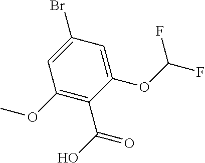 [SMILES: COc1cc(Br)cc(OC(F)F)c1C(=O)O]

The title compound of step 1 (888 mg, 100% yield) was prepared in a manner similar to that described in Example 25 step 8 from tert-butyl 4-bromo-2-(difluoromethoxy)-6-methoxybenzoate. LC-MS [M+H]⁺=297.0.

### Step 2: 4-bromo-2-(difluoromethoxy)-N-((1R,2S)-2-fluorocyclopropyl)-6-methoxybenzamide

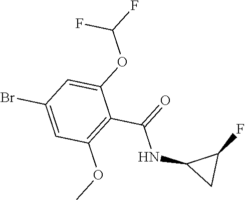 [SMILES: COc1cc(Br)cc(OC(F)F)c1C(=O)N[C@@H]1C[C@@H]1F]

The title compound of step 2 (1.0 g, 100% yield) was prepared in a manner similar to that described in Example 25 step 9 from 4-bromo-2-(difluoromethoxy)-6-methoxybenzoic acid and (1R,2S)-2-fluorocyclopropan-1-amine 4-methylbenzenesulfonate. LC-MS [M+H]⁺=354.1.

### Step 3: 3-(4,4,5,5-tetramethyl-1,3,2-dioxaborolan-2-yl)benzofuran-6-yl acetate

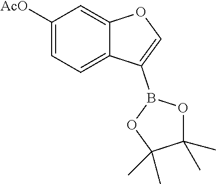 [SMILES: CC(=O)Oc1ccc2c(B3OC(C)(C)C(C)(C)O3)coc2c1]

The title compound of step 3 (1.1 g, 100% yield) was prepared in a manner similar to that described in Example 1 step 4 from 3-(((trifluoromethyl) sulfonyl)oxy)benzofuran-6-yl acetate and 4,4,4′,4′,5,5,5′,5′-octamethyl-2,2′-bi (1,3,2-dioxaborolane). LC-MS [M+H]⁺=303.0.

### Step 4: 3-(3-(difluoromethoxy)-4-(((1R,2S)-2-fluorocyclopropyl) carbamoyl)-5-methoxyphenyl)benzofuran-6-yl acetate

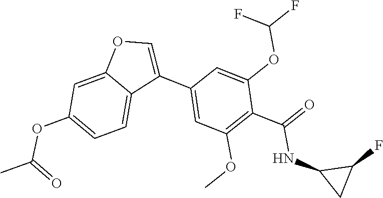 [SMILES: COc1cc(-c2coc3cc(OC(C)=O)ccc23)cc(OC(F)F)c1C(=O)N[C@@H]1C[C@@H]1F]

The title compound of step 4 (1.26 g, 85% yield) was prepared in a manner similar to that described in Example 1 step 6 from 3-(4,4,5,5-tetramethyl-1,3,2-dioxaborolan-2-yl)benzofuran-6-yl acetate and 4-bromo-2-(difluoromethoxy)-N-((1R,2S)-2-fluorocyclopropyl)-6-methoxybenzamide. LC-MS [M+H]⁺=450.0.

### Step 5: 2-(difluoromethoxy)-N-((1R,2S)-2-fluorocyclopropyl)-4-(6-hydroxybenzofuran-3-yl)-6-methoxybenzamide

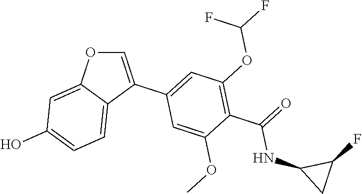 [SMILES: COc1cc(-c2coc3cc(O)ccc23)cc(OC(F)F)c1C(=O)N[C@@H]1C[C@@H]1F]

The title compound of step 5 (720 mg, 63% yield) was prepared in a manner similar to that described in Example 13 step 7 from 3-(3-(difluoromethoxy)-4-(((1R,2S)-2-fluorocyclopropyl) carbamoyl)-5-methoxyphenyl)benzofuran-6-yl acetate. LC-MS [M+H]⁺=408.0.

### Step 6: 3-(3-(difluoromethoxy)-4-(((1R,2S)-2-fluorocyclopropyl) carbamoyl)-5-methoxyphenyl)benzofuran-6-yl trifluoromethanesulfonate

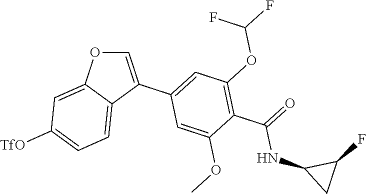 [SMILES: COc1cc(-c2coc3cc(OS(=O)(=O)C(F)(F)F)ccc23)cc(OC(F)F)c1C(=O)N[C@@H]1C[C@@H]1F]

The title compound of step 6 (900 mg, 94% yield) was prepared in a manner similar to that described in Example 13 step 8 from 2-(difluoromethoxy)-N-((1R,2S)-2-fluorocyclopropyl)-4-(6-hydroxybenzofuran-3-yl)-6-methoxybenzamide and triflic anhydride. LC-MS [M+H]⁺=540.4.

### Step 7: tert-butyl 3-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) azetidine-1-carboxylate

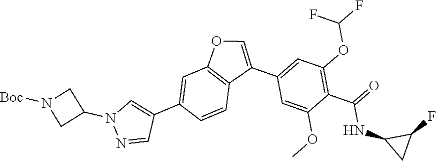 [SMILES: COc1cc(-c2coc3cc(-c4cnn(C5CN(C(=O)OC(C)(C)C)C5)c4)ccc23)cc(OC(F)F)c1C(=O)N[C@@H]1C[C@@H]1F]

The title compound of step 7 (61 mg, 50% yield) was prepared in a manner similar to that described in Example 1 step 5 from 3-(3-(difluoromethoxy)-4-(((1R,2S)-2-fluorocyclopropyl) carbamoyl)-5-methoxyphenyl)benzofuran-6-yl trifluoromethanesulfonate and tert-butyl 3-(4-(4,4,5,5-tetramethyl-1,3,2-dioxaborolan-2-yl)-1H-pyrazol-1-yl) azetidine-1-carboxylate. LC-MS [M+H]⁺=613.4.

### Step 8: 4-(6-(1-(azetidin-3-yl)-1H-pyrazol-4-yl)benzofuran-3-yl)-2-(difluoromethoxy)-N-((1R,2S)-2-fluorocyclopropyl)-6-methoxybenzamide

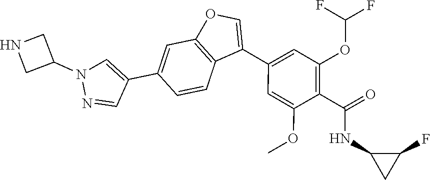 [SMILES: COc1cc(-c2coc3cc(-c4cnn(C5CNC5)c4)ccc23)cc(OC(F)F)c1C(=O)N[C@@H]1C[C@@H]1F]

The title compound of step 8 (51 mg, 100% yield) was prepared in a manner similar to that described in Example 28 step 2 from tert-butyl 3-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) azetidine-1-carboxylate. LC-MS [M+H]⁺=513.4.

### Step 9: 2-(difluoromethoxy)-N-((1R,2S)-2-fluorocyclopropyl)-6-methoxy-4-(6-(1-(1-methylazetidin-3-yl)-1H-pyrazol-4-yl)benzofuran-3-yl)benzamide (compound 37)

To a solution of 4-(6-(1-(azetidin-3-yl)-1H-pyrazol-4-yl)benzofuran-3-yl)-2-(difluoromethoxy)-N-((1R,2S)-2-fluorocyclopropyl)-6-methoxybenzamide (51 mg, 0.1 mmol) in MeOH (5 mL) and formaldehyde solution (37 wt. % in H₂O) cooled to 0° C. was added sodium triacetoxyborohydride (63 mg, 0.3 mmol) in portions. The mixture was stirred at room temperature for 1 h, diluted with water and extracted with EA (20 mL*3); the combined organic layers were washed with brine (20 mL), dried over anhydrous Na₂SO₄ and concentrated under reduced pressure. The residue was purified by Pre-HPLC to afford compound 37 (5 mg, 9% yield). ¹H NMR (500 MHz, DMSO-d₆) δ 8.51 (d, J=4.5 Hz, 1H), 8.47 (s, 1H), 8.46 (s, 1H), 8.07 (s, 1H), 7.95 (s, 1H), 7.88 (d. J=8.2 Hz, 1H), 7.66-7.64 (m, 1H), 7.26 (s, 1H), 7.22 (t, J=75.0 Hz, 1H), 7.12 (s, 1H), 4.99-4.93 (m, 1H), 4.86-4.56 (m, 1H), 3.90 (s, 3H), 3.73 (t, J=7.5 Hz, 2H), 3.41 (t, J=7.5 Hz, 2H), 2.87-2.83 (m, 1H), 2.35 (s, 3H), 1.14-1.06 (m, 1H), 0.99-0.85 (m, 1H). LC-MS [M+H]=527.4.

### Example 38: 2-(difluoromethoxy)-N-((1R,2S)-2-fluorocyclopropyl)-6-methoxy-4-(6-(1-(1-methylpyrrolidin-3-yl)-1H-pyrazol-4-yl)benzofuran-3-yl)benzamide (compound 38)

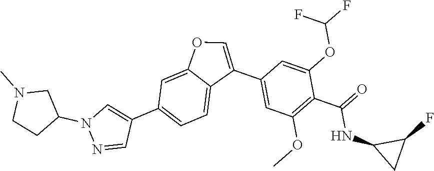 [SMILES: COc1cc(-c2coc3cc(-c4cnn(C5CCN(C)C5)c4)ccc23)cc(OC(F)F)c1C(=O)N[C@@H]1C[C@@H]1F]

### Step 1: tert-butyl 3-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) pyrrolidine-1-carboxylate

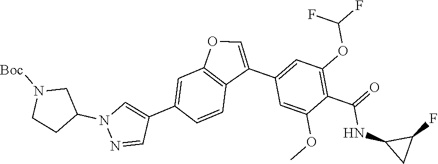 [SMILES: COc1cc(-c2coc3cc(-c4cnn(C5CCN(C(=O)OC(C)(C)C)C5)c4)ccc23)cc(OC(F)F)c1C(=O)N[C@@H]1C[C@@H]1F]

The title compound of step 1 (63 mg, 50% yield) was prepared in a manner similar to that described in Example 1 step 5 from 3-(3-(difluoromethoxy)-4-(((1R,2S)-2-fluorocyclopropyl) carbamoyl)-5-methoxyphenyl)benzofuran-6-yl trifluoromethanesulfonate and tert-butyl 3-(4-(4,4,5,5-tetramethyl-1,3,2-dioxaborolan-2-yl)-1H-pyrazol-1-yl) pyrrolidine-1-carboxylate. LC-MS [M+H]⁺=627.4.

### Step 2: 2-(difluoromethoxy)-N-((1R,2S)-2-fluorocyclopropyl)-6-methoxy-4-(6-(1-(pyrrolidin-3-yl)-1H-pyrazol-4-yl)benzofuran-3-yl)benzamide

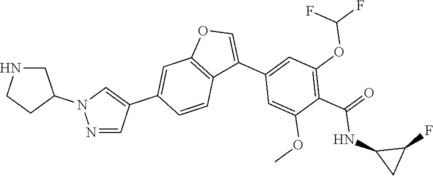 [SMILES: COc1cc(-c2coc3cc(-c4cnn(C5CCNC5)c4)ccc23)cc(OC(F)F)c1C(=O)N[C@@H]1C[C@@H]1F]

The title compound of step 2 (53 mg, 100% yield) was prepared in a manner similar to that described in Example 28 step 2 from tert-butyl 3-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) pyrrolidine-1-carboxylate. LC-MS [M+H]⁺=527.4.

### Step 3: 2-(difluoromethoxy)-N-((1R,2S)-2-fluorocyclopropyl)-6-methoxy-4-(6-(1-(1-methylpyrrolidin-3-yl)-1H-pyrazol-4-yl)benzofuran-3-yl)benzamide (compound 38)

Compound 38 (16 mg, 30% yield) was prepared in a manner similar to that described in Example 37 step 9 from 2-(difluoromethoxy)-N-((1R,2S)-2-fluorocyclopropyl)-6-methoxy-4-(6-(1-(pyrrolidin-3-yl)-1H-pyrazol-4-yl)benzofuran-3-yl)benzamide and formaldehyde solution. ¹H NMR (500 MHz, DMSO-d₆) δ 8.51 (d, J=4.4 Hz, 1H), 8.48 (s, 1H), 8.35 (s, 1H), 8.00 (s, 1H), 7.93 (s, 1H), 7.87 (d, J=8.2 Hz, 1H), 7.64 (d, J=8.2 Hz, 1H), 7.26 (s, 1H), 7.22 (t, J=75.0 Hz, 1H), 7.12 (s, 1H), 4.93-4.89 (m, 1H), 4.79-4.65 (m, 1H), 3.90 (s, 3H), 2.94-2.88 (m, 1H), 2.85-2.82 (m, 1H), 2.82-2.74 (m, 2H), 2.55-2.53 (m, 1H), 2.41-2.35 (m, 1H), 2.32 (s, 3H), 2.19-2.14 (m, 1H), 1.12-1.06 (m, 1H), 1.01-0.83 (m, 1H). LC-MS [M+H]⁺=541.4.

### Example 39: 2-(difluoromethoxy)-N-((1R,2S)-2-fluorocyclopropyl)-6-methoxy-4-(6-(1-(1-methylpiperidin-4-yl)-1H-pyrazol-4-yl)benzofuran-3-yl)benzamide (compound 39)

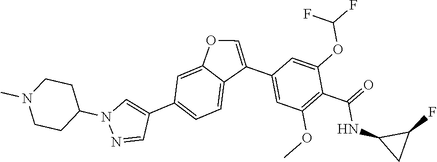 [SMILES: COc1cc(-c2coc3cc(-c4cnn(C5CCN(C)CC5)c4)ccc23)cc(OC(F)F)c1C(=O)N[C@@H]1C[C@@H]1F]

### Step 1: tert-butyl 4-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) piperidine-1-carboxylate

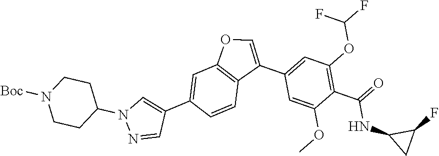 [SMILES: COc1cc(-c2coc3cc(-c4cnn(C5CCN(C(=O)OC(C)(C)C)CC5)c4)ccc23)cc(OC(F)F)c1C(=O)N[C@@H]1C[C@@H]1F]

The title compound of step 1 (64 mg, 100% yield) was prepared in a manner similar to that described in Example 1 step 5 from 3-(3-(difluoromethoxy)-4-(((1R,2S)-2-fluorocyclopropyl) carbamoyl)-5-methoxyphenyl)benzofuran-6-yl trifluoromethanesulfonate and tert-butyl 4-(4-(4,4,5,5-tetramethyl-1,3,2-dioxaborolan-2-yl)-1H-pyrazol-1-yl) piperidine-1-carboxylate. LC-MS [M+H]⁺=641.4.

### Step 2: 2-(difluoromethoxy)-N-((1R,2S)-2-fluorocyclopropyl)-6-methoxy-4-(6-(1-(piperidin-4-yl)-1H-pyrazol-4-yl)benzofuran-3-yl)benzamide

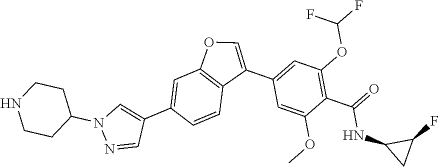 [SMILES: COc1cc(-c2coc3cc(-c4cnn(C5CCNCC5)c4)ccc23)cc(OC(F)F)c1C(=O)N[C@@H]1C[C@@H]1F]

The title compound of step 2 (54 mg, 100% yield) was prepared in a manner similar to that described in Example 28 step 2 from tert-butyl 4-(4-(3-(3-(difluoromethoxy)-4-(((1R,2S)-2-fluorocyclopropyl) carbamoyl)-5-methoxyphenyl)benzofuran-6-yl)-1H-pyrazol-1-yl) piperidine-1-carboxylate. LC-MS [M+H]⁺=541.4.

### Step 3: 2-(difluoromethoxy)-N-((1R,2S)-2-fluorocyclopropyl)-6-methoxy-4-(6-(1-(1-methylpiperidin-4-yl)-1H-pyrazol-4-yl)benzofuran-3-yl)benzamide (compound 39)

Compound 39 (16 mg, 30% yield) was prepared in a manner similar to that described in Example 37 step 9 from 2-(difluoromethoxy)-N-((1R,2S)-2-fluorocyclopropyl)-6-methoxy-4-(6-(1-(piperidin-4-yl)-1H-pyrazol-4-yl)benzofuran-3-yl)benzamide and formaldehyde solution. ¹H NMR (500 MHz, DMSO-d₆) δ 8.51 (d, J=4.5 Hz, 1H), 8.46 (s, 1H), 8.36 (s, 1H), 8.00 (s, 1H), 7.92 (s, 1H), 7.87 (d, J=8.2 Hz, 1H), 7.65-7.63 (m, 1H), 7.26 (s, 1H), 7.22 (t. J=75.0 Hz, 1H), 7.12 (s, 1H), 4.88-4.51 (m, 1H), 4.22-4.07 (m, 1H), 3.93-3.83 (m, 3H), 2.91-2.89 (m, 2H), 2.86-2.81 (m, 2H), 2.24 (s, 3H), 2.13-2.08 (m, 2H), 2.07-1.94 (m, 4H), 1.12-1.09 (m, 1H), 1.00-0.86 (m, 1H). LC-MS [M+H]⁺=555.4.

### Example 40: 8-(difluoromethoxy)-6-(6-(1-(tetrahydrofuran-3-yl)-1H-pyrazol-4-yl)benzofuran-3-yl)-2-(2,2,2-trifluoroethyl)-3,4-dihydroisoquinolin-1 (2H)-one (compound 40)

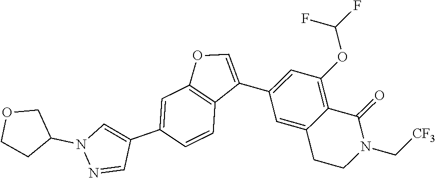 [SMILES: O=C1c2c(cc(-c3coc4cc(-c5cnn(C6CCOC6)c5)ccc34)cc2OC(F)F)CCN1CC(F)(F)F]

### Step 1: 6-(6-chlorobenzofuran-3-yl)-8-(difluoromethoxy)-2-(2,2,2-trifluoroethyl)-3,4-dihydroisoquinolin-1 (2H)-one

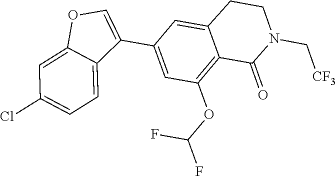 [SMILES: O=C1c2c(cc(-c3coc4cc(Cl)ccc34)cc2OC(F)F)CCN1CC(F)(F)F]

The tittle compound of step 1 (1.1 g, 67% yield) was prepared in a manner similar to that described in Example 1 step 5 from 6-chlorobenzofuran-3-yl trifluoromethanesulfonate and 8-(difluoromethoxy)-6-(4,4,5,5-tetramethyl-1,3,2-dioxaborolan-2-yl)-2-(2,2,2-trifluoroethyl)-3,4-dihydroisoquinolin-1 (2H)-one. LC-MS [M+H]⁺=445.3.

### Step 2: 8-(difluoromethoxy)-6-(6-(1-(tetrahydrofuran-3-yl)-1H-pyrazol-4-yl)benzofuran-3-yl)-2-(2,2,2-trifluoroethyl)-3,4-dihydroisoquinolin-1 (2H)-one (compound 40)

Compound 40 (4 mg, 6% yield) was prepared in a manner similar to that described in Example 1 step 6 from 6-(6-chlorobenzofuran-3-yl)-8-(difluoromethoxy)-2-(2,2,2-trifluoroethyl)-3,4-dihydroisoquinolin-1 (2H)-one and 1-(tetrahydrofuran-3-yl)-4-(4,4,5,5-tetramethyl-1,3,2-dioxaborolan-2-yl)-1H-pyrazole. ¹H NMR (500 MHz, DMSO-d₆) § 8.56 (s, 1H), 8.37 (s, 1H), 7.98 (s, 1H), 7.94 (d, J=8.2 Hz, 1H), 7.91 (s, 1H), 7.70 (s, 1H), 7.63 (d, J=8.2 Hz, 1H), 7.51 (s, 1H), 7.17 (t, J=75.4 Hz, 1H), 5.07-5.03 (m, 1H), 4.39 (q, J=9.5 Hz, 2H), 4.05-4.00 (m, 2H), 3.96-3.94 (m, 1H), 3.87-3.83 (m, 1H), 3.70 (t, J=6.1 Hz, 2H), 3.11 (t, J=6.0 Hz, 2H), 2.45-2.31 (m, 2H). LC-MS [M+H]⁺=548.3.

### Example 41: 8-(difluoromethoxy)-6-(6-(1-(tetrahydro-2H-pyran-4-yl)-1H-pyrazol-4-yl)benzofuran-3-yl)-2-(2,2,2-trifluoroethyl)-3,4-dihydroisoquinolin-1 (2H)-one (compound 41)

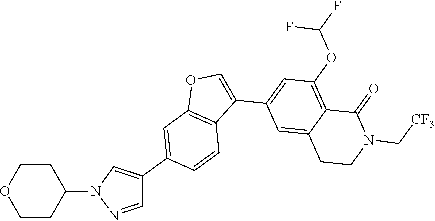 [SMILES: O=C1c2c(cc(-c3coc4cc(-c5cnn(C6CCOCC6)c5)ccc34)cc2OC(F)F)CCN1CC(F)(F)F]

### Step 1: 8-(difluoromethoxy)-6-(6-(1-(tetrahydro-2H-pyran-4-yl)-1H-pyrazol-4-yl)benzofuran-3-yl)-2-(2,2,2-trifluoroethyl)-3,4-dihydroisoquinolin-1 (2H)-one (compound 41)

Compound 41 (3 mg, 4 yield) was prepared in a manner similar to that described in Example 1 step 6 from 6-(6-chlorobenzofuran-3-yl)-8-(difluoromethoxy)-2-(2,2,2-trifluoroethyl)-3,4-dihydroisoquinolin-1 (2H)-one and 1-(tetrahydro-2H-pyran-4-yl)-4-(4,4,5,5-tetramethyl-1,3,2-dioxaborolan-2-yl)-1H-pyrazole. ¹H NMR (500 MHz, DMSO-d₆) § 8.56 (s, 1H), 8.39 (s, 1H), 8.02 (s, 1H), 7.95 (d, J=5.9 Hz, 2H), 7.70 (s, 1H), 7.66 (d, J=8.3 Hz, 1H), 7.51 (s, 1H), 7.17 (t, J=75.5 Hz, 1H), 4.48-4.33 (m, 3H), 3.99 (d, J=10.4 Hz, 2H), 3.71 (t, J=5.4 Hz, 2H), 3.50 (t, J=11.3 Hz, 2H), 3.12 (t, J=5.3 Hz, 2H), 2.09-1.92 (m, 4H). LC-MS [M+H]⁺=562.3.

### Example 42: 6-(6-(1-(azetidin-3-yl)-1H-pyrazol-4-yl)benzofuran-3-yl)-8-(difluoromethoxy)-2-(2,2,2-trifluoroethyl)-3,4-dihydroisoquinolin-1 (2H)-one (compound 42)

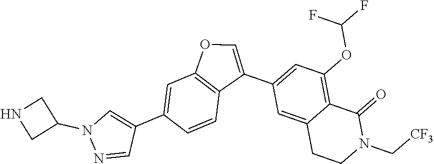 [SMILES: O=C1c2c(cc(-c3coc4cc(-c5cnn(C6CNC6)c5)ccc34)cc2OC(F)F)CCN1CC(F)(F)F]

### Step 1: tert-butyl 3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) azetidine-1-carboxylate

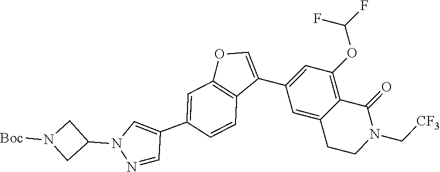 [SMILES: CC(C)(C)OC(=O)N1CC(n2cc(-c3ccc4c(-c5cc6c(c(OC(F)F)c5)C(=O)N(CC(F)(F)F)CC6)coc4c3)cn2)C1]

The title compound of step 1 (260 mg, 61% yield) was prepared in a manner similar to that described in Example 1 step 6 from 6-(6-chlorobenzofuran-3-yl)-8-(difluoromethoxy)-2-(2,2,2-trifluoroethyl)-3,4-dihydroisoquinolin-1 (2H)-one and tert-butyl 3-(4-(4,4,5,5-tetramethyl-1,3,2-dioxaborolan-2-yl)-1H-pyrazol-1-yl) azetidine-1-carboxylate. LC-MS [M+H]⁺=633.3.

### Step 2: 6-(6-(1-(azetidin-3-yl)-1H-pyrazol-4-yl)benzofuran-3-yl)-8-(difluoromethoxy)-2-(2,2,2-trifluoroethyl)-3,4-dihydroisoquinolin-1 (2H)-one (compound 42)

Compound 42 (1.7 mg, 10% yield) was prepared in a manner similar to that described in Example 28 step 2 from tert-butyl 3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) azetidine-1-carboxylate. ¹H NMR (500 MHz, DMSO-d₆) δ 8.57 (s, 1H), 8.46 (s, 1H), 8.26 (s, 1H), 8.12 (s, 1H), 7.98-7.90 (m, 2H), 7.70 (s, 1H), 7.66 (d, J=8.4 Hz, 1H), 7.51 (s, 1H), 7.17 (t, J=75.4 Hz, 1H), 5.29-5.18 (m, 1H), 4.39 (q. J=9.5 Hz, 2H), 4.06 (t. J=7.7 Hz, 2H), 3.92 (t, J=8.1 Hz, 2H), 3.70 (t, J=6.0 Hz, 2H), 3.11 (t, J=6.0 Hz, 2H). LC-MS [M+H]⁺=533.3.

### Example 43: 8-(difluoromethoxy)-6-(6-(1-(piperidin-4-yl)-1H-pyrazol-4-yl)benzofuran-3-yl)-2-(2,2,2-trifluoroethyl)-3,4-dihydroisoquinolin-1 (2H)-one (compound 43)

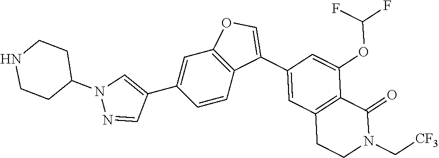 [SMILES: O=C1c2c(cc(-c3coc4cc(-c5cnn(C6CCNCC6)c5)ccc34)cc2OC(F)F)CCN1CC(F)(F)F]

### Step 1: tert-butyl 4-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) piperidine-1-carboxylate

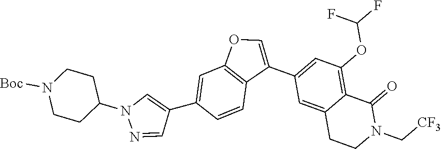 [SMILES: CC(C)(C)OC(=O)N1CCC(n2cc(-c3ccc4c(-c5cc6c(c(OC(F)F)c5)C(=O)N(CC(F)(F)F)CC6)coc4c3)cn2)CC1]

The title compound of step 1 (130 mg, 44% yield) was prepared in a manner similar to that described in Example 1 step 6 from 6-(6-chlorobenzofuran-3-yl)-8-(difluoromethoxy)-2-(2,2,2-trifluoroethyl)-3,4-dihydroisoquinolin-1 (2H)-one and tert-butyl 4-(4-(4,4,5,5-tetramethyl-1,3,2-dioxaborolan-2-yl)-1H-pyrazol-1-yl) piperidine-1-carboxylate. LC-MS [M+H]⁺=661.3.

### Step 2: 8-(difluoromethoxy)-6-(6-(1-(piperidin-4-yl)-1H-pyrazol-4-yl)benzofuran-3-yl)-2-(2,2,2-trifluoroethyl)-3,4-dihydroisoquinolin-1 (2H)-one (compound 43)

Compound 43 (3.5 mg, 20% yield) was prepared in a manner similar to that described in Example 28 step 2 from tert-butyl 4-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) piperidine-1-carboxylate. ¹H NMR (500 MHz, DMSO-d₆) δ 8.56 (s, 1H), 8.35 (s, 1H), 8.02 (s, 1H), 7.94 (d, J=6.7 Hz, 2H), 7.70 (s, 1H), 7.66 (d, J=8.3 Hz, 1H), 7.51 (s, 1H), 7.17 (t, J=75.4 Hz, 1H), 4.39 (q, J=9.5 Hz, 2H), 4.31 (t, J=11.2 Hz, 1H), 3.72-3.68 (m, 2H), 3.17 (d, J=12.1 Hz, 2H), 3.13-3.08 (m, 2H), 2.75 (t, J=11.4 Hz, 2H), 2.08 (d, J=12.0 Hz, 2H), 1.98-1.90 (m, 2H). LC-MS [M+H]⁺=561.3.

### Example 44: 8-(difluoromethoxy)-6-(6-(1-(1-methylazetidin-3-yl)-1H-pyrazol-4-yl)benzofuran-3-yl)-2-(2,2,2-trifluoroethyl)-3,4-dihydroisoquinolin-1 (2H)-one (compound 44)

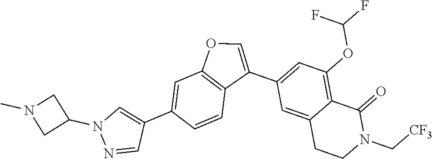 [SMILES: CN1CC(n2cc(-c3ccc4c(-c5cc6c(c(OC(F)F)c5)C(=O)N(CC(F)(F)F)CC6)coc4c3)cn2)C1]

### Step 1: 8-(difluoromethoxy)-6-(6-(1-(1-methylazetidin-3-yl)-1H-pyrazol-4-yl)benzofuran-3-yl)-2-(2,2,2-trifluoroethyl)-3,4-dihydroisoquinolin-1 (2H)-one (compound 44)

Compound 44 (2 mg, 6% yield) was prepared in a manner similar to that described in Example 37 step 9 from 6-(6-(1-(azetidin-3-yl)-1H-pyrazol-4-yl)benzofuran-3-yl)-8-(difluoromethoxy)-2-(2,2,2-trifluoroethyl)-3,4-dihydroisoquinolin-1 (2H)-one and formaldehyde solution. ¹H NMR (500 MHz, d-DMSO) δ 8.57 (s, 1H), 8.47 (s, 1H), 8.31 (s, 1H), 8.07 (s, 1H), 7.96 (s, 1H), 7.70 (s, 1H), 7.66 (d, J=9.4 Hz, 1H), 7.51 (s, 1H), 7.17 (t, J=75.4 Hz, 1H), 4.99-4.93 (m, 1H), 4.39 (q, J=9.7 Hz, 2H), 3.75-3.68 (m, 4H), 3.13-3.09 (m, 4H), 2.35 (s, 3H). LC-MS [M+H]⁺=547.3.

### Example 45: 3-(3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) azetidin-1-yl) propanenitrile (compound 45)

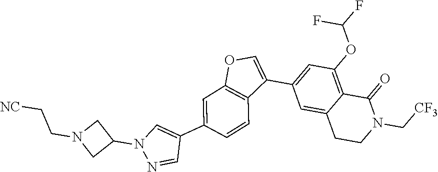 [SMILES: N#CCCN1CC(n2cc(-c3ccc4c(-c5cc6c(c(OC(F)F)c5)C(=O)N(CC(F)(F)F)CC6)coc4c3)cn2)C1]

### Step 1: 3-(3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) azetidin-1-yl) propanenitrile (compound 45)

A mixture of 6-(6-(1-(azetidin-3-yl)-1H-pyrazol-4-yl)benzofuran-3-yl)-8-(difluoromethoxy)-2-(2,2,2-trifluoroethyl)-3,4-dihydroisoquinolin-1 (2H)-one (30 mg, 0.056 mmol), DIPEA (21.7 mg, 0.168 mmol), 3-bromopropanenitrile (15 mg, 0.113 mmol) in THF (3 mL) was stirred for overnight at room temperature. It was diluted with water and EtOAc (15 mL*3); the combined organic layers were washed with brine, dried over anhydrous Na₂SO₄ and evaporated in vacuum. The residue was purified by prep-HPLC to afford compound 45 (3 mg, 9% yield). ¹H NMR (500 MHz, DMSO-d₆) δ 8.57 (s, 1H), 8.48 (s, 1H), 8.09 (s, 1H), 7.95 (t, J=3.9 Hz, 2H), 7.70 (s, 1H), 7.66 (d, J=8.3 Hz, 1H), 7.51 (s, 1H), 7.17 (t, J=75.4 Hz, 1H), 5.05-5.01 (m, 1H), 4.39 (q, J=9.4 Hz, 2H), 3.80 (t, J=7.5 Hz, 2H), 3.70 (t, J=5.9 Hz, 2H), 3.49 (t, J=7.4 Hz, 2H), 3.11 (t, J=5.8 Hz, 2H), 2.76 (t, J=6.5 Hz, 2H), 2.59 (t, J=6.5 Hz, 2H). LC-MS [M+H]⁺=586.3.

### Example 46: 8-(difluoromethoxy)-6-(6-(1-(1-methylpiperidin-4-yl)-1H-pyrazol-4-yl)benzofuran-3-yl)-2-(2,2,2-trifluoroethyl)-3,4-dihydroisoquinolin-1 (2H)-one (compound 46)

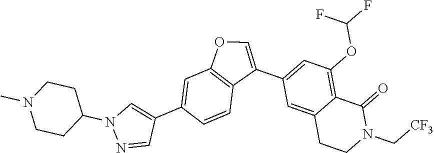 [SMILES: CN1CCC(n2cc(-c3ccc4c(-c5cc6c(c(OC(F)F)c5)C(=O)N(CC(F)(F)F)CC6)coc4c3)cn2)CC1]

### Step 1: 8-(difluoromethoxy)-6-(6-(1-(1-methylpiperidin-4-yl)-1H-pyrazol-4-yl)benzofuran-3-yl)-2-(2,2,2-trifluoroethyl)-3,4-dihydroisoquinolin-1 (2H)-one (compound 46)

Compound 46 (9 mg, 17% yield) was prepared in a manner similar to that described in Example 37 step 9 from 8-(difluoromethoxy)-6-(6-(1-(piperidin-4-yl)-1H-pyrazol-4-yl)benzofuran-3-yl)-2-(2,2,2-trifluoroethyl)-3,4-dihydroisoquinolin-1 (2H)-one and formaldehyde solution. ¹H NMR (500 MHz, DMSO-d₆) δ 8.56 (s, 1H), 8.37 (s, 1H), 8.01 (s, 1H), 7.94 (d, J=8.1 Hz, 2H), 7.70 (s, 1H), 7.65 (d, J=9.2 Hz, 1H), 7.50 (s, 1H), 7.17 (t, J=75.4 Hz, 1H), 4.39 (q. J=9.6 Hz, 2H), 4.19-4.09 (m, 1H), 3.70 (t, J=6.0 Hz, 2H), 3.11 (t, J=6.0 Hz, 2H), 2.88 (d, J=11.2 Hz, 2H), 2.22 (s, 3H), 2.14-1.96 (m, 6H). LC-MS [M+H]⁺=575.3.

### Example 47: 8-(difluoromethoxy)-6-(6-(1-(1-methylpyrrolidin-3-yl)-1H-pyrazol-4-yl)benzofuran-3-yl)-2-(2,2,2-trifluoroethyl)-3,4-dihydroisoquinolin-1 (2H)-one (compound 47)

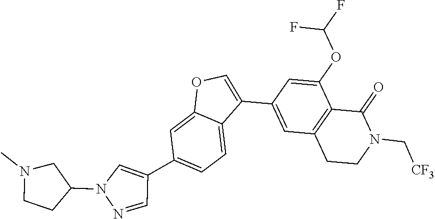 [SMILES: CN1CCC(n2cc(-c3ccc4c(-c5cc6c(c(OC(F)F)c5)C(=O)N(CC(F)(F)F)CC6)coc4c3)cn2)C1]

### Step 1: 6-(6-(1H-pyrazol-4-yl)benzofuran-3-yl)-8-(difluoromethoxy)-2-(2,2,2-trifluoroethyl)-3,4-dihydroisoquinolin-1 (2H)-one

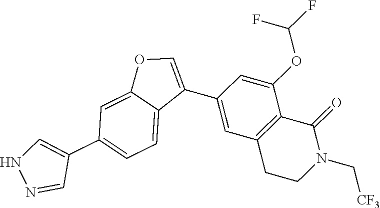 [SMILES: O=C1c2c(cc(-c3coc4cc(-c5cn[nH]c5)ccc34)cc2OC(F)F)CCN1CC(F)(F)F]

The title compound of step 1 (173 mg, 70% yield) was prepared in a manner similar to that described in Example 1 step 6 from 6-(6-chlorobenzofuran-3-yl)-8-(difluoromethoxy)-2-(2,2,2-trifluoroethyl)-3,4-dihydroisoquinolin-1 (2H)-one and 4-(4,4,5,5-tetramethyl-1,3,2-dioxaborolan-2-yl)-1H-pyrazole. LC-MS [M+H]⁺=478.2.

### Step 2: 1-methylpyrrolidin-3-yl methanesulfonate

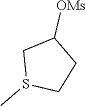

To the mixture of 1-methylpyrrolidin-3-ol (470 mg, 4.65 mmol) and Et₃N (1.17 g, 11.63 mmol, 2.5 eq) in DCM (20 mL) was added methanesulfonyl chloride (799 mg, 6.98 mmol) dropwise at 0° C. under N₂, the resulting solution was stirred at r.t for 2 hours. Water (10 mL) was added, extracted with DCM (10 mL*3); the combined organic layers were dried over Na₂SO₄, filtered and concentrated in vacuo. The residue was purified by silica gel column chromatography, eluting with DCM/MeOH (v/v=15/1), to afford the title product (618 mg, 74% yield). LC-MS [M+H]⁺=180.2.

### Step 3: 8-(difluoromethoxy)-6-(6-(1-(1-methylpyrrolidin-3-yl)-1H-pyrazol-4-yl)benzofuran-3-yl)-2-(2,2,2-trifluoroethyl)-3,4-dihydroisoquinolin-1 (2H)-one (compound 47)

A solution of 6-(6-(1H-pyrazol-4-yl)benzofuran-3-yl)-8-(difluoromethoxy)-2-(2,2,2-trifluoroethyl)-3,4-dihydroisoquinolin-1 (2H)-one (66 mg, 0.138 mmol) in DMF (5 mL) was added NaH (60% dispersion in mineral oil, 11 mg, 0.276 mmol) at 0° C. The mixture was stirred at 0° C. for 20 min, then 1-methylpyrrolidin-3-yl methanesulfonate (37 mg, 0.207 mmol) in DMF (1 mL) was added dropwise, the resulting solution was stirred at r.t for 12 hours. the solution was quenched by water (5 mL), extracted with EA (5 mL*3); the combined organic layers were dried over Na₂SO₄, filtered, and concentrated, the residue was purified by Prep-HPLC to give compound 47 (14 mg, 18% yield). ¹H NMR (500 MHz, DMSO-d₆) § 8.56 (s, 1H), 8.36 (s, 1H), 8.00 (s, 1H), 7.96-7.90 (m, 2H), 7.70 (s, 1H), 7.67-7.63 (m, 1H), 7.50 (s, 1H), 7.17 (t, J=75.0 Hz, 1H), 4.98-4.87 (m, 1H), 4.39 (q, J=9.6 Hz, 2H), 3.70 (t, J=6.1 Hz, 2H), 3.11 (t, J=5.9 Hz, 2H), 2.94-2.85 (m, 1H), 2.83-2.72 (m, 2H), 2.57-2.52 (m, 1H), 2.43-2.35 (m, 1H), 2.32 (s, 3H), 2.21-2.10 (m, 1H). LC-MS [M+H]⁺=561.4.

### Example 48: 8-(difluoromethoxy)-6-(6-(1-(pyrrolidin-3-yl)-1H-pyrazol-4-yl)benzofuran-3-yl)-2-(2,2,2-trifluoroethyl)-3,4-dihydroisoquinolin-1 (2H)-one (compound 48)

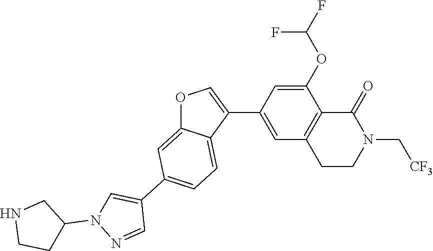 [SMILES: O=C1c2c(cc(-c3coc4cc(-c5cnn(C6CCNC6)c5)ccc34)cc2OC(F)F)CCN1CC(F)(F)F]

### Step 1: tert-butyl 3-((methylsulfonyl)oxy) pyrrolidine-1-carboxylate

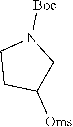

The title compound of step 1 (986 mg, 94% yield) was prepared in a manner similar to that described in Example 47 step 2 from tert-butyl 3-hydroxypyrrolidine-1-carboxylate and MsCl. LC-MS [M+H]⁺=266.4.

### Step 2: tert-butyl 3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) pyrrolidine-1-carboxylate

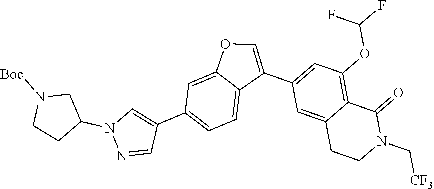 [SMILES: CC(C)(C)OC(=O)N1CCC(n2cc(-c3ccc4c(-c5cc6c(c(OC(F)F)c5)C(=O)N(CC(F)(F)F)CC6)coc4c3)cn2)C1]

The title compound of step 2 (30 mg, 23% yield) was prepared in a manner similar to that described in Example 47 step 3 from 6-(6-(1H-pyrazol-4-yl)benzofuran-3-yl)-8-(difluoromethoxy)-2-(2,2,2-trifluoroethyl)-3,4-dihydroisoquinolin-1 (2H)-one and tert-butyl 3-((methylsulfonyl)oxy) pyrrolidine-1-carboxylate. LC-MS [M+H]⁺=647.4.

### Step 3: 8-(difluoromethoxy)-6-(6-(1-(pyrrolidin-3-yl)-1H-pyrazol-4-yl)benzofuran-3-yl)-2-(2,2,2-trifluoroethyl)-3,4-dihydroisoquinolin-1 (2H)-one (compound 48)

Compound 48 (6 mg, 23% yield) was prepared in a manner similar to that descried in Example 28 step 2 from tert-butyl 3-(4-(3-(8-(difluoromethoxy)-1-oxo-2-(2,2,2-trifluoroethyl)-1,2,3,4-tetrahydroisoquinolin-6-yl)benzofuran-6-yl)-1H-pyrazol-1-yl) pyrrolidine-1-carboxylate. ¹H NMR (500 MHz, DMSO-d₆) δ 8.56 (s, 1H), 8.38 (s, 1H), 8.26 (s, 1H), 8.03 (s, 1H), 7.98-7.91 (m, 2H), 7.70 (s, 1H), 7.67-7.63 (m, 1H), 7.50 (s, 1H), 7.17 (t. J=75.0 Hz, 1H), 4.94-4.84 (m, 1H), 4.39 (q, J=9.5 Hz, 2H), 3.70 (t, J=6.1 Hz, 2H), 3.28-3.23 (m, 1H), 3.16-3.07 (m, 4H), 3.01-2.91 (m, 1H), 2.31-2.20 (m, 1H), 2.17-2.07 (m, 1H). LC-MS [M+H]⁺=547.4.

### Example 49: 8-methoxy-6-(6-(1-(tetrahydrofuran-3-yl)-1H-pyrazol-4-yl)benzofuran-3-yl)-2-(2,2,2-trifluoroethyl)-3,4-dihydroisoquinolin-1 (2H)-one (compound 49)

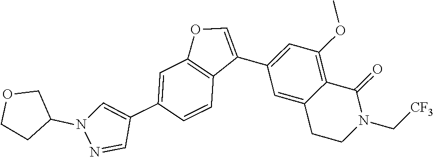 [SMILES: COc1cc(-c2coc3cc(-c4cnn(C5CCOC5)c4)ccc23)cc2c1C(=O)N(CC(F)(F)F)CC2]

### Step 1: 8-methoxy-6-(6-(1-(tetrahydrofuran-3-yl)-1H-pyrazol-4-yl)benzofuran-3-yl)-2-(2,2,2-trifluoroethyl)-3,4-dihydroisoquinolin-1 (2H)-one (compound 49)

Compound 49 (15 mg, 30% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and 1-(tetrahydrofuran-3-yl)-4-(4,4,5,5-tetramethyl-1,3,2-dioxaborolan-2-yl)-1H-pyrazole. ¹H NMR (500 MHz, DMSO-d₆) δ 8.50 (s, 1H), 8.36 (s, 1H), 8.04 (s, 1H), 7.95 (dd, J₁=15.6 Hz, J₂=4.6 Hz, 2H), 7.64 (dd, J₁=8.2 Hz, J₂=1.3 Hz, 1H), 7.29 (d, J=12.2 Hz, 2H), 5.07-5.03 (m, 1H), 4.35 (q, J=9.6 Hz, 2H), 4.05-4.00 (m, 2H), 3.97-3.94 (m, 1H), 3.91 (s, 3H), 3.88-3.83 (m, 1H), 3.64 (t, J=6.1 Hz, 2H), 3.01 (t, J=6.1 Hz, 2H), 2.47-2.31 (m, 2H). LC-MS [M+H]⁺=512.3.

### Example 50: 8-methoxy-6-(6-(1-(tetrahydro-2H-pyran-4-yl)-1H-pyrazol-4-yl)benzofuran-3-yl)-2-(2,2,2-trifluoroethyl)-3,4-dihydroisoquinolin-1 (2H)-one (compound 50)

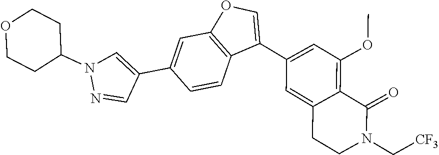 [SMILES: COc1cc(-c2coc3cc(-c4cnn(C5CCOCC5)c4)ccc23)cc2c1C(=O)N(CC(F)(F)F)CC2]

### Step 1: 8-methoxy-6-(6-(1-(tetrahydro-2H-pyran-4-yl)-1H-pyrazol-4-yl)benzofuran-3-yl)-2-(2,2,2-trifluoroethyl)-3,4-dihydroisoquinolin-1 (2H)-one (compound 50)

Compound 50 (17 mg, 33% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and 1-(tetrahydro-2H-pyran-4-yl)-4-(4,4,5,5-tetramethyl-1,3,2-dioxaborolan-2-yl)-1H-pyrazole. ¹H NMR (500 MHz, DMSO-d₆) δ 8.50 (s, 1H), 8.38 (s, 1H), 8.02 (s, 1H), 7.97 (d, J=8.2 Hz, 1H), 7.93 (s, 1H), 7.63 (dd, J₁=8.2 Hz, J₂=1.1 Hz, 1H), 7.29 (d, J=12.5 Hz, 2H), 4.48-4.39 (m, 1H), 4.35 (q, J=9.6 Hz, 2H), 3.99 (dd, J₁=10.8 Hz, J₂=2.5 Hz, 2H), 3.91 (s, 3H), 3.64 (t, J=6.0 Hz, 2H), 3.50 (td, J₁=11.5 Hz, J₂=2.6 Hz, 2H), 3.01 (t, J=6.0 Hz, 2H), 2.09-1.94 (m, 4H). LC-MS [M+H]⁺=526.3.

### Example 51: 8-methoxy-6-(6-(1-(1-methylpyrrolidin-3-yl)-1H-pyrazol-4-yl)benzofuran-3-yl)-2-(2,2,2-trifluoroethyl)-3,4-dihydroisoquinolin-1 (2H)-one (compound 51)

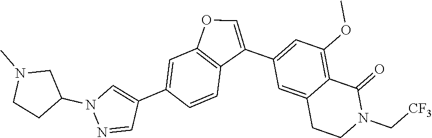 [SMILES: COc1cc(-c2coc3cc(-c4cnn(C5CCN(C)C5)c4)ccc23)cc2c1C(=O)N(CC(F)(F)F)CC2]

### Step 1: 6-(6-(1H-pyrazol-4-yl)benzofuran-3-yl)-8-methoxy-2-(2,2,2-trifluoroethyl)-3,4-dihydroisoquinolin-1 (2H)-one

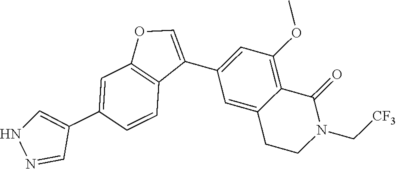 [SMILES: COc1cc(-c2coc3cc(-c4cn[nH]c4)ccc23)cc2c1C(=O)N(CC(F)(F)F)CC2]

The title compound of step 1 (60 mg, 79% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and 4-(4,4,5,5-tetramethyl-1,3,2-dioxaborolan-2-yl)-1H-pyrazole. LC-MS [M+H]⁺=442.3.

### Step 2: 8-methoxy-6-(6-(1-(1-methylpyrrolidin-3-yl)-1H-pyrazol-4-yl)benzofuran-3-yl)-2-(2,2,2-trifluoroethyl)-3,4-dihydroisoquinolin-1 (2H)-one (compound 51)

Compound 51 (1 mg, 1.5% yield) was prepared in a manner similar to that described in Example 47 step 3 from 6-(6-(1H-pyrazol-4-yl)benzofuran-3-yl)-8-methoxy-2-(2,2,2-trifluoroethyl)-3,4-dihydroisoquinolin-1 (2H)-one and 1-methylpyrrolidin-3-yl methanesulfonate. ¹H NMR (500 MHz, DMSO-d₆) δ 8.50 (s, 1H), 8.34 (d, J=8.5 Hz, 1H), 7.99-7.93 (m, 3H), 7.62 (d, J=8.1 Hz, 1H), 7.29 (d, J=12.4 Hz, 2H), 5.34-5.30 (m, 1H), 4.93-4.90 (m, 1H), 4.35 (d, J=8.9 Hz, 2H), 3.90 (s, 3H), 3.63 (s, 3H), 3.03-3.00 (m, 2H), 2.31 (s, 3H), 2.16 (s, 1H), 1.99 (s, 3H). LC-MS [M+H]⁺=525.3.

### Example 52: 8-methoxy-6-(6-(1-(1-methylpiperidin-4-yl)-1H-pyrazol-4-yl)benzofuran-3-yl)-2-(2,2,2-trifluoroethyl)-3,4-dihydroisoquinolin-1 (2H)-one (compound 52)

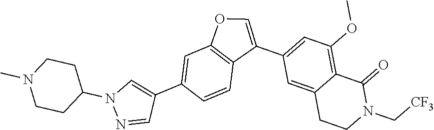 [SMILES: COc1cc(-c2coc3cc(-c4cnn(C5CCN(C)CC5)c4)ccc23)cc2c1C(=O)N(CC(F)(F)F)CC2]

### Step 1: tert-butyl 4-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) piperidine-1-carboxylate

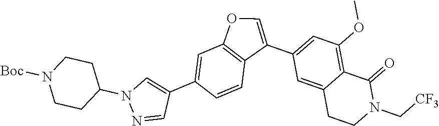 [SMILES: COc1cc(-c2coc3cc(-c4cnn(C5CCN(C(=O)OC(C)(C)C)CC5)c4)ccc23)cc2c1C(=O)N(CC(F)(F)F)CC2]

The title compound of step 1 (70 mg, 65% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and tert-butyl 4-(4-(4,4,5,5-tetramethyl-1,3,2-dioxaborolan-2-yl)-1H-pyrazol-1-yl) piperidine-1-carboxylate. LC-MS [M+H]⁺=625.4.

### Step 2: 8-methoxy-6-(6-(1-(piperidin-4-yl)-1H-pyrazol-4-yl)benzofuran-3-yl)-2-(2,2,2-trifluoroethyl)-3,4-dihydroisoquinolin-1 (2H)-one

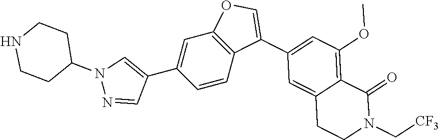 [SMILES: COc1cc(-c2coc3cc(-c4cnn(C5CCNCC5)c4)ccc23)cc2c1C(=O)N(CC(F)(F)F)CC2]

The title compound of step 2 (40 mg, 68% yield) was prepared in a manner similar to that described in Example 28 step 2 from tert-butyl 4-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) piperidine-1-carboxylate. LC-MS [M+H]⁺=525.3.

### Step 3: 8-methoxy-6-(6-(1-(1-methylpiperidin-4-yl)-1H-pyrazol-4-yl)benzofuran-3-yl)-2-(2,2,2-trifluoroethyl)-3,4-dihydroisoquinolin-1 (2H)-one (compound 52)

Compound 52 (35 mg, 85% yield) was prepared in a manner similar to that described in Example 37 step 9 from 8-methoxy-6-(6-(1-(piperidin-4-yl)-1H-pyrazol-4-yl)benzofuran-3-yl)-2-(2,2,2-trifluoroethyl)-3,4-dihydroisoquinolin-1 (2H)-one and formaldehyde solution. ¹H NMR (500 MHz, DMSO-d₆) δ 8.50 (s, 1H), 8.36 (s, 1H), 8.01 (s, 1H), 7.97 (d, J=8.2 Hz, 1H), 7.92 (s, 1H), 7.63 (dd, J₁=8.2 Hz, J₂=1.1 Hz, 1H), 7.29 (d, J=12.8 Hz, 2H), 4.35 (q, J=9.6 Hz, 2H), 4.16-4.10 (m, 1H), 3.91 (s, 3H), 3.64 (t, J=6.0 Hz, 2H), 3.01 (t, J=6.0 Hz, 2H), 2.93 (d, J=11.5 Hz, 2H), 2.27 (s, 3H), 2.19-2.13 (m, 2H), 2.11-1.95 (m, 4H). LC-MS [M+H]⁺=539.4.

### Example 53: 8-methoxy-6-(6-(1-(1-methylazetidin-3-yl)-1H-pyrazol-4-yl)benzofuran-3-yl)-2-(2,2,2-trifluoroethyl)-3,4-dihydroisoquinolin-1 (2H)-one (compound 53)

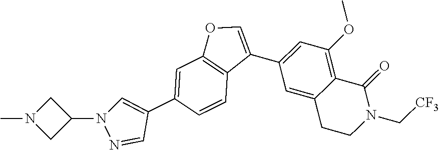 [SMILES: COc1cc(-c2coc3cc(-c4cnn(C5CN(C)C5)c4)ccc23)cc2c1C(=O)N(CC(F)(F)F)CC2]

### Step 1: tert-butyl 3-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) azetidine-1-carboxylate

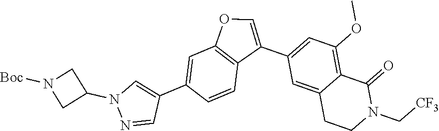 [SMILES: COc1cc(-c2coc3cc(-c4cnn(C5CN(C(=O)OC(C)(C)C)C5)c4)ccc23)cc2c1C(=O)N(CC(F)(F)F)CC2]

The title compound of step 1 (160 mg, 70% yield) was prepared in a manner similar to that described in Example 1 step 5 from 3-(8-methoxy-1-oxo-2-(2,2,2-trifluoroethyl)-1,2,3,4-tetrahydroisoquinolin-6-yl)benzofuran-6-yl trifluoromethanesulfonate and tert-butyl 3-(4-(4,4,5,5-tetramethyl-1,3,2-dioxaborolan-2-yl)-1H-pyrazol-1-yl) azetidine-1-carboxylate. LC-MS [M+H]⁺=597.2.

### Step 2: 6-(6-(1-(azetidin-3-yl)-1H-pyrazol-4-yl)benzofuran-3-yl)-8-methoxy-2-(2,2,2-trifluoroethyl)-3,4-dihydroisoquinolin-1 (2H)-one

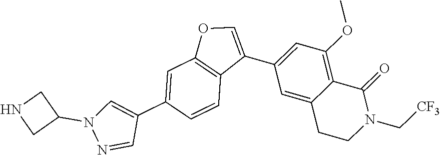 [SMILES: COc1cc(-c2coc3cc(-c4cnn(C5CNC5)c4)ccc23)cc2c1C(=O)N(CC(F)(F)F)CC2]

The title compound of step 2 (100 mg, 75% yield) was prepared in a manner similar to that described in Example 28 step 2 from tert-butyl 3-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) azetidine-1-carboxylate. LC-MS [M+H]⁺=497.3.

### Step 3: 8-methoxy-6-(6-(1-(1-methylazetidin-3-yl)-1H-pyrazol-4-yl)benzofuran-3-yl)-2-(2,2,2-trifluoroethyl)-3,4-dihydroisoquinolin-1 (2H)-one (compound 53)

Compound 53 (3 mg, 6% yield) was prepared in a manner similar to that described in Example 37 step 9 from 6-(6-(1-(azetidin-3-yl)-1H-pyrazol-4-yl)benzofuran-3-yl)-8-methoxy-2-(2,2,2-trifluoroethyl)-3,4-dihydroisoquinolin-1 (2H)-one and formaldehyde solution. ¹H NMR (500 MHz, DMSO-d₆) δ 8.51 (s, 1H), 8.46 (s, 1H), 8.06 (s, 1H), 8.01-7.91 (m, 2H), 7.64 (dd, J₁=8.2 Hz, J₂=1.1 Hz, 1H), 7.29 (d, J=11.9 Hz, 2H), 4.98-4.94 (m, 1H), 4.35 (q, J=9.7 Hz, 2H), 3.91 (s, 3H), 3.73 (t, J=7.5 Hz, 2H), 3.64 (t. J=6.0 Hz, 2H), 3.35 (t, J=7.5 Hz, 2H), 3.01 (t, J=6.0 Hz, 2H), 2.35 (s, 3H). LC-MS [M+H]⁺=511.3.

### Example 54: 3-(3-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) azetidin-1-yl) propanenitrile (compound 54)

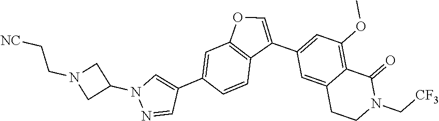 [SMILES: COc1cc(-c2coc3cc(-c4cnn(C5CN(CCC#N)C5)c4)ccc23)cc2c1C(=O)N(CC(F)(F)F)CC2]

### Step 1: 3-(3-(4-(3-(8-methoxy-1-oxo-2-(2,2,2-trifluoroethyl)-1,2,3,4-tetrahydroisoquinolin-6-yl)benzofuran-6-yl)-1H-pyrazol-1-yl) azetidin-1-yl) propanenitrile (compound 54)

Compound 54 (2 mg, 4% yield) was prepared in a manner similar to that described in Example 45 step 1 from 6-(6-(1-(azetidin-3-yl)-1H-pyrazol-4-yl)benzofuran-3-yl)-8-methoxy-2-(2,2,2-trifluoroethyl)-3,4-dihydroisoquinolin-1 (2H)-one and 3-bromopropanenitrile. ¹H NMR (500 MHz, DMSO-d₆) § 8.51 (s, 1H), 8.47 (s, 1H), 8.08 (s, 1H), 7.98 (d, J=8.2 Hz, 1H), 7.94 (d, J=0.9 Hz, 1H), 7.63 (dd, J₁=8.2 Hz, J₂=1.3 Hz, 1H), 7.29 (d, J=11.3 Hz, 2H), 5.05-5.00 (m, 1H), 4.35 (q, J=9.7 Hz, 2H), 3.91 (s, 3H), 3.80 (t, J=7.6 Hz, 2H), 3.63 (t, J=6.1 Hz, 2H), 3.49 (t, J=8.0 Hz, 2H), 3.01 (t, J=6.0 Hz, 2H), 2.76 (t, J=6.5 Hz, 2H), 2.59 (t. J=6.5 Hz, 2H). LC-MS [M+H]⁺=550.3.

### Example 55: 2-(8-(difluoromethoxy)-6-(6-(1-(1-methylpiperidin-4-yl)-1H-pyrazol-4-yl)benzofuran-3-yl)-1-oxo-3,4-dihydroisoquinolin-2 (1H)-yl) acetonitrile (compound 55)

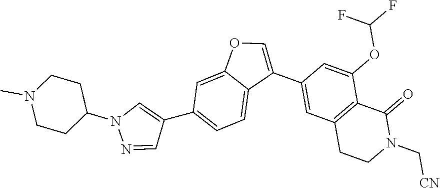 [SMILES: CN1CCC(n2cc(-c3ccc4c(-c5cc6c(c(OC(F)F)c5)C(=O)N(CC#N)CC6)coc4c3)cn2)CC1]

### Step 1: 1-methylpiperidin-4-yl methanesulfonate

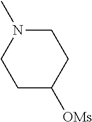

The title compound of step 1 (105 mg, 13% yield) was prepared in a manner similar to that described in Example 47 step 2 from 1-methylpiperidin-4-ol and MsCl. LC-MS [M+H]⁺=194.3.

### Step 2: 4-(4-iodo-1H-pyrazol-1-yl)-1-methylpiperidine

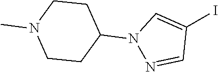

A solution of 4-iodo-1H-pyrazole (105 mg, 0.543 mmol) and 1-methylpiperidin-4-yl methanesulfonate (105 mg, 0.543 mmol) in DMF (5 mL) was added Cs₂CO₃ (353 mg, 1.09 mmol), the resulting solution was stirred at 100° C. for 12 hours. The mixture was added water (10 mL), extracted with EtOAc (10 mL*3); the combined organic layers were dried over Na₂SO₄, filtered and concentrated in vacuo, the residue was purified by silica gel column chromatography, eluting with PE/EtOAc (v/v=1/1), to give the title product (90 mg, 60% yield). LC-MS [M+H]⁺=292.1.

### Step 3: 2-(8-(difluoromethoxy)-6-(6-(1-(1-methylpiperidin-4-yl)-1H-pyrazol-4-yl)benzofuran-3-yl)-1-oxo-3,4-dihydroisoquinolin-2 (1H)-yl) acetonitrile (compound 55)

Compound 55 (4 mg, 14% yield) was prepared in a manner similar to that described in Example 1 step 6 from 4-(4-iodo-1H-pyrazol-1-yl)-1-methylpiperidine and 2-(8-(difluoromethoxy)-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile. ¹H NMR (500 MHz, DMSO-d₆) δ 8.56 (s, 1H), 8.37 (s, 1H), 8.01 (s, 1H), 7.97-7.90 (m, 2H), 7.70 (s, 1H), 7.65 (d, J=8.1 Hz, 1H), 7.50 (s, 1H), 7.28 (t, J=75.0 Hz, 1H), 4.62 (s, 2H), 4.18-4.08 (m, 1H), 3.70 (t, J=6.3 Hz, 2H), 3.15 (t, J=6.2 Hz, 2H), 2.93-2.83 (m, 2H), 2.23 (s, 3H), 2.13-1.94 (m, 6H). LC-MS [M+H]⁺=532.4.

### Example 56: 2-(8-(difluoromethoxy)-6-(6-(1-(1-methylpyrrolidin-3-yl)-1H-pyrazol-4-yl)benzofuran-3-yl)-1-oxo-3,4-dihydroisoquinolin-2 (1H)-yl) acetonitrile (compound 56)

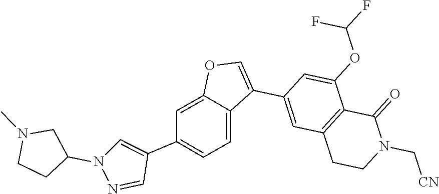 [SMILES: CN1CCC(n2cc(-c3ccc4c(-c5cc6c(c(OC(F)F)c5)C(=O)N(CC#N)CC6)coc4c3)cn2)C1]

### Step 1: 4-iodo-1-(1-methylpyrrolidin-3-yl)-1H-pyrazole

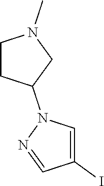

The title compound of step 1 (100 mg, 26% yield) was prepared in a manner similar to that described in Example 55 step 2 from 4-iodo-1H-pyrazole and 1-methylpyrrolidin-3-yl methanesulfonate. LC-MS (M+H)+=278.1.

### Step 2: 2-(8-(difluoromethoxy)-6-(6-(1-(1-methylpyrrolidin-3-yl)-1H-pyrazol-4-yl)benzofuran-3-yl)-1-oxo-3,4-dihydroisoquinolin-2 (1H)-yl) acetonitrile (compound 56)

Compound 56 (5 mg, 9% yield) was prepared in a manner similar to that described in Example 1 step 6 from 4-iodo-1-(1-methylpyrrolidin-3-yl)-1H-pyrazole and 2-(8-(difluoromethoxy)-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile. ¹H NMR (500 MHz, DMSO-d₆) δ 8.56 (s, 1H), 8.36 (s, 1H), 8.00 (s, 1H), 7.97-7.90 (m, 2H), 7.70 (s, 1H), 7.68-7.62 (m, 1H), 7.50 (s, 1H), 7.18 (t, J=75.0 Hz, 1H), 4.96-4.86 (m, 1H), 4.62 (s, 2H), 3.70 (t, J=6.3 Hz, 2H), 3.15 (t, J=6.2 Hz, 2H), 2.93-2.85 (m, 1H), 2.82-2.74 (m, 2H), 2.57-2.53 (m, 1H), 2.44-2.35 (m, 1H), 2.31 (s, 3H), 2.21-2.10 (m, 1H). LC-MS [M+H]⁺=518.3.

### Example 57: 2-(8-(difluoromethoxy)-6-(6-(1-(1-methylazetidin-3-yl)-1H-pyrazol-4-yl)benzofuran-3-yl)-1-oxo-3,4-dihydroisoquinolin-2 (1H)-yl) acetonitrile (compound 57)

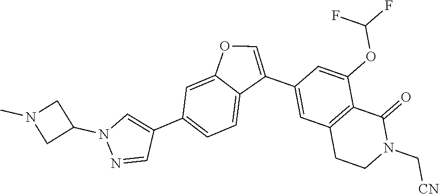 [SMILES: CN1CC(n2cc(-c3ccc4c(-c5cc6c(c(OC(F)F)c5)C(=O)N(CC#N)CC6)coc4c3)cn2)C1]

### Step 1: tert-butyl 3-(4-iodo-1H-pyrazol-1-yl)azetidine-1-carboxylate

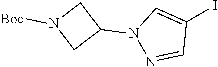 [SMILES: CC(C)(C)OC(=O)N1CC(n2cc(I)cn2)C1]

The title compound of step 1 (790 mg, 100% yield) was prepared in a manner similar to that described in Example 55 step 2 from 4-iodo-1H-pyrazole and tert-butyl 3-bromoazetidine-1-carboxylate. LC-MS [M+H−56]⁺=294.0.

### Step 2: 1-(azetidin-3-yl)-4-iodo-1H-pyrazole

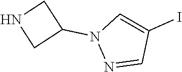

The title compound of step 2 (160 mg, 28% yield) was prepared in a manner similar to that described in Example 28 step 2 from tert-butyl 3-(4-iodo-1H-pyrazol-1-yl) azetidine-1-carboxylate. LC-MS [M+H]⁺=250.0.

### Step 3: 4-iodo-1-(1-methylazetidin-3-yl)-1H-pyrazole

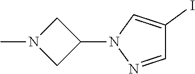

The title compound of step 3 (100 mg, 59% yield) was prepared in a manner similar to that described in Example 37 step 9 from 1-(azetidin-3-yl)-4-iodo-1H-pyrazole and formaldehyde solution. LC-MS [M+H]⁺=264.0.

### Step 4: 2-(8-(difluoromethoxy)-6-(6-(1-(1-methylazetidin-3-yl)-1H-pyrazol-4-yl)benzofuran-3-yl)-1-oxo-3,4-dihydroisoquinolin-2 (1H)-yl) acetonitrile (compound 57)

Compound 57 (3 mg, 5% yield) was prepared in a manner similar to that described in Example 1 step 6 from 2-(8-(difluoromethoxy)-1-oxo-6-(6-(4,4,5,5-tetramethyl-1,3,2-dioxaborolan-2-yl)benzofuran-3-yl)-3,4-dihydroisoquinolin-2 (1H)-yl) acetonitrile and 4-iodo-1-(1-methylazetidin-3-yl)-1H-pyrazole. ¹H NMR (500 MHz, DMSO-d₆) δ 8.57 (s, 1H), 8.47 (s, 1H), 8.07 (s, 1H), 7.98-7.92 (m, 2H), 7.70 (s, 1H), 7.68-7.63 (m, 1H), 7.50 (s, 1H), 7.18 (t, J=75.0 Hz, 1H), 5.01-4.91 (m, 1H), 4.62 (s, 2H), 3.76-3.67 (m, 4H), 3.41 (t, J=7.2 Hz, 2H), 3.15 (t, J=6.5 Hz, 2H), 2.35 (s, 3H). LC-MS [M+H]⁺=504.4.

### Example 58: 8-methoxy-6-(6-(2-morpholinoethoxy)benzofuran-3-yl)-2-(2,2,2-trifluoroethyl)-3,4-dihydroisoquinolin-1 (2H)-one (compound 58)

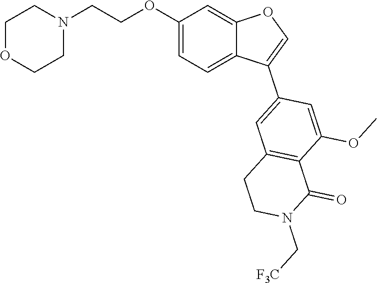 [SMILES: COc1cc(-c2coc3cc(OCCN4CCOCC4)ccc23)cc2c1C(=O)N(CC(F)(F)F)CC2]

### Step 1: 8-methoxy-6-(6-(2-morpholinoethoxy)benzofuran-3-yl)-2-(2,2,2-trifluoroethyl)-3,4-dihydroisoquinolin-1 (2H)-one (compound 58)

To a solution of 6-(6-hydroxybenzofuran-3-yl)-8-methoxy-2-(2,2,2-trifluoroethyl)-3,4-dihydroisoquinolin-1 (2H)-one (100 mg, 0.25 mmol), 2-morpholinoethan-1-ol (39 mg, 0.3 mmol), PPh₃ (96 mg, 0.37 mmol) in dried THF (10 mL) was added DIAD (74 mg, 0.37 mmol) dropwise at 0° C. The mixture was stirred at room temperature for overnight. The mixture was concentrated and purified by prep-HPLC to give compound 58 (18.8 mg, 14% yield). ¹H NMR (500 MHz, DMSO-d₆) δ 8.42 (s, 1H), 7.86 (d, J=8.4 Hz, 1H), 7.31 (d, J=2.0 Hz, 1H), 7.25 (d, J=8.0 Hz, 2H), 6.99 (dd, J₁=8.8 Hz, J₂=2.0 Hz, 1H), 4.34 (q, J=9.6 Hz, 2H), 4.17 (t, J=6.0 Hz, 2H), 3.89 (s, 3H), 3.64-3.58 (m, 6H), 2.99 (t, J=5.6 Hz, 2H), 2.73 (t, J=5.6 Hz, 2H). LC-MS [M+H]⁺=505.5.

### Example 59: 8-methoxy-6-(6-(2-(pyrrolidin-1-yl) ethoxy)benzofuran-3-yl)-2-(2,2,2-trifluoroethyl)-3,4-dihydroisoquinolin-1 (2H)-one (compound 59)

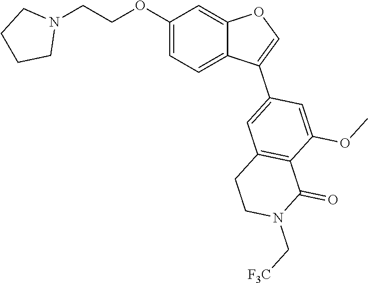 [SMILES: COc1cc(-c2coc3cc(OCCN4CCCC4)ccc23)cc2c1C(=O)N(CC(F)(F)F)CC2]

### Step 1: 8-methoxy-6-(6-(2-(pyrrolidin-1-yl) ethoxy)benzofuran-3-yl)-2-(2,2,2-trifluoroethyl)-3,4-dihydroisoquinolin-1 (2H)-one (compound 59)

Compound 59 (35 mg, 27% yield) was prepared in a manner similar to that described in Example 58 step 1 from 6-(6-hydroxybenzofuran-3-yl)-8-methoxy-2-(2,2,2-trifluoroethyl)-3,4-dihydroisoquinolin-1 (2H)-one and 2-(pyrrolidin-1-yl) ethan-1-ol. ¹H NMR (500 MHz, DMSO-d₆) δ 8.42 (s, 1H), 7.88 (d, J=8.7 Hz, 1H), 7.31 (d, J=1.6 Hz, 1H), 7.26 (d, J=12.2 Hz, 2H), 7.00 (dd, J₁=8.6 Hz, J₂=1.8 Hz, 1H), 4.34 (q, J=9.6 Hz, 2H), 4.18 (t, J=5.6 Hz, 2H), 3.89 (s, 3H), 3.63 (t, J=5.9 Hz, 2H), 3.00 (t, J=5.9 Hz, 2H), 2.92 (t, J=5.6 Hz, 2H), 2.65 (s, 4H), 1.73 (s, 4H). LC-MS [M+H]⁺=489.4.

### Example 60: 8-methoxy-6-(6-((1-methyl-1H-imidazol-5-yl) methoxy)benzofuran-3-yl)-2-(2,2,2-trifluoroethyl)-3,4-dihydroisoquinolin-1 (2H)-one (compound 60)

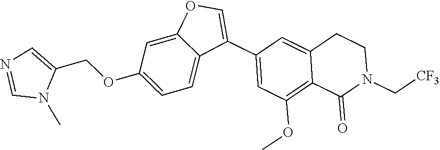 [SMILES: COc1cc(-c2coc3cc(OCc4cncn4C)ccc23)cc2c1C(=O)N(CC(F)(F)F)CC2]

Compound 60 (14 mg, 11% yield) was prepared in a manner similar to that described in Example 58 step 1 from 6-(6-hydroxybenzofuran-3-yl)-8-methoxy-2-(2,2,2-trifluoroethyl)-3,4-dihydroisoquinolin-1 (2H)-one and (1-methyl-1H-imidazol-5-yl) methanol. ¹H NMR (500 MHz, DMSO-d₆) δ 8.38 (s, 1H), 7.82 (d, J=8.7 Hz, 1H), 7.59 (s, 1H), 7.40 (d, J=2.0 Hz, 1H), 7.19 (d, J=10.7 Hz, 2H), 7.05-6.92 (m, 2H), 5.12 (s, 2H), 4.27 (q, J=9.6 Hz, 2H), 3.82 (s, 3H), 3.60 (s, 3H), 3.56 (t. J=6.0 Hz, 2H), 2.93 (t, J=5.9 Hz, 2H). LC-MS [M+H]⁺=486.3.

### Example 61: 8-methoxy-6-(6-((1-methylpyrrolidin-2-yl) methoxy)benzofuran-3-yl)-2-(2,2,2-trifluoroethyl)-3,4-dihydroisoquinolin-1 (2H)-one (compound 61)

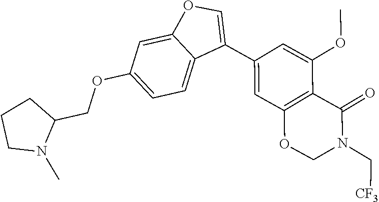 [SMILES: COc1cc(-c2coc3cc(OCC4CCCN4C)ccc23)cc2c1C(=O)N(CC(F)(F)F)CO2]

Compound 61 (24 mg, 19% yield) was prepared in a manner similar to that described in Example 58 step 1 from 6-(6-hydroxybenzofuran-3-yl)-8-methoxy-2-(2,2,2-trifluoroethyl)-3,4-dihydroisoquinolin-1 (2H)-one and (1-methylpyrrolidin-2-yl) methanol. ¹H NMR (500 MHz, DMSO-d₆) δ 8.43 (s, 1H), 7.87 (d, J=8.7 Hz, 1H), 7.31 (d, J=2.0 Hz, 1H), 7.26 (d, J=11.0 Hz, 2H), 7.00 (dd, J₁=8.7 Hz, J₂=2.1 Hz, 1H), 4.34 (q, J=9.6 Hz, 2H), 4.09-4.05 (m, 1H), 3.95-3.92 (m, 1H), 3.89 (s, 3H), 3.63 (t, J=5.9 Hz, 2H), 3.00 (t, J=5.9 Hz, 2H), 2.70-2.64 (m, 1H), 2.42 (s, 3H), 2.30-2.19 (m, 1H), 2.03-1.94 (m, 1H), 1.78-1.68 (m, 2H), 1.68-1.56 (m, 1H). LC-MS [M+H]⁺=489.4.

### Example 62: N-cyclopropyl-2-(difluoromethoxy)-6-methoxy-4-(6-(2-(pyrrolidin-1-yl) ethoxy)benzofuran-3-yl)benzamide (compound 62)

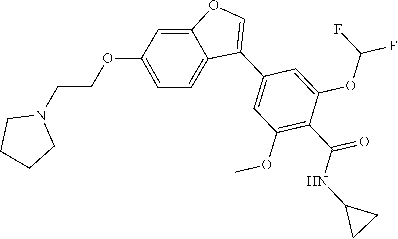 [SMILES: COc1cc(-c2coc3cc(OCCN4CCCC4)ccc23)cc(OC(F)F)c1C(=O)NC1CC1]

### Step 1: 4-bromo-N-cyclopropyl-2-(difluoromethoxy)-6-methoxybenzamide

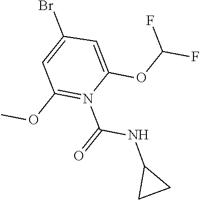

The title compound of step 1 (2.0 g, 80% yield) was prepared in a manner similar to that described in Example 25 step 9 from 4-bromo-2-(difluoromethoxy)-6-methoxybenzoic acid and cyclopropanamine. LC-MS [M+H]⁺=337.1.

### Step 2: N-cyclopropyl-2-(difluoromethoxy)-6-methoxy-4-(4,4,5,5-tetramethyl-1,3,2-dioxaborolan-2-yl)benzamide

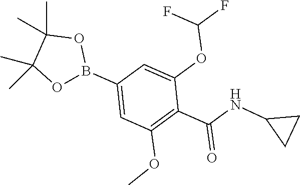 [SMILES: COc1cc(B2OC(C)(C)C(C)(C)O2)cc(OC(F)F)c1C(=O)NC1CC1]

The title compound of step 2 (2.0 g, 90% yield) was prepared in a manner similar to that described in Example 1 step 4 from 4-bromo-N-cyclopropyl-2-(difluoromethoxy)-6-methoxybenzamide and 4,4,4′,4′,5,5,5′,5′-octamethyl-2,2′-bi (1,3,2-dioxaborolane). LC-MS [M+H]⁺=384.2.

### Step 3: 3-(4-(cyclopropylcarbamoyl)-3-(difluoromethoxy)-5-methoxyphenyl)benzofuran-6-yl acetate

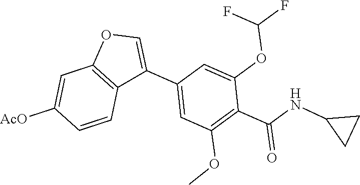 [SMILES: COc1cc(-c2coc3cc(OC(C)=O)ccc23)cc(OC(F)F)c1C(=O)NC1CC1]

The title compound of step 3 (400 mg, crude) was prepared in a manner similar to that described in Example 1 step 5 from N-cyclopropyl-2-(difluoromethoxy)-6-methoxy-4-(4,4,5,5-tetramethyl-1,3,2-dioxaborolan-2-yl)benzamide and 3-(((trifluoromethyl) sulfonyl)oxy)benzofuran-6-yl acetate. LC-MS [M+H]⁺=432.4.

### Step 4: N-cyclopropyl-2-(difluoromethoxy)-4-(6-hydroxybenzofuran-3-yl)-6-methoxybenzamide

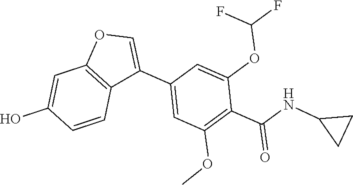 [SMILES: COc1cc(-c2coc3cc(O)ccc23)cc(OC(F)F)c1C(=O)NC1CC1]

The title compound of step 4 (370 mg, 95% yield for 2 steps) was prepared in a manner similar to that described in Example 13 step 7 from 3-(4-(cyclopropylcarbamoyl)-3-(difluoromethoxy)-5-methoxyphenyl)benzofuran-6-yl acetate. LC-MS [M+H]⁺=390.4.

### Step 5: N-cyclopropyl-2-(difluoromethoxy)-6-methoxy-4-(6-(2-(pyrrolidin-1-yl) ethoxy)benzofuran-3-yl)benzamide (compound 62)

Compound 62 (6 mg, 3% yield) was prepared in a manner similar to that described in Example 58 step 1 from N-cyclopropyl-2-(difluoromethoxy)-4-(6-hydroxybenzofuran-3-yl)-6-methoxybenzamide and 2-(pyrrolidin-1-yl) ethan-1-ol. ¹H NMR (500 MHz, DMSO-d₆) § 8.38 (s, 1H), 8.33 (s, 1H), 8.22 (s, 1H), 7.78 (d, J=8.6 Hz, 1H), 7.32 (s, 1H), 7.21 (s, 1H), 7.10-7.05 (m, 1H), 7.02 (d, J=8.4 Hz, 1H), 4.22-4.12 (m, 2H), 3.91 (s, 3H), 2.95-2.85 (m, 2H), 2.83-2.73 (m, 1H), 2.68-2.58 (m, 4H), 1.78-1.67 (m, 4H), 0.74-0.62 (m, 2H), 0.51-0.43 (m, 2H). LC-MS [M+H]⁺=487.4.

### Example 63: 8-(difluoromethoxy)-6-(6-(2-(pyrrolidin-1-yl) ethoxy)benzofuran-3-yl)-2-(2,2,2-trifluoroethyl)-3,4-dihydroisoquinolin-1 (2H)-one (compound 63)

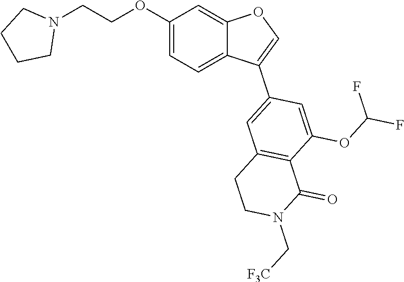 [SMILES: O=C1c2c(cc(-c3coc4cc(OCCN5CCCC5)ccc34)cc2OC(F)F)CCN1CC(F)(F)F]

### Step 1: 8-(difluoromethoxy)-6-(6-(2-(pyrrolidin-1-yl) ethoxy)benzofuran-3-yl)-2-(2,2,2-trifluoroethyl)-3,4-dihydroisoquinolin-1 (2H)-one (compound 63)

Compound 63 (12 mg, 18% yield) was prepared in a manner similar to that described in Example 58 step 1 from 8-(difluoromethoxy)-6-(6-hydroxybenzofuran-3-yl)-2-(2,2,2-trifluoroethyl)-3,4-dihydroisoquinolin-1 (2H)-one and 2-(pyrrolidin-1-yl) ethan-1-ol. ¹H NMR (500 MHz, DMSO-d₆) δ 8.49 (s, 1H), 7.85 (d, J=8.7 Hz, 1H), 7.67 (s, 1H), 7.47 (s, 1H), 7.33 (d, J=1.9 Hz, 1H), 7.15 (t, J=75.0 Hz, 1H), 7.02 (d, J=5.0 Hz, 1H), 4.38 (q, J=9.5 Hz, 2H), 4.16 (t, J=5.8 Hz, 2H), 3.69 (t, J=6.0 Hz, 2H), 3.10 (t, J=6.0 Hz, 2H), 2.87 (t, J=5.7 Hz, 2H), 2.62-2.55 (m, 4H), 1.76-1.64 (m, 4H). LC-MS [M+H]⁺=525.3.

### Example 64: 8-methoxy-6-(6-(2-(piperidin-1-yl) ethoxy)benzofuran-3-yl)-2-(2,2,2-trifluoroethyl)-3,4-dihydroisoquinolin-1 (2H)-one (compound 64)

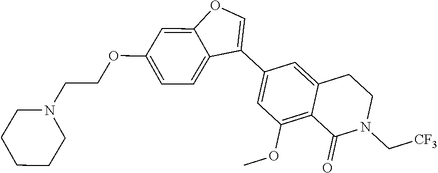 [SMILES: COc1cc(-c2coc3cc(OCCN4CCCCC4)ccc23)cc2c1C(=O)N(CC(F)(F)F)CC2]

### Step 1: 8-methoxy-6-(6-(2-(piperidin-1-yl) ethoxy)benzofuran-3-yl)-2-(2,2,2-trifluoroethyl)-3,4-dihydroisoquinolin-1 (2H)-one (compound 64)

Compound 64 (13 mg, 23% yield) was prepared in a manner similar to that described in Example 58 step 1 from 6-(6-hydroxybenzofuran-3-yl)-8-methoxy-2-(2,2,2-trifluoroethyl)-3,4-dihydroisoquinolin-1 (2H)-one and 2-(piperidin-1-yl) ethan-1-ol. ¹H NMR (500 MHz, DMSO-d₆) δ 8.42 (s, 1H), 7.87 (d, J=8.7 Hz, 1H), 7.32 (d, J=1.9 Hz, 1H), 7.26 (d, J=9.5 Hz, 2H), 6.99 (dd, J₁=8.7 Hz, J₂=2.0 Hz, 1H), 4.34 (q, J=9.6 Hz, 2H), 4.15 (t, J=5.9 Hz, 2H), 3.89 (s, 3H), 3.63 (t, J=5.9 Hz, 3H), 3.00 (t, J=5.9 Hz, 2H), 2.70 (t, J=5.8 Hz, 2H), 2.46 (s, 4H), 1.52-1.49 (m, 4H), 1.42-1.36 (m, 2H). LC-MS [M+H]⁺=503.4.

### Example 65: 2-(8-methoxy-1-oxo-6-(6-(2-(pyrrolidin-1-yl) ethoxy)benzofuran-3-yl)-3,4-dihydroisoquinolin-2 (1H)-yl) acetonitrile (compound 65)

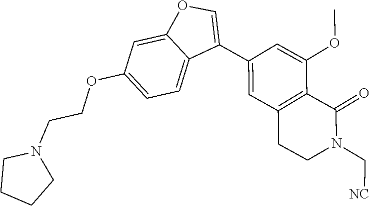 [SMILES: COc1cc(-c2coc3cc(OCCN4CCCC4)ccc23)cc2c1C(=O)N(CC#N)CC2]

### Step 1: 6-hydroxybenzofuran-3-yl trifluoromethanesulfonate

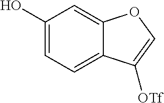 [SMILES: O=S(=O)(Oc1coc2cc(O)ccc12)C(F)(F)F]

The title compound of step 1 (900 mg, 90% yield) was prepared in a manner similar to that described in Example 13 step 7 from 3-(((trifluoromethyl) sulfonyl)oxy)benzofuran-6-yl acetate. LC-MS [M+H]⁺=283.2.

### Step 2: 6-(2-(pyrrolidin-1-yl) ethoxy)benzofuran-3-yl trifluoromethanesulfonate

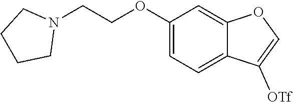 [SMILES: O=S(=O)(Oc1coc2cc(OCCN3CCCC3)ccc12)C(F)(F)F]

The title compound of step 2 (1.1 g, 91% yield) was prepared in a manner similar to that described in Example 58 step 1 from 6-hydroxybenzofuran-3-yl trifluoromethanesulfonate and 2-(pyrrolidin-1-yl) ethan-1-ol. LC-MS [M+H]⁺=380.3.

### Step 3: 2-(8-methoxy-1-oxo-6-(6-(2-(pyrrolidin-1-yl) ethoxy)benzofuran-3-yl)-3,4-dihydroisoquinolin-2 (1H)-yl) acetonitrile (compound 65)

Compound 65 (6 mg, 8% yield) was prepared in a manner similar to that described in Example 1 step 5 from 6-(2-(pyrrolidin-1-yl) ethoxy)benzofuran-3-yl trifluoromethanesulfonate and 2-(8-methoxy-1-oxo-6-(4,4,5,5-tetramethyl-1,3,2-dioxaborolan-2-yl)-3,4-dihydroisoquinolin-2 (1H)-yl) acetonitrile. ¹H NMR (500 MHz, DMSO-d₆) δ 8.43 (s, 1H), 7.88 (d, J=8.7 Hz, 1H), 7.32 (d, J=2.0 Hz, 1H), 7.26 (d, J=9.5 Hz, 2H), 7.00 (dd, J₁=8.7 Hz, J₂=2.2 Hz, 1H), 4.56 (s, 2H), 4.20-4.11 (m, 2H), 3.90 (s, 3H), 3.62 (t, J=6.3 Hz, 2H), 3.04 (t, J=6.3 Hz, 2H), 2.93-2.75 (m, 2H), 2.60-2.52 (m, 4H), 1.77-1.64 (m, 4H). LC-MS [M+H]⁺=446.3.

### Example 66: N-(cyanomethyl)-2-(difluoromethoxy)-6-methoxy-4-(6-(2-(pyrrolidin-1-yl) ethoxy)benzofuran-3-yl)benzamide (compound 66)

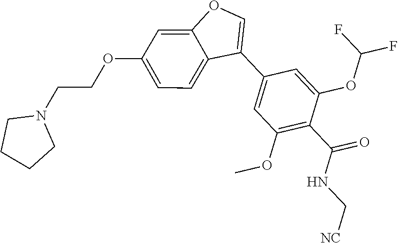 [SMILES: COc1cc(-c2coc3cc(OCCN4CCCC4)ccc23)cc(OC(F)F)c1C(=O)NCC#N]

### Step 1: tert-butyl 4-(6-acetoxybenzofuran-3-yl)-2-(difluoromethoxy)-6-methoxybenzoate

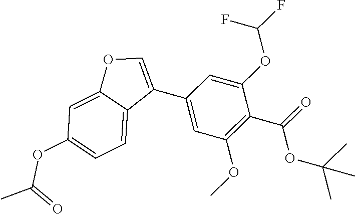 [SMILES: COc1cc(-c2coc3cc(OC(C)=O)ccc23)cc(OC(F)F)c1C(=O)OC(C)(C)C]

The title compound of step 1 (635 mg, 100% yield) was prepared in a manner similar to that described in Example 1 step 5 from 3-(((trifluoromethyl) sulfonyl)oxy)benzofuran-6-yl acetate and tert-butyl 2-(difluoromethoxy)-6-methoxy-4-(4,4,5,5-tetramethyl-1,3,2-dioxaborolan-2-yl)benzoate. LC-MS [M+H]⁺=449.0.

### Step 2: tert-butyl 2-(difluoromethoxy)-4-(6-hydroxybenzofuran-3-yl)-6-methoxybenzoate

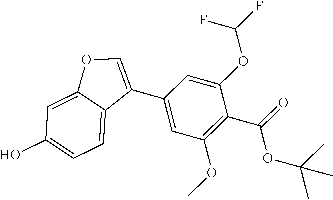 [SMILES: COc1cc(-c2coc3cc(O)ccc23)cc(OC(F)F)c1C(=O)OC(C)(C)C]

The title compound of step 2 (425 mg, 73% yield) was prepared in a manner similar to that described in Example 13 step 7 from tert-butyl 4-(6-acetoxybenzofuran-3-yl)-2-(difluoromethoxy)-6-methoxybenzoate. LC-MS [M+H]⁺=407.0.

### Step 3: tert-butyl 2-(difluoromethoxy)-6-methoxy-4-(6-(2-(pyrrolidin-1-yl) ethoxy)benzofuran-3-yl)benzoate

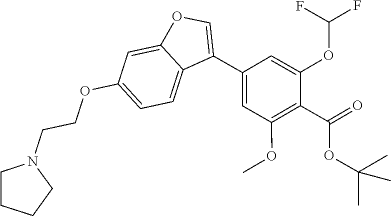 [SMILES: COc1cc(-c2coc3cc(OCCN4CCCC4)ccc23)cc(OC(F)F)c1C(=O)OC(C)(C)C]

The title compound of step 3 (324 mg, 66% yield) was prepared in a manner similar to that described in Example 58 step 1 from tert-butyl 2-(difluoromethoxy)-4-(6-hydroxybenzofuran-3-yl)-6-methoxybenzoate and 2-(pyrrolidin-1-yl) ethan-1-ol. LC-MS [M+H]⁺=504.0.

### Step 4: 2-(difluoromethoxy)-6-methoxy-4-(6-(2-(pyrrolidin-1-yl) ethoxy)benzofuran-3-yl)benzoic acid

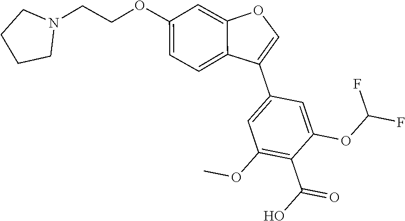 [SMILES: COc1cc(-c2coc3cc(OCCN4CCCC4)ccc23)cc(OC(F)F)c1C(=O)O]

The title compound of step 4 (288 mg, 100% yield) was prepared in a manner similar to that described in Example 25 step 8 from tert-butyl 2-(difluoromethoxy)-6-methoxy-4-(6-(2-(pyrrolidin-1-yl) ethoxy)benzofuran-3-yl)benzoate. LC-MS [M+H]⁺=448.0.

### Step 5: N-(cyanomethyl)-2-(difluoromethoxy)-6-methoxy-4-(6-(2-(pyrrolidin-1-yl) ethoxy)benzofuran-3-yl)benzamide (compound 66)

Compound 66 (6 mg, 15% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and 2-aminoacetonitrile. ¹H NMR (500 MHz, DMSO-d₆) δ 9.06 (t, J=5.6 Hz, 1H), 8.41 (s, 1H), 7.81 (d. J=8.7 Hz, 1H), 7.37-6.97 (m, 5H), 4.31 (d, J=5.7 Hz, 2H), 4.16 (t, J=5.8 Hz, 2H), 3.90 (s, 3H), 2.85 (t, J=5.8 Hz, 2H), 2.63-2.54 (m, 4H), 1.74-1.67 (m, 4H). LC-MS [M+H]⁺=486.0.

### Example 67: 2-(difluoromethoxy)-N-((1R,2S)-2-fluorocyclopropyl)-6-methoxy-4-(6-(2-(pyrrolidin-1-yl) ethoxy)benzofuran-3-yl)benzamide (compound 67)

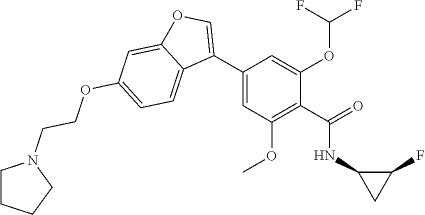 [SMILES: COc1cc(-c2coc3cc(OCCN4CCCC4)ccc23)cc(OC(F)F)c1C(=O)N[C@@H]1C[C@@H]1F]

### Step 1: 4-(6-(2-bromoethoxy)benzofuran-3-yl)-2-(difluoromethoxy)-N-((1R,2S)-2-fluorocyclopropyl)-6-methoxybenzamide

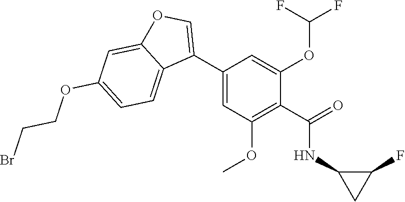 [SMILES: COc1cc(-c2coc3cc(OCCBr)ccc23)cc(OC(F)F)c1C(=O)N[C@@H]1C[C@@H]1F]

2-(difluoromethoxy)-N-((1R,2S)-2-fluorocyclopropyl)-4-(6-hydroxybenzofuran-3-yl)-6-methoxybenzamide (255 mg, 0.626 mmol) and 1,2-dibromoethane (1.18 g, 6.26 mmol) were dissolved in acetonitrile (20 mL). K₂CO₃ (518.4 mg, 3.76 mmol) was added and the mixture was heated to 80° C. for overnight. The mixture was filtered, dried and concentrated. the residue (322 mg, crude) was directly used in next step without further purification. LC-MS [M+H]=514.0.

### Step 2: 2-(difluoromethoxy)-N-((1R,2S)-2-fluorocyclopropyl)-6-methoxy-4-(6-(2-(pyrrolidin-1-yl) ethoxy)benzofuran-3-yl)benzamide (compound 67)

4-(6-(2-bromoethoxy)benzofuran-3-yl)-2-(difluoromethoxy)-N-((1R,2S)-2-fluorocyclopropyl)-6-methoxybenzamide (70 mg, 0.13 mmol) and pyrrolidine (19.4 mg, 0.26 mmol) were dissolved acetonitrile (6 mL). K₂CO₃ (56.4 mg, 0.41 mmol) and KI (2.3 mg, 0.013 mmol) were added and the mixture was heated to 80° C. for overnight. The mixture was filtered, dried and concentrated. The residue was purified by prep-HPLC to afford compound 67 (5.4 mg, 8% yield). ¹H NMR (500 MHz, DMSO-d₆) δ 8.50 (d, J=4.5 Hz, 1H), 8.39 (s, 1H), 7.78 (d, J=8.0 Hz, 1H), 7.36-7.01 (m, 5H), 4.79-4.66 (m, 1H), 4.19 (dd, J₁=12.4 Hz, J₂=6.7 Hz, 2H), 3.89 (s, 3H), 3.00 (s, 2H), 2.83 (dt, J₁=9.2, J₂=4.5 Hz, 2H), 2.72 (s, 4H), 1.75 (s, 4H), 1.13-1.06 (m, 1H), 0.99-0.87 (m, 1H). LC-MS [M+H]=505.0.

### Example 68: 2-(difluoromethoxy)-N-((1R,2S)-2-fluorocyclopropyl)-6-methoxy-4-(6-((1-(pyrrolidin-1-yl) propan-2-yl)oxy)benzofuran-3-yl)benzamide (compound 68)

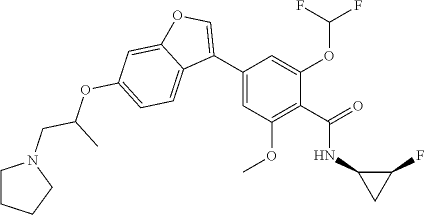 [SMILES: COc1cc(-c2coc3cc(OC(C)CN4CCCC4)ccc23)cc(OC(F)F)c1C(=O)N[C@@H]1C[C@@H]1F]

### Step 1: 2-(difluoromethoxy)-N-((1R,2S)-2-fluorocyclopropyl)-6-methoxy-4-(6-((1-(pyrrolidin-1-yl) propan-2-yl)oxy)benzofuran-3-yl)benzamide (compound 68)

Compound 68 (2.3 mg, 4% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and 1-(pyrrolidin-1-yl) propan-2-ol. ¹H NMR (500 MHz, DMSO-d₆) δ 8.50 (d, J=4.5 Hz, 1H), 8.45-8.35 (m, 3H), 7.79-7.76 (m, 1H), 7.31 (d, J=2.0 Hz, 1H), 7.22 (s, 1H), 7.20 (t, J=75.0 Hz, 1H), 7.09 (s, 1H), 7.03-6.98 (m, 1H), 4.80-4.77 (m, 0.5H), 4.71-4.63 (m, 1H), 4.16-4.13 (m, 0.6H), 3.94-3.83 (m, 4H), 2.84-2.82 (m, 1H), 2.76-2.69 (m, 1H), 2.68-2.64 (m, 1H), 2.60 (s, 3H), 1.68-1.66 (m, 4H), 1.28 (d, J=6.1 Hz, 1H), 1.17 (d, J=6.5 Hz, 2H), 1.13-1.05 (m, 1H), 0.99-0.85 (m, 1H). LC-MS [M+H]⁺=519.4.

### Example 69: 2-(difluoromethoxy)-N-((1R,2S)-2-fluorocyclopropyl)-6-methoxy-4-(6-(2-morpholinoethoxy)benzofuran-3-yl)benzamide (compound 69)

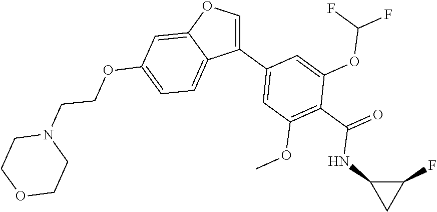 [SMILES: COc1cc(-c2coc3cc(OCCN4CCOCC4)ccc23)cc(OC(F)F)c1C(=O)N[C@@H]1C[C@@H]1F]

Compound 69 (15 mg, 23% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and 2-morpholinoethan-1-ol. ¹H NMR (500 MHz, DMSO-d₆) δ 8.50 (d, J=4.5 Hz, 1H), 8.39 (s, 1H), 7.78 (d, J=8.7 Hz, 1H), 7.32 (d, J=2.0 Hz, 1H), 7.22 (s, 1H), 7.20 (t, J=75.0 Hz, 1H), 7.08 (s, 1H), 7.01 (dd, J₁=9.0 Hz, J₂=2.0 Hz, 1H), 4.90-4.61 (m, 1H), 4.17 (t, J=5.7 Hz, 2H), 3.86 (s, 3H), 3.59 (t. J=5.0 Hz, 4H), 2.84 (dt, J₁=13.8 Hz, J₂=4.6 Hz, 1H), 2.73 (t, J=5.7 Hz, 2H), 2.51 (s, 4H), 1.11-1.07 (m, 1H), 1.02-0.86 (m, 1H). LC-MS [M+H]⁺=521.0.

### Example 70: 2-(difluoromethoxy)-N-((1R,2S)-2-fluorocyclopropyl)-6-methoxy-4-(6-(2-(piperidin-1-yl) ethoxy)benzofuran-3-yl)benzamide (compound 70)

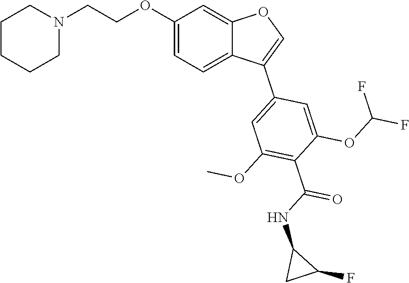 [SMILES: COc1cc(-c2coc3cc(OCCN4CCCCC4)ccc23)cc(OC(F)F)c1C(=O)N[C@@H]1C[C@@H]1F]

Compound 70 (10 mg, 14% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and 2-(piperidin-1-yl) ethan-1-ol. ¹H NMR (500 MHz, DMSO-d₆) δ 8.50 (d. J=4.4 Hz, 1H), 8.38 (s, 1H), 7.78 (d, J=8.7 Hz, 1H), 7.31 (d, J=2.5 Hz, 1H), 7.22 (s, 1H), 7.20 (t, J=75.0 Hz, 1H), 7.09 (s, 1H), 7.00 (dd, J₁=8.5 Hz, J₂=2.5 Hz, 1H), 4.89-4.59 (m, 1H), 4.14 (t, J=5.9 Hz, 2H), 3.88 (s, 3H), 2.86-2.81 (m, 1H), 2.69 (t, J=5.9 Hz, 2H), 2.45 (s, 4H), 1.56-1.44 (m, 4H), 1.41-1.35 (m, 2H), 1.13-1.05 (m, 1H), 1.01-0.87 (m, 1H). LC-MS [M+H]⁺=519.0.

### Example 71: 2-(difluoromethoxy)-N-((1R,2S)-2-fluorocyclopropyl)-4-(6-(2-((R)-3-fluoropyrrolidin-1-yl) ethoxy)benzofuran-3-yl)-6-methoxybenzamide (compound 71)

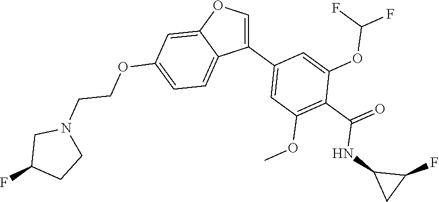 [SMILES: COc1cc(-c2coc3cc(OCCN4CC[C@@H](F)C4)ccc23)cc(OC(F)F)c1C(=O)N[C@@H]1C[C@@H]1F]

Compound 71 (10 mg, 15% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and (R)-2-(3-fluoropyrrolidin-1-yl) ethan-1-ol. ¹H NMR (500 MHz, DMSO-d₆) δ 8.50 (d, J=4.5 Hz, 1H), 8.39 (s, 1H), 7.79 (d, J=8.7 Hz, 1H), 7.32 (d, J=2.5 Hz, 1H), 7.22 (s, 1H), 7.20 (t, J=75.0 Hz, 1H), 7.09 (s, 1H), 7.01 (dd, J₁=9.0 Hz, J₂=2.5 Hz, 1H), 5.20 (dt, J₁=56.0 Hz, J₂=5.9 Hz, 1H), 4.84-4.59 (m, 1H), 4.16 (t, J=5.7 Hz, 2H), 3.88 (s, 3H), 2.95-2.80 (m, 5H), 2.75-2.63 (m, 1H), 2.44-2.36 (m, 1H), 2.22-2.04 (m, 1H), 1.97-1.75 (m, 1H), 1.13-1.05 (m, 1H), 0.97-0.85 (m, 1H). LC-MS [M+H]⁺=523.0.

### Example 72: 2-(difluoromethoxy)-N-((1R,2S)-2-fluorocyclopropyl)-6-methoxy-4-(6-(3-(pyrrolidin-1-yl) propoxy)benzofuran-3-yl)benzamide (compound 72)

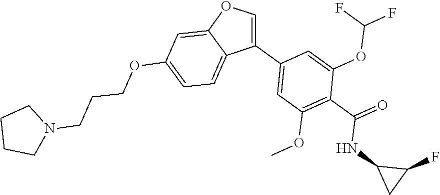 [SMILES: COc1cc(-c2coc3cc(OCCCN4CCCC4)ccc23)cc(OC(F)F)c1C(=O)N[C@@H]1C[C@@H]1F]

Compound 72 (0.38 mg, 0.6% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and 3-(pyrrolidin-1-yl) propan-1-ol. ¹H NMR (500 MHz, DMSO-d₆) δ 8.50 (d, J=4.4 Hz, 1H), 8.38 (s, 1H), 7.78 (d, J=8.7 Hz, 1H), 7.29 (d, J=5.0 Hz, 1H), 7.22 (s, 1H), 7.20 (t, J=75.0 Hz, 1H), 7.09 (s, 1H), 7.00 (dd, J₁=9.0 Hz, J₂=2.5 Hz, 1H), 4.80-4.64 (m, 1H), 4.09 (t, J=6.4 Hz, 2H), 3.88 (s, 3H), 2.86-2.81 (m, 1H), 2.57-2.54 (m, 2H), 2.46 (s, 4H), 1.98-1.88 (m, 2H), 1.69 (d, J=3.2 Hz, 4H), 1.14-1.05 (m, 1H), 0.98-0.88 (m, 1H). LC-MS [M+H]⁺=519.0.

### Example 73: 2-(difluoromethoxy)-4-(6-(2-(dimethylamino) ethoxy)benzofuran-3-yl)-N-((1R,2S)-2-fluorocyclopropyl)-6-methoxybenzamide (compound 73)

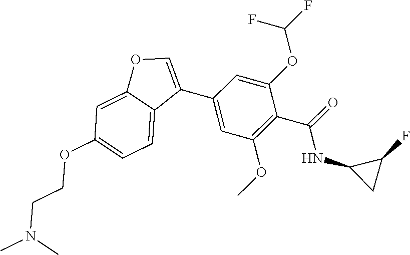 [SMILES: COc1cc(-c2coc3cc(OCCN(C)C)ccc23)cc(OC(F)F)c1C(=O)N[C@@H]1C[C@@H]1F]

Compound 73 (10 mg, 35% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and dimethylamine hydrochloride. ¹H NMR (500 MHz, DMSO-d₆) δ 8.50 (d, J=4.4 Hz, 1H), 8.39 (s, 1H), 7.79 (d, J=8.7 Hz, 1H), 7.33 (s, 1H), 7.22 (s, 1H), 7.20 (t, J=75.0 Hz, 1H), 7.09 (s, 1H), 7.03 (dd, J₁=9.0 Hz, J₂=2.5 Hz, 1H), 4.80-4.65 (m, 1H), 4.16 (t, J=5.5 Hz, 2H), 3.88 (s, 3H), 2.98-2.68 (m, 3H), 2.32 (s, 6H), 1.12-1.05 (m, 1H), 1.02-0.80 (m, 1H). LC-MS [M+H]=479.0.

### Example 74: 2-(difluoromethoxy)-N-((1R,2S)-2-fluorocyclopropyl)-4-(6-(2-((S)-3-fluoropyrrolidin-1-yl) ethoxy)benzofuran-3-yl)-6-methoxybenzamide (compound 74)

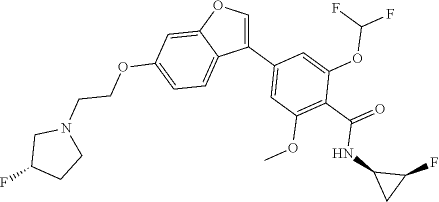 [SMILES: COc1cc(-c2coc3cc(OCCN4CC[C@H](F)C4)ccc23)cc(OC(F)F)c1C(=O)N[C@@H]1C[C@@H]1F]

Compound 74 (18 mg, 40% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-3-fluoropyrrolidine hydrochloride. ¹H NMR (500 MHz, DMSO-d₆) δ 8.50 (d. J=4.4 Hz, 1H), 8.40 (d, J=8.7 Hz, 1H), 7.79 (d. J=8.7 Hz, 1H), 7.33 (s, 1H), 7.22 (s, 1H), 7.20 (t, J=75.0 Hz, 1H), 7.09 (s, 1H), 7.02 (d, J=5.0 Hz, 1H), 5.27-5.15 (m, 1H), 4.80-4.64 (m, 1H), 4.17 (s, 2H), 3.88 (s, 3H), 2.96-2.82 (m, 6H), 2.20-2.07 (m, 1H), 1.95-1.80 (m, 1H), 1.13-1.05 (m, 1H), 1.02-0.84 (m, 1H). LC-MS [M+H]⁺=523.0.

### Example 75: 4-(6-(2-(azetidin-1-yl) ethoxy)benzofuran-3-yl)-2-(difluoromethoxy)-N-((1R,2S)-2-fluorocyclopropyl)-6-methoxybenzamide (compound 75)

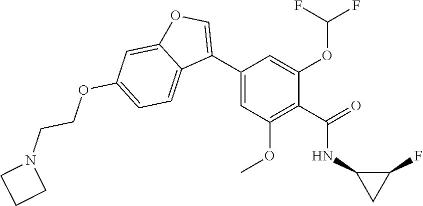 [SMILES: COc1cc(-c2coc3cc(OCCN4CCC4)ccc23)cc(OC(F)F)c1C(=O)N[C@@H]1C[C@@H]1F]

Compound 75 (3 mg, 10% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and azetidine hydrochloride. ¹H NMR (500 MHz, DMSO-d₆) δ 8.50 (d, J=4.5 Hz, 1H), 8.39 (d, J=9.2 Hz, 1H), 7.78 (d, J=8.7 Hz, 1H), 7.28 (s, 1H), 7.24 (s, 1H), 7.20 (t, J=75.0 Hz, 1H), 7.09 (s, 1H), 6.99 (dd, J₁=10.0 Hz, J₂=5.0 Hz, 1H), 4.80-4.64 (m, 1H), 4.00 (t, J=5.6 Hz, 2H), 3.88 (s, 3H), 3.23 (t, J=7.0 Hz, 4H), 2.85-2.82 (m, 1H), 2.77 (t, J=5.5 Hz, 2H), 1.99 (quint, J=6.9 Hz, 2H), 1.13-1.05 (m, 1H), 1.02-0.87 (m, 1H). LC-MS [M+H]⁺=491.4.

### Example 76: 2-(difluoromethoxy)-N-((1R,2S)-2-fluorocyclopropyl)-6-methoxy-4-(6-(2-(3-(trifluoromethyl) pyrrolidin-1-yl) ethoxy)benzofuran-3-yl)benzamide (compound 76)

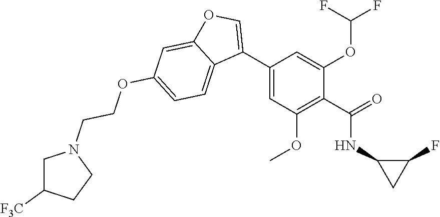 [SMILES: COc1cc(-c2coc3cc(OCCN4CCC(C(F)(F)F)C4)ccc23)cc(OC(F)F)c1C(=O)N[C@@H]1C[C@@H]1F]

### Step 1: 2-(difluoromethoxy)-N-((1R,2S)-2-fluorocyclopropyl)-6-methoxy-4-(6-(2-(3-(trifluoromethyl) pyrrolidin-1-yl) ethoxy)benzofuran-3-yl)benzamide (compound 76)

Compound 76 (18 mg, 40% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and 3-(trifluoromethyl) pyrrolidine hydrochloride. ¹H NMR (500 MHz, DMSO-d₆) § 8.50 (d, J=4.5 Hz, 1H), 8.40 (d, J=8.9 Hz, 1H), 7.78 (d, J=8.7 Hz, 1H), 7.33 (s, 1H), 7.22 (s, 1H), 7.20 (t, J=75.0 Hz, 1H), 7.09 (s, 1H), 7.01 (dd, J₁=10.0 Hz, J₂=2.0 Hz, 1H), 4.90-4.52 (m, 1H), 4.16 (t. J=5.7 Hz, 2H), 3.88 (s, 3H), 3.10-3.05 (m, 1H), 2.90-2.81 (m, 4H), 2.71-2.58 (m, 4H), 2.06-1.97 (m, 1H), 1.78-1.70 (m, 1H), 1.11-1.06 (m, 1H), 0.99-0.87 (m, 1H). LC-MS [M+H]⁺=573.0.

### Example 77: 2-(difluoromethoxy)-N-((1R,2S)-2-fluorocyclopropyl)-6-methoxy-4-(6-(2-((R)-2-methylpyrrolidin-1-yl) ethoxy)benzofuran-3-yl)benzamide (compound 77)

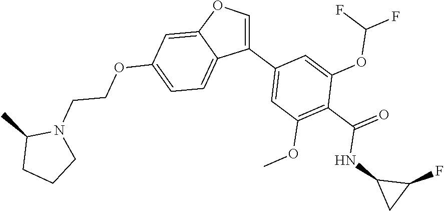 [SMILES: COc1cc(-c2coc3cc(OCCN4CCC[C@H]4C)ccc23)cc(OC(F)F)c1C(=O)N[C@@H]1C[C@@H]1F]

Compound 77 (7 mg, 35% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-2-methylpyrrolidine hydrochloride. ¹H NMR (500 MHz, DMSO-d₆) δ 8.50 (d, J=4.3 Hz, 1H), 8.39 (s, 1H), 7.79 (d, J=8.6 Hz, 1H), 7.32 (s, 1H), 7.22 (s, 1H), 7.20 (t, J=75.0 Hz, 1H), 7.09 (s, 1H), 7.01 (d, J=8.0 Hz, 1H), 4.80-4.64 (m, 1H), 4.15 (t, J=5.5 Hz, 2H), 3.88 (s, 3H), 3.29-3.26 (m, 2H), 3.18 (s, 2H), 2.84 (d, J=4.0 Hz, 1H), 2.27 (s, 1H), 1.93-1.86 (m, 1H), 1.71-1.63 (m, 2H), 1.35-1.28 (m, 1H), 1.17-1.02 (m, 4H), 0.97-0.89 (m, 1H). LC-MS [M+H]⁺=519.4.

### Example 78: 2-(difluoromethoxy)-N-((1R,2S)-2-fluorocyclopropyl)-6-methoxy-4-(6-(2-((R)-3-methylpyrrolidin-1-yl) ethoxy)benzofuran-3-yl)benzamide (compound 78)

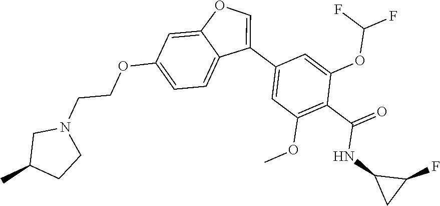 [SMILES: COc1cc(-c2coc3cc(OCCN4CC[C@@H](C)C4)ccc23)cc(OC(F)F)c1C(=O)N[C@@H]1C[C@@H]1F]

### Step 1: 2-(difluoromethoxy)-N-((1R,2S)-2-fluorocyclopropyl)-6-methoxy-4-(6-(2-((R)-3-methylpyrrolidin-1-yl) ethoxy)benzofuran-3-yl)benzamide (compound 78)

Compound 78 (3.5 mg, 17% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-3-methylpyrrolidine hydrochloride. ¹H NMR (500 MHz, DMSO-d₆) δ 8.50 (d. J=4.4 Hz, 1H), 8.39 (s, 1H), 7.79 (d, J=8.6 Hz, 1H), 7.33 (s, 1H), 7.22 (s, 1H), 7.20 (t, J=75.0 Hz, 1H), 7.09 (s, 1H), 7.02 (dd, J₁=8.5 Hz, J₂=1.5 Hz, 1H), 4.80-4.65 (m, 1H), 4.17 (t, J=5.0 Hz, 2H), 3.88 (s, 3H), 3.29 (s, 2H), 2.95 (s, 3H), 2.25-2.17 (m, 2H), 2.01-1.94 (m, 1H), 1.35-1.28 (m, 1H), 1.09-1.04 (m, 1H), 1.00 (d, J=6.50 Hz, 3H), 0.95-0.88 (m, 1H). LC-MS [M+H]⁺=519.4.

### Example 79: 2-(difluoromethoxy)-N-((1R,2S)-2-fluorocyclopropyl)-6-methoxy-4-(6-(2-((R)-3-methoxypyrrolidin-1-yl) ethoxy)benzofuran-3-yl)benzamide (compound 79)

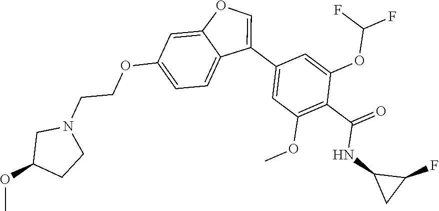 [SMILES: COc1cc(-c2coc3cc(OCCN4CC[C@@H](OC)C4)ccc23)cc(OC(F)F)c1C(=O)N[C@@H]1C[C@@H]1F]

Compound 79 (9 mg, 52% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-3-methoxypyrrolidine hydrochloride. ¹H NMR (500 MHz, DMSO-d₆) § 8.50 (d. J=4.5 Hz, 1H), 8.39 (s, 1H), 7.80 (d, J 8.7 Hz, 1H), 7.34 (s, 1H), 7.22 (s, 1H), 7.20 (t, J=75.0 Hz, 1H), 7.09 (s, 1H), 7.02 (dd, J₁=8.5 Hz, J₂=1.0 Hz, 1H), 4.95-4.60 (m, 1H), 4.19 (s, 2H), 3.94-3.90 (m, 1H), 3.88 (s, 3H), 3.29 (s, 2H), 3.19 (s, 3H), 2.85-2.80 (m, 5H), 2.07-1.95 (m, 1H), 1.78-1.66 (m, 1H), 1.13-1.05 (m, 1H), 0.99-0.83 (m, 1H). LC-MS [M+H]⁺=535.4.

### Example 80: 2-(difluoromethoxy)-N-((1R,2S)-2-fluorocyclopropyl)-6-methoxy-4-(6-(2-((S)-2-methylpyrrolidin-1-yl) ethoxy)benzofuran-3-yl)benzamide (compound 80)

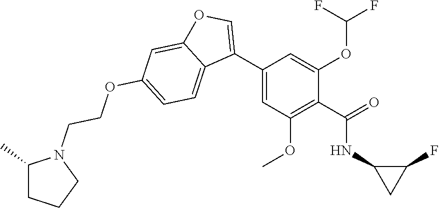 [SMILES: COc1cc(-c2coc3cc(OCCN4CCC[C@@H]4C)ccc23)cc(OC(F)F)c1C(=O)N[C@@H]1C[C@@H]1F]

Compound 80 (10 mg, 53% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-2-methylpyrrolidine hydrochloride. ¹H NMR (500 MHz, DMSO-d₆) δ 8.50 (d, J=4.5 Hz, 1H), 8.39 (s, 1H), 7.79 (d, J=8.7 Hz, 1H), 7.32 (d, J=2.0 Hz, 1H), 7.22 (s, 1H), 7.20 (t, J=75.0 Hz, 1H), 7.09 (s, 1H), 7.00 (dd, J₁=8.5 Hz, J₂=2.0 Hz, 1H), 4.95-4.64 (m, 1H), 4.16 (t, J=6.0 Hz, 2H), 3.88 (s, 3H), 3.22-3.11 (m, 2H), 2.84 (td, J₁=9.2 Hz, J₂=4.5 Hz, 1H), 2.68-2.60 (m, 1H), 2.48-2.41 (m, 2H), 2.29 (d, J=7.3 Hz, 1H), 1.94-1.86 (m, 1H), 1.77-1.59 (m, 2H), 1.18-1.03 (m, 5H), 0.98-0.86 (m, 1H). LC-MS [M+H]⁺=519.4.

### Example 81: 2-(difluoromethoxy)-N-((1R,2S)-2-fluorocyclopropyl)-6-methoxy-4-(6-(2-((S)-3-methylpyrrolidin-1-yl) ethoxy)benzofuran-3-yl)benzamide (compound 81)

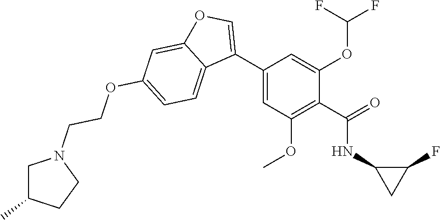 [SMILES: COc1cc(-c2coc3cc(OCCN4CC[C@H](C)C4)ccc23)cc(OC(F)F)c1C(=O)N[C@@H]1C[C@@H]1F]

Compound 81 (8 mg, 40% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-3-methylpyrrolidine hydrochloride. ¹H NMR (500 MHz, DMSO-d₆) δ 8.50 (d, J=4.5 Hz, 1H), 8.40 (s, 1H), 7.79 (d, J=8.7 Hz, 1H), 7.34 (d, J=2.0 Hz, 1H), 7.22 (s, 1H), 7.20 (t, J=75.0 Hz, 1H), 7.09 (s, 1H), 7.02 (dd, J₁=8.5 Hz, J₂=2.0 Hz, 1H), 4.80-4.64 (m, 1H), 4.18 (t, J=5.4 Hz, 2H), 3.88 (s, 3H), 2.97 (s, 3H), 2.86-2.79 (m, 2H), 2.71 (s, 1H), 2.31-2.15 (m, 2H), 2.09-1.95 (m, 1H), 1.36-1.28 (m, 1H), 1.13-1.05 (m, 1H), 0.99 (d, J=6.5 Hz, 3H), 0.92-0.88 (m, 1H). LC-MS [M+H]⁺=519.3.

### Example 82: 2-(difluoromethoxy)-N-((1R,2S)-2-fluorocyclopropyl)-6-methoxy-4-(6-(2-((S)-3-methoxypyrrolidin-1-yl) ethoxy)benzofuran-3-yl)benzamide (compound 82)

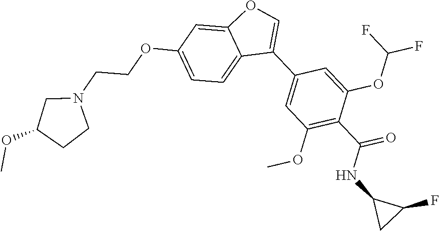 [SMILES: COc1cc(-c2coc3cc(OCCN4CC[C@H](OC)C4)ccc23)cc(OC(F)F)c1C(=O)N[C@@H]1C[C@@H]1F]

Compound 82 (8.5 mg, 42% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-3-methoxypyrrolidine hydrochloride. ¹H NMR (500 MHz, DMSO-d₆) δ 8.50 (d, J=4.4 Hz, 1H), 8.39 (s, 1H), 7.79 (d, J=8.7 Hz, 1H), 7.32 (J=2.0 Hz, 1H), 7.22 (s, 1H), 7.20 (t. J=75.0 Hz, 1H), 7.09 (s, 1H), 7.01 (dd, J₁=8.5 Hz, J₂=2.5 Hz, 1H), 4.87-4.48 (m, 1H), 4.16 (d, J=5.6 Hz, 2H), 3.88 (s, 3H), 3.29 (s, 1H), 3.17 (s, 3H), 3.00-2.75 (m, 4H), 2.70 (s, 2H), 2.03-1.95 (m, 1H), 1.67 (s, 1H), 1.13-1.04 (m, 1H), 0.99-0.84 (m, 1H). LC-MS [M+H]⁺=535.4.

### Example 83: 2-(difluoromethoxy)-N-((1R,2S)-2-fluorocyclopropyl)-6-methoxy-4-(6-(2-(4-methylpiperazin-1-yl) ethoxy)benzofuran-3-yl)benzamide (compound 83)

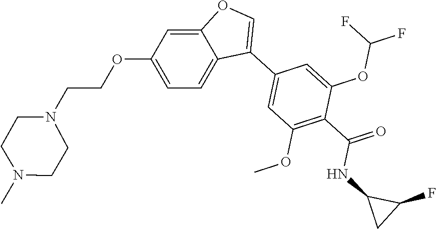 [SMILES: COc1cc(-c2coc3cc(OCCN4CCN(C)CC4)ccc23)cc(OC(F)F)c1C(=O)N[C@@H]1C[C@@H]1F]

Compound 83 (3.5 mg, 2% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and 1-methylpiperazine. ¹H NMR (500 MHz, DMSO-d₆) δ 8.50 (d, J=4.5 Hz, 1H), 8.38 (s, 1H), 7.79 (t. J=9.9 Hz, 1H), 7.32 (d, J=2.0 Hz, 1H), 7.22 (s, 1H), 7.20 (t, J=75.0 Hz, 1H), 7.09 (s, 1H), 7.01 (dd, J₁=8.5 Hz, J₂=2.5 Hz, 1H), 4.84-4.48 (m, 1H), 4.15 (t, J=5.8 Hz, 2H), 3.89 (s, 3H), 2.90-2.79 (m, 2H), 2.72 (t, J=5.8 Hz, 3H), 2.62 (t, J=17.2 Hz, 2H), 2.35 (d, J=13.7 Hz, 3H), 2.15 (s, 3H), 1.13-1.04 (m, 1H), 1.01-0.82 (m, 1H). LC-MS [M+H]⁺=534.5.

### Example 84: 2-(difluoromethoxy)-N-((1R,2S)-2-fluorocyclopropyl)-6-methoxy-4-(6-(3-(piperidin-1-yl) propoxy)benzofuran-3-yl)benzamide (compound 84)

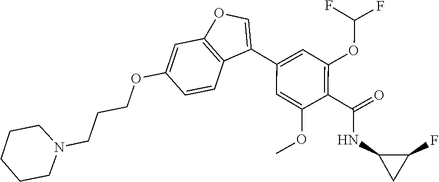 [SMILES: COc1cc(-c2coc3cc(OCCCN4CCCCC4)ccc23)cc(OC(F)F)c1C(=O)N[C@@H]1C[C@@H]1F]

### Step 1: 4-(6-(3-bromopropoxy)benzofuran-3-yl)-2-(difluoromethoxy)-N-((1R,2S)-2-fluorocyclopropyl)-6-methoxybenzamide

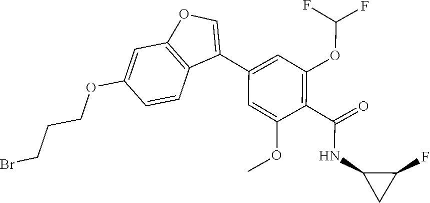 [SMILES: COc1cc(-c2coc3cc(OCCCBr)ccc23)cc(OC(F)F)c1C(=O)N[C@@H]1C[C@@H]1F]

The title compound of step 1 (90 mg, 78% yield) was prepared in a manner similar to that described in Example 67 step 1 from 2-(difluoromethoxy)-N-((1R,2S)-2-fluorocyclopropyl)-4-(6-hydroxybenzofuran-3-yl)-6-methoxybenzamide and 1,3-dibromopropane. LC-MS [M+H]⁺=529.3.

### Step 2: 2-(difluoromethoxy)-N-((1R,2S)-2-fluorocyclopropyl)-6-methoxy-4-(6-(3-(piperidin-1-yl) propoxy)benzofuran-3-yl)benzamide (compound 84)

Compound 84 (7.5 mg, 28% yield) was prepared in a manner similar to that described in Example 67 step 2 from 4-(6-(3-bromopropoxy)benzofuran-3-yl)-2-(difluoromethoxy)-N-((1R,2S)-2-fluorocyclopropyl)-6-methoxybenzamide and piperidine. ¹H NMR (500 MHz, DMSO-d₆) δ 8.50 (d, J=4.5 Hz, 1H), 8.39 (s, 1H), 7.79 (t, J=9.9 Hz, 1H), 7.30 (d, J=2.0 Hz, 1H), 7.22 (s, 1H), 7.20 (t, J=75.0 Hz, 1H), 7.09 (s, 1H), 7.01 (dd, J₁=8.5 Hz, J₂=2.5 Hz, 1H), 4.88-4.37 (m, 1H), 4.09 (t, J=6.3 Hz, 2H), 3.88 (s, 3H), 2.86-2.81 (m, 1H), 2.55 (s, 2H), 2.43-2.38 (m, 4H), 2.01-1.84 (m, 2H), 1.56-1.51 (m, 4H), 1.41 (d, J=5.0 Hz, 2H), 1.17-1.03 (m, 1H), 1.00-0.81 (m, 1H). LC-MS [M+H]⁺=533.5.

### Example 85: 2-(difluoromethoxy)-N-((1R,2S)-2-fluorocyclopropyl)-6-methoxy-4-(6-(3-(4-methylpiperazin-1-yl) propoxy)benzofuran-3-yl)benzamide (compound 85)

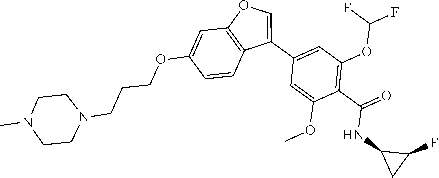 [SMILES: COc1cc(-c2coc3cc(OCCCN4CCN(C)CC4)ccc23)cc(OC(F)F)c1C(=O)N[C@@H]1C[C@@H]1F]

Compound 85 (8 mg, 40% yield) was prepared in a manner similar to that described in Example 67 step 2 from 4-(6-(3-bromopropoxy)benzofuran-3-yl)-2-(difluoromethoxy)-N-((1R,2S)-2-fluorocyclopropyl)-6-methoxybenzamide and 1-methylpiperazine. ¹H NMR (500 MHz, DMSO-d₆) δ 8.50 (d, J=4.5 Hz, 1H), 8.38 (s, 1H), 7.79 (t, J=10.0 Hz, 1H), 7.29 (d, J=2.0 Hz, 1H), 7.22 (s, 1H), 7.20 (t, J=75.0 Hz, 1H), 7.09 (s, 1H), 7.01 (dd, J₁=8.5 Hz, J₂=2.5 Hz, 1H), 4.81-4.58 (m, 1H), 4.08 (t. J=6.3 Hz, 2H), 3.88 (s, 3H), 2.87-2.75 (m, 1H), 2.46-2.23 (m, 10H), 2.18 (s, 3H), 1.96-1.75 (m, 2H), 1.13-1.05 (m, 1H), 1.02-0.78 (m, 1H). LC-MS [M+H]⁺=548.5.

### Example 86: 2-(difluoromethoxy)-N-((1R,2S)-2-fluorocyclopropyl)-6-methoxy-4-(6-(3-morpholinopropoxy)benzofuran-3-yl)benzamide (compound 86)

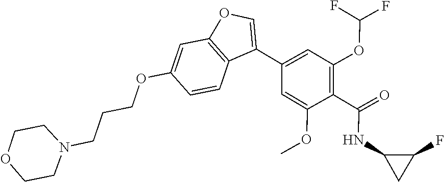 [SMILES: COc1cc(-c2coc3cc(OCCCN4CCOCC4)ccc23)cc(OC(F)F)c1C(=O)N[C@@H]1C[C@@H]1F]

Compound 86 (7 mg, 40% yield) was prepared in a manner similar to that described in Example 67 step 2 from 4-(6-(3-bromopropoxy)benzofuran-3-yl)-2-(difluoromethoxy)-N-((1R,2S)-2-fluorocyclopropyl)-6-methoxybenzamide and morpholine. ¹H NMR (500 MHz, DMSO-d₆) δ 8.50 (d, J=4.4 Hz, 1H), 8.39 (s, 1H), 7.78 (d, J=8.7 Hz, 1H), 7.29 (d, J=2.0 Hz, 1H), 7.22 (s, 1H), 7.20 (t, J=75.0 Hz, 1H), 7.09 (s, 1H), 7.01 (dd, J₁=8.5 Hz, J₂=2.5 Hz, 1H), 4.96-4.37 (m, 1H), 4.09 (t, J=6.5 Hz, 2H), 3.88 (s, 3H), 3.59 (s, 4H), 2.86-2.81 (m, 1H), 2.44-2.29 (m, 6H), 1.95-1.88 (m, 2H), 1.13-1.05 (m, 1H), 0.99-0.80 (m, 1H). LC-MS [M+H]⁺=535.5.

### Example 87: 2-(difluoromethoxy)-4-(6-(2-(3,3-difluoropyrrolidin-1-yl) ethoxy)benzofuran-3-yl)-N-((1R,2S)-2-fluorocyclopropyl)-6-methoxybenzamide (compound 87)

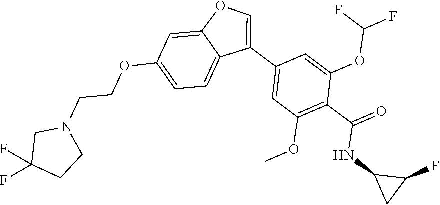 [SMILES: COc1cc(-c2coc3cc(OCCN4CCC(F)(F)C4)ccc23)cc(OC(F)F)c1C(=O)N[C@@H]1C[C@@H]1F]

Compound 87 (5 mg, 16% yield) was prepared in a manner similar to that described in Example 58 step 1 from 2-(difluoromethoxy)-N-((1R,2S)-2-fluorocyclopropyl)-4-(6-hydroxybenzofuran-3-yl)-6-methoxybenzamide and 2-(3,3-difluoropyrrolidin-1-yl) ethan-1-ol. ¹H NMR (500 MHz, DMSO-d₆) δ 8.50 (d, J=4.4 Hz, 1H), 8.39 (s, 1H), 7.79 (d, J=8.7 Hz, 1H), 7.33 (d, J=5.0 Hz, 1H), 7.22 (s, 1H), 7.20 (t, J=75.0 Hz, 1H), 7.09 (s, 1H), 7.02 (dd, J₁=8.5 Hz. J₂=2.5 Hz, 1H), 4.79-7.66 (m, 1H), 4.16 (t, J=5.6 Hz, 2H), 3.88 (s, 3H), 2.98 (t, J=5.6 Hz, 2H), 2.87-2.80 (m, 5H), 2.31-2.13 (m, 2H), 1.11-1.07 (m, 1H), 0.96-0.89 (m, 1H). LC-MS [M+H]⁺=541.4.

### Example 88: 2-(difluoromethoxy)-N-((1R,2S)-2-fluorocyclopropyl)-6-methoxy-4-(6-(2-((S)-3-methylpiperidin-1-yl) ethoxy)benzofuran-3-yl)benzamide (compound 88)

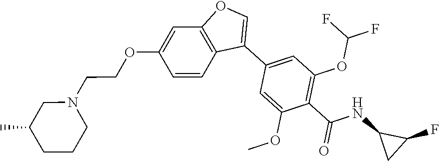 [SMILES: COc1cc(-c2coc3cc(OCCN4CCC[C@H](C)C4)ccc23)cc(OC(F)F)c1C(=O)N[C@@H]1C[C@@H]1F]

Compound 88 (17 mg, 55% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-3-methylpiperidine. ¹H NMR (500 MHz, DMSO-d₆) δ 8.50 (d. J=4.7 Hz, 1H), 8.42-8.34 (m, 1H), 7.78 (dd, J₁=14.2 Hz, J₂=8.0 Hz, 1H), 7.34-6.95 (m, 5H), 4.72 (d, J=64.7 Hz, 1H), 4.15 (d, J=6.0 Hz, 2H), 3.93-3.81 (m, 3H), 2.85 (s, 3H), 2.71 (d, J=6.1 Hz, 2H), 1.96 (s, 1H), 1.71-1.52 (m, 4H), 1.47 (d, J=9.3 Hz, 1H), 1.14-1.05 (m, 1H), 0.98-0.89 (m, 1H), 0.87-0.79 (m, 3H). LC-MS [M+H]⁺=533.5.

### Example 89: 2-(difluoromethoxy)-N-((1R,2S)-2-fluorocyclopropyl)-6-methoxy-4-(6-(2-((R)-3-methylpiperidin-1-yl) ethoxy)benzofuran-3-yl)benzamide (compound 89)

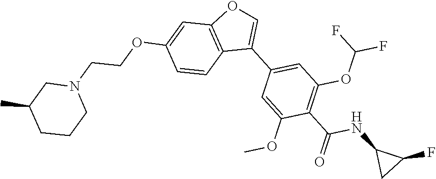 [SMILES: COc1cc(-c2coc3cc(OCCN4CCC[C@@H](C)C4)ccc23)cc(OC(F)F)c1C(=O)N[C@@H]1C[C@@H]1F]

Compound 89 (15 mg, 48% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-3-methylpiperidine. ¹H NMR (500 MHz, DMSO-d₆) δ 8.50 (d, J=4.5 Hz, 1H), 8.40 (s, 1H), 7.78 (d, J=8.7 Hz, 1H), 7.33 (s, 1H), 7.22 (s, 1H), 7.20 (t, J=75.0 Hz, 1H), 7.09 (s, 1H), 7.00 (dd, J₁=8.0 Hz, J₂=2.0 Hz, 1H), 4.80-4.65 (m, 1H), 4.17 (t, J=5.8 Hz, 2H), 3.88 (s, 3H), 2.92 (t, J=11.9 Hz, 2H), 2.86-2.81 (m, 1H), 2.76 (t, J=5.6 Hz, 2H), 2.02 (t, J=10.7 Hz, 1H), 1.75 (t, J=10.6 Hz, 1H), 1.69-1.54 (m, 3H), 1.53-1.40 (m, 1H), 1.15-1.03 (m, 1H), 0.97-0.79 (m, 5H). LC-MS [M+H]⁺=533.4.

### Example 90: 2-(difluoromethoxy)-N-((1R,2S)-2-fluorocyclopropyl)-4-(6-(2-((R)-3-isopropylpyrrolidin-1-yl) ethoxy)benzofuran-3-yl)-6-methoxybenzamide (compound 90)

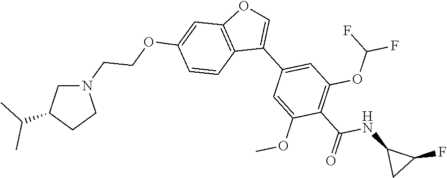 [SMILES: COc1cc(-c2coc3cc(OCCN4CC[C@H](C(C)C)C4)ccc23)cc(OC(F)F)c1C(=O)N[C@@H]1C[C@@H]1F]

Compound 90 (9 mg, 48% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-3-isopropylpyrrolidine hydrochloride. ¹H NMR (500 MHz, DMSO-d₆) δ 8.50 (d, J=4.5 Hz, 1H), 8.40 (s, 1H), 7.79 (d, J=8.7 Hz, 1H), 7.33 (s, 1H), 7.23 (s, 1H), 7.20 (t, J=75.0 Hz, 1H), 7.09 (s, 1H), 7.00 (dd, J₁=7.5 Hz, J₂=2.0 Hz, 1H), 4.84-4.62 (m, 1H), 4.15 (t, J=5.8 Hz, 2H), 3.88 (s, 3H), 2.96-2.75 (m, 5H), 2.21 (t, J=8.6 Hz, 1H), 1.91-1.72 (m, 2H), 1.51-1.34 (m, 2H), 1.13-1.06 (m, 1H), 1.01-0.81 (m, 8H). LC-MS [M+H]⁺=547.5.

### Example 91: 2-(difluoromethoxy)-N-((1R,2S)-2-fluorocyclopropyl)-6-methoxy-4-(6-(2-((S)-2-methylpiperidin-1-yl) ethoxy)benzofuran-3-yl)benzamide (compound 91)

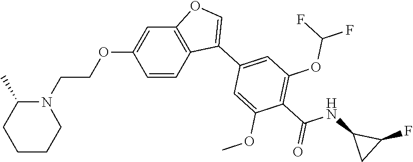 [SMILES: COc1cc(-c2coc3cc(OCCN4CCCC[C@@H]4C)ccc23)cc(OC(F)F)c1C(=O)N[C@@H]1C[C@@H]1F]

Compound 91 (12 mg, 48% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-2-methylpiperidine. ¹H NMR (500 MHz, DMSO-d₆) δ 8.50 (d, J=4.0 Hz, 1H), 8.38 (d, J=2.0 Hz, 1H), 7.78 (d, J=8.6 Hz, 1H), 7.32 (s, 1H), 7.22 (s, 1H), 7.20 (t, J=75.0 Hz, 1H), 7.09 (s, 1H), 7.00 (dd, J₁=8.5 Hz, J₂=2.5 Hz, 1H), 4.85-4.54 (m, 1H), 4.13 (d, J=5.8 Hz, 2H), 3.88 (s, 3H), 3.05 (dt, J₁=12.0 Hz, J₂=6.0 Hz, 1H), 2.96-2.79 (m, 2H), 2.77-2.66 (m, 1H), 2.43 (s, 1H), 2.32 (t, J=11.0 Hz, 1H), 1.60-1.39 (m, 4H), 1.24 (t, J=18.5 Hz, 2H), 1.14-1.03 (m, 4H), 1.00-0.87 (m, 1H). LC-MS [M+H]⁺=533.4.

### Example 92: 2-(difluoromethoxy)-N-((1R,2S)-2-fluorocyclopropyl)-6-methoxy-4-(6-(2-((R)-2-methylpiperidin-1-yl) ethoxy)benzofuran-3-yl)benzamide (compound 92)

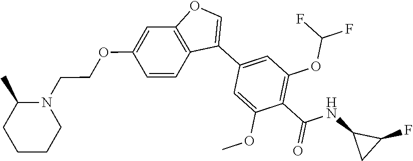 [SMILES: COc1cc(-c2coc3cc(OCCN4CCCC[C@H]4C)ccc23)cc(OC(F)F)c1C(=O)N[C@@H]1C[C@@H]1F]

Compound 92 (9 mg, 29% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and (R)-2-methylpiperidine. ¹H NMR (500 MHz, DMSO-d₆) δ 8.50 (s, 1H), 8.40 (s, 1H), 7.80 (dd, J₁=8.7 Hz, J₂=1.9 Hz, 1H), 7.34 (s, 1H), 7.22 (s, 1H), 7.20 (t, J=75.0 Hz, 1H), 7.09 (s, 1H), 7.00 (dd, J₁=8.5 Hz, J₂=2.5 Hz, 1H), 4.86-4.60 (m, 1H), 4.19 (t, J=5.6 Hz, 2H), 3.88 (s, 3H), 3.25-3.11 (m, 1H), 3.03 (d, J=11.3 Hz, 1H), 2.95-2.79 (m, 2H), 2.64 (s, 1H), 1.74-0.56 (m, 3H), 1.50 (d, J=10.2 Hz, 1H), 1.29-1.25 (m, 2H), 1.19-1.04 (m, 4H), 1.01-0.86 (m, 1H). LC-MS [M+H]=533.4.

### Example 93: 2-(difluoromethoxy)-4-(6-(2-(3,3-dimethylpyrrolidin-1-yl) ethoxy)benzofuran-3-yl)-N-((1R,2S)-2-fluorocyclopropyl)-6-methoxybenzamide (compound 93)

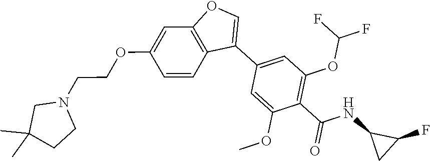 [SMILES: COc1cc(-c2coc3cc(OCCN4CCC(C)(C)C4)ccc23)cc(OC(F)F)c1C(=O)N[C@@H]1C[C@@H]1F]

Compound 93 (13 mg, 48% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and 3,3-dimethylpyrrolidine hydrochloride. ¹H NMR (500 MHz, DMSO-d₆) δ 8.50 (d, J=4.4 Hz, 1H), 8.39 (s, 1H), 7.79 (d, J=8.7 Hz, 1H), 7.34 (s, 1H), 7.22 (s, 1H), 7.20 (t, J=75.0 Hz, 1H), 7.09 (s, 1H), 7.00 (dd, J₁=8.0 Hz, J₂=2.0 Hz, 1H), 4.79-4.65 (m, 1H), 4.17 (t, J=5.7 Hz, 2H), 3.88 (s, 3H), 2.94 (s, 2H), 2.88-2.73 (m, 3H), 1.57 (t, J=7.1 Hz, 2H), 1.15-1.03 (m, 8H), 0.99-0.87 (m, 1H). LC-MS [M+H]⁺=533.4.

### Example 94: 2-(difluoromethoxy)-N-((1R,2S)-2-fluorocyclopropyl)-6-methoxy-4-(6-(2-(4-methylpiperidin-1-yl) ethoxy)benzofuran-3-yl)benzamide (compound 94)

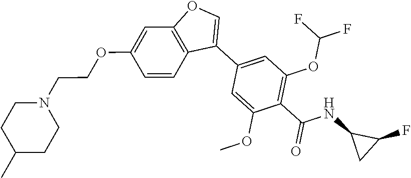 [SMILES: COc1cc(-c2coc3cc(OCCN4CCC(C)CC4)ccc23)cc(OC(F)F)c1C(=O)N[C@@H]1C[C@@H]1F]

Compound 94 (19 mg, 62% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and 4-methylpiperidine. ¹H NMR (500 MHz, DMSO-d₆) δ 8.50 (d, J=4.3 Hz, 1H), 8.39 (s, 1H), 7.79 (d, J=8.7 Hz, 1H), 7.34 (s, 1H), 7.22 (s, 1H), 7.20 (t, J=75.0 Hz, 1H), 7.09 (s, 1H), 7.00 (dd, J₁=8.5 Hz, J₂=2.0 Hz, 1H), 4.81-7.65 (m, 1H), 4.18 (t, J=5.6 Hz, 2H), 3.89 (s, 3H), 2.99 (d. J=11.1 Hz, 2H), 2.91-2.78 (m, 3H), 2.16 (t, J=11.0 Hz, 2H), 1.61 (d, J=12.3 Hz, 2H), 1.37 (d, J=6.1 Hz, 1H), 1.26-1.05 (m, 3H), 0.96-0.90 (m, 4H). LC-MS [M+H]⁺=533.4.

### Example 95: 2-(difluoromethoxy)-N-((1R,2S)-2-fluorocyclopropyl)-6-methoxy-4-(6-(2-(2-(trifluoromethyl) pyrrolidin-1-yl) ethoxy)benzofuran-3-yl)benzamide (compound 95)

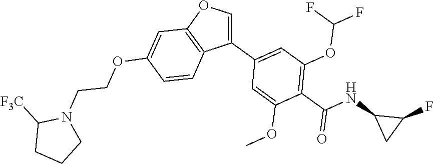 [SMILES: COc1cc(-c2coc3cc(OCCN4CCCC4C(F)(F)F)ccc23)cc(OC(F)F)c1C(=O)N[C@@H]1C[C@@H]1F]

Compound 95 (26.5 mg, 34% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and 2-(trifluoromethyl) pyrrolidine. ¹H NMR (500 MHz, DMSO-d₆) δ 8.50 (d, J=4.4 Hz, 1H), 8.39 (s, 1H), 7.78 (d, J=8.7 Hz, 1H), 7.30 (s, 1H), 7.22 (s, 1H), 7.20 (t, J=75.0 Hz, 1H), 7.09 (s, 1H), 7.00 (dd, J₁=10.0 Hz, J₂=5.0 Hz, 1H), 4.87-4.51 (m, 1H), 4.16 (t, J=5.9 Hz, 2H), 3.88 (s, 3H), 3.60-3.45 (m, 1H), 3.20 (dt, J₁=15.9 Hz, J₂=6.3 Hz, 2H), 3.11-2.98 (m, 1H), 2.83 (dt, J₁=9.1 Hz, J₂=4.6 Hz, 1H), 2.65-2.56 (m, 1H), 2.09-1.97 (m, 1H), 1.80 (dd, J₁=12.0 Hz, J₂=8.6 Hz, 2H), 1.78-1.70 (m, 1H), 1.12-1.07 (m, 1H), 1.02-0.84 (m, 1H). LC-MS [M+H]⁺=573.0.

### Example 96: 2-(difluoromethoxy)-4-(6-(2-((S)-3-ethylpyrrolidin-1-yl) ethoxy)benzofuran-3-yl)-N-((1R,2S)-2-fluorocyclopropyl)-6-methoxybenzamide (compound 96)

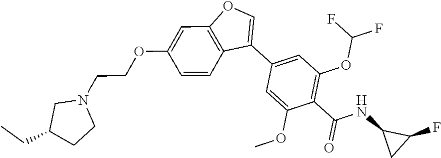 [SMILES: CC[C@H]1CCN(CCOc2ccc3c(-c4cc(OC)c(C(=O)N[C@@H]5C[C@@H]5F)c(OC(F)F)c4)coc3c2)C1]

Compound 96 (17 mg, 48% yield) was prepared in a manner similar to that described in Example 67 step 2 from 4-(6-(2-bromoethoxy)benzofuran-3-yl)-2-(difluoromethoxy)-N-((1R,2S)-2-fluorocyclopropyl)-6-methoxybenzamide and(S)-3-ethylpyrrolidine hydrochloride. ¹H NMR (500 MHz, DMSO-d₆) δ 8.50 (d, J=4.5 Hz, 1H), 8.40 (s, 1H), 7.80 (d, J=8.7 Hz, 1H), 7.34 (s, 1H), 7.22 (s, 1H), 7.20 (t. J=75.0 Hz, 1H), 7.09 (s, 1H), 7.00 (dd, J₁=10.0 Hz, J₂=5.0 Hz, 1H), 4.83-4.62 (m, 1H), 4.21 (t, J=5.6 Hz, 2H), 3.88 (s, 3H), 3.07 (s, 3H), 2.97-2.88 (m, 1H), 2.86-2.79 (m, 2H), 2.44-2.34 (m, 1H), 2.14-2.02 (m, 1H), 2.01-1.92 (m, 1H), 1.44-1.30 (m, 3H), 1.12-1.06 (m, 1H), 1.00-0.91 (m, 1H), 0.87 (t, J=7.4 Hz, 3H). LC-MS [M+H]⁺=533.5.

### Example 97: 2-(difluoromethoxy)-N-((1R,2R)-2-fluorocyclopropyl)-6-methoxy-4-(6-(2-(pyrrolidin-1-yl) ethoxy)benzofuran-3-yl)benzamide (compound 97)

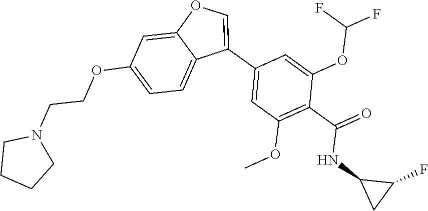 [SMILES: COc1cc(-c2coc3cc(OCCN4CCCC4)ccc23)cc(OC(F)F)c1C(=O)N[C@@H]1C[C@H]1F]

Compound 97 (11 mg, 24% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and (1R,2R)-2-fluorocyclopropan-1-amine. ¹H NMR (500 MHz, DMSO-d₆) δ 8.43-8.40 (m, 1H), 8.39 (s, 1H), 8.17 (s, 1H), 7.79 (d, J=8.7 Hz, 1H), 7.34 (s, 1H), 7.22 (s, 1H), 7.20 (t. J=75.0 Hz, 1H), 7.09 (s, 1H), 7.00 (dd, J₁=10.0 Hz, J₂=5.0 Hz, 1H), 4.71-4.54 (m, 1H), 4.21-4.15 (m, 2H), 3.88 (s, 3H), 3.22-3.11 (m, 1H), 2.97-2.89 (m, 2H), 2.69-2.62 (m, 4H), 1.81-1.67 (m, 4H), 1.46-1.32 (m, 1H), 0.98-0.87 (m, 1H). LC-MS [M+H]=505.3.

### Example 98: 2-(difluoromethoxy)-N-((1S,2R)-2-fluorocyclopropyl)-6-methoxy-4-(6-(2-(pyrrolidin-1-yl) ethoxy)benzofuran-3-yl)benzamide (compound 98)

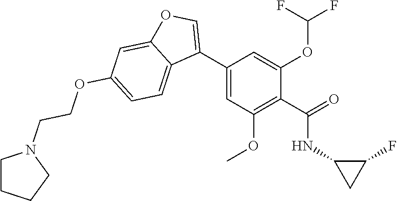 [SMILES: COc1cc(-c2coc3cc(OCCN4CCCC4)ccc23)cc(OC(F)F)c1C(=O)N[C@H]1C[C@H]1F]

Compound 98 (10 mg, 22% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and (1S,2R)-2-fluorocyclopropan-1-amine. ¹H NMR (500 MHz, DMSO-d₆) δ 8.50 (d, J=4.5 Hz, 1H), 8.41 (s, 1H), 7.80 (d, J=8.7 Hz, 1H), 7.34 (s, 1H), 7.22 (s, 1H), 7.20 (t, J=75.0 Hz, 1H), 7.09 (s, 1H), 7.00 (dd, J₁=8.5 Hz, J₂=2.0 Hz, 1H), 4.84-4.62 (m, 1H), 4.29-4.16 (m, 2H), 3.90 (s, 3H), 3.31-3.28 (m, 1H), 3.12 (s, 2H), 2.91-2.73 (m, 4H), 1.79 (s, 4H), 1.10-1.06 (m, 1H), 0.99-0.87 (m, 1H). LC-MS [M+H]⁺=505.3.

### Example 99: 2-(difluoromethoxy)-N-((1S,2S)-2-fluorocyclopropyl)-6-methoxy-4-(6-(2-(pyrrolidin-1-yl) ethoxy)benzofuran-3-yl)benzamide (compound 99)

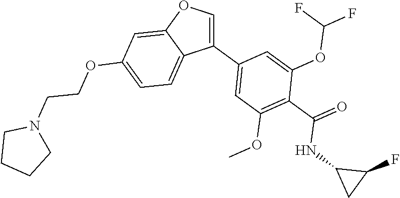 [SMILES: COc1cc(-c2coc3cc(OCCN4CCCC4)ccc23)cc(OC(F)F)c1C(=O)N[C@H]1C[C@@H]1F]

The trans racemate (30 mg) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and trans-2-fluorocyclopropanamine hydrochloride. The trans racemate was separated by chiral HPLC with the following conditions [column: i-cellulose-5 4.6*250 mm 5 um; gradient: Hex/EtOH (0.1% 2M NH₃MeOH, v/v=70/30); flow rate: 1.0 mL/min] to afford compound 97 (11 mg, 9% yield, retention time: 8.9 min) and compound 99 (12 mg, 10% yield, retention time: 10.5 min).

Compound 99: ¹H NMR (500 MHz, DMSO-d₆) δ 8.44-8.40 (m, 1H), 8.39 (s, 1H), 7.78 (d, J=8.7 Hz, 1H), 7.32 (s, 1H), 7.24 (s, 1H), 7.22 (t, J=75.0 Hz, 1H), 7.09 (s, 1H), 7.01 (dd, J₁=8.5 Hz, J₂=2.0 Hz, 1H), 4.74-4.52 (m, 1H), 4.24-4.11 (m, 2H), 3.89 (s, 3H), 3.22-3.09 (m, 1H), 2.96-2.83 (m, 2H), 2.69-2.56 (m, 4H), 1.78-1.64 (m, 4H), 1.45-1.32 (m, 1H), 0.98-0.88 (m, 1H). LC-MS [M+H]⁺=505.4.

### Example 100: N-(2,2-difluorocyclopropyl)-2-(difluoromethoxy)-6-methoxy-4-(6-(2-(pyrrolidin-1-yl) ethoxy)benzofuran-3-yl)benzamide (compound 100)

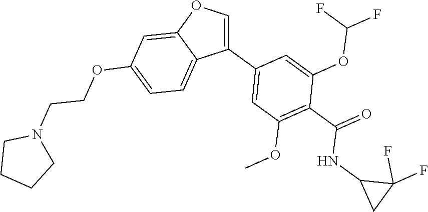 [SMILES: COc1cc(-c2coc3cc(OCCN4CCCC4)ccc23)cc(OC(F)F)c1C(=O)NC1CC1(F)F]

Compound 100 (5 mg, 14% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and 2,2-difluorocyclopropan-1-amine. ¹H NMR (500 MHz, DMSO-d₆) δ 8.78 (s, 1H), 8.40 (s, 1H), 7.79 (d, J=8.7 Hz, 1H), 7.33 (s, 1H), 7.24 (s, 1H), 7.22 (t, J=75.0 Hz, 1H), 7.10 (s, 1H), 7.01 (dd, J₁=8.5 Hz, J₂=2.0 Hz, 1H), 4.16 (t, J=5.8 Hz, 2H), 3.90 (s, 3H), 3.53-3.42 (m, 1H), 2.88 (t, J=5.6 Hz, 2H), 2.62-2.56 (m, 4H), 1.99-1.88 (m, 1H), 1.79-1.64 (m, 4H), 1.54-1.41 (m, 1H). LC-MS [M+H]⁺=523.2.

### Example 101: N-cyclopropyl-2-(difluoromethoxy)-6-(((1r,4r)-4-hydroxycyclohexyl)oxy)-4-(6-(2-(pyrrolidin-1-yl) ethoxy)benzofuran-3-yl)benzamide (compound 101)

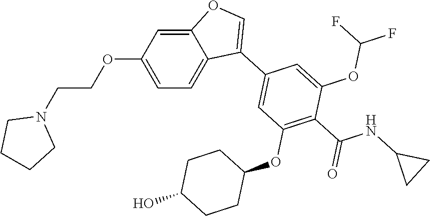 [SMILES: O=C(NC1CC1)c1c(OC(F)F)cc(-c2coc3cc(OCCN4CCCC4)ccc23)cc1O[C@H]1CC[C@H](O)CC1]

### Step 1: 4-bromo-N-cyclopropyl-2-(difluoromethoxy)-6-hydroxybenzamide

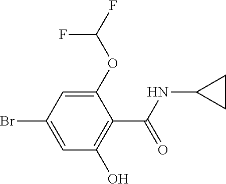 [SMILES: O=C(NC1CC1)c1c(O)cc(Br)cc1OC(F)F]

The title compound of step 1 (600 mg, 62% yield) was prepared in a manner similar to that described in Example 13 step 1 from 4-bromo-N-cyclopropyl-2-(difluoromethoxy)-6-methoxybenzamide and BBr₃. LC-MS [M+H]⁺=322.3.

### Step 2: N-cyclopropyl-2-(difluoromethoxy)-6-hydroxy-4-(4,4,5,5-tetramethyl-1,3,2-dioxaborolan-2-yl)benzamide

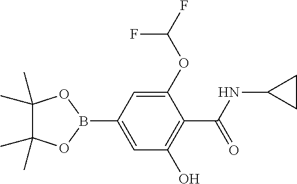 [SMILES: CC1(C)OB(c2cc(O)c(C(=O)NC3CC3)c(OC(F)F)c2)OC1(C)C]

The title compound of step 2 (400 mg, 65% yield) was prepared in a manner similar to that described in Example 1 step 4 from 4-bromo-N-cyclopropyl-2-(difluoromethoxy)-6-hydroxybenzamide and 4,4,4′,4′,5,5,5′,5′-octamethyl-2,2′-bi (1,3,2-dioxaborolane). LC-MS [M+H]⁺=370.3.

### Step 3: N-cyclopropyl-2-(difluoromethoxy)-6-hydroxy-4-(6-(2-(pyrrolidin-1-yl) ethoxy)benzofuran-3-yl)benzamide

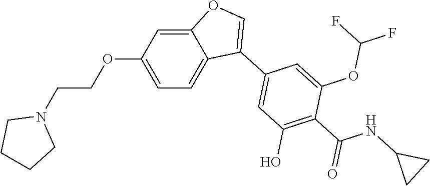 [SMILES: O=C(NC1CC1)c1c(O)cc(-c2coc3cc(OCCN4CCCC4)ccc23)cc1OC(F)F]

The title compound of step 3 (300 mg, 59% yield) was prepared in a manner similar to that described in Example 1 step 5 from N-cyclopropyl-2-(difluoromethoxy)-6-hydroxy-4-(4,4,5,5-tetramethyl-1,3,2-dioxaborolan-2-yl)benzamide and 6-(2-(pyrrolidin-1-yl) ethoxy)benzofuran-3-yl trifluoromethanesulfonate. LC-MS [M+H]⁺=473.2.

### Step 4: N-cyclopropyl-2-(difluoromethoxy)-6-(((1r,4r)-4-hydroxycyclohexyl)oxy)-4-(6-(2-(pyrrolidin-1-yl) ethoxy)benzofuran-3-yl)benzamide (compound 101)

Compound 101 (1.6 mg, 1% yield) was prepared in a manner similar to that described in Example 58 step 1 from N-cyclopropyl-2-(difluoromethoxy)-6-hydroxy-4-(6-(2-(pyrrolidin-1-yl) ethoxy)benzofuran-3-yl)benzamide and trans-cyclohexane-1,4-diol. ¹H NMR (500 MHz, DMSO-d₆) δ 8.36 (s, 1H), 8.27 (d, J=4.2 Hz, 1H), 7.71 (d, J=8.6 Hz, 1H), 7.39-7.18 (m, 3H), 7.08-6.97 (m, 2H), 5.32 (t, J=4.7 Hz, 1H), 4.54 (d, J=3.8 Hz, 2H), 4.15 (t. J=5.7 Hz, 2H), 3.59 (d, J=3.7 Hz, 1H), 2.85 (s, 2H), 2.75 (td, J=7.2, 3.6 Hz, 1H), 2.00 (dd, J=16.6, 7.4 Hz, 3H), 1.82 (s, 3H), 1.70 (s, 4H), 1.46 (d, J=10.1 Hz, 3H), 0.85 (t, J=6.9 Hz, 2H), 0.74-0.63 (m, 2H), 0.49-0.41 (m, 2H). LC-MS [M+H]⁺=570.9.

### Example 102: N-cyclopropyl-2-(difluoromethoxy)-6-(((1s,4s)-4-hydroxycyclohexyl)oxy)-4-(6-(2-(pyrrolidin-1-yl) ethoxy)benzofuran-3-yl)benzamide (compound 102)

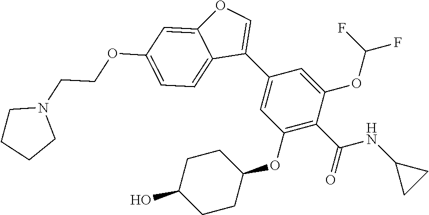 [SMILES: O=C(NC1CC1)c1c(OC(F)F)cc(-c2coc3cc(OCCN4CCCC4)ccc23)cc1O[C@H]1CC[C@@H](O)CC1]

Compound 102 (8 mg, 6% yield) was prepared in a manner similar to that described in Example 58 step 1 from N-cyclopropyl-2-(difluoromethoxy)-6-hydroxy-4-(6-(2-(pyrrolidin-1-yl) ethoxy)benzofuran-3-yl)benzamide and cis-cyclohexane-1,4-diol. ¹H NMR (500 MHz, DMSO-d₆) δ 8.35 (s, 1H), 8.30 (d, J=4.0 Hz, 1H), 7.70 (d, J=8.7 Hz, 1H), 7.32 (s, 1H), 7.24 (s, 1H), 7.22 (t, J=75.0 Hz, 1H), 7.08 (s, 1H), 7.01 (dd, J₁=8.5 Hz, J₂=2.0 Hz, 1H), 4.60 (s, 1H), 4.15 (t, J=5.8 Hz, 2H), 3.59-3.54 (m, 1H), 2.86 (t, J=5.8 Hz, 2H), 2.75 (td, J₁=7.2 Hz, J₂=3.7 Hz, 2H), 2.58 (s, 2H), 1.86 (s, 3H), 1.70 (t, J=3.2 Hz, 5H), 1.64-1.60 (m, 2H), 1.58-1.53 (m, 4H), 0.69-0.64 (m, 2H), 0.54-0.48 (m, 2H). LC-MS [M+H]⁺=570.9.

### Example 103: N-cyclopropyl-2-(difluoromethoxy)-6-(2-hydroxyethoxy)-4-(6-(2-(pyrrolidin-1-yl) ethoxy)benzofuran-3-yl)benzamide (compound 103)

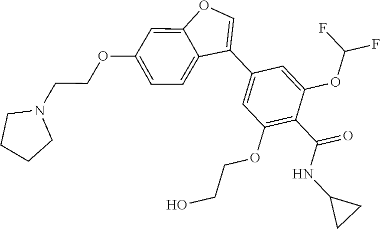 [SMILES: O=C(NC1CC1)c1c(OCCO)cc(-c2coc3cc(OCCN4CCCC4)ccc23)cc1OC(F)F]

### Step 1: methyl 4-bromo-2-(difluoromethoxy)-6-fluorobenzoate

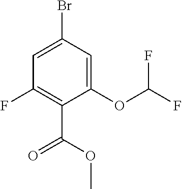 [SMILES: COC(=O)c1c(F)cc(Br)cc1OC(F)F]

To a solution of methyl 4-bromo-2-fluoro-6-hydroxybenzoate (14 g, 56.22 mmol) in ACN (100 mL) and H₂O (100 mL) was added KOH (63.09 g, 1.12 mol) at 20° C. After addition, then diethyl (bromodifluoromethyl)phosphonate (30.02 g, 112.43 mmol) was added dropwise at −40° C. The resulting mixture was stirred at 20° C. for 0.5 hrs. TLC indicated the reactant was consumed completely. The combined reaction mixture was poured into water (100 mL) and extracted with EtOAc (100 mL*2). The combined organic layers were washed with brine, dried over anhydrous Na₂SO₄, filtered and concentrated under reduced pressure. The residue was purified by silica gel column chromatography, eluting with PE/EtOAc (v/v=1/0 to 1/1), to give the title product (12 g, 65% yield). ¹H NMR (400 MHz, CDCl₃) δ 7.31-7.23 (m, 2H), 6.55 (t, J=76.0 Hz, 1H), 3.96 (s, 3H).

### Step 2: methyl 4-bromo-2-(2-((tert-butyldimethylsilyl)oxy) ethoxy)-6-(difluoromethoxy)benzoate

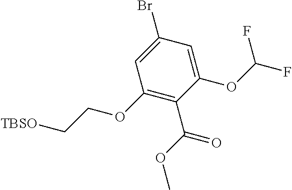 [SMILES: COC(=O)c1c(OCCO[Si](C)(C)C(C)(C)C)cc(Br)cc1OC(F)F]

To a solution of 2-((tert-butyldimethylsilyl)oxy) ethan-1-ol (352 mg, 2 mmol) in anhydrous THF (10 mL) cooled to 0° C. was added NaH (60% dispersion in mineral oil, 96 mg, 2.4 mmol) in four equivalent portions. The mixture was stirred at 0° C. for 30 minutes, and then methyl 4-bromo-2-(difluoromethoxy)-6-fluorobenzoate (299 mg, 1 mmol) was added slowly. The reaction was stirred at room temperature for 16 hours, diluted with EA (20 mL), and washed with brine (20 mL). The organic layers were dried over anhydrous Na₂SO₄ and concentrated under reduced pressure to afford the title product (272 mg, 60% yield). LC-MS [M+H]⁺=455.1.

### Step 3:4-bromo-2-(difluoromethoxy)-6-(2-hydroxyethoxy)benzoic acid

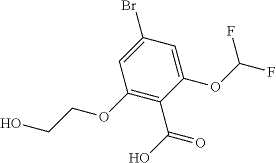 [SMILES: O=C(O)c1c(OCCO)cc(Br)cc1OC(F)F]

A mixture of methyl 4-bromo-2-(2-((tert-butyldimethylsilyl)oxy) ethoxy)-6-(difluoromethoxy)benzoate (272 mg, 0.6 mmol) and LiOH (50 mg, 1.2 mmol) in MeOH (10 mL) and water (10 mL) was stirred at room temperature for 4 h. The pH of mixture was adjusted to 2-3 by 1N HCl solution. Extraction was carried out using EtOAc (20 mL*2); the combined organic layers were washed with brine (30 mL), dried over anhydrous Na₂SO₄, filtered and concentrated under reduced pressure to give the title product as a white solid (196 mg, 100% yield). LC-MS [M+H]⁺=327.1.

### Step 4: 4-bromo-N-cyclopropyl-2-(difluoromethoxy)-6-(2-hydroxyethoxy)benzamide

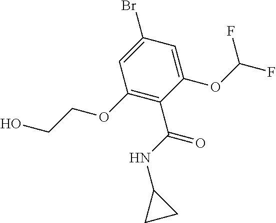 [SMILES: O=C(NC1CC1)c1c(OCCO)cc(Br)cc1OC(F)F]

The title compound of step 4 (182 mg, 67% yield) was prepared in a manner similar to that described in Example 25 step 9 from 4-bromo-2-(difluoromethoxy)-6-(2-hydroxyethoxy)benzoic acid and cyclopropanamine. LC-MS [M+H]⁺=366.1.

### Step 5: N-cyclopropyl-2-(difluoromethoxy)-6-(2-hydroxyethoxy)-4-(4,4,5,5-tetramethyl-1,3,2-dioxaborolan-2-yl)benzamide

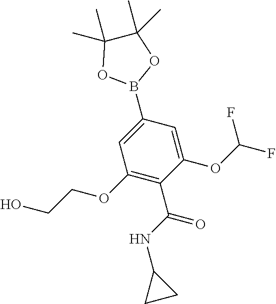 [SMILES: CC1(C)OB(c2cc(OCCO)c(C(=O)NC3CC3)c(OC(F)F)c2)OC1(C)C]

The title compound of step 5 (207 mg, 100% yield) was prepared in a manner similar to that described in Example 1 step 4 from 4-bromo-N-cyclopropyl-2-(difluoromethoxy)-6-(2-hydroxyethoxy)benzamide and 4,4,4′,4′,5,5,5′,5′-octamethyl-2,2′-bi (1,3,2-dioxaborolane). LC-MS [M+H]⁺=414.1.

### Step 6: N-cyclopropyl-2-(difluoromethoxy)-6-(2-hydroxyethoxy)-4-(6-(2-(pyrrolidin-1-yl) ethoxy)benzofuran-3-yl)benzamide (compound 103)

Compound 103 (12 mg, 5% yield) was prepared in a manner similar to that described in Example 1 step 5 from N-cyclopropyl-2-(difluoromethoxy)-6-(2-hydroxyethoxy)-4-(4,4,5,5-tetramethyl-1,3,2-dioxaborolan-2-yl)benzamide and 6-(2-(pyrrolidin-1-yl) ethoxy)benzofuran-3-yl trifluoromethanesulfonate. ¹H NMR (500 MHz, DMSO-d₆) δ 8.38 (s, 1H), 8.28 (d, J=4.3 Hz, 1H), 7.77 (d, J=8.7 Hz, 1H), 7.32 (s, 1H), 7.24 (s, 1H), 7.22 (t, J=75.0 Hz, 1H), 7.09 (s, 1H), 7.01 (dd, J₁=8.5 Hz, J₂=2.0 Hz, 1H), 4.84 (brs, 1H), 4.16 (t, J=4.8 Hz, 4H), 3.70 (t, J=5.1 Hz, 2H), 2.88 (t, J=5.6 Hz, 2H), 2.80-2.76 (m, 1H), 2.60 (s, 4H), 1.71 (s, 4H), 0.75-0.63 (m, 2H), 0.53-0.44 (m, 2H). LC-MS [M+H]⁺=517.4.

### Example 104: N-cyclopropyl-2-(difluoromethoxy)-6-((2-hydroxyethyl)amino)-4-(6-(2-(pyrrolidin-1-yl) ethoxy)benzofuran-3-yl)benzamide (compound 104)

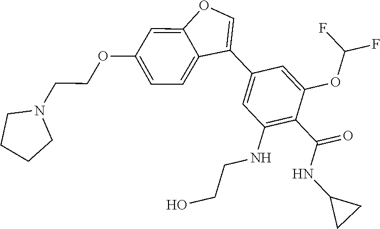 [SMILES: O=C(NC1CC1)c1c(NCCO)cc(-c2coc3cc(OCCN4CCCC4)ccc23)cc1OC(F)F]

### Step 1: methyl 4-bromo-2-(difluoromethoxy)-6-((2-hydroxyethyl)amino) benzoate

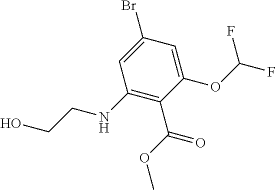 [SMILES: COC(=O)c1c(NCCO)cc(Br)cc1OC(F)F]

A solution of methyl 4-bromo-2-(difluoromethoxy)-6-fluorobenzoate (299 mg, 1 mmol) and 2-aminoethan-1-ol (260 mg, 5 mmol) in DME (6 mL) was stirred at 90° C. for 8 hours, diluted with EA (20 mL), and washed with brine (20 mL). The combined organic layers were dried over anhydrous Na₂SO₄, filtered and concentrated in vacuo. The residue was purified by silica gel column chromatography, eluting with PE/EtOAc (v/v=2/1), to afford the title product as a white solid (180 mg, 53% yield). LC-MS [M+H]⁺=339.1.

### Step 2: 4-bromo-2-(difluoromethoxy)-6-((2-hydroxyethyl)amino)benzoic acid

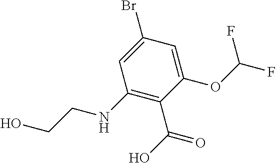 [SMILES: O=C(O)c1c(NCCO)cc(Br)cc1OC(F)F]

The title compound of step 2 (220 mg, 100% yield) was prepared in a manner similar to that described in Example 103 step 3 form methyl 4-bromo-2-(difluoromethoxy)-6-((2-hydroxyethyl)amino)benzoate and lithium hydroxide. LC-MS [M+H]⁺=326.1.

### Step 3: 4-bromo-N-cyclopropyl-2-(difluoromethoxy)-6-((2-hydroxyethyl)amino)benzamide

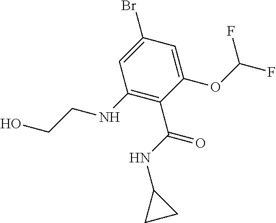 [SMILES: O=C(NC1CC1)c1c(NCCO)cc(Br)cc1OC(F)F]

The title compound of step 2 (180 mg, 67% yield) was prepared in a manner similar to that described in Example 25 step 9 from 4-bromo-2-(difluoromethoxy)-6-((2-hydroxyethyl)amino)benzoic acid and cyclopropanamine. LC-MS [M+H]⁺=365.1.

### Step 3: N-cyclopropyl-2-(difluoromethoxy)-6-((2-hydroxyethyl)amino)-4-(4,4,5,5-tetramethyl-1,3,2-dioxaborolan-2-yl)benzamide

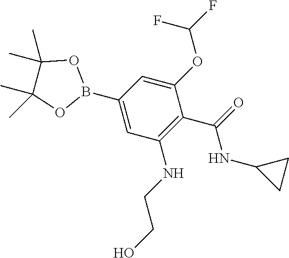 [SMILES: CC1(C)OB(c2cc(NCCO)c(C(=O)NC3CC3)c(OC(F)F)c2)OC1(C)C]

The title compound of step 3 (205 mg, 100% yield) was prepared in a manner similar to that described in Example 1 step 4 from 4-bromo-N-cyclopropyl-2-(difluoromethoxy)-6-((2-hydroxyethyl)amino)benzamide and 4,4,4′,4′,5,5,5′,5′-octamethyl-2,2′-bi (1,3,2-dioxaborolane). LC-MS [M+H]⁺=413.1.

### Step 4: N-cyclopropyl-2-(difluoromethoxy)-6-((2-hydroxyethyl)amino)-4-(6-(2-(pyrrolidin-1-yl) ethoxy)benzofuran-3-yl)benzamide (compound 104)

Compound 104 (6 mg, 2% yield) was prepared in a manner similar to that described in Example 1 step 5 from N-cyclopropyl-2-(difluoromethoxy)-6-((2-hydroxyethyl)amino)-4-(4,4,5,5-tetramethyl-1,3,2-dioxaborolan-2-yl)benzamide and 6-(2-(pyrrolidin-1-yl) ethoxy)benzofuran-3-yl trifluoromethanesulfonate. ¹H NMR (500 MHz, DMSO-d₆) δ 8.33-8.29 (m, 2H), 7.73 (d, J=8.7 Hz, 1H), 7.30 (d, J=2.1 Hz, 1H), 7.12 (t, J=75.0 Hz, 1H), 7.00 (dd, J₁=8.5 Hz, J₂=2.0 Hz, 1H), 6.81 (s, 1H), 6.65 (s, 1H), 5.91 (t, J=5.3 Hz, 1H), 4.85 (bro, 1H), 4.16 (t, J=5.8 Hz, 2H), 3.61 (t. J=5.5 Hz, 2H), 3.24-3.21 (m, 2H), 2.92-2.81 (m, 3H), 2.59 (s, 4H), 1.71 (t, J=3.2 Hz, 4H), 0.74-0.66 (m, 2H), 0.55-0.47 (m, 2H). LC-MS [M+H]⁺=516.4.

### Example 105: N-cyclopropyl-2-(difluoromethoxy)-6-fluoro-4-(6-(2-(pyrrolidin-1-yl) ethoxy)benzofuran-3-yl)benzamide (compound 105)

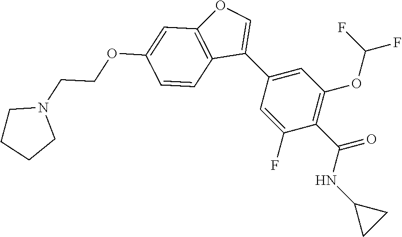 [SMILES: O=C(NC1CC1)c1c(F)cc(-c2coc3cc(OCCN4CCCC4)ccc23)cc1OC(F)F]

### Step 1: 4-bromo-2-(difluoromethoxy)-6-fluorobenzoic acid

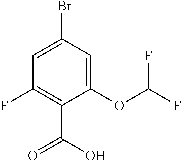 [SMILES: O=C(O)c1c(F)cc(Br)cc1OC(F)F]

To a solution of methyl 4-bromo-2-(difluoromethoxy)-6-fluorobenzoate (12.5 g, 41.80 mmol) in THF (60 mL), MeOH (60 mL) and H₂O (20 mL) was added LiOH·H₂O (5.26 g, 125.40 mmol). The mixture was stirred at 40° C. for 1 hrs. TLC indicated the reactant was consumed completely. The pH was adjusted to around 6 by 1M HCl, extracted with EtOAc (100 mL*3). The combined organic layers were washed with brine, dried over Na₂SO₄, filtered and concentrated under reduced pressure. The residue was purified by silica gel column chromatography, eluting with PE/EtOAc (v/v=1/0 to 1/1), to give the title product (9 g, 75% yield). ¹H NMR (400 MHz, CDCl₃) δ 11.12-10.59 (bro, 1H), 7.32-7.14 (m, 2H), 6.50 (t, J=72.8 Hz, 1H).

### Step 2: 4-bromo-N-cyclopropyl-2-(difluoromethoxy)-6-fluorobenzamide

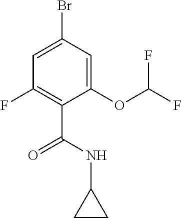 [SMILES: O=C(NC1CC1)c1c(F)cc(Br)cc1OC(F)F]

The title compound of step 2 (8.5 g, 83% yield) was prepared in a manner similar to that described in Example 25 step 9 from 4-bromo-2-(difluoromethoxy)-6-fluorobenzoic acid and cyclopropanamine. LC-MS [M+H]⁺=324.0.

### Step 3: N-cyclopropyl-2-(difluoromethoxy)-6-fluoro-4-(4,4,5,5-tetramethyl-1,3,2-dioxaborolan-2-yl)benzamide

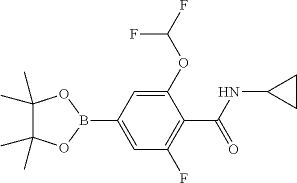 [SMILES: CC1(C)OB(c2cc(F)c(C(=O)NC3CC3)c(OC(F)F)c2)OC1(C)C]

The title compound of step 3 (5.5 g, 96% yield) was prepared in a manner similar to that described in Example 1 step 4 from 4-bromo-N-cyclopropyl-2-(difluoromethoxy)-6-fluorobenzamide and 4,4,4′,4′,5,5,5′,5′-octamethyl-2,2′-bi (1,3,2-dioxaborolane). ¹H NMR (400 MHz, CDCl₃) δ 7.43-7.35 (m, 2H), 6.61 (t, J=74.0 Hz, 1H), 5.99 (s, 1H), 2.90 (qt, J=3.2, 6.8 Hz, 1H), 1.35 (s, 12H), 0.91-0.85 (m, 2H), 0.68-0.62 (m, 2H).

### Step 4: 6-((2-methoxyethoxy) methoxy)benzofuran-3 (2H)-one

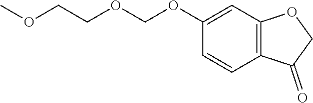 [SMILES: COCCOCOc1ccc2c(c1)OCC2=O]

To a solution of 6-hydroxybenzofuran-3 (2H)-one (5 g, 33.30 mmol) in DCM (50 mL) was added 1-(chloromethoxy)-2-methoxyethane (4.98 g, 39.97 mmol, 4.56 mL) and DIEA (10.76 g, 83.26 mmol, 14.50 mL) at 0° C. The mixture was stirred at 25° C. for 2 hrs. LCMS showed the reactant was consumed completely. The reaction mixture was added water 50 mL and extracted with DCM 100 mL (50 mL*2). The combined organic layers were washed with brine, dried over Na₂SO₄, filtered and concentrated under reduced pressure to give the title product (7.9 g, crude), which was directly used in the next step without further purification.

### Step 5: 6-((2-methoxyethoxy) methoxy)benzofuran-3-yl trifluoromethanesulfonate

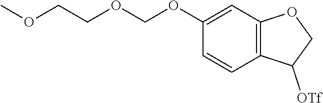 [SMILES: COCCOCOc1ccc2c(c1)OCC2OS(=O)(=O)C(F)(F)F]

The title compound of step 5 (3 g, 24% yield) was prepared in a manner similar to that described in Example 1 step 1 from 6-((2-methoxyethoxy) methoxy)benzofuran-3 (2H)-one and triflic anhydride.

¹H NMR (400 MHz, CDCl₃) δ 7.76-7.73 (m, 1H), 7.50-7.42 (m, 1H), 7.11-7.08 (m, 1H), 6.98-6.89 (m, 1H), 5.32 (s, 2H), 3.87-3.84 (m, 2H), 3.60-3.57 (m, 2H), 3.40 (s, 3H).

### Step 6: N-cyclopropyl-2-(difluoromethoxy)-6-fluoro-4-(6-((2-methoxyethoxy) methoxy)benzofuran-3-yl)benzamide

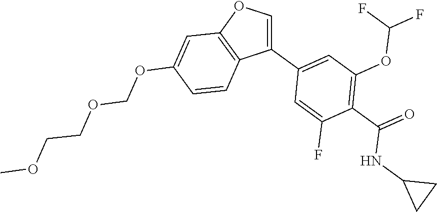 [SMILES: COCCOCOc1ccc2c(-c3cc(F)c(C(=O)NC4CC4)c(OC(F)F)c3)coc2c1]

The title compound of step 6 (500 mg, 73% yield) was prepared in a manner similar to that described in Example 1 step 5 from 6-((2-methoxyethoxy) methoxy)benzofuran-3-yl trifluoromethanesulfonate and N-cyclopropyl-2-(difluoromethoxy)-6-fluoro-4-(4,4,5,5-tetramethyl-1,3,2-dioxaborolan-2-yl)benzamide . LC-MS: [M+H]⁺=466.2.

### Step 7: N-cyclopropyl-2-(difluoromethoxy)-6-fluoro-4-(6-hydroxybenzofuran-3-yl)benzamide

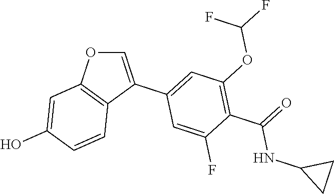 [SMILES: O=C(NC1CC1)c1c(F)cc(-c2coc3cc(O)ccc23)cc1OC(F)F]

A solution of N-cyclopropyl-2-(difluoromethoxy)-6-fluoro-4-(6-((2-methoxyethoxy) methoxy)benzofuran-3-yl)benzamide (0.5 g, 1.07 mmol) in 4N HCl in EtOAc (5 mL) and EtOAc (5 mL) was stirred at 20° C. for 2 hrs. LCMS showed the reactant was consumed completely. The mixture was added H₂O (10 mL), extracted with EtOAc (5 mL*2). The combined organic layers were washed with saturated NaHCO₃ and brine, dried over Na₂SO₄, filtered and concentrated under reduced pressure to give the title product (0.5 g, crude) which was directly used in next step without further purification. LC-MS: [M+H]⁺=378.1.

### Step 8: N-cyclopropyl-2-(difluoromethoxy)-6-fluoro-4-(6-(2-(pyrrolidin-1-yl) ethoxy)benzofuran-3-yl)benzamide (compound 105)

Compound 105 (3.5 mg, 6% yield) was prepared in a manner similar to that described in Example 58 step 1 from N-cyclopropyl-2-(difluoromethoxy)-6-fluoro-4-(6-hydroxybenzofuran-3-yl)benzamide and 2-(pyrrolidin-1-yl) ethan-1-ol. ¹H NMR (400 MHz, DMSO-d₆) δ 8.68 (d, J=4.0 Hz, 1H), 8.45 (s, 1H), 7.81 (d, J=8.8 Hz, 1H), 7.54 (d, J=10.4 Hz, 1H), 7.41-7.13 (m, 3 H), 7.08-6.98 (m, 1H), 4.15 (t, J=6.0 Hz, 2H), 2.90-2.78 (m, 3H), 2.55 (s, 4H), 1.70 (s, 4H), 0.77-0.67 (m, 2H), 0.53-0.43 (m, 2H). LC-MS: [M+H]⁺=475.1.

### Example 106: N-cyclopropyl-2-(difluoromethoxy)-6-((1-methylazetidin-3-yl)oxy)-4-(6-(2-(pyrrolidin-1-yl) ethoxy)benzofuran-3-yl)benzamide (compound 106)

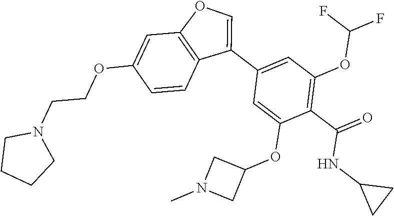 [SMILES: CN1CC(Oc2cc(-c3coc4cc(OCCN5CCCC5)ccc34)cc(OC(F)F)c2C(=O)NC2CC2)C1]

### Step 1: N-cyclopropyl-2-(difluoromethoxy)-6-((1-methylazetidin-3-yl)oxy)-4-(6-(2-(pyrrolidin-1-yl) ethoxy)benzofuran-3-yl)benzamide (compound 106)

To a solution of 1-methylazetidin-3-ol (18.36 mg, 210.76 μmol) in THF (2 mL) was added NaH (8.43 mg, 210.76 μmol, 60% dispersion in mineral oil) at 0° C. The mixture was stirred at 0° C. for 1 hr. N-cyclopropyl-2-(difluoromethoxy)-6-fluoro-4-(6-(2-(pyrrolidin-1-yl) ethoxy)benzofuran-3-yl)benzamide (50 mg, 105.38 μmol) was added at 0° C. The mixture was stirred at 80° C. for 16 hrs. LCMS showed 50% of reactant was remained and 50% of desired MS was detected. The reaction was quenched by saturated NH₄Cl (5 mL) and concentrated under reduced pressure. The residue was purified by prep-HPLC [column: Phenomenex Luna C18 75*30 mm*3 um, mobile phase: [H₂O (0.2% FA)-ACN]; gradient: 1%-40% ACN over 8.0 min] to give compound 106 as a white solid (1.03 mg, 3% yield). ¹H NMR (400 MHz, DMSO-d₆) δ 8.37 (s, 1H), 8.33 (d, J=4.4 Hz, 1H), 8.23 (s, 2H), 7.71 (d, J=8.8 Hz, 1H), 7.44-7.22 (m, 2H), 7.09 (s, 1H), 7.06-7.00 (m, 1H), 6.93 (s, 1H), 5.00-4.88 (m, 1H), 4.14 (t, J=6.0 Hz, 2H), 3.76 (t, J=6.4 Hz, 2H), 2.95 (t, J=6.4 Hz, 2H), 2.83 (t, J=6.0 Hz, 2H), 2.79-2.76 (m, 1H), 2.54 (s, 4H), 2.29 (s, 3H), 1.69 (s, 4H), 0.74-0.65 (m, 2H), 0.52-0.46 (m, 2H). LC-MS: [M+H]⁺=542.2.

### Example 107: (R)—N-cyclopropyl-2-(difluoromethoxy)-6-((1-methylpyrrolidin-2-yl) methoxy)-4-(6-(2-(pyrrolidin-1-yl) ethoxy)benzofuran-3-yl)benzamide (compound 107)

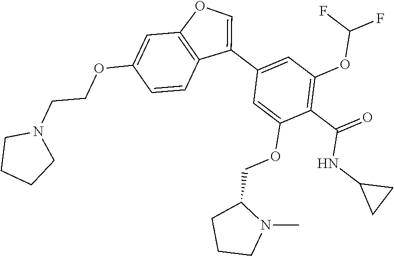 [SMILES: CN1CCC[C@@H]1COc1cc(-c2coc3cc(OCCN4CCCC4)ccc23)cc(OC(F)F)c1C(=O)NC1CC1]

Compound 107 (2.5 mg, 4% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and (R)-(1-methylpyrrolidin-2-yl) methanol. ¹H NMR (400 MHz, DMSO-d₆) δ 8.37 (s, 1H), 8.33 (d, J=4.0 Hz, 1H), 7.76 (d. J=8.4 Hz, 1H), 7.42-7.03 (m, 4H), 7.01 (dd, J₁=8.8 Hz, J₂=2.4 Hz, 1H), 4.14 (t, J₁=6.0 Hz, 2H), 4.07 (dd, J₁=9.6 Hz, J₂=5.6 Hz, 1H), 3.96 (dd, J₁=9.6 Hz, J₂=5.6 Hz, 1H), 3.00-2.91 (m, 1H), 2.82 (t, J=6.0 Hz, 2H), 2.78-2.70 (m, 1H), 2.59-2.51 (m, 5H), 2.37 (s, 3H), 2.20 (q, J=8.8 Hz, 1H), 1.98-1.87 (m, 1H), 1.74-1.58 (m, 7H), 0.72-0.62 (m, 2H), 0.50-0.42 (m, 2H). LC-MS: [M+H]⁺=570.3.

### Example 108: N-cyclopropyl-2-(difluoromethoxy)-6-(2-hydroxy-2-methylpropoxy)-4-(6-(2-(pyrrolidin-1-yl) ethoxy)benzofuran-3-yl)benzamide (compound 108)

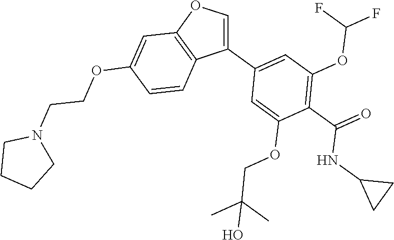 [SMILES: CC(C)(O)COc1cc(-c2coc3cc(OCCN4CCCC4)ccc23)cc(OC(F)F)c1C(=O)NC1CC1]

Compound 108 (2.7 mg, 4% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and 2-methylpropane-1,2-diol. ¹H NMR (400 MHz, DMSO-d₆) δ 8.41-8.35 (m, 2H), 7.75 (d, J=8.8 Hz, 1H), 7.43-7.23 (m, 2H), 7.18 (s, 1H), 7.08 (s, 1H), 7.01 (dd, J₁=8.8 Hz, J₂=2.4 Hz, 1H), 4.63 (s, 1H), 4.14 (t, J=6.0 Hz, 2H), 3.84 (s, 2H), 2.82 (t, J=6.0 Hz, 2H), 2.76 (dt, J₁=7.2 Hz, J₂=3.6 Hz, 1H), 2.53 (s, 4H), 1.72-1.67 (m, 4H), 1.19 (s, 6H), 0.73-0.64 (m, 2H), 0.51-0.45 (m, 2H). LC-MS: [M+H]⁺=545.3.

### Example 109:2-(3-aminocyclobutoxy)-N-cyclopropyl-6-(difluoromethoxy)-4-(6-(2-(pyrrolidin-1-yl) ethoxy)benzofuran-3-yl)benzamide (compound 109)

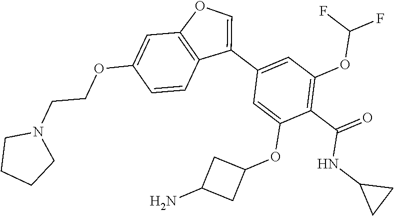 [SMILES: NC1CC(Oc2cc(-c3coc4cc(OCCN5CCCC5)ccc34)cc(OC(F)F)c2C(=O)NC2CC2)C1]

### Step 1: tert-butyl (3-(2-(cyclopropylcarbamoyl)-3-(difluoromethoxy)-5-(6-(2-(pyrrolidin-1-yl) ethoxy)benzofuran-3-yl) phenoxy)cyclobutyl) carbamate

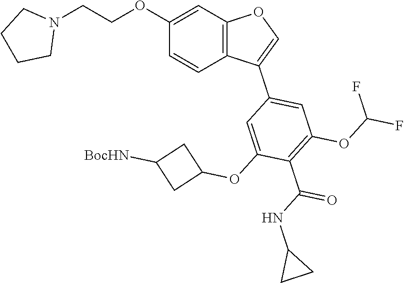 [SMILES: CC(C)(C)OC(=O)NC1CC(Oc2cc(-c3coc4cc(OCCN5CCCC5)ccc34)cc(OC(F)F)c2C(=O)NC2CC2)C1]

The title compound of step 1 (25 mg, 36% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and tert-butyl (3-hydroxycyclobutyl) carbamate. LC-MS: [M+H]⁺=642.3.

### Step 2: 2-(3-aminocyclobutoxy)-N-cyclopropyl-6-(difluoromethoxy)-4-(6-(2-(pyrrolidin-1-yl) ethoxy)benzofuran-3-yl)benzamide (compound 109)

Compound 109 (0.5 mg, 2% yield) was prepared in a manner similar to that described in Example 28 step 2 from tert-butyl (3-(2-(cyclopropylcarbamoyl)-3-(difluoromethoxy)-5-(6-(2-(pyrrolidin-1-yl) ethoxy)benzofuran-3-yl) phenoxy)cyclobutyl) carbamate. ¹H NMR (400 MHz, DMSO-d₆) δ 7.77-7.68 (m, 1H), 7.62-7.49 (m, 1H), 7.10 (s, 1H), 7.07-7.03 (m, 1H), 7.02-6.96 (m, 1H), 6.90-6.43 (m, 2H), 6.11-5.95 (m, 1H), 4.47-4.34 (m, 1H), 4.30-4.17 (m, 2H), 3.28-3.15 (m, 1H), 3.03 (s, 2H), 2.97-2.87 (m, 3H), 2.75 (s, 4H), 2.64-2.42 (m, 1H), 2.30-2.02 (m, 1H), 1.99-1.91 (m, 2H), 1.88 (s, 4H), 0.96-0.84 (m, 2H), 0.70-0.60 (m, 2H). LC-MS: [M+H]⁺=542.3.

### Example 110: (R)—N-cyclopropyl-2-(difluoromethoxy)-6-((1-methylpyrrolidin-3-yl)oxy)-4-(6-(2-(pyrrolidin-1-yl) ethoxy)benzofuran-3-yl)benzamide (compound 110)

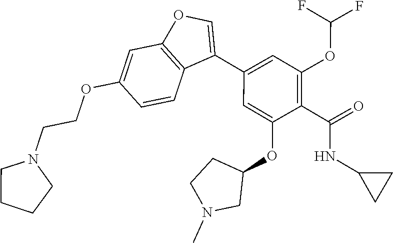 [SMILES: CN1CC[C@@H](Oc2cc(-c3coc4cc(OCCN5CCCC5)ccc34)cc(OC(F)F)c2C(=O)NC2CC2)C1]

Compound 110 (1.98 mg, 3% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and (R)-1-methylpyrrolidin-3-ol. ¹H NMR (400 MHz, CD₃OD) δ 8.04 (s, 1H), 7.68 (d, J=8.8 Hz, 1H), 7.21-7.17 (m, 1H), 7.13-7.08 (m, 2H), 7.05-6.68 (m, 2H), 5.09-5.02 (m, 1H), 4.21 (t, J=5.6 Hz, 2H), 3.07-2.95 (m, 3H), 2.88-2.78 (m, 3H), 2.74 (s, 4H), 2.67-2.59 (m, 1H), 2.46-2.34 (m, 4H), 2.12-1.99 (m, 1H), 1.91-1.83 (m, 4H), 0.85-0.77 (m, 2H), 0.67-0.58 (m, 2H). LC-MS: [M+H]⁺=556.3.

### Example 111: (S)—N-cyclopropyl-2-(difluoromethoxy)-6-((1-methylpyrrolidin-3-yl)oxy)-4-(6-(2-(pyrrolidin-1-yl) ethoxy)benzofuran-3-yl)benzamide (compound 111)

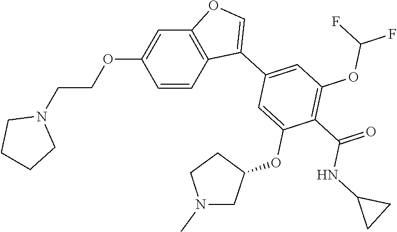 [SMILES: CN1CC[C@H](Oc2cc(-c3coc4cc(OCCN5CCCC5)ccc34)cc(OC(F)F)c2C(=O)NC2CC2)C1]

Compound 111 (14.1 mg, 13% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and(S)-1-methylpyrrolidin-3-ol. ¹H NMR (400 MHz, DMSO-d₆) § 8.37 (s, 1H), 8.28 (d, J=4.0 Hz, 1H), 7.73 (d, J=8.4 Hz, 1H), 7.42-7.23 (m, 1H), 7.10 (s, 1H), 7.08-6.96 (m, 3H), 5.04 (d, J=6.4 Hz, 1H), 4.14 (t, J=5.6 Hz, 2H), 2.81 (t, J=5.6 Hz, 3H), 2.78-2.73 (m, 1H), 2.60 (d. J=7.6 Hz, 1H), 2.53 (s, 4H), 2.47-2.39 (m, 2 H), 2.34-2.28 (m. 1H), 2.26 (s, 3H), 1.69 (s, 4H), 0.72-0.66 (m, 2H), 0.51-0.46 (m, 2H). LC-MS: [M+H]⁺=556.3.

### Example 112: (R)—N-cyclopropyl-2-(difluoromethoxy)-6-((1-methylpyrrolidin-3-yl) methoxy)-4-(6-(2-(pyrrolidin-1-yl) ethoxy)benzofuran-3-yl)benzamide (compound 112)

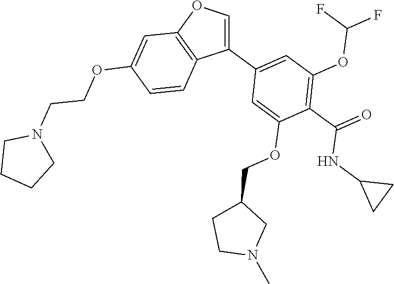 [SMILES: CN1CC[C@@H](COc2cc(-c3coc4cc(OCCN5CCCC5)ccc34)cc(OC(F)F)c2C(=O)NC2CC2)C1]

Compound 112 (1.01 mg, 1% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and (R)-(1-methylpyrrolidin-3-yl) methanol. ¹H NMR (400 MHz, CD₃OD) δ 8.04 (s, 1H), 7.70 (d, J=8.8 Hz, 1H), 7.23-7.16 (m, 2H), 7.10 (s, 1H), 7.08-6.68 (m, 2H), 4.21 (t, J=5.6 Hz, 2H), 4.14-4.00 (m, 2H), 3.01 (t, J=5.6 Hz, 2H), 2.91-2.82 (m, 2H), 2.79-2.53 (m. 8H), 2.45 (s, 3H), 2.19-2.07 (m, 1H), 1.94-1.83 (m, 4H), 1.80-1.69 (m, 1H), 0.86-0.78 (m, 2H), 0.67-0.56 (m, 2H). LC-MS: [M+H]⁺=570.3.

### Example 113: (S)—N-cyclopropyl-2-(difluoromethoxy)-6-((1-methylpyrrolidin-2-yl) methoxy)-4-(6-(2-(pyrrolidin-1-yl) ethoxy)benzofuran-3-yl)benzamide (compound 113)

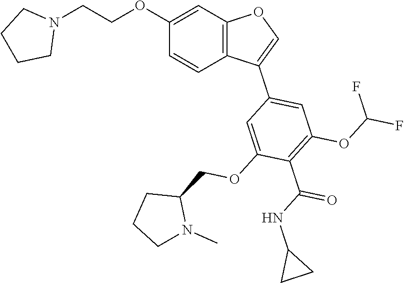 [SMILES: CN1CCC[C@H]1COc1cc(-c2coc3cc(OCCN4CCCC4)ccc23)cc(OC(F)F)c1C(=O)NC1CC1]

Compound 113 (19.3 mg, 17% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and(S)-(1-methylpyrrolidin-2-yl) methanol. ¹H NMR (400 MHz, DMSO-d₆) δ 8.38 (s, 1H), 8.35 (d, J=4.0 Hz, 1H), 7.76 (d, J=8.4 Hz, 1H), 7.43-7.04 (m, 4H), 7.01 (dd, J₁=8.8 Hz, J₂=2.0 Hz, 1H), 4.14 (t, J=6.0 Hz, 2H), 4.07 (dd, J₁=8.4 Hz J₂=5.2 Hz, 1H), 3.96 (dd, J₁=8.8 Hz, J₂=4.8 Hz, 1H), 2.99-2.91 (m, 1H), 2.82 (t, J=6.0 Hz, 2H), 2.77-2.69 (m, 1H), 2.53 (brs, 5H), 2.36 (s, 3H), 2.20 (q, J=8.0 Hz, 1H), 1.98-1.87 (m, 1H), 1.73-1.61 (m, 7H), 0.70-0.63 (m, 2H), 0.50-0.43 (m, 2H). LC-MS: [M+H]⁺=570.3.

### Example 114: (R)—N-cyclopropyl-2-(difluoromethoxy)-6-((1-methylazetidin-2-yl) methoxy)-4-(6-(2-(pyrrolidin-1-yl) ethoxy)benzofuran-3-yl)benzamide (compound 114)

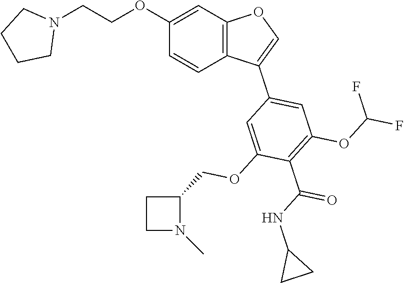 [SMILES: CN1CC[C@@H]1COc1cc(-c2coc3cc(OCCN4CCCC4)ccc23)cc(OC(F)F)c1C(=O)NC1CC1]

Compound 114 (11.0 mg, 9% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and (R)-(1-methylazetidin-2-yl) methanol. ¹H NMR (400 MHz, DMSO-d₆) δ 8.38-8.31 (m, 2H), 7.78 (d. J=8.8 Hz, 1H), 7.42-7.06 (m, 4H), 7.04-6.98 (m, 1H), 4.14 (t, J=6.0 Hz, 2H), 4.10-4.04 (m, 2H), 3.27 (d, J=6.4 Hz, 2H), 2.82 (t, J=6.0 Hz, 2H), 2.78-2.70 (m, 2H), 2.53 (s, 4H), 2.27 (s, 3H), 2.02-1.89 (m, 2H), 1.71-1.67 (m, 4H), 0.70-0.63 (m, 2H), 0.50-0.44 (m, 2H). LC-MS: [M+H]⁺=556.3.

### Example 115: N-cyclopropyl-2-(difluoromethoxy)-6-((1-methylazetidin-3-yl) methoxy)-4-(6-(2-(pyrrolidin-1-yl) ethoxy)benzofuran-3-yl)benzamide (compound 115)

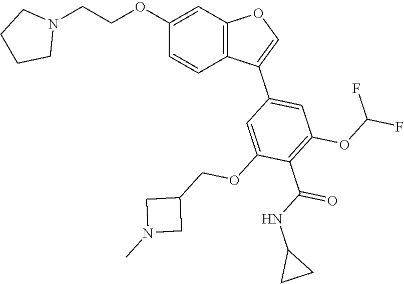 [SMILES: CN1CC(COc2cc(-c3coc4cc(OCCN5CCCC5)ccc34)cc(OC(F)F)c2C(=O)NC2CC2)C1]

Compound 115 (6.2 mg, 5% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and (1-methylazetidin-3-yl) methanol. ¹H NMR (400 MHz, DMSO-d₆) δ 8.37 (s, 1H), 8.33 (d. J=4.4 Hz, 1H), 7.76 (d. J=8.8 Hz, 1H), 7.42-7.04 (m, 4H), 7.01 (dd, J₁=8.8 Hz, J₂=2.0 Hz, 1H), 4.19 (d, J=6.4 Hz, 2H), 4.14 (t, J=6.0 Hz, 2H), 3.24 (t, J=7.2 Hz, 2H), 2.99 (t, J=6.4 Hz, 2H), 2.82 (t, J=6.0 Hz, 2H), 2.79-2.69 (m, 2H), 2.53 (s, 4H), 2.20 (s, 3H), 1.71-1.67 (m, 4H), 0.71-0.64 (m, 2H), 0.48-0.43 (m, 2H). LC-MS: [M+H]⁺=556.3.

### Example 116: (S)—N-cyclopropyl-2-(difluoromethoxy)-6-((1-methylpyrrolidin-3-yl) methoxy)-4-(6-(2-(pyrrolidin-1-yl) ethoxy)benzofuran-3-yl)benzamide (compound 116)

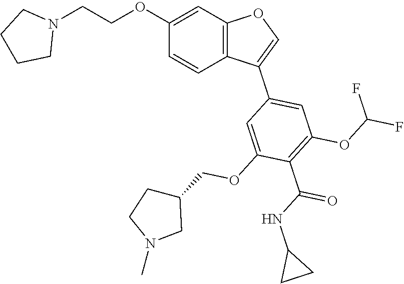 [SMILES: CN1CC[C@H](COc2cc(-c3coc4cc(OCCN5CCCC5)ccc34)cc(OC(F)F)c2C(=O)NC2CC2)C1]

Compound 116 (23.3 mg, 19% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and(S)-(1-methylpyrrolidin-3-yl) methanol. ¹H NMR (400 MHz, DMSO-d₆) δ 8.36 (s, 1H), 8.34 (d, J=4.2 Hz, 1H), 8.23 (s, 1H), 7.75 (d, J=8.8 Hz, 1H), 7.43-7.04 (m, 4H), 7.01 (dd, J₁=8.4 Hz, J₂=2.0 Hz, 1H), 4.15 (t, J=5.6 Hz, 2H), 4.07-3.94 (m, 3H), 2.85 (t, J=5.6 Hz, 2H), 2.76 (dt, J₁=7.2 Hz, J₂=3.6 Hz, 1H), 2.68-2.53 (m, 8H), 2.49-2.45 (m, 2H), 2.31 (s, 3H), 2.03-1.87 (m, 1H), 1.76-1.67 (m, 4H), 1.64-1.49 (m, 1H), 0.74-0.64 (m, 2H), 0.50-0.44 (m, 2H). LC-MS: [M+H]⁺=570.3.

### Example 117: (S)—N-cyclopropyl-2-(difluoromethoxy)-6-((1-methylazetidin-2-yl) methoxy)-4-(6-(2-(pyrrolidin-1-yl) ethoxy)benzofuran-3-yl)benzamide (compound 117)

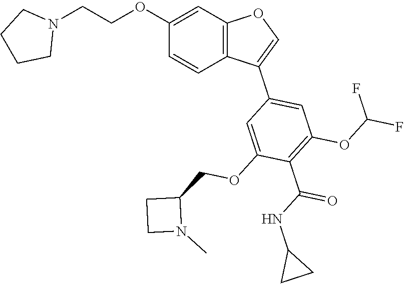 [SMILES: CN1CC[C@H]1COc1cc(-c2coc3cc(OCCN4CCCC4)ccc23)cc(OC(F)F)c1C(=O)NC1CC1]

Compound 117 (7.5 mg, 6% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and(S)-(1-methylazetidin-2-yl) methanol. ¹H NMR (400 MHz, DMSO-d₆) δ 8.37 (s, 1H), 8.34 (d, J=4.0 Hz, 1H), 7.78 (d, J=8.4 Hz, 1H), 7.41-7.03 (m, 4H), 7.01 (dd, J₁=8.8 Hz, J₂=2.4 Hz, 1H), 4.14 (t, J=5.6 Hz, 2H), 4.12-4.03 (m, 2H), 3.30-3.25 (m, 2H), 2.82 (t, J=5.6 Hz, 2H), 2.78-2.69 (m, 2H), 2.53 (s, 4H), 2.27 (s, 3H), 2.02-1.88 (m, 2H), 1.72-1.66 (m, 4H), 0.71-0.62 (m, 2H), 0.50-0.43 (m, 2H). LC-MS: [M+H]⁺=556.3.

### Example 118: N-cyclopropyl-2-(difluoromethoxy)-6-(2-(dimethylamino) ethoxy)-4-(6-(2-(pyrrolidin-1-yl) ethoxy)benzofuran-3-yl)benzamide (compound 118)

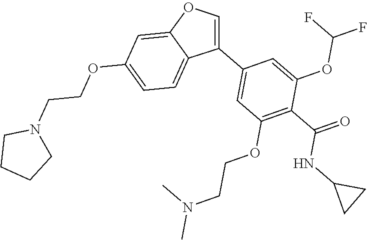 [SMILES: CN(C)CCOc1cc(-c2coc3cc(OCCN4CCCC4)ccc23)cc(OC(F)F)c1C(=O)NC1CC1]

### Step 1: methyl 4-bromo-2-(difluoromethoxy)-6-(2-(dimethylamino) ethoxy)benzoate

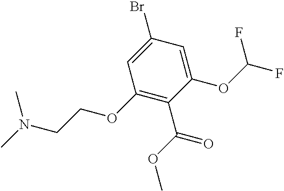 [SMILES: COC(=O)c1c(OCCN(C)C)cc(Br)cc1OC(F)F]

The title compound of step 1 (240 mg, 33% yield) was prepared in a manner similar to that described in Example 103 step 2 from methyl 4-bromo-2-(difluoromethoxy)-6-fluorobenzoate and 2-(dimethylamino) ethan-1-ol. LC-MS: [M+H]⁺=368.1.

### Step 2: 4-bromo-2-(difluoromethoxy)-6-(2-(dimethylamino) ethoxy)benzoic acid

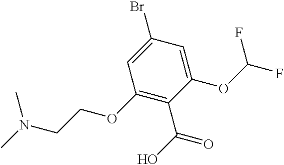 [SMILES: CN(C)CCOc1cc(Br)cc(OC(F)F)c1C(=O)O]

The title compound of step 2 (220 mg, 96% yield) was prepared in a manner similar to that described in Example 103 step 3 from methyl 4-bromo-2-(difluoromethoxy)-6-(2-(dimethylamino) ethoxy)benzoate and lithium hydroxide. LC-MS: [M+H]⁺=354.1.

### Step 3: 4-bromo-N-cyclopropyl-2-(difluoromethoxy)-6-(2-(dimethylamino) ethoxy)benzamide

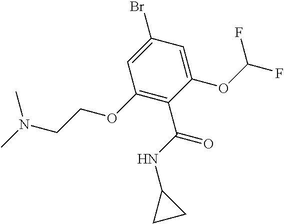 [SMILES: CN(C)CCOc1cc(Br)cc(OC(F)F)c1C(=O)NC1CC1]

The title compound of step 3 (98 mg, 40% yield) was prepared in a manner similar to that described in Example 25 step 9 from 4-bromo-2-(difluoromethoxy)-6-(2-(dimethylamino) ethoxy)benzoic acid and cyclopropanamine. LC-MS: [M+H]⁺=393.1.

### Step 4: N-cyclopropyl-2-(difluoromethoxy)-6-(2-(dimethylamino) ethoxy)-4-(4,4,5,5-tetramethyl-1,3,2-dioxaborolan-2-yl)benzamide

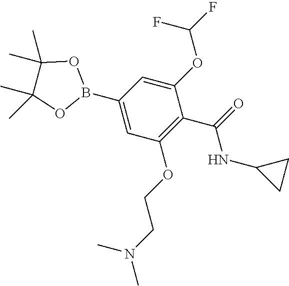 [SMILES: CN(C)CCOc1cc(B2OC(C)(C)C(C)(C)O2)cc(OC(F)F)c1C(=O)NC1CC1]

The title compound of step 4 (60 mg, 45% yield) was prepared in a manner similar to that described in Example 1 step 4 from 4-bromo-N-cyclopropyl-2-(difluoromethoxy)-6-(2-(dimethylamino) ethoxy)benzamide and 4,4,4′,4′,5,5,5′,5′-octamethyl-2,2′-bi (1,3,2-dioxaborolane). LC-MS: [M+H]⁺=441.1.

### Step 5: N-cyclopropyl-2-(difluoromethoxy)-6-(2-(dimethylamino) ethoxy)-4-(6-(2-(pyrrolidin-1-yl) ethoxy)benzofuran-3-yl)benzamide (compound 118)

Compound 118 (0.55 mg, 0.5% yield) was prepared in a manner similar to that described in Example 1 step 5 from N-cyclopropyl-2-(difluoromethoxy)-6-(2-(dimethylamino) ethoxy)-4-(4,4,5,5-tetramethyl-1,3,2-dioxaborolan-2-yl)benzamide and 6-(2-(pyrrolidin-1-yl) ethoxy)benzofuran-3-yl trifluoromethanesulfonate. ¹H NMR (500 MHz, CD3OD) δ 8.52 (s, 1H), 8.08 (s, 1H), 7.74 (d, J=8.7 Hz, 1H), 7.25-7.24 (m, 1H), 7.22-7.00 (m, 4H), 4.33-4.30 (m, 4H), 3.35 (s, 2H), 3.12 (s, 2H), 2.95 (s, 2H), 2.84-2.83 (m, 1H), 2.50 (s, 6H), 2.02 (brs, 4H), 1.29 (s, 2H), 0.85-0.76 (m, 2H), 0.66-0.57 (m, 2H). LC-MS [M+H]⁺=544.3.

### Example 119: N-cyclopropyl-2-(difluoromethoxy)-6-ethoxy-4-(6-(2-(pyrrolidin-1-yl) ethoxy)benzofuran-3-yl)benzamide (compound 119)

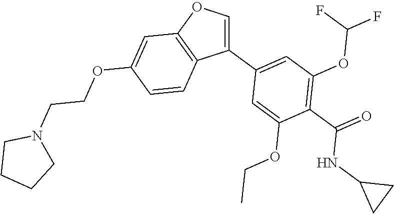 [SMILES: CCOc1cc(-c2coc3cc(OCCN4CCCC4)ccc23)cc(OC(F)F)c1C(=O)NC1CC1]

To a solution of N-cyclopropyl-2-(difluoromethoxy)-6-fluoro-4-(6-(2-(pyrrolidin-1-yl) ethoxy)benzofuran-3-yl)benzamide (0.1 g, 210.76 μmol) in DMF (2 mL) was added EtONa (71.71 mg, 210.76 μmol) at 0° C. The mixture was stirred at 70° C. for 2 hrs. LCMS showed the reactant was consumed completely. The reaction mixture was filtered. The filtrate was concentrated and purified by prep-HPLC (column: Waters Xbridge BEH C18 100*25 mm*10 um; mobile phase: [H₂O (10 mM NH₄HCO₃)-ACN]; gradient: 20%-60% ACN over 8.0 min) to give compound 119 (30.2 mg, 28% yield). ¹H NMR (400 MHz, DMSO-d₆) δ 8.36 (s, 1H), 8.29 (d, J=4.4 Hz, 1H), 7.75 (d, J=8.8 Hz, 1H), 7.41-7.04 (m, 4H), 7.02-6.98 (m, 1H), 4.20-4.11 (m, 4H), 2.84-2.75 (m, 3H), 2.53 (s, 4H), 1.71-1.66 (m, 4H), 1.32 (t, J=6.8 Hz, 3H), 0.73-0.64 (m, 2H), 0.50-0.43 (m, 2H). LC-MS: [M+H]⁺=501.3.

### Example 120: N-cyclopropyl-2-(difluoromethoxy)-6-isopropoxy-4-(6-(2-(pyrrolidin-1-yl) ethoxy)benzofuran-3-yl)benzamide (compound 120)

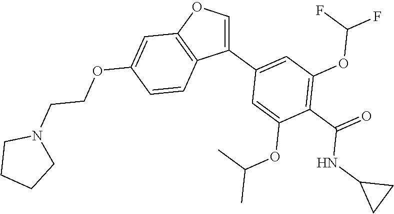 [SMILES: CC(C)Oc1cc(-c2coc3cc(OCCN4CCCC4)ccc23)cc(OC(F)F)c1C(=O)NC1CC1]

Compound 120 (4.8 mg, 4% yield) was prepared in a manner similar to that described in Example 106 step 1 from N-cyclopropyl-2-(difluoromethoxy)-6-fluoro-4-(6-(2-(pyrrolidin-1-yl) ethoxy)benzofuran-3-yl)benzamide and isopropanol. ¹H NMR (400 MHz, DMSO-d₆) δ 8.36 (s, 1H), 8.25 (d, J=4.8 Hz, 1H), 7.72 (d, J=8.4 Hz, 1H), 7.31-7.04 (m, 4H), 7.01 (d, J=9.2 Hz, 1H), 4.79-4.70 (m, 1H), 4.16-4.12 (m, 2H), 2.81 (t, J=5.6 Hz, 2H), 2.78-2.73 (m, 1H), 2.53 (s, 4H), 1.69 (s, 4H), 1.28 (d, J=5.6 Hz, 6H), 0.68-0.60 (m, 2H), 0.50-0.41 (m, 2H). LC-MS: [M+H]⁺=515.3.

### Example 121: 2-cyclopropoxy-N-cyclopropyl-6-(difluoromethoxy)-4-(6-(2-(pyrrolidin-1-yl) ethoxy)benzofuran-3-yl)benzamide (compound 121)

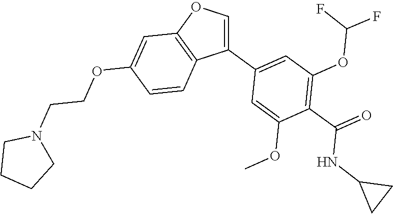 [SMILES: COc1cc(-c2coc3cc(OCCN4CCCC4)ccc23)cc(OC(F)F)c1C(=O)NC1CC1]

Compound 121 (1.7 mg, 1% yield) was prepared in a manner similar to that described in Example 106 step 1 from N-cyclopropyl-2-(difluoromethoxy)-6-fluoro-4-(6-(2-(pyrrolidin-1-yl)ethoxy)benzofuran-3-yl)benzamide and cyclopropanol. ¹H NMR (400 MHz, DMSO-d₆) δ 8.37 (s, 1H), 8.27 (d, J=4.0 Hz, 1H), 7.77 (d, J=8.8 Hz, 1H), 7.55-7.48 (m, 1H), 7.41-7.31 (m, 1H), 7.25-7.15 (m, 1H), 7.14-7.06 (m, 1H), 7.04-7.00 (m, 1H), 4.15 (t, J=5.6 Hz, 2H), 4.11-4.02 (m, 1H), 3.51 (s, 2H), 2.83 (t, J=5.6 Hz, 2H), 2.78-2.74 (m, 1H), 2.54 (s, 2H), 1.70 (s, 4H), 0.95-0.35 (m, 8H). LC-MS: [M+H]⁺=513.3.

### Example 122: N-cyclopropyl-2-(difluoromethoxy)-6-(methylamino)-4-(6-(2-(pyrrolidin-1-yl) ethoxy)benzofuran-3-yl)benzamide (compound 122)

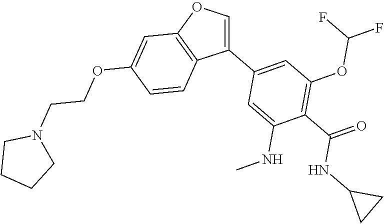 [SMILES: CNc1cc(-c2coc3cc(OCCN4CCCC4)ccc23)cc(OC(F)F)c1C(=O)NC1CC1]

To a solution of N-cyclopropyl-2-(difluoromethoxy)-6-fluoro-4-(6-(2-(pyrrolidin-1-yl) ethoxy)benzofuran-3-yl)benzamide (0.05 g, 105.38 μmol) in THF (5 mL) was added K₂CO₃ (43.69 mg, 316.14 μmol) and MeNH₂ (1.05 mL, 2 M in THF) at 0° C. The mixture was stirred at 90° C. for 48 hrs in a sealed tube. LCMS showed the reaction was completed. The mixture was filtered and the filter cake was washed by MeOH (5 mL). The filtrate was concentrated under reduced pressure. The crude product was purified by prep-HPLC (column: Waters Xbridge BEH C18 100*30 mm*10 um; mobile phase: [H₂O (10 mM NH₄HCO₃)-ACN]; gradient: 40%-70% ACN over 8.0 min) to give compound 122 (11.4 mg, 22% yield). ¹H NMR (400 MHz, DMSO-d₆) δ 8.32-8.30 (m, 2H), 7.73 (d, J=8.8 Hz, 1H), 7.31-6.61 (m, 5H), 5.81 (q, J=4.8 Hz, 1H), 4.13 (t, J=6.0 Hz, 2H), 2.88-2.76 (m, 6H), 2.53 (s, 4H), 1.69 (t, J=3.2 Hz, 4H), 0.72-0.64 (m, 2H), 0.56-0.47 (m, 2H). LC-MS: [M+H]⁺=486.2.

### Example 123: N-cyclopropyl-2,6-bis(difluoromethoxy)-4-(6-(2-(pyrrolidin-1-yl) ethoxy)benzofuran-3-yl)benzamide (compound 123)

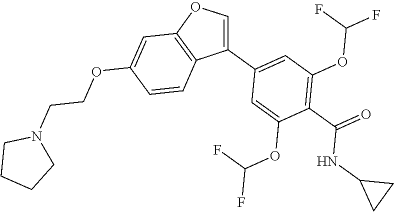 [SMILES: O=C(NC1CC1)c1c(OC(F)F)cc(-c2coc3cc(OCCN4CCCC4)ccc23)cc1OC(F)F]

### Step 1: 4-bromo-N-cyclopropyl-2,6-difluorobenzamide

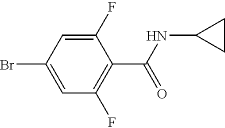 [SMILES: O=C(NC1CC1)c1c(F)cc(Br)cc1F]

The title compound of step 1 (1.9 g, 81% yield) was prepared in a manner similar to that described in Example 25 step 9 from 4-bromo-2,6-difluorobenzoic acid and cyclopropane. ¹H NMR (400 MHz, CDCl₃) § 7.17-7.10 (m, 2H), 6.17-6.01 (m. 1H), 2.91 (dt, J=3.6, 7.2 Hz, 1H), 0.92-0.85 (m, 2H), 0.68-0.61 (m, 2H).

### Step 2: 4-bromo-N-cyclopropyl-2,6-dimethoxybenzamide

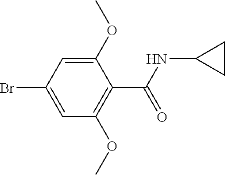 [SMILES: COc1cc(Br)cc(OC)c1C(=O)NC1CC1]

To a solution of 4-bromo-N-cyclopropyl-2,6-difluorobenzamide (0.5 g. 1.81 mmol) in THF (5 mL) was added NaOMe (978.42 mg, 5.43 mmol) at 20° C. under N₂. Then the mixture was stirred at 50° C. for 12 hrs. LCMS showed the reactant was consumed completely. The combined reaction mixture was poured into H₂O (10 mL) and extracted with EtOAc (10 mL*2). The combined organic layers were washed with brine, dried over anhydrous Na₂SO₄, filtered and concentrated under reduced pressure. The residue was purified by silica gel column chromatography, eluting with PE/EtOAc (v/v=1/0 to 0/1), to give the title product (0.45 g, 82% yield). ¹H NMR (400 MHz, CDCl₃) δ 6.76-6.67 (m, 2H), 3.85-3.77 (m, 6H), 2.91 (qt, J=3.6, 7.2 Hz, 1H), 0.87-0.80 (m, 2H), 0.63-0.56 (m, 2H).

### Step 3: 4-bromo-N-cyclopropyl-2,6-dihydroxybenzamide

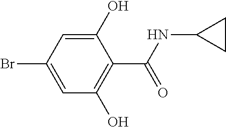 [SMILES: O=C(NC1CC1)c1c(O)cc(Br)cc1O]

To a solution of 4-bromo-N-cyclopropyl-2,6-dimethoxybenzamide (0.45 g. 1.50 mmol) in DCM (10 mL) was added BBr₃ (2.25 g, 9.00 mmol) at 0° C. The mixture was stirred at 20° C. for 12 hrs under N₂. LCMS showed the reactant was consumed completely. The combined reaction mixture was poured into H₂O (20 mL) and extracted with EtOAc (15 mL*2). The combined organic layers were washed with brine, dried over anhydrous Na₂SO₄, filtered and concentrated under reduced pressure. The residue was purified by silica gel column chromatography, eluting with PE/EtOAc (v/v=1/0 to 0/1), to give the title product (70 mg, 17% yield). LC-MS: [M+H]⁺=273.1.

### Step 4: 4-bromo-N-cyclopropyl-2,6-bis(difluoromethoxy)benzamide

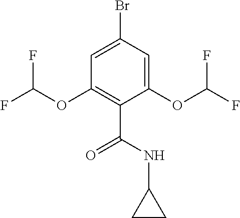 [SMILES: O=C(NC1CC1)c1c(OC(F)F)cc(Br)cc1OC(F)F]

The title compound of step 4 (70 mg, 63% yield) was prepared in a manner similar to that described in Example 13 step 2 from 4-bromo-N-cyclopropyl-2,6-dihydroxybenzamide and diethyl (bromodifluoromethyl)phosphonate. ¹H NMR (400 MHz, CDCl₃) & 7.32-7.27 (m, 2H), 6.77-6.35 (m, 2H), 5.94 (s, 1H), 2.87 (qt, J=3.6, 7.2 Hz, 1H), 0.94-0.84 (m, 2H), 0.67-0.58 (m, 2H). LC-MS: [M+H]⁺=372.0.

### Step 5: N-cyclopropyl-2,6-bis(difluoromethoxy)-4-(4,4,5,5-tetramethyl-1,3,2-dioxaborolan-2-yl)benzamide

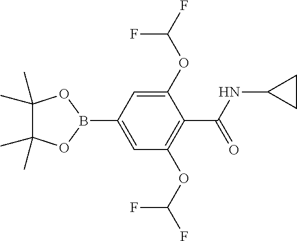 [SMILES: CC1(C)OB(c2cc(OC(F)F)c(C(=O)NC3CC3)c(OC(F)F)c2)OC1(C)C]

The title compound of step 5 (70 mg, 77% yield) was prepared in a manner similar to that described in Example 1 step 4 from 4-bromo-N-cyclopropyl-2,6-bis(difluoromethoxy)benzamide and 4,4,4′,4′,5,5,5′,5′-octamethyl-2,2′-bi (1,3,2-dioxaborolane). ¹H NMR (400 MHz, CDCl₃) δ 7.50-7.41 (m, 2H), 6.81-6.39 (m, 2H), 5.94 (br s, 1H), 2.88 (qt, J=3.6, 6.8 Hz, 1H), 1.37-1.33 (m, 12H), 0.92-0.83 (m, 2H), 0.69-0.61 (m, 2H). LC-MS: [M+H]⁺=420.1.

### Step 6: N-cyclopropyl-2,6-bis(difluoromethoxy)-4-(6-((2-methoxyethoxy) methoxy)benzofuran-3-yl)benzamide

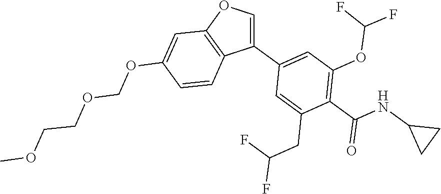 [SMILES: COCCOCOc1ccc2c(-c3cc(CC(F)F)c(C(=O)NC4CC4)c(OC(F)F)c3)coc2c1]

The title compound of step 6 (50 mg, 58% yield) was prepared in a manner similar to that described in Example 1 step 5 from N-cyclopropyl-2,6-bis(difluoromethoxy)-4-(4,4,5,5-tetramethyl-1,3,2-dioxaborolan-2-yl)benzamide and 6-((2-methoxyethoxy) methoxy)benzofuran-3-yl trifluoromethanesulfonate. LC-MS: [M+H]⁺=514.3.

### Step 7: N-cyclopropyl-2,6-bis(difluoromethoxy)-4-(6-hydroxybenzofuran-3-yl)benzamide

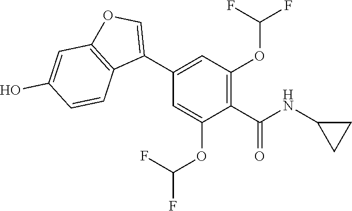 [SMILES: O=C(NC1CC1)c1c(OC(F)F)cc(-c2coc3cc(O)ccc23)cc1OC(F)F]

The title compound of step 6 (20 mg, crude) was prepared in a manner similar to that described in Example 105 step 7 from N-cyclopropyl-2,6-bis(difluoromethoxy)-4-(6-((2-methoxyethoxy) methoxy)benzofuran-3-yl)benzamide . LC-MS: [M+H]⁺=426.3.

### Step 8: N-cyclopropyl-2,6-bis(difluoromethoxy)-4-(6-(2-(pyrrolidin-1-yl) ethoxy)benzofuran-3-yl)benzamide (compound 123)

To a solution of N-cyclopropyl-2,6-bis(difluoromethoxy)-4-(6-hydroxybenzofuran-3-yl)benzamide (20 mg, 47.02 μmol) and 2-pyrrolidin-1-ylethanol (6.50 mg, 56.43 μmol) in toluene (1 mL) was added (cyanomethylene)tributylphosphorane (22.70 mg, 94.04 μmol) at 25° C. under N₂. The mixture was stirred at 90° C. for 12 hrs. LCMS showed the reactant was consumed completely. The mixture was filtered through a Celite pad, and the filtrate was concentrated under reduced pressure. The residue was purified by prep-HPLC (column: Phenomenex Luna C18 100*30 mm*3 um; mobile phase: [H₂O (0.2% FA)-ACN]; gradient: 20%-60% ACN over 8.0 min) to give compound 123 (3.8 mg, 15% yield). ¹H NMR (400 MHz, DMSO-d₆) δ 8.53 (d, J=4.4 Hz, 1H), 8.41 (s, 1H), 7.76 (d, J=8.8 Hz, 1H), 7.54-7.12 (m, 5H), 7.03 (dd, J₁=8.8 Hz, J₂=2.0 Hz, 1H), 4.14 (t. J=6.0 Hz, 2H), 2.85-2.76 (m, 3H), 2.53 (s, 4H), 1.71-1.67 (m, 4H), 0.74-0.67 (m, 2H), 0.50-0.45 (m, 2H). LC-MS: [M+H]⁺=523.2.

### Example 124: N-(2-aminocyclohexyl)-2-(difluoromethoxy)-6-methoxy-4-(6-(2-(pyrrolidin-1-yl) ethoxy)benzofuran-3-yl)benzamide (cis racemate, compound 124)

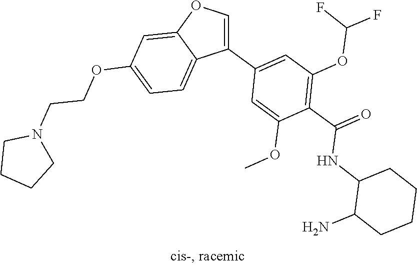 [SMILES: COc1cc(-c2coc3cc(OCCN4CCCC4)ccc23)cc(OC(F)F)c1C(=O)NC1CCCCC1N]

### Step 1: N-(2-aminocyclohexyl)-2-(difluoromethoxy)-6-methoxy-4-(6-(2-(pyrrolidin-1-yl) ethoxy)benzofuran-3-yl)benzamide (compound 124)

Compound 124 (6 mg, 12% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cis-1,2-cyclohexanediamine. ¹H NMR (500 MHz, DMSO-d₆) δ 8.45-8.35 (m, 1H), 8.20-8.12 (m, 1H), 7.80-7.86 (m, 1H), 7.35-7.23 (m, 3H), 7.12-7.02 (m, 2H), 4.16 (d, J=6.2 Hz, 2H), 4.09 (s, 1H), 3.97-3.84 (m, 3H), 3.10 (s, 2H), 2.84 (d, J=5.7 Hz, 6H), 1.80-1.30 (m, 12H). LC-MS: [M+H]⁺=544.0.

### Example 125:2-(difluoromethoxy)-N-((1S,2R)-2-hydroxycyclohexyl)-6-methoxy-4-(6-(2-(pyrrolidin-1-yl) ethoxy)benzofuran-3-yl)benzamide (compound 125)

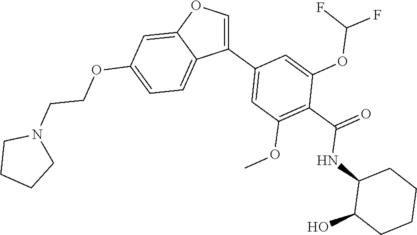 [SMILES: COc1cc(-c2coc3cc(OCCN4CCCC4)ccc23)cc(OC(F)F)c1C(=O)N[C@H]1CCCC[C@H]1O]

Compound 125 (13 mg, 30% yield) was prepared in a manner similar to that described in Example 25 step 9 from 2-(difluoromethoxy)-6-methoxy-4-(6-(2-(pyrrolidin-1-yl)ethoxy)benzofuran-3-yl)benzoic acid and (1R,2S)-2-aminocyclohexan-1-ol. ¹H NMR (500 MHz, DMSO-d₆) δ 8.39 (s, 1H), 7.82-7.73 (m, 2H), 7.36-6.98 (m, 5H), 4.37 (d, J=3.3 Hz, 1H), 4.16 (t, J=5.8 Hz, 2H), 3.88 (s, 3H), 3.82-3.78 (m, 2H), 2.87 (t, J=5.7 Hz, 2H), 2.59 (s, 4H), 1.74-1.68 (m, 5H), 1.67-1.58 (m, 2H), 1.58-1.42 (m, 3H), 1.32-1.28 (m, 2H). LC-MS: [M+H]⁺=545.0.

### Example 126: 2-(difluoromethoxy)-N-((1S,2R)-2-hydroxycyclopentyl)-6-methoxy-4-(6-(2-(pyrrolidin-1-yl) ethoxy)benzofuran-3-yl)benzamide (compound 126)

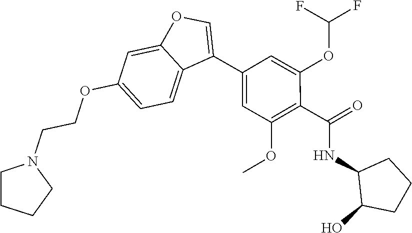 [SMILES: COc1cc(-c2coc3cc(OCCN4CCCC4)ccc23)cc(OC(F)F)c1C(=O)N[C@H]1CCC[C@H]1O]

Compound 126 (7 mg, 16% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and (1R,2S)-2-aminocyclopentan-1-ol. ¹H NMR (500 MHz, DMSO-d₆) δ 8.39 (s, 1H), 7.79 (d, J=8.7 Hz, 1H), 7.74 (d, J=7.5 Hz, 1H), 7.35-6.95 (m, 5H), 4.38 (s, 1H), 4.15 (t, J=5.9 Hz, 2H), 4.08-3.95 (m, 2H), 3.90 (d, J=8.3 Hz, 3H), 2.82 (t, J=5.8 Hz, 2H), 2.53 (d, J=5.4 Hz, 4H), 1.90-1.86 (m, 1H), 1.81-1.64 (m, 6H), 1.62-1.47 (m, 3H). LC-MS: [M+H]⁺=531.0.

### Example 127: N-cyclohexyl-2-(difluoromethoxy)-6-methoxy-4-(6-(2-(pyrrolidin-1-yl) ethoxy)benzofuran-3-yl)benzamide (compound 127)

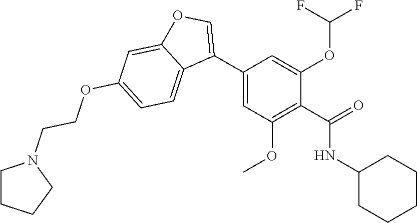 [SMILES: COc1cc(-c2coc3cc(OCCN4CCCC4)ccc23)cc(OC(F)F)c1C(=O)NC1CCCCC1]

Compound 127 (11 mg, 26% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cyclohexanamine. ¹H NMR (500 MHz, DMSO-d₆) δ 8.37 (s, 1H), 8.15 (d, J=8.1 Hz, 1H), 7.78 (d, J=8.7 Hz, 1H), 7.33-7.00 (m, 5H), 4.15 (t, J=5.8 Hz, 2H), 3.89 (d. J=11.8 Hz, 3H), 3.78-3.60 (m, 1H), 2.84 (t, J=5.8 Hz, 2H), 2.67 (d, J=32.3 Hz, 4H), 1.81 (d, J=10.0 Hz, 2H), 1.75-1.68 (m, 6H), 1.36-1.06 (m, 6H). LC-MS: [M+H]⁺=529.0.

### Example 128: N-cyclopentyl-2-(difluoromethoxy)-6-methoxy-4-(6-(2-(pyrrolidin-1-yl) ethoxy)benzofuran-3-yl)benzamide (compound 128)

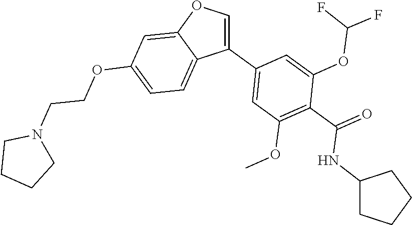 [SMILES: COc1cc(-c2coc3cc(OCCN4CCCC4)ccc23)cc(OC(F)F)c1C(=O)NC1CCCC1]

Compound 128 (9 mg, 22% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cyclopentanamine. ¹H NMR (500 MHz, DMSO-d₆) δ 8.46-8.35 (m, 1H), 8.31-8.20 (m, 1H), 7.87-7.70 (m, 1H), 7.43-7.15 (m, 3H), 7.15-6.97 (m, 2H), 4.17 (d, J=5.5 Hz, 3H), 4.00-3.80 (m, 3H), 2.91 (s, 2H), 2.62 (s, 4H), 1.84 (s, 2H), 1.73-1.69 (m, 6H), 1.52 (s, 4H). LC-MS: [M+H]⁺=515.0.

### Example 129: N-cyclobutyl-2-(difluoromethoxy)-6-methoxy-4-(6-(2-(pyrrolidin-1-yl) ethoxy)benzofuran-3-yl)benzamide (compound 129)

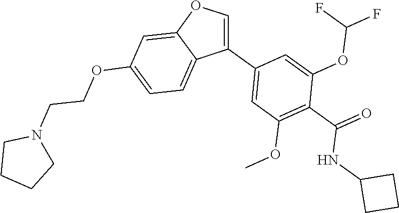 [SMILES: COc1cc(-c2coc3cc(OCCN4CCCC4)ccc23)cc(OC(F)F)c1C(=O)NC1CCC1]

Compound 129 (9 mg, 23% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cyclobutanamine. ¹H NMR (500 MHz, DMSO-d₆) δ 8.52 (d, J=7.7 Hz, 1H), 8.39 (d, J=8.2 Hz, 1H), 7.78 (d, J=8.7 Hz, 1H), 7.34-7.00 (m, 5H), 4.33 (dd, J₁=16.1 Hz, J₂=8.0 Hz, 1H), 4.16 (t, J=5.8 Hz, 2H), 3.90 (d, J=15.3 Hz, 3H), 2.87 (t, J=5.6 Hz, 2H), 2.59 (s, 4H), 2.30-2.17 (m, 2H), 2.02-1.89 (m, 2H), 1.73-1.63 (m, 6H). LC-MS: [M+H]⁺=501.0.

### Example 130: N-(2-aminocyclopentyl)-2-(difluoromethoxy)-6-methoxy-4-(6-(2-(pyrrolidin-1-yl) ethoxy)benzofuran-3-yl)benzamide (cis racemate, compound 130)

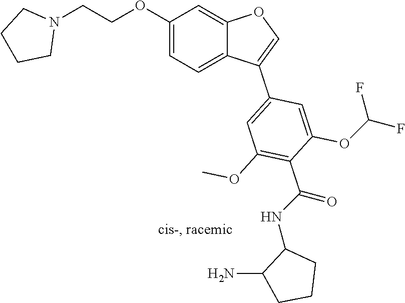 [SMILES: COc1cc(-c2coc3cc(OCCN4CCCC4)ccc23)cc(OC(F)F)c1C(=O)NC1CCCC1N]

Compound 130 (8 mg, 11% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cis-1,2-cyclopentanediamine hydrochloride. 1H NMR (500 MHz, DMSO-d₆) δ 8.70 (d, J=7.8 Hz, 1H), 8.41 (s, 1H), 7.80 (d, J=8.7 Hz, 1H), 7.28 (s, 1H), 7.23 (t. J=75.0 Hz, 1H), 7.13 (s, 1H), 7.09 (s, 1H), 7.01 (dd, J₁=8.5 Hz, J₂=2.0 Hz, 1H), 4.30-4.24 (m, 1H), 4.17 (t, J=5.8 Hz, 2H), 3.87 (s, 3H), 3.54-3.40 (m, 1H), 2.90 (t, J=5.8 Hz, 2H), 2.68-2.58 (m, 4H), 1.97-1.86 (m, 2H), 1.79-1.66 (m, 6H), 1.64-1.56 (m, 1H), 1.56-1.46 (m, 1H). LC-MS: [M+H]⁺=530.3.

### Example 131: N-(2-aminocyclopropyl)-2-(difluoromethoxy)-6-methoxy-4-(6-(2-(pyrrolidin-1-yl) ethoxy)benzofuran-3-yl)benzamide (cis racemate, compound 131)

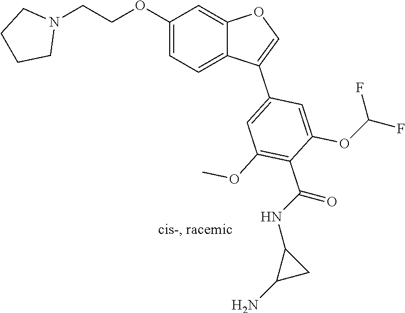 [SMILES: COc1cc(-c2coc3cc(OCCN4CCCC4)ccc23)cc(OC(F)F)c1C(=O)NC1CC1N]

Compound 131 (4 mg, 5% yield) was prepared in a manner similar to that described in Example 25 step 9 from 2-(difluoromethoxy)-6-methoxy-4-(6-(2-(pyrrolidin-1-yl) ethoxy)benzofuran-3-yl)benzoic acid and cis-1,2-cyclopropanediamine hydrochloride. 1H NMR (500 MHz, DMSO-d₆) δ 8.38 (s, 1H), 8.05 (d, J=4.7 Hz, 1H), 7.79 (d, J=8.7 Hz, 1H), 7.33 (s, 1H), 7.25 (s, 1H), 7.23 (t, J=75.0 Hz, 1H), 7.09 (s, 1H), 7.01 (dd, J₁=8.5 Hz, J₂=2.0 Hz, 1H), 4.15 (t, J=5.8 Hz, 2H), 3.89 (s, 3H), 2.84 (t, J=5.8 Hz, 2H), 2.76-2.68 (m, 1H), 2.59-2.53 (m, 4H), 2.35-2.28 (m, 1H), 1.75-1.66 (m, 4H), 0.95-0.87 (m, 1H), 0.36-0.26 (m, 1H). LC-MS: [M+H]⁺=502.4.

### Example 132: N-cyclopropyl-2-(difluoromethoxy)-6-methoxy-4-(6-(1-methyl-1H-pyrazol-4-yl)benzofuran-3-yl)benzamide (compound 132)

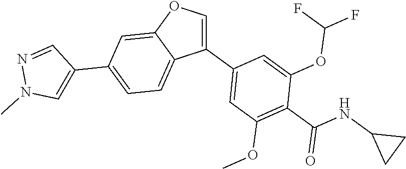 [SMILES: COc1cc(-c2coc3cc(-c4cnn(C)c4)ccc23)cc(OC(F)F)c1C(=O)NC1CC1]

### Step 1:3-(4-(cyclopropylcarbamoyl)-3-(difluoromethoxy)-5-methoxyphenyl)benzofuran-6-yl trifluoromethanesulfonate

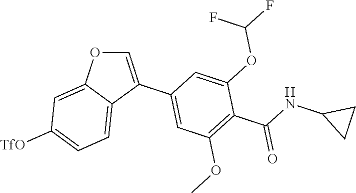 [SMILES: COc1cc(-c2coc3cc(OS(=O)(=O)C(F)(F)F)ccc23)cc(OC(F)F)c1C(=O)NC1CC1]

The title compound of step 1 (210 mg, 82% yield) was prepared in a manner similar to that described in Example 13 step 8 from N-cyclopropyl-2-(difluoromethoxy)-4-(6-hydroxybenzofuran-3-yl)-6-methoxybenzamide and triflic anhydride. LC-MS: [M+H]⁺=522.2.

### Step 2: N-cyclopropyl-2-(difluoromethoxy)-6-methoxy-4-(6-(1-methyl-1H-pyrazol-4-yl)benzofuran-3-yl)benzamide (compound 132)

Compound 132 (26 mg, 30% yield) was prepared in a manner similar to that described in Example 1 step 5 from 3-(4-(cyclopropylcarbamoyl)-3-(difluoromethoxy)-5-methoxyphenyl)benzofuran-6-yl trifluoromethanesulfonate and (1-methyl-1H-pyrazol-4-yl) boronic acid. ¹H NMR (500 MHz, DMSO-d₆) δ 8.45 (s, 1H), 8.34 (d, J=4.3 Hz, 1H), 8.23 (s, 1H), 7.97 (s, 1H), 7.91-7.86 (m, 2H), 7.61 (d, J=8.2 Hz, 1H), 7.24 (s, 1H), 7.23 (t, J=75.0 Hz, 1H), 7.11 (s, 1H), 3.89 (s, 3H), 3.88 (s, 3H), 2.84-2.74 (m, 1H), 0.70-0.65 (m, 2H), 0.51-0.43 (m, 2H). LC-MS [M+H]⁺=454.2.

### Example 133: N-cyclopropyl-2-(difluoromethoxy)-6-methoxy-4-(6-(6-methylpyridazin-3-yl)benzofuran-3-yl)benzamide (compound 133)

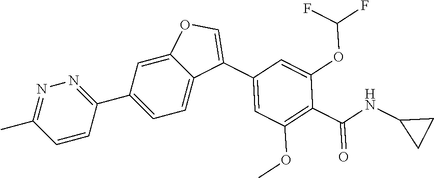 [SMILES: COc1cc(-c2coc3cc(-c4ccc(C)nn4)ccc23)cc(OC(F)F)c1C(=O)NC1CC1]

### Step 1: N-cyclopropyl-2-(difluoromethoxy)-6-methoxy-4-(6-(4,4,5,5-tetramethyl-1,3,2-dioxaborolan-2-yl)benzofuran-3-yl)benzamide

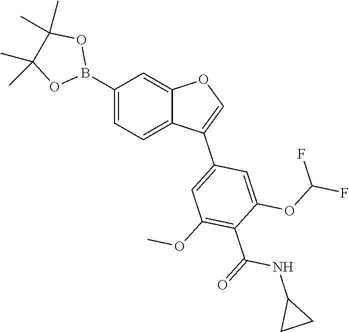 [SMILES: COc1cc(-c2coc3cc(B4OC(C)(C)C(C)(C)O4)ccc23)cc(OC(F)F)c1C(=O)NC1CC1]

The title compound of step 1 (30 mg, 52% yield) was prepared in a manner similar to that described in Example 1 step 4 from 3-(4-(cyclopropylcarbamoyl)-3-(difluoromethoxy)-5-methoxyphenyl)benzofuran-6-yl trifluoromethanesulfonate and 4,4,4′,4′,5,5,5′,5′-octamethyl-2,2′-bi (1,3,2-dioxaborolane). LC-MS [M+H]⁺=500.4.

### Step 2: N-cyclopropyl-2-(difluoromethoxy)-6-methoxy-4-(6-(6-methylpyridazin-3-yl)benzofuran-3-yl)benzamide (compound 133)

Compound 133 (19 mg, 44% yield) was prepared in a manner similar to that described in Example 1 step 6 from N-cyclopropyl-2-(difluoromethoxy)-6-methoxy-4-(6-(4,4,5,5-tetramethyl-1,3,2-dioxaborolan-2-yl)benzofuran-3-yl)benzamide and 3-bromo-6-methylpyridazine. ¹H NMR (500 MHz, DMSO-d₆) δ 8.62 (s, 1H), 8.47 (s, 1H), 8.36 (d, J=4.3 Hz, 1H), 8.27-8.22 (m, 2H), 8.08 (d, J=8.3 Hz, 1H), 7.69 (d, J=8.8 Hz, 1H), 7.29 (s, 1H), 7.25 (t, J=75.0 Hz, 1H), 7.16 (s, 1H), 3.92 (s, 3H), 2.82-2.75 (m, 1H), 2.68 (s, 3H), 0.72-0.65 (m, 2H), 0.51-0.44 (m, 2H). LC-MS [M+H]⁺=466.2.

### Example 134: 1-(2-(6-(2-morpholinoethoxy)benzofuran-3-yl) quinolin-8-yl) piperidin-4-amine (compound 134)

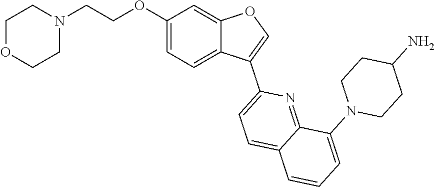 [SMILES: NC1CCN(c2cccc3ccc(-c4coc5cc(OCCN6CCOCC6)ccc45)nc23)CC1]

### Step 1: 3-(4,4,5,5-tetramethyl-1,3,2-dioxaborolan-2-yl)benzofuran-6-yl acetate

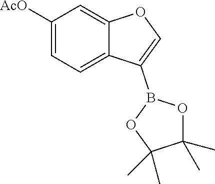 [SMILES: CC(=O)Oc1ccc2c(B3OC(C)(C)C(C)(C)O3)coc2c1]

The title compound of step 1 (5 g, crude) was prepared in a manner similar to that described in Example 1 step 4 from 3-(((trifluoromethyl) sulfonyl)oxy)benzofuran-6-yl acetate and 4,4,4′,4′,5,5,5′,5′-octamethyl-2,2′-bi (1,3,2-dioxaborolane). LC-MS [M+H]⁺=303.1.

### Step 2: 3-(8-(benzyloxy) quinolin-2-yl)benzofuran-6-yl acetate

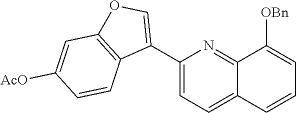 [SMILES: CC(=O)Oc1ccc2c(-c3ccc4cccc(OCc5ccccc5)c4n3)coc2c1]

The title compound of step 2 (4 g, 97% yield) was prepared in a manner similar to that described in Example 1 step 6 from 3-(4,4,5,5-tetramethyl-1,3,2-dioxaborolan-2-yl)benzofuran-6-yl acetate and 8-(benzyloxy)-2-chloroquinoline. LC-MS [M+H]⁺=410.4.

### Step 3: 3-(8-(benzyloxy) quinolin-2-yl)benzofuran-6-ol

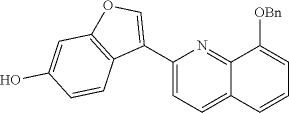 [SMILES: Oc1ccc2c(-c3ccc4cccc(OCc5ccccc5)c4n3)coc2c1]

The title compound of step 3 (3.2 g, 87% yield) was prepared in a manner similar to that described in Example 13 step 7 from 3-(8-(benzyloxy) quinolin-2-yl)benzofuran-6-yl acetate. LC-MS [M+H]⁺=367.4.

### Step 4: 4-(2-((3-(8-(benzyloxy) quinolin-2-yl)benzofuran-6-yl)oxy)ethyl) morpholine

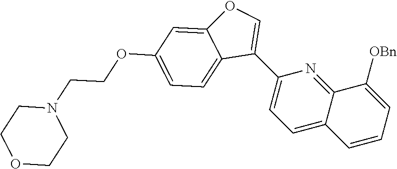 [SMILES: c1ccc(COc2cccc3ccc(-c4coc5cc(OCCN6CCOCC6)ccc45)nc23)cc1]

The title compound of step 4 (652 mg, 100% yield) was prepared in a manner similar to that described in Example 58 step 1 from 3-(8-(benzyloxy) quinolin-2-yl)benzofuran-6-ol and 2-morpholinoethan-1-ol. LC-MS [M+H]⁺=481.4.

### Step 5: 2-(6-(2-morpholinoethoxy)benzofuran-3-yl) quinolin-8-ol

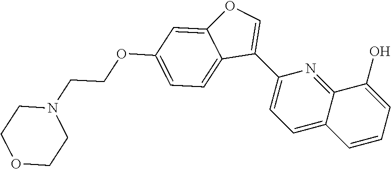 [SMILES: Oc1cccc2ccc(-c3coc4cc(OCCN5CCOCC5)ccc34)nc12]

A mixture of 4-(2-((3-(8-(benzyloxy) quinolin-2-yl)benzofuran-6-yl)oxy)ethyl) morpholine (652 mg, 1.36 mmol), Pd(OH)₂ (70 mg) and Ammonium formate (857 mg, 13.6 mmol) in MeOH (10 mL) was stirred at refluxed temperature for 2 h. The solvent was evaporated and the residue was treated with saturated aqueous NaHCO₃ solution. The precipitate was filtered, washed with water and dried to give the crude title product (390 mg) which was directly used in the next step without further purification. LC-MS [M+H]=391.4.

### Step 6: 2-(6-(2-morpholinoethoxy)benzofuran-3-yl) quinolin-8-yl trifluoromethanesulfonate

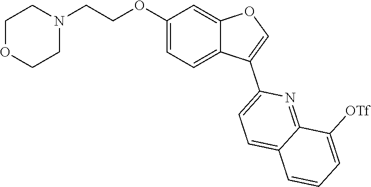 [SMILES: O=S(=O)(Oc1cccc2ccc(-c3coc4cc(OCCN5CCOCC5)ccc34)nc12)C(F)(F)F]

The title compound of step 6 (430 mg, 80% yield) was prepared in a manner similar to that described in Example 13 step 8 from 2-(6-(2-morpholinoethoxy)benzofuran-3-yl) quinolin-8-ol and triflic anhydride. LC-MS [M+H]⁺=523.4.

### Step 7: tert-butyl (1-(2-(6-(2-morpholinoethoxy)benzofuran-3-yl) quinolin-8-yl) piperidin-4-yl) carbamate

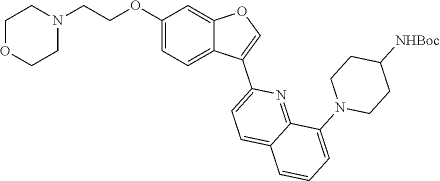 [SMILES: CC(C)(C)OC(=O)NC1CCN(c2cccc3ccc(-c4coc5cc(OCCN6CCOCC6)ccc45)nc23)CC1]

The reaction mixture of 2-(6-(2-morpholinoethoxy)benzofuran-3-yl) quinolin-8-yl trifluoromethanesulfonate (52 mg, 0.1 mmol), tert-butyl piperidin-4-ylcarbamate (30 mg, 0.15 mmol), Pd₂(dba)₃ (9 mg, 0.01 mmol), BINAP (6 mg, 0.01 mmol) and Cs₂CO₃ (65 mg, 0.2 mmol) in 1,4-dioxane (5 mL) under nitrogen was stirred at 90° C. for overnight. The mixture was filtered and the filtrate was evaporated. The residue was purified by silica gel column chromatography, eluting with PE/EtOAc (v/v=1/1), to afford the title product (15 mg, 26% yield). LC-MS [M+H]⁺=573.4.

### Step 8: 1-(2-(6-(2-morpholinoethoxy)benzofuran-3-yl) quinolin-8-yl) piperidin-4-amine (compound 134)

Compound 134 (12 mg, 100% yield) was prepared in a manner similar to that described in Example 28 step 2 from tert-butyl (1-(2-(6-(2-morpholinoethoxy)benzofuran-3-yl) quinolin-8-yl) piperidin-4-yl) carbamate. ¹H NMR (500 MHz, DMSO-d₆) δ 8.85 (s, 1H), 8.78 (d, J=8.8 Hz, 1H), 8.34 (d, J=8.7 Hz, 2H), 8.04 (d, J=8.6 Hz, 1H), 7.56 (d, J=8.1 Hz, 1H), 7.47 (t, J=7.7 Hz, 1H), 7.34 (s, 1H), 7.25 (d, J=7.5 Hz, 2H), 4.19 (t, J=5.7 Hz, 2H), 3.95-3.93 (m, 2H), 3.64-3.56 (m, 5H), 3.13 (s, 1H), 2.83-2.79 (m, 2H), 2.77-2.74 (m, 2H), 2.07-2.05 (m, 2H), 1.95-1.93 (m, 2H). LC-MS [M+H]⁺=473.4.

### Biological Assays

### 1. Biochemical Activities Determination for SIK1 Protein

Compounds were tested for inhibition of SIK1 kinase (aa1-303, Thermo Fisher) in assays based on the time-resolved fluorescence-resonance energy transfer (TR-FRET) methodology. The assays were carried out in 384-well low volume white plates in a reaction mixture containing SIK1 kinase, 110 uM ATP, 0.5 μM STK1 substrate and 0-10 μM compound in buffer containing 25 mM Tris, 0.01% Trition X-100, 0.5 mM EGTA, 5 mM MgCl₂, 2.5 mM DTT, pH=7.5. The kinase was incubated with compounds for 60 minutes at room temperature and the reaction was initiated by the addition of ATP and STK1 substrate. After reaction at room temperature for 180 minutes, an equal volume of stop/detection solution was added according to the manufacture's instruction (CisBio Bioassays). The stop/detection solution contained STK Antibody-Cryptate and XL665-conjugated streptavidin (Cat: 62ST1PEC, Cisbio) in Detection Buffer (Cat: 62SDBRDF, Cisbio). Plates were sealed and incubated at room temperature for overnight, and the TR-FRET signals (ratio of fluorescence emission at 665 nm over emission at 620 nm with excitation at 337 nm wavelength) were recorded on a PHERAstar FS plate reader (BMG Labtech). Phosphorylation of STK1 substrate led to the binding of STK Antibody-Cryptate to the biotinylated SIK1 substrate, which places fluorescent donor (Eu³⁺ crypate) in close proximity to the accepter (Streptavidin-XL665), thus resulting in a high degree of fluorescence resonance energy transfer from the donor fluorophore (at 620 nm) to the acceptor fluorophore (at 665 nm). Inhibition of SIK1 kinase activity resulted in decrease of the TR-FRET signal. The IC₅₀ for each compound was derived from fitting the data to the four-parameter logistic equation by Dotmatics.

### 2. Biochemical Activities Determination for SIK2 Protein

Compounds were tested for inhibition of SIK2 kinase (full length, Thermo Fisher) in assays based on the time-resolved fluorescence-resonance energy transfer (TR-FRET) methodology. The assays were carried out in 384-well low volume white plates in a reaction mixture containing SIK2 kinase, 80 uM ATP, 0.5 μM SIK1 substrate and 0-10 μM compound in buffer containing 25 mM Tris, 0.01% Trition X-100, 0.5 mM EGTA, 5 mM MgCl₂, 2.5 mM DTT, pH=7.5. The kinase was incubated with compounds for 60 minutes at room temperature and the reaction was initiated by the addition of ATP and STK1 substrate. After reaction at room temperature for 180 minutes, an equal volume of stop/detection solution was added according to the manufacture's instruction (CisBio Bioassays). The stop/detection solution contained STK Antibody-Cryptate and XL665-conjugated streptavidin (Cat: 62ST1PEC, Cisbio) in Detection Buffer (Cat: 62SDBRDF, Cisbio). Plates were sealed and incubated at room temperature for overnight, and the TR-FRET signals (ratio of fluorescence emission at 665 nm over emission at 620 nm with excitation at 337 nm wavelength) were recorded on a PHERAstar FS plate reader (BMG Labtech). Phosphorylation of STK1 substrate led to the binding of STK Antibody-Cryptate to the biotinylated STK1 substrate, which places fluorescent donor (Eu³⁺ crypate) in close proximity to the accepter (Streptavidin-XL665), thus resulting in a high degree of fluorescence resonance energy transfer from the donor fluorophore (at 620 nm) to the acceptor fluorophore (at 665 nm). Inhibition of SIK2 kinase activity resulted in decrease of the TR-FRET signal. The IC₅₀ for each compound was derived from fitting the data to the four-parameter logistic equation by Dotmatics.

### 3. Biochemical Activities Determination for SIK3 Protein

Compounds were tested for inhibition of SIK3 kinase (aa59-365, Thermo Fisher) in assays based on the time-resolved fluorescence-resonance energy transfer (TR-FRET) methodology. The assays were carried out in 384-well low volume white plates in a reaction mixture containing SIK3 kinase, 125 uM ATP, 0.5 μM STK1 substrate and 0-10 μM compound in buffer containing 25 mM Tris, 0.01% Trition X-100, 0.5 mM EGTA, 5 mM MgCl₂, 2.5 mM DTT, PH=7.5. The kinase was incubated with compound for 60 minutes at room temperature and the reaction was initiated by the addition of ATP and STK1 substrate. After reaction at room temperature for 180 minutes, an equal volume of stop/detection solution was added according to the manufacture's instruction (CisBio Bioassays). The stop/detection solution contained STK Antibody-Cryptate and XL665-conjugated streptavidin (Cat: 62ST1PEC, Cisbio) in Detection Buffer (Cat: 62SDBRDF, Cisbio). Plates were sealed and incubated at room temperature for overnight, and the TR-FRET signals (ratio of fluorescence emission at 665 nm over emission at 620 nm with excitation at 337 nm wavelength) were recorded on a PHERAstar FS plate reader (BMG Labtech). Phosphorylation of STK1 substrate led to the binding of STK Antibody-Cryptate to the biotinylated STK1 substrate, which places fluorescent donor (Eu³⁺ crypate) in close proximity to the accepter (Streptavidin-XL665), thus resulting in a high degree of fluorescence resonance energy transfer from the donor fluorophore (at 620 nm) to the acceptor fluorophore (at 665 nm). Inhibition of SIK3 kinase activity resulted in decrease of the TR-FRET signal. The IC₅₀ for each compound was derived from fitting the data to the four-parameter logistic equation by Dotmatics.

TABLE 1
biochemical activities (IC₅₀, nM) for the compounds disclosed herein:

| Example | Biochemical IC₅₀ (nM) | | | Example | Biochemical IC₅₀ (nM) | | |
|---|---|---|---|---|---|---|---|
| No. | SIK1 | SIK2 | SIK3 | No. | SIK1 | SIK2 | SIK3 |
| 1 | 303 | 10 | 19 | 2 | 375 | 15 | 28 |
| 3 | 476 | 28 | 42 | 4 | 728 | 37 | 53 |
| 5 | 538 | 33 | 41 | 6 | >10000 | 7462 | 1431 |
| 7 | 671 | 14 | 60 | 8 | >10000 | 197 | 304 |
| 9 | >10000 | 296 | 334 | 10 | >10000 | 418 | 187 |
| 11 | 1546 | 49 | 155 | 12 | 2277 | 59 | 186 |
| 13 | 647 | 25 | 54 | 14 | >10000 | 310 | 329 |
| 15 | 6040 | 95 | 174 | 16 | >10000 | 3355 | 5843 |
| 17 | 2308 | 28 | 79 | 18 | >10000 | 214 | 237 |
| 19 | 3135 | 80 | 132 | 20 | 362 | 12 | 14 |
| 21 | 289 | 11 | 15 | 22 | 5923 | 79 | 127 |
| 23 | >10000 | 223 | 387 | 24 | 3342 | 281 | 320 |
| 25 | 195 | 15 | 26 | 26 | 33 | 3.4 | 5.0 |
| 27 | 1131 | 34 | 72 | 28 | 3937 | 140 | 281 |
| 29A | 216 | 15 | 42 | 29B | 5976 | 178 | 374 |
| 30 | 2612 | 531 | 1115 | 31 | 5454 | 673 | 882 |
| 32 | 1884 | 96 | 829 | 33 | 9096 | 82 | 156 |
| 34 | 2678 | 239 | 420 | 35 | 2076 | 132 | 255 |
| 36 | 2094 | 144 | 305 | 37 | 25 | 1.4 | 4.2 |
| 38 | 24 | 1.7 | 3.9 | 39 | 25 | 1.7 | 4.1 |
| 40 | 537 | 14 | 31 | 41 | 1707 | 33 | 69 |
| 42 | 1586 | 25 | 115 | 43 | 676 | 8 | 57 |
| 44 | 538 | 5.2 | 45 | 45 | 560 | 9.8 | 28 |
| 46 | 430 | 7.8 | 36.1 | 47 | 335 | 3.0 | 33 |
| 48 | 524 | 11 | 63 | 49 | 337 | 11 | 25 |
| 50 | 347 | 17 | 34 | 51 | 317 | 3.7 | 26 |
| 52 | 212 | 4.9 | 23 | 53 | 294 | 2.8 | 19 |
| 54 | 326 | 6.9 | 19 | 55 | 301 | 3.9 | 18 |
| 56 | 315 | 2.6 | 15 | 57 | 405 | 2.7 | 17 |
| 58 | >10000 | 167 | 391 | 59 | >10000 | 77 | 348 |
| 60 | >10000 | 635 | 421 | 61 | >10000 | 147 | 322 |
| 62 | >10000 | 76 | 122 | 63 | >10000 | 67 | 300 |
| 64 | >10000 | 73 | 415 | 65 | >10000 | 103 | 313 |
| 66 | 6695 | 58 | 132 | 67 | 2450 | 23 | 79 |
| 68 | 8179 | 90 | 139 | 69 | 1274 | 41 | 45 |
| 70 | 2949 | 14 | 42 | 71 | 1375 | 48 | 39 |
| 72 | 461 | 6.0 | 31 | 73 | 3264 | 24 | 65 |
| 74 | 1133 | 26 | 34 | 75 | 1739 | 16 | 93 |
| 76 | 1008 | 34 | 34 | 77 | 3372 | 27 | 80 |
| 78 | 1888 | 19 | 42 | 79 | 2194 | 19 | 57 |
| 80 | 2849 | 20 | 54 | 81 | 1891 | 11 | 32 |
| 82 | 1466 | 15 | 57 | 83 | 737 | 13 | 75 |
| 84 | 559 | 9.0 | 44 | 85 | 236 | 8.1 | 32.8 |
| 86 | 562 | 18 | 43 | 87 | 1088 | 78 | 22 |
| 88 | 4536 | 17 | 68 | 89 | 4213 | 24 | 74 |
| 90 | 2354 | 13 | 33 | 91 | 2495 | 31 | 74 |
| 92 | 3011 | 32 | 104 | 93 | 2529 | 30 | 60 |
| 94 | 2869 | 33 | 74 | 95 | >10000 | 507 | 117 |
| 96 | 2037 | 22 | 43 | 97 | 8353 | 85 | 289 |
| 98 | >10000 | 128 | 525 | 99 | >10000 | 269 | 759 |
| 100 | 3592 | 31 | 80 | 101 | >10000 | 8968 | >10000 |
| 102 | >10000 | 2477 | >10000 | 103 | 9498 | 114 | 410 |
| 104 | 4579 | 52 | 211 | 105 | >10000 | 398 | 1770 |
| 106 | >10000 | 2956 | >10000 | 107 | >10000 | 5612 | >10000 |
| 108 | >10000 | 2777 | >10000 | 109 | >10000 | 329 | 2522 |
| 110 | >10000 | 3854 | >10000 | 111 | >10000 | 1189 | 5705 |
| 112 | >10000 | 1303 | 7619 | 113 | >10000 | 7317 | >10000 |
| 114 | >10000 | 745 | 5491 | 115 | >10000 | 582 | 4240 |
| 116 | >10000 | 1121 | >10000 | 117 | >10000 | 2941 | >10000 |
| 118 | >10000 | 781 | 6308 | 119 | 7176 | 136 | 452 |
| 120 | >10000 | 619 | 2540 | 121 | >10000 | 438 | 2121 |
| 122 | 3963 | 45 | 158 | 123 | 8005 | 114 | 506 |
| 124 | 1220 | 18 | 161 | 125 | >10000 | 385 | 2490 |
| 126 | >10000 | 224 | 878 | 127 | >10000 | 619 | 2904 |
| 128 | >10000 | 156 | 812 | 129 | >10000 | 146 | 683 |
| 130 | >10000 | 103 | 359 | 131 | >10000 | 653 | 1563 |
| 132 | 124 | 13 | 15 | 133 | >10000 | 199 | 199 |
| 134 | 958 | 66 | 156 | | | | |

It is to be understood that, if any prior art publication is referred to herein; such references does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (I): 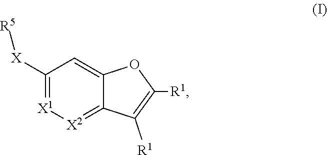
or a pharmaceutically acceptable salt, stereoisomer, or tautomer thereof,
wherein:
X is a divalent heteroaryl ring, a divalent heterocycloalkyl ring, —O—, —CH₂—, —NH— or —O—C₁₋₈alkylene-, wherein each of said divalent heteroaryl ring, heterocycloalkyl ring, —CH₂—, —NH— or —O—C₁₋₈alkylene is independently optionally substituted with at least one R^{a};
R^{a} is each independently selected from halogen, —C₁₋₈alkyl, —C₁₋₈haloalkyl, —CN, and —C₁₋₈alkoxy;
R⁵ is selected from hydrogen, —C₁₋₈alkyl, —C₁₋₈alkoxy, —C2-8alkynyl, —C₁₋₈haloalkyl, —C₃-C₈cycloalkyl, 4- to 8-membered heterocycloalkyl, aryl, 5- to 12-membered heteroaryl, —NR⁵ⁱR^{5j}, —CONR⁵ⁱR^{5j}, —SR⁵ⁱ, and —NR⁵ⁱCOR^{5j}; wherein said —C₁₋₈alkyl, —C₁₋₈alkoxy, —C₂₋₈alkynyl, —C₁₋₈haloalkyl, —C₃-C₈cycloalkyl, 4- to 8-membered heterocycloalkyl, aryl, or 5- to 12-membered heteroaryl is independently optionally substituted with at least one R^{5a},
wherein said R^{5a} is each independently selected from halogen, oxo, —OH, —CN, —C₁₋₈alkyl, —C₁₋₈haloalkyl, —C₁₋₈alkoxy, cycloalkyl, heterocycloalkyl, aryl, heteroaryl, —NR^{5b}R^{5c}, and —COR^{5d}; wherein said —C₁₋₈alkyl, —C₁₋₈haloalkyl, —C₁₋₈alkoxy, cycloalkyl, heterocycloalkyl, aryl, or heteroaryl is independently optionally substituted with at least one R^{5g};
R^{5b}, R^{5c} and R^{5d} are each independently selected from hydrogen, —C₁₋₈alkyl, cycloalkyl, heterocycloalkyl, aryl, and heteroaryl;
R^{5g} is each independently selected from halogen, oxo, —OH, —CN, —C₁₋₈alkyl, —C₁₋₈haloalkyl, —C₁₋₈alkoxy, C₁₋₈alkyl-CO—, and —NO₂,
wherein said R⁵ⁱ and R^{5j} are each independently selected from hydrogen, —C₁₋₈alkyl, cycloalkyl, heterocycloalkyl, aryl, and heteroaryl; wherein said —C₁₋₈alkyl, cycloalkyl, heterocycloalkyl, aryl, or heteroaryl is independently optionally substituted with at least one R^{5m};
wherein said R^{5m} is each independently selected from halogen, oxo, —OH, —CN, —C₁₋₈alkyl, —C₁₋₈haloalkyl, —C₁₋₈alkoxy, C₁₋₈alkyl-CO—, and —NO₂;
R¹ is selected from hydrogen, halogen, —C₁₋₈alkyl, —C₁₋₈haloalkyl, and —C₁₋₈alkoxy;
X¹ is N or CR³ and X² is N or CR⁴, with the proviso that X¹ and X² cannot both be N;
R³ and R⁴ are each independently selected from hydrogen, halogen (F, Cl, Br or I), —C₁₋₈alkyl (preferably —CH₃), —C₁₋₈haloalkyl, —C₁₋₈alkoxy (preferably —OCH₃), —CN, and —NR^{1a}R^{1b}(preferably —NHCH₃);
R^{1a} and R^{1b} are each independently selected from hydrogen and —C₁₋₈alkyl (preferably —CH₃);
R² is 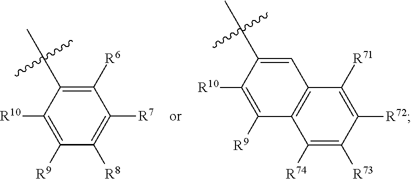
R⁶ and R¹⁰ are each independently selected from hydrogen, halogen, —C₁₋₈alkyl, —C₁₋₈haloalkyl, and —C₁₋₈alkoxy; wherein either
(A) R⁷ and R⁹ are each independently selected from hydrogen, halogen, —C₁₋₈alkyl, cycloalkyl, heterocycloalkyl, —OR^{7a}, —COR^{7a}, —NR^{7d}R^{7e}, —CONR^{7d}R^{7e}, —OH, and —CN; wherein said —C₁₋₈alkyl, cycloalkyl, or heterocycloalkyl is optionally substituted with at least one R^{7g};
wherein R^{7g} is each independently selected from hydrogen, halogen, —C₁₋₈alkoxy, —COC₁₋₈alkyl, —NH₂, —OH, and —CN;
wherein R^{7a} is each independently selected from —C₁₋₈alkyl, C₃₋₈cycloalkyl, and 3- to 10-membered heterocycloalkyl, wherein said —C₁₋₈alkyl, C₃₋₈cycloalkyl, or 3- to 10-membered heterocycloalkyl is optionally substituted with at least one R^{7b};
wherein R^{7b} is each independently selected from halogen, —OH, —C₁₋₈alkyl, cycloalkyl, heterocycloalkyl, —NH₂, and —C₁₋₈alkoxy, wherein said cycloalkyl, heterocycloalkyl or —C₁₋₈alkoxy is optionally substituted with at least one R^{7c};
wherein R^{7c} is each independently selected from halogen, —C₁₋₈alkyl, —CN, and —OH;
wherein R^{7d} and R^{7e} are each independently selected from hydrogen and —C₁₋₈alkyl, wherein said —C₁₋₈alkyl is optionally substituted with at least one R^{7f};
wherein R^{7f} is each independently halogen, —CN, or —OH;
R⁷², R⁷³, and R⁷⁴ are each independently selected from hydrogen, halogen, —C₁₋₈alkyl, —C₁₋₈haloalkyl, and —C₁₋₈alkoxy;
R⁷¹ is selected from 4- to 15-membered monocyclic, spirocyclic, and bridged polycyclic heterocycloalkyl comprising one, two, or three heteroatoms independently selected from N, O, and S, wherein each of said heterocycloalkyl is independently optionally substituted with at least one R^{71a};
R^{71a} is each independently selected from hydrogen, halogen, —C₁₋₈alkyl, —C₁₋₈haloalkyl, —C₁₋₈alkoxy and —NH₂;
R⁸ is selected from:
(i) —CN,
(ii) 5- to 6-membered monocyclic heteroaryl comprising one, two, or three heteroatoms independently selected from N, O, and S; wherein said heteroaryl is optionally substituted with at least one R^{8a}; wherein R^{8a} is each independently selected from —C₁₋₈alkyl and cycloalkyl; and
(iii) —CONR^{8b}R^{8c}, wherein R^{8b} and R^{8c} are each independently selected from hydrogen, —C₁₋₈alkyl, 5- to 6-membered heterocycloalkyl and C₃-C₈cycloalkyl, wherein said —C₁₋₈alkyl, 5- to 6-membered heterocycloalkyl or C₃-C₈cycloalkyl is optionally substituted with at least one R^{8d}, wherein R^{8d} is each independently selected from halogen,
—OH, —NH₂, —CN, —CONH₂, aryl, and heteroaryl; or
(B) R⁸ and R⁹ together with the atoms onto which they are attached form a fused 5-, 6- or 7-membered heterocycloalkyl 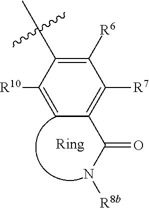
containing —CONR^{8b}— moiety, wherein said heterocycloalkyl is optionally substituted with at least one R^{8e};
R^{8b} is selected from hydrogen, —C₁₋₈alkyl, 5- to 6-membered heterocycloalkyl and C₃-C₈cycloalkyl, wherein said —C₁₋₈alkyl 5- to 6-membered heterocycloalkyl or C₃-C₈cycloalkyl is optionally substituted with at least one R^{8d};
R^{8d} is each independently selected from halogen, —OH, —NH₂, —CN, —CONH₂, aryl, and heteroaryl;
wherein R^{8e} is each independently selected from —C₁₋₈alkyl or cycloalkyl; and
R⁷ is selected from hydrogen, halogen, —C₁₋₈alkyl, cycloalkyl, heterocycloalkyl, —OR^{7a}, —COR^{7a}, —NR^{7d}R^{7e}, —CONR^{7d}R^{7e}, —OH, and —CN; wherein said —C₁₋₈alkyl, cycloalkyl, or heterocycloalkyl is optionally substituted with at least one R^{7g};
wherein R^{7g} is each independently selected from hydrogen, halogen, —C₁₋₈alkoxy, COC₁₋₈alkyl, —NH₂, —OH, and —CN;
wherein R^{7a} is selected from —C₁₋₈alkyl, C₃₋₈cycloalkyl, and 3- to 10-membered heterocycloalkyl, wherein said —C₁₋₈alkyl, C₃₋₈cycloalkyl, or 3- to 10-membered heterocycloalkyl is optionally substituted with at least one R^{7b};
wherein R^{7b} is each independently selected from halogen, —OH, —C₁₋₈alkyl, cycloalkyl, heterocycloalkyl, —NH₂, and —C₁₋₈alkoxy, wherein said cycloalkyl, heterocycloalkyl or —C₁₋₈alkoxy is optionally substituted with at least one R^{7c};
wherein R^{7c} is each independently selected from halogen, —C₁₋₈alkyl, —CN, and —OH;
wherein R^{7d} and R^{7e} are each independently selected from hydrogen and —C₁₋₈alkyl,
wherein said —C₁₋₈alkyl is optionally substituted with at least one R^{7f},
wherein R^{7f} is each independently halogen, —CN, or —OH.

2. The compound according to claim 1, or a pharmaceutically acceptable salt, stereoisomer, or tautomer thereof, wherein:
R⁵ is selected from
(i) —C₁₋₈alkyl, aryl and 5- to 12-membered heteroaryl, wherein said —C₁₋₈alkyl, aryl or 5- to 12-membered heteroaryl is optionally substituted with at least one R^{5a};
wherein R^{5a} is each independently selected from halogen, —OH, —CN, —C₁₋₈alkyl, —C₁₋₈haloalkyl, —C₁₋₈alkoxy, —NR^{5b}R^{5c}, and —COR^{5d};
R^{5b} and R^{5c} are each independently hydrogen or C₁₋₈alkyl;
R^{5d} is each independently hydrogen, —C₁₋₈alkyl, or cycloalkyl;
(ii) —C₁₋₈alkoxy;
(iii) —C₁₋₈haloalkyl;
(iv)-C₃₋₈cycloalkyl, wherein said —C₃₋₈cycloalkyl is optionally substituted with at least one R^{5a};
wherein R^{5a} is each independently selected from halogen, —C₁₋₈alkyl, —C₁₋₈haloalkyl, and —C₁₋₈alkoxy;
(v) 3- to 8-membered heterocycloalkyl comprising one, two, or three heteroatoms independently selected from N, O, and S, wherein said 3- to 8-membered heterocycloalkyl is independently optionally substituted with at least one R^{5a};
wherein R^{5a} is each independently selected from halogen, oxo, —C₁₋₄alkyl, —C₁₋₄alkoxy, C₁₋₄haloalkyl, and —COC₁₋₄alkyl; wherein each of said —C₁₋₄alkyl, —C₁₋₄alkoxy, and —C₁₋₄haloalkyl is independently optionally substituted with at least one R^{5g}; wherein R^{5g} is each independently selected from halogen, —CN, and —C₁₋₈alkyl;
(vi) 5- to 6-membered monocyclic heteroaryl or 8- to 10-membered bicyclic heteroaryl comprising one, two, or three heteroatoms independently selected from N, O, and S; wherein said heteroaryl is each independently optionally substituted with at least one R^{5a}, wherein R^{5a} is each independently selected from halogen, oxo, —C₁₋₈alkyl, —C₁₋₈alkoxy, and —COC₁₋₈alkyl; and
(vii) —NR⁵ⁱR^{5j}; wherein R⁵ⁱ and R^{5j} are each independently selected from hydrogen, —C₁₋₈alkyl, aryl, and heteroaryl.

3. The compound according to claim 1 or 2, or a pharmaceutically acceptable salt, stereoisomer, or tautomer thereof, wherein:
R² is 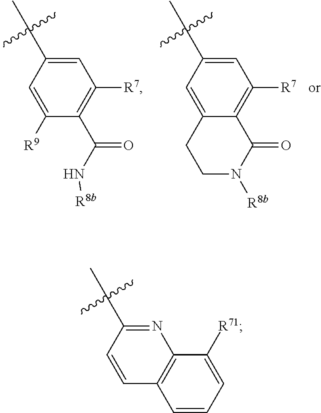
wherein 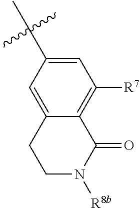
is optionally substituted with at least one R^{8e};
wherein R^{8b} is each independently selected from hydrogen, —C₁₋₈alkyl, 5- to 6-membered heterocycloalkyl and C₃₋₆cycloalkyl, wherein said —C₁₋₈alkyl, 5- to 6-membered heterocycloalkyl or C₃₋₆cycloalkyl is optionally substituted with at least one R^{8d};
wherein R^{8d} is each independently selected from halogen (F, Cl, Br and I), —OH, —CN, —NH₂, —CONH₂, aryl, and heteroaryl;
wherein R^{8e} is each independently selected from —C₁₋₄alkyl and C₃₋₆cycloalkyl;
R⁷¹ is selected from 4- to 15-membered monocyclic, spirocyclic, and bridged polycyclic heterocycloalkyl comprising one, two, or three heteroatoms independently selected from N, O, and S, which each of said heterocycloalkyl is independently optionally substituted with at least one R^{71a}; and
R^{71a} is each independently selected from hydrogen, —C₁₋₈alkyl, and —NH₂.

4. The compound according to any one of claims 1-3, or a pharmaceutically acceptable salt, stereoisomer, or tautomer thereof, wherein the compound is formula (II-1), (II-2), (II-3), or (II-4),

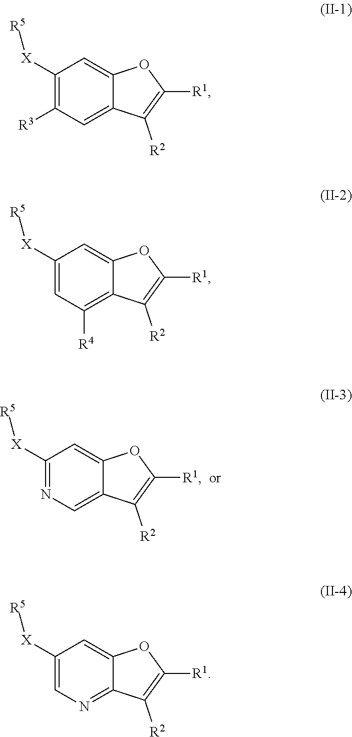

5. The compound according to any one of claims 1-4, or a pharmaceutically acceptable salt, stereoisomer, or tautomer thereof, wherein
X is selected from:
a1) 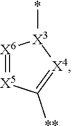
wherein X³, X⁴, X⁵, and X⁶ are each independently selected from N and CR^{A}; R^{A} is each independently selected from hydrogen, halogen, —C₁₋₈alkyl, —C₁₋₈haloalkyl, —CN, and —C₁₋₈alkoxy; and two or three of X³, X⁴, X⁵, and X⁶ are N;
a2) 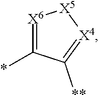
wherein X⁴ and X⁶ are each independently selected from N and CR^{A}; X⁵ is each independently selected from —NR^{B}— or —CR^{C}R^{D}—; two or three of X⁴, X⁵, and X⁶ contain N; and when X⁴ or X⁶ is CR^{A}, R^{A} is each independently selected from hydrogen, halogen, —C₁₋₈alkyl, —C₁₋₈haloalkyl, —CN, and —C₁₋₈alkoxy; when X⁵ is —NR^{B}—, R^{B} is each independently selected from hydrogen, —C₁₋₈alkyl, and —C₁₋₈haloalkyl; and when X⁵ is —CR^{C}R^{D}, R^{C} and R^{D} are each independently selected from hydrogen, halogen, —C₁₋₈alkyl, —C₁₋₈haloalkyl, —CN, and —C₁₋₈alkoxy;
b) 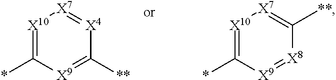
wherein X⁷, X⁸, X⁹, and X¹⁰ are each independently selected from N and CR^{A}, R^{A} is each independently selected from hydrogen, halogen, —C₁₋₈alkyl, —C₁₋₈haloalkyl, —CN, and —C₁₋₈alkoxy; and one, two or three of X⁷, X⁸, X⁹, and X¹⁰ are N;
c) 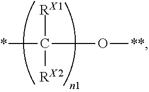
wherein n¹ is 0, 1, 2, 3, or 4; R^{X1} and R^{X2} are each independently selected from hydrogen, halogen, —C₁₋₈alkyl, —C₁₋₈haloalkyl, —CN, and —C₁₋₈alkoxy;
d) 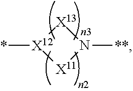
wherein n² and n³ are each independently selected from 0, 1, 2, or 3; wherein X¹² is N or CR^{A}, R^{A} is each independently selected from hydrogen, halogen, —C₁₋₈alkyl, —C₁₋₈haloalkyl, —CN, and —C₁₋₈alkoxy; X¹¹ and X¹³ are each independently selected from —NR^{B}— and —CR^{C}R^{D}—; when X¹¹ or X¹³ is —NR^{B}, R^{B} is each independently selected from hydrogen, —C₁₋₈alkyl, and —C₁₋₈haloalkyl; when X¹¹ or X¹³ is —CR^{C}R^{D}, R^{C} and R^{D} are each independently selected from hydrogen, halogen, —C₁₋₈alkyl, —C₁₋₈haloalkyl, —CN, and —C₁₋₈alkoxy; and
e) 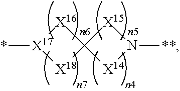
wherein n⁴, n⁵, n⁶ and n⁷ are each independently selected from 0, 1, 2, and 3; wherein X¹⁷ is N or CR^{A}, R^{A} is each independently selected from hydrogen, halogen, —C₁₋₈alkyl, —C₁₋₈haloalkyl, —CN, and —C₁₋₈alkoxy; X¹⁴, X¹⁵, X¹⁶ and X¹⁸ are each independently-NR^{B}—, or —CR^{C}R^{D}—; wherein R^{B} is each independently selected from hydrogen, —C₁₋₈alkyl, and —C₁₋₈haloalkyl; R^{C} and R^{D} are each independently selected from hydrogen, halogen, —C₁₋₈alkyl, —C₁₋₈haloalkyl, —CN, and —C₁₋₈alkoxy;
wherein * refers to the position attached to R⁵, and ** refers to the position attached to the 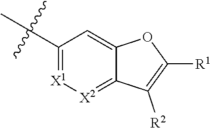
moiety.

6. The compound according to any one of claims 1-4, or a pharmaceutically acceptable salt, stereoisomer, or tautomer thereof, wherein
X is selected from 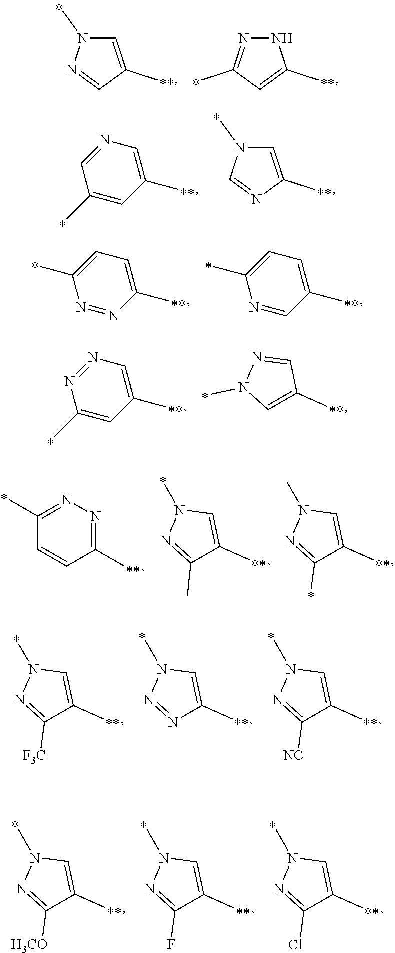
*—O—** *—CH₂—O—**, *—CH₂—**, *—NH—**, *—CH₂—CH₂—O—**, *—CH₂—CH₂—CH₂—O—**, *—CH₂—CH₂ (CH₃)—O—**,

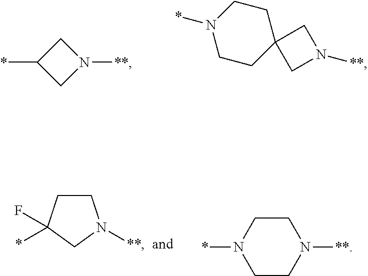

7. The compound according to any one of claims 1-6, or a pharmaceutically acceptable salt, stereoisomer, or tautomer thereof, wherein
R⁵ is selected from:
(i) —C₁₋₄alkyl, aryl and 5- to 12-membered heteroaryl, wherein said —C₁₋₄alkyl, aryl or 5- to 12-membered heteroaryl is optionally substituted with at least one R^{5a};
wherein R^{5a} is each independently selected from halogen, —OH, —CN, —C₁₋₈alkyl, —C₁₋₈haloalkyl, —C₁₋₈alkoxy, —NR^{5b}R^{5c}, or —COR^{5d};
R^{5b} and R^{5c} are each independently hydrogen, or —C₁₋₈alkyl;
R^{5d} is selected from hydrogen, —C₁₋₈alkyl, or —C₃₋₆cycloalkyl;
(ii) —C₁₋₄haloalkyl;
(iii) —C₁₋₄alkoxy;
(iv)-C₃₋₆ monocyclic cycloalkyl, wherein each of said —C₃₋₆ monocyclic cycloalkyl is independently optionally substituted with at least one R^{5a};
wherein R^{5a} is each independently selected from halogen, —C₁₋₈alkyl, —C₁₋₈haloalkyl, and —C₁₋₈alkoxy;
(v) 3- to 6-membered monocyclic heterocycloalkyl comprising one, two, or three heteroatoms independently selected from N, O, and S, which each of said 3- to 6-membered monocyclic heterocycloalkyl is independently optionally substituted with at least one R^{5a};
wherein R^{5a} is each independently selected from selected halogen, oxo, —C₁₋₄alkyl, —C₁₋₄alkoxy, and —COC₁₋₄alkyl; wherein each of said —C₁₋₄alkyl, —C₁₋₄alkoxy, or —COC₁₋₄alkyl is independently optionally substituted with at least one R^{5g};
wherein R^{5g} is each independently selected from hydrogen, halogen, —CN, and —C₁₋₈alkyl,
(vi) 5- to 6-membered monocyclic, and 8- to 10-membered fused bicyclic heteroaryl comprising one, two, or three heteroatoms independently selected from N, O, and S; wherein said monocyclic, or 8- to 10-membered fused bicyclic heteroaryl is each independently optionally substituted with at least one R^{5a};
wherein R^{5a} is each independently selected from halogen, oxo, —C₁₋₈alkyl, —C₁₋₈alkoxy, or —COC₁₋₈alkyl, and
(vii) —NR⁵ⁱR^{5j}; wherein R⁵ⁱ and R^{5j} are each independently selected from hydrogen, —C₁₋₈alkyl, 5- to 6-membered monocyclic aryl, or 5- to 6-membered monocyclic heteroaryl.

8. The compound according to any one of claims 1-7, or a pharmaceutically acceptable salt, stereoisomer, or tautomer thereof, wherein
R⁵ is selected from —CH₃, —CH₂CH₃, —CH(CH₃)₂, 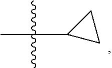 [SMILES: *C1CC1]
—OCH₂CH₃, —OCH₃, —CF₃,

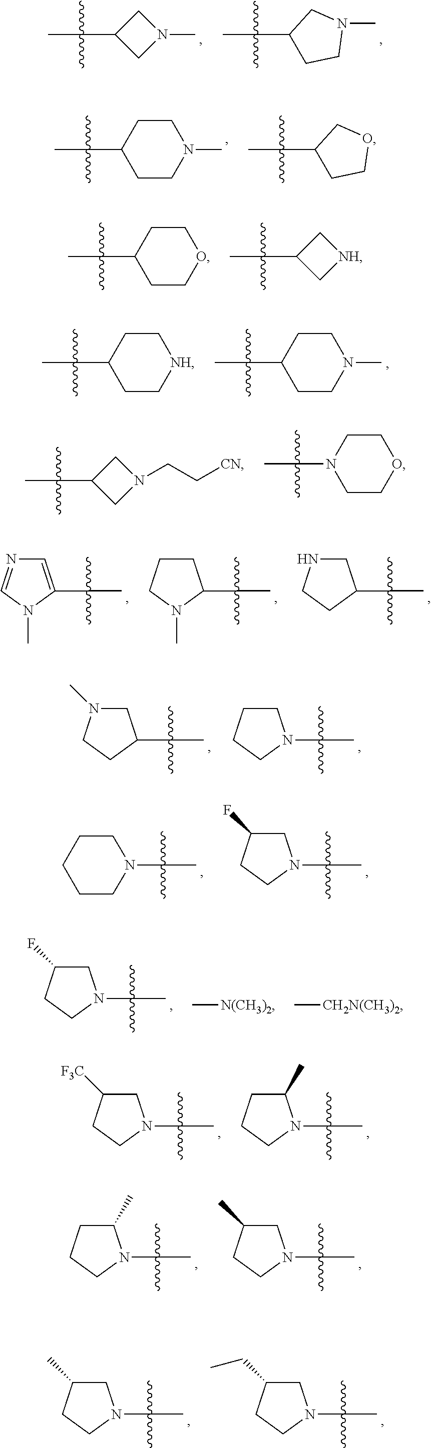

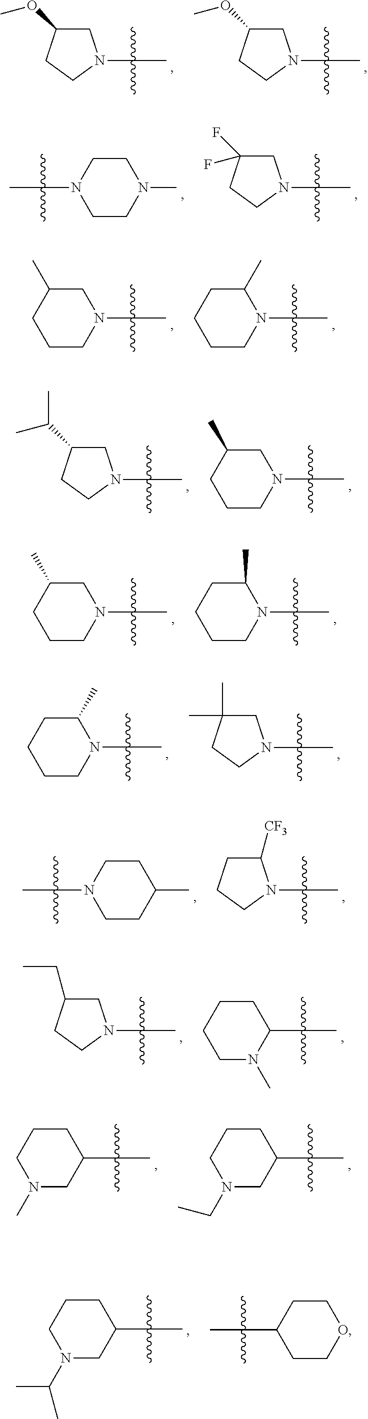

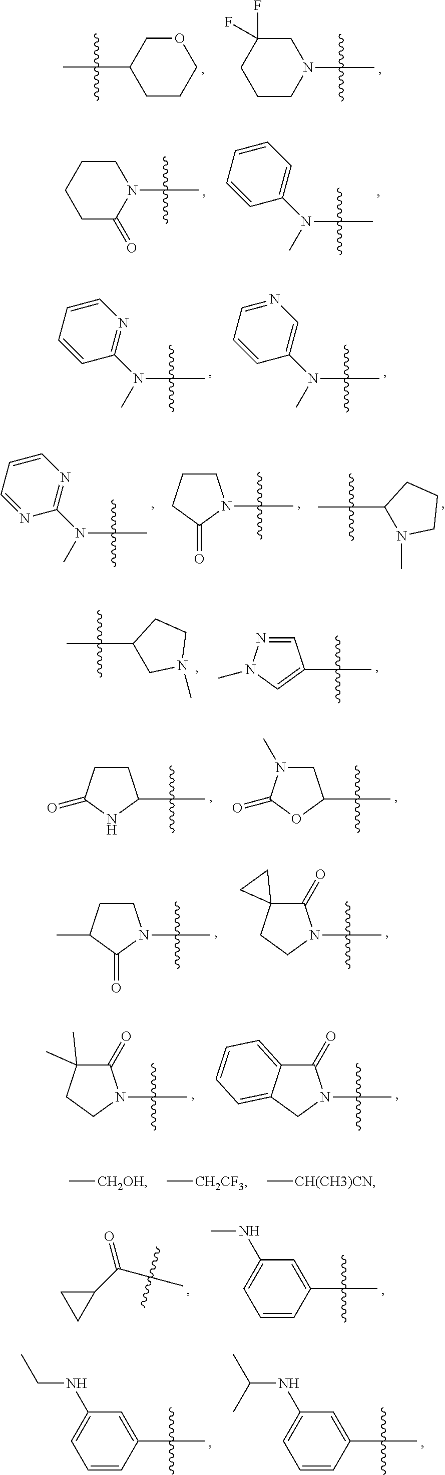

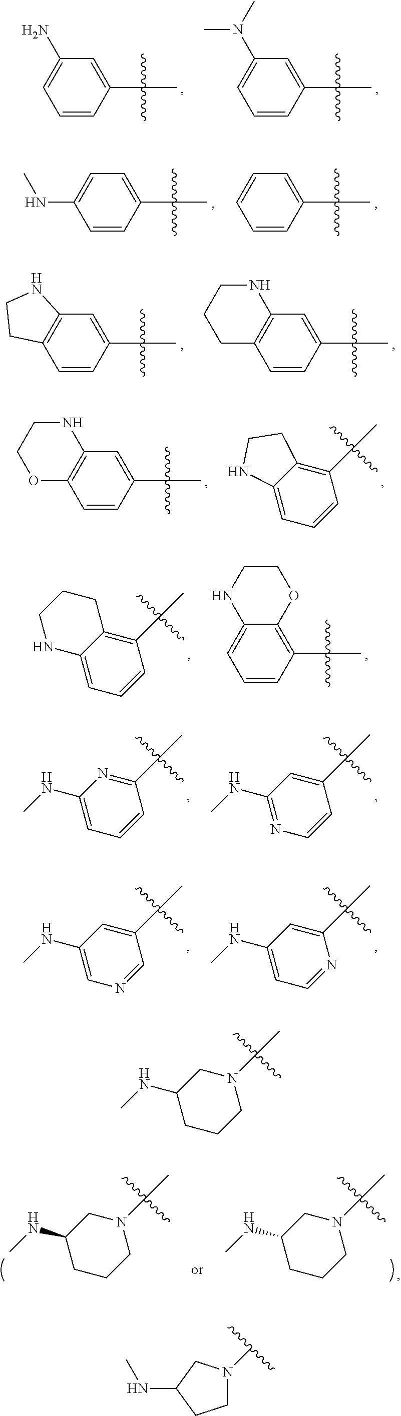

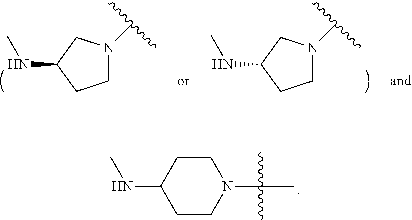

9. The compound according to any one of claims 1-2, or a pharmaceutically acceptable salt, stereoisomer, or tautomer thereof, wherein
R² is selected from:
a) 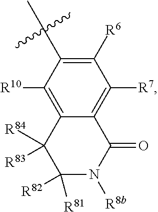
wherein R⁶ is H; R¹⁰ is H; R⁷ is hydrogen or —OR^{7a}, wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b}; wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c}; wherein R^{7c} is each independently-C₁₋₈alkyl, or OH; R⁸¹, R⁸², R⁸³, R⁸⁴ are each independently selected from hydrogen, and —C₁₋₈alkyl; and R^{8b} is selected from hydrogen, —C₁₋₈alkyl, and C₃-C₈cycloalkyl, wherein said —C₁₋₈alkyl, or C₃-C₈cycloalkyl is optionally substituted with at least one R^{8d}; R^{8d} is each independently selected from halogen, —OH, —NH₂, —CN, —CONH₂, aryl, and heteroaryl;
b) 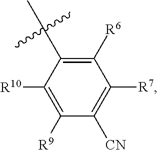
wherein R⁶, R⁹, and R¹⁰ are each independently selected from hydrogen, —C₁₋₈alkyl, and —C₁₋₈alkoxy; R⁷ is selected from hydrogen, —C₁₋₈alkyl, C₃-C₈cycloalkyl, and —OR^{7a}, wherein said —C₁₋₈alkyl, or C₃-C₈cycloalkyl is optionally substituted with at least one substituent selected from halogen, —C₁₋₈alkoxy, —COC₁₋₈alkyl, —NH₂, —OH, and —CN; R^{7a} is selected from —C₁₋₈alkyl, C₃₋₈cycloalkyl, and 3- to 10-membered heterocycloalkyl; wherein said —C₁₋₈alkyl, C₃₋₈cycloalkyl, or 3- to 10-membered heterocycloalkyl is optionally substituted with at least one R^{7b}, wherein R^{7b} is halogen;
c) 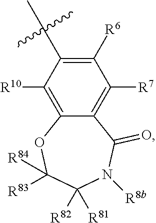
wherein R⁶ is H; R¹⁰ is H; R⁷ is hydrogen or —OR^{7a}, wherein R^{7a} is selected from —C₁₋₈alkyl, cycloalkyl, or heterocycloalkyl, wherein said —C₁₋₈alkyl, cycloalkyl, or heterocycloalkyl is optionally substituted with at least one R^{7b}, wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c}; wherein R^{7c} is selected from —C₁₋₈alkyl, and OH; R⁸¹, R⁸², R⁸³, R⁸⁴ are each independently selected from hydrogen, or —C₁₋₈alkyl; and R^{8b} is selected from hydrogen, —C₁₋₈alkyl, and C₃-C₈cycloalkyl, wherein said —C₁₋₈alkyl, or C₃-C₈cycloalkyl is optionally substituted with at least one R^{8d}; R^{8d} is each independently selected from halogen, —OH, —NH₂, —CN, —CONH₂, aryl, and heteroaryl;
d) 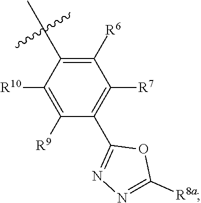
wherein R⁷ and R⁹ are each independently hydrogen or —OR^{7a}, wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b}, wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c}; wherein R^{7c} is each independently selected from —C₁₋₈alkyl, and OH; R⁶ and R¹⁰ are both H; R^{8a} is selected from —C₁₋₈alkyl, and cycloalkyl;
e) 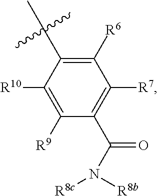
wherein R⁷ and R⁹ are each independently hydrogen or —OR^{7a}, wherein R^{7a} is selected from —C₁₋₈alkyl, cycloalkyl, and heterocycloalkyl, wherein said —C₁₋₈alkyl, cycloalkyl, or heterocycloalkyl is optionally substituted with at least one R^{7b}, wherein R^{7b} is each independently selected from halogen, —OH, —C₁₋₈alkyl, cycloalkyl, heterocycloalkyl, —NH₂, and C₁₋₈alkoxy, wherein said cycloalkyl or heterocycloalkyl is optionally substituted with at least one R^{7c}; wherein R^{7c} is each independently selected from —C₁₋₈alkyl, and OH; wherein R^{8b} and R^{8c} are each independently selected from hydrogen, —C₁₋₈alkyl, and C₃-C₈cycloalkyl, wherein said —C₁₋₈alkyl, or C₃-C₈cycloalkyl is optionally substituted with at least one R^{8d}; wherein R^{8d} is each independently selected from halogen, —OH, —NH₂, —CN, —CONH₂, aryl, and heteroaryl; R⁶ and R¹⁰ are both H; and
f) 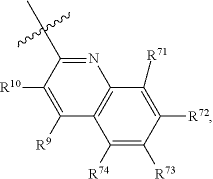
wherein R⁹ and R¹⁰ are each independently selected from hydrogen, halogen, —C₁₋₈alkyl, and —C₁₋₈alkoxy; R⁷², R⁷³, and R⁷⁴ are each independently selected from hydrogen, halogen, —C₁₋₈alkyl, —C₁₋₈haloalkyl, and —C₁₋₈alkoxy; R⁷¹ is selected from 4- to 15-membered monocyclic, spirocyclic, and bridged polycyclic heterocycloalkyl comprising one, two, or three heteroatoms independently selected from N, O, and S, which each of said heterocycloalkyl is independently optionally substituted with at least one R^{71a}; R^{71a} is each independently selected from hydrogen, —C₁₋₈alkyl, or —NH₂.

10. The compound according to claim 9, a pharmaceutically acceptable salt, stereoisomer, or tautomer thereof, wherein
R² is e) 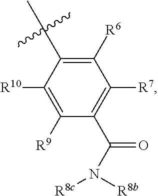
wherein R⁶ and R¹⁰ are both H; R⁷ and R⁹ are each independently-OR^{7a}, wherein R^{7a} is —C₁₋₈alkyl, wherein said —C₁₋₈alkyl is optionally substituted with at least one halogen; R^{8c} is H; R^{8b} is selected from —(CH₂)ₘ₁CHₘ₂F₃₋ₘ₂, —(CH₂)ₘ₁CN, and 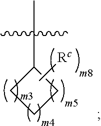
wherein m1 is 1, 2 or 3; m2 is 0, 1, or 2;
m3, m4, and m5 are each independently selected from 0, 1, 2 or 3; and m3+m4+m5>2;
R^{C} is each independently selected from —F, —OH, —CN, and —CONH₂.

11. The compound according to claim 9, or a pharmaceutically acceptable salt, stereoisomer, or tautomer thereof, wherein
R² is e) 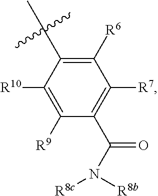
wherein R^{8c} is H; R^{8b} is selected from —(CH₂)ₘ₁CHₘ₂F₃₋ₘ₂, —(CH₂)ₘ₁CN,

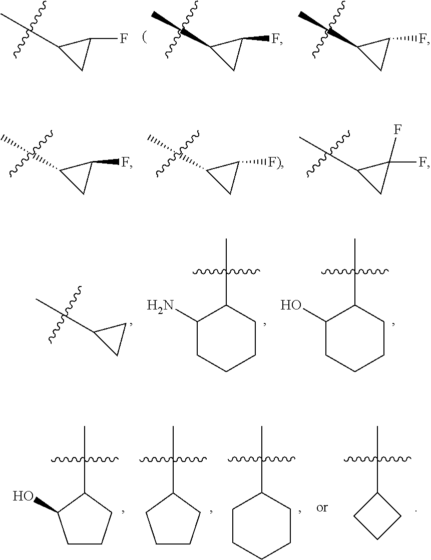

12. The compound according to claim 9, or a pharmaceutically acceptable salt, stereoisomer, or tautomer thereof, wherein R² is e) 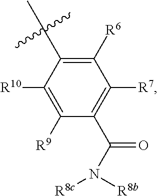
R⁶ and R¹⁰ are both H; R⁷ and R⁹ are each independently selected from hydrogen, —OCH₃, —OCHF₂, —F,

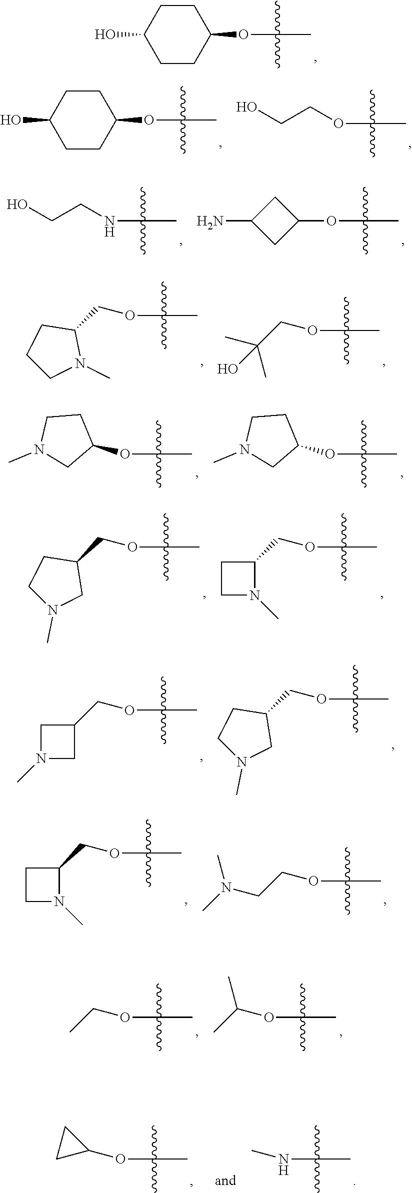

13. The compound according to claim 9, or a pharmaceutically acceptable salt, stereoisomer, or tautomer thereof, wherein R² is selected from
a) 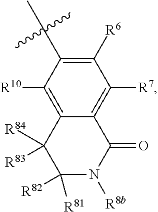
wherein R^{8b} is selected from

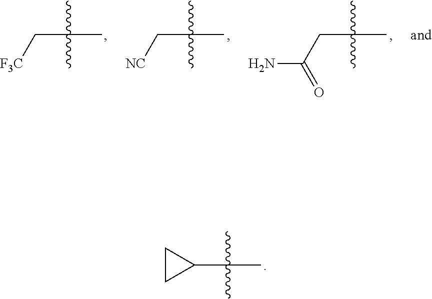

14. The compound according to claim 9, or a pharmaceutically acceptable salt, stereoisomer,
or tautomer thereof, wherein R² is d) 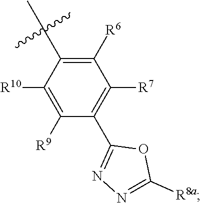
wherein R^{8b} is selected from —CH₃, —CH₂CH₃, and

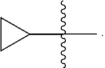 [SMILES: *C1CC1]

15. The compound according to claim 9, or a pharmaceutically acceptable salt, stereoisomer, or tautomer thereof, wherein R² is c) 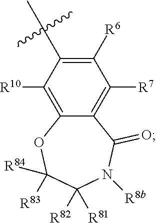
wherein R^{8b} is

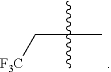 [SMILES: *CC(F)(F)F]

16. The compound according to any one of claim 9, or a pharmaceutically acceptable salt, stereoisomer, or tautomer thereof, wherein the compound is formula (III-1) or (III-2), 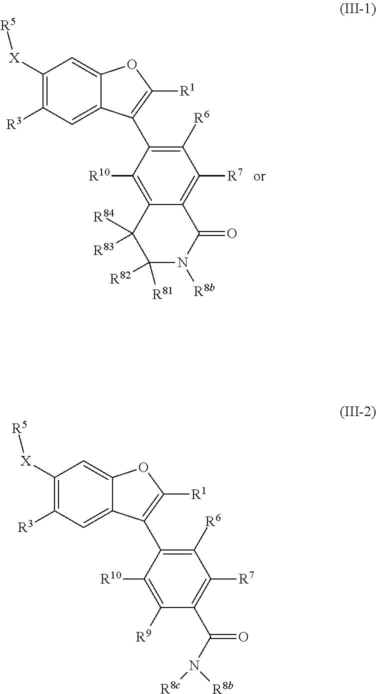
wherein X, R¹, R³, R⁵, R⁶, R⁷, R⁹, R¹⁰, R^{8b}, R^{8c} R⁸¹, R⁸², R⁸³, R⁸⁴, R^{8b}, and R^{8c} are defined as claim 9.

17. The compound according to claim 16, or a pharmaceutically acceptable salt, stereoisomer, or tautomer thereof, X is *—CH₂—O—**, *—CH₂—CH₂—O—**, *—CH₂—CH₂—CH₂—O—**, or *—CH₂—CH₂ (CH₃)—O—**; wherein * refers to the position attached to R⁵, ** refers to the position attached to the 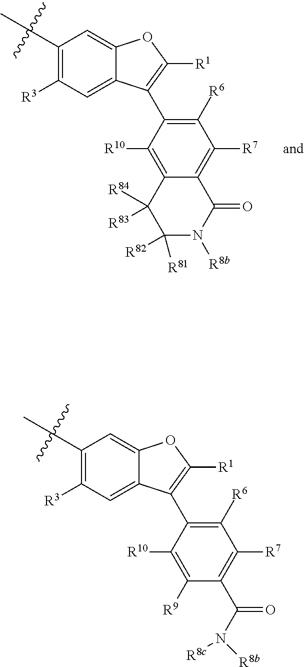
moiety.

18. The compound according to claim 17, or a pharmaceutically acceptable salt, stereoisomer, or tautomer thereof, the compound is formula (IV), (IV-2), (IV-3), (IV-4) or (IV-5) 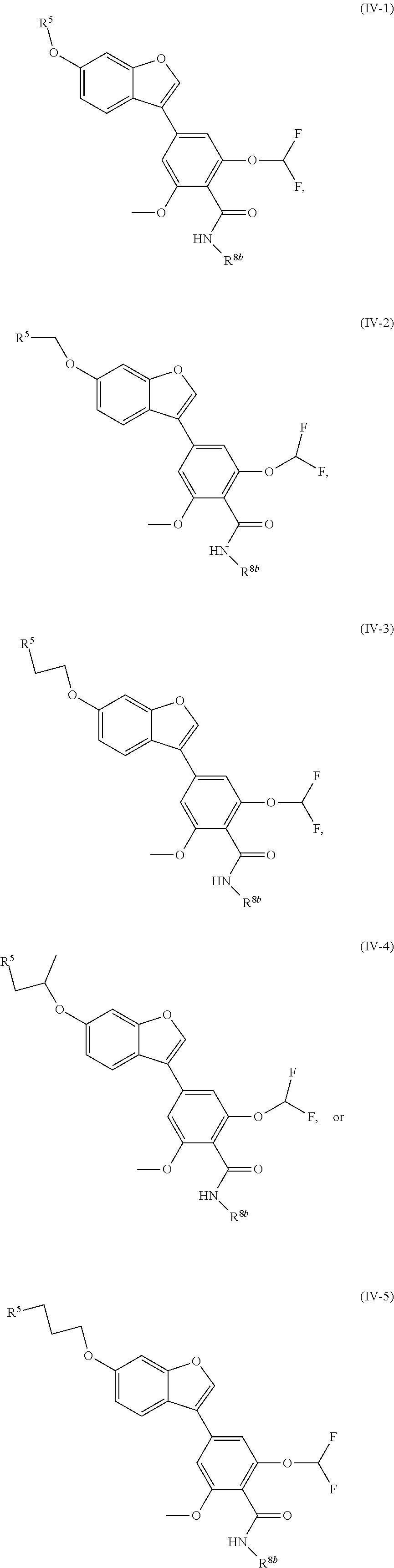
wherein R⁵, and R^{8b} are defined as claim 17; R⁶ and R¹⁰ are both H.

19. The compound according to claim 18, or a pharmaceutically acceptable salt, stereoisomer, or tautomer thereof, wherein R⁵ is selected from
(i) —C₁₋₈alkyl, aryl and 5- to 12-membered heteroaryl, wherein said —C₁₋₈alkyl, aryl or 5- to 12-membered heteroaryl is optionally substituted with at least one R^{5a};
wherein R^{5a} is each independently selected from halogen, —OH, —CN, —C₁₋₈alkyl, —C₁₋₈haloalkyl, —C₁₋₈alkoxy, —NR^{5b}R^{5c}, and —COR^{5d};
R^{5b} and R^{5c} are each independently hydrogen, or —C₁₋₈alkyl;
R^{5d} is hydrogen, —C₁₋₈alkyl, or cycloalkyl;
(ii) —C₁₋₈alkoxy;
(iii) —C₁₋₈haloalkyl;
(iv) —C₃₋₈cycloalkyl, wherein each of said —C₃₋₈cycloalkyl is independently optionally substituted with at least one R^{5a},
wherein R^{5a} is each independently selected from halogen, —C₁₋₈alkyl, —C₁₋₈haloalkyl, and —C₁₋₈alkoxy;
(v) 3- to 8-membered heterocycloalkyl comprising one, two, or three heteroatoms independently selected from N, O, and S, which each of said 3- to 8-membered heterocycloalkyl is independently optionally substituted with at least one R^{5a}; wherein R^{5a} is each independently selected from halogen, oxo, —C₁₋₈alkyl, —C₁₋₄alkoxy, C₁₋₄haloalkyl, and —COC₁₋₄alkyl; wherein each of said —C₁₋₄alkyl, —C₁₋₄alkoxy, and C₁₋₄haloalkyl is independently optionally substituted with at least one R^{5g}; wherein R^{5g} is each independently selected from halogen, —CN, and —C₁₋₈alkyl;
(vi) 5- to 6-membered monocyclic heteroaryl or 8- to 10-membered bicyclic heteroaryl comprising one, two, or three heteroatoms independently selected from N, O, and S; wherein said heteroaryl is each independently optionally substituted with at least one R^{5a}; wherein R^{5a} is each independently selected from selected halogen, oxo, —C₁₋₈alkyl, —C₁₋₈alkoxy, and —COC₁₋₈alkyl; and
(vii) —NR⁵ⁱR^{5j}; wherein R⁵ⁱ and R^{5j} are each independently selected from hydrogen, —C₁₋₈alkyl, aryl, and heteroaryl.

20. The compound according to claim 18 or 19, or a pharmaceutically acceptable salt, stereoisomer, or tautomer thereof, R^{8b} is selected from

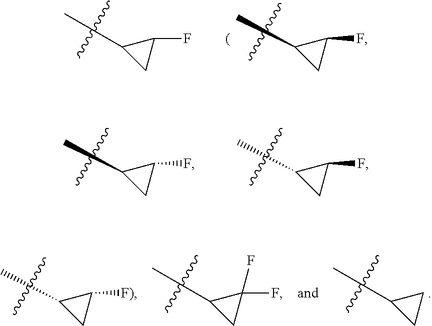

21. The compound according to any one of claims 1-20, or a pharmaceutically acceptable salt, stereoisomer, or tautomer thereof, wherein R¹ is selected from hydrogen, halogen, and —C₁₋₈alkyl.

22. The compound according to any one of claims 1-21, or a pharmaceutically acceptable salt, stereoisomer, or tautomer thereof, wherein R¹ is selected from hydrogen, F, and —CH₃.

23. A compound or a pharmaceutically acceptable salt, stereoisomer thereof, or tautomer thereof, wherein the compound is selected from

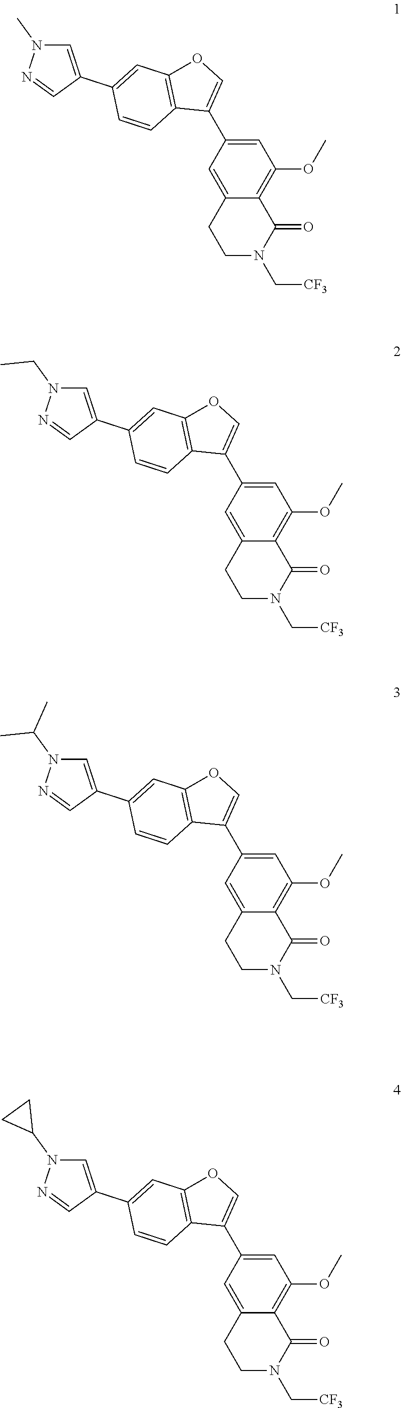

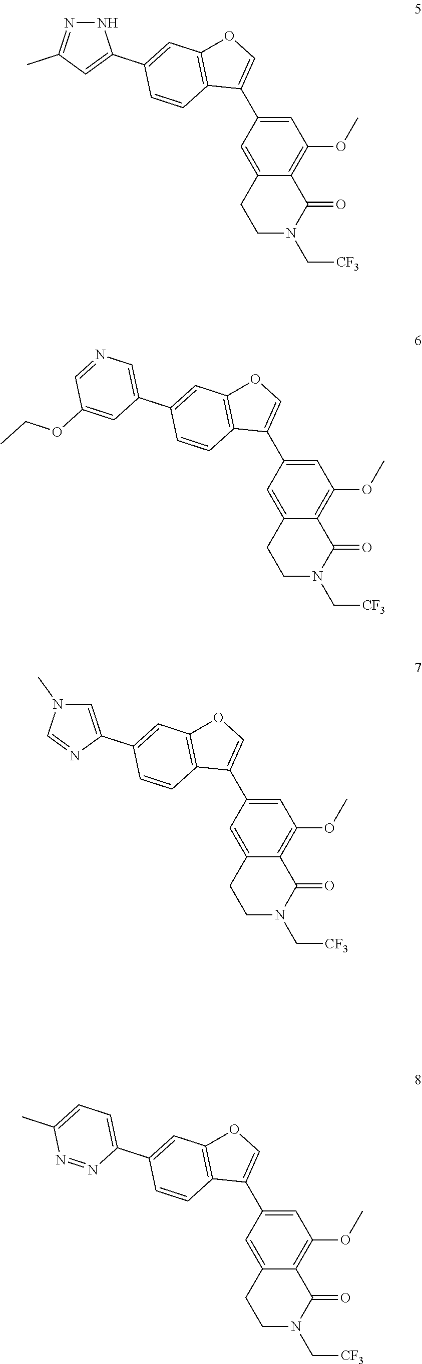

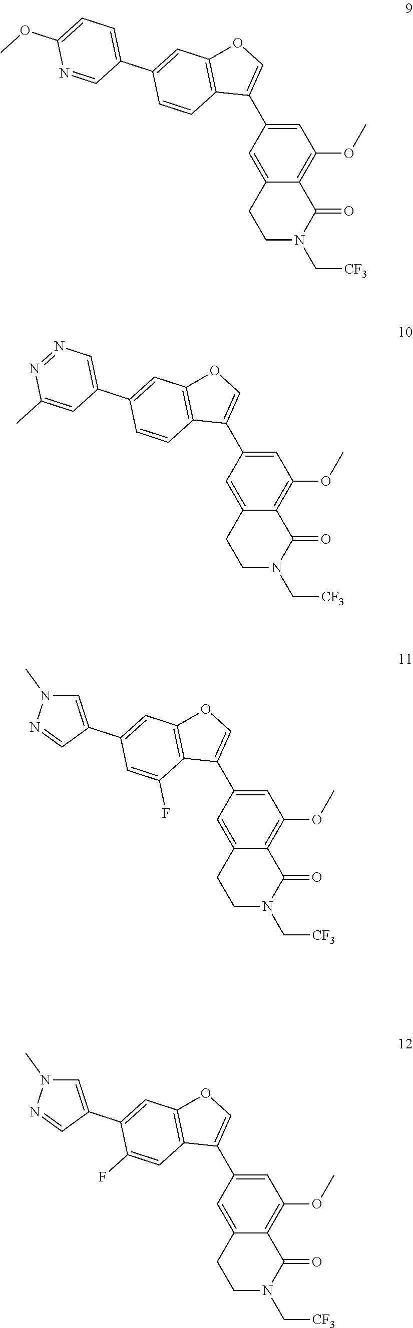

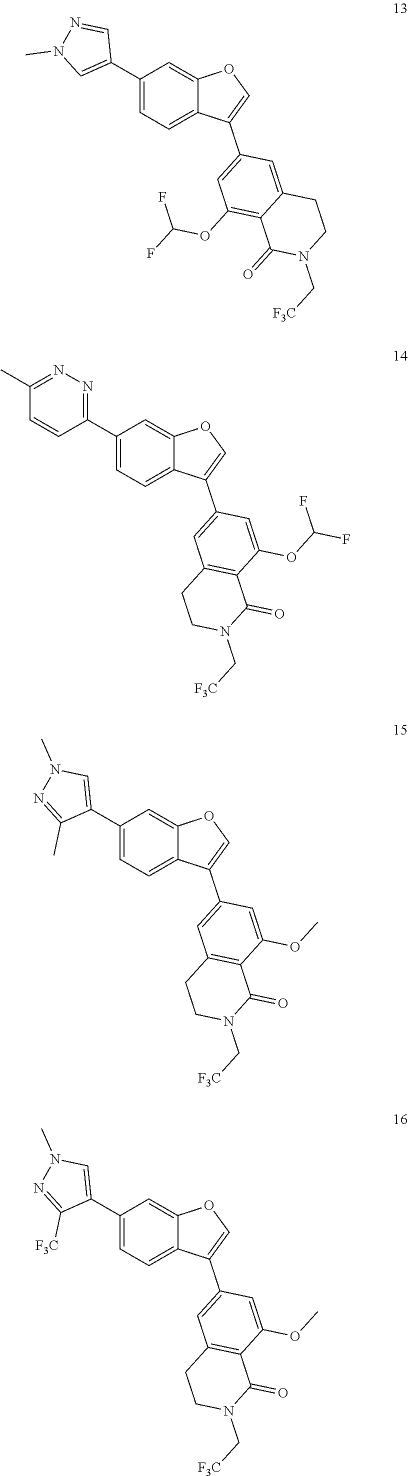 [SMILES: Cn1cc(-c2ccc3c(-c4cc5c(c(OC(F)F)c4)C(=O)N(CC(F)(F)F)CC5)coc3c2)cn1 ; Cc1ccc(-c2ccc3c(-c4cc5c(c(OC(F)F)c4)C(=O)N(CC(F)(F)F)CC5)coc3c2)nn1 ; COc1cc(-c2coc3cc(-c4cn(C)nc4C)ccc23)cc2c1C(=O)N(CC(F)(F)F)CC2 ; COc1cc(-c2coc3cc(-c4cn(C)nc4C(F)(F)F)ccc23)cc2c1C(=O)N(CC(F)(F)F)CC2]

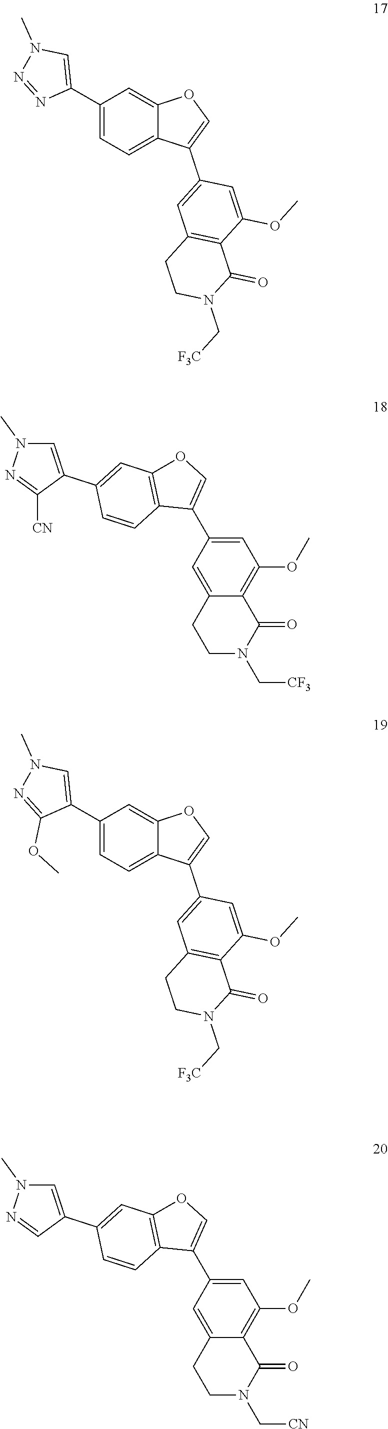

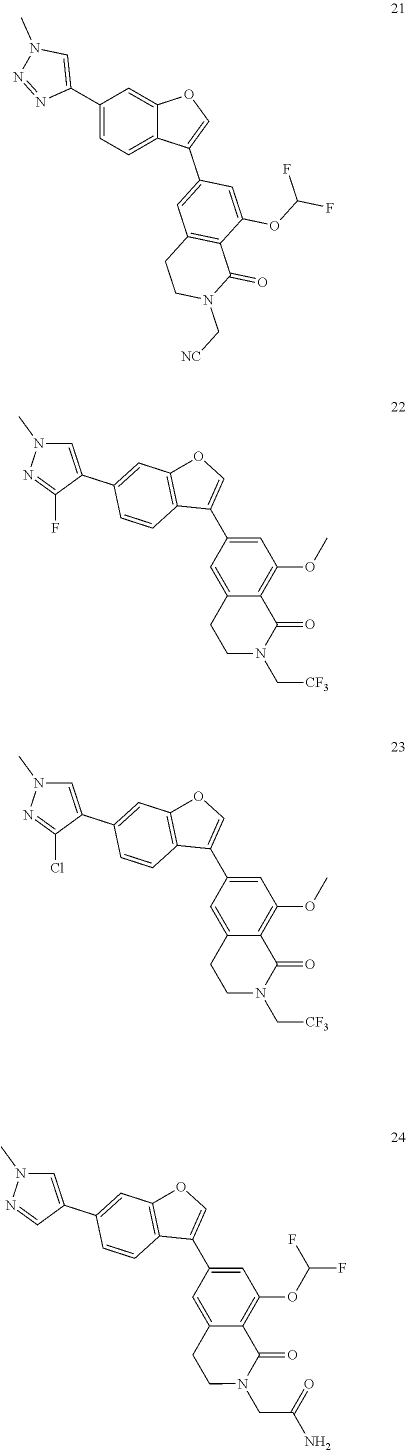

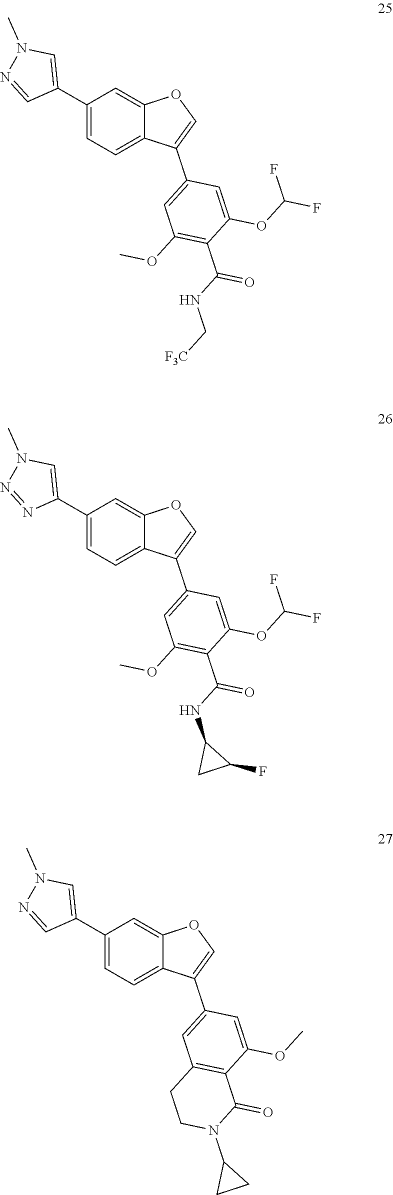 [SMILES: COc1cc(-c2coc3cc(-c4cnn(C)c4)ccc23)cc(OC(F)F)c1C(=O)NCC(F)(F)F ; COc1cc(-c2coc3cc(-c4cn(C)nn4)ccc23)cc(OC(F)F)c1C(=O)N[C@@H]1C[C@@H]1F ; COc1cc(-c2coc3cc(-c4cnn(C)c4)ccc23)cc2c1C(=O)N(C1CC1)CC2]

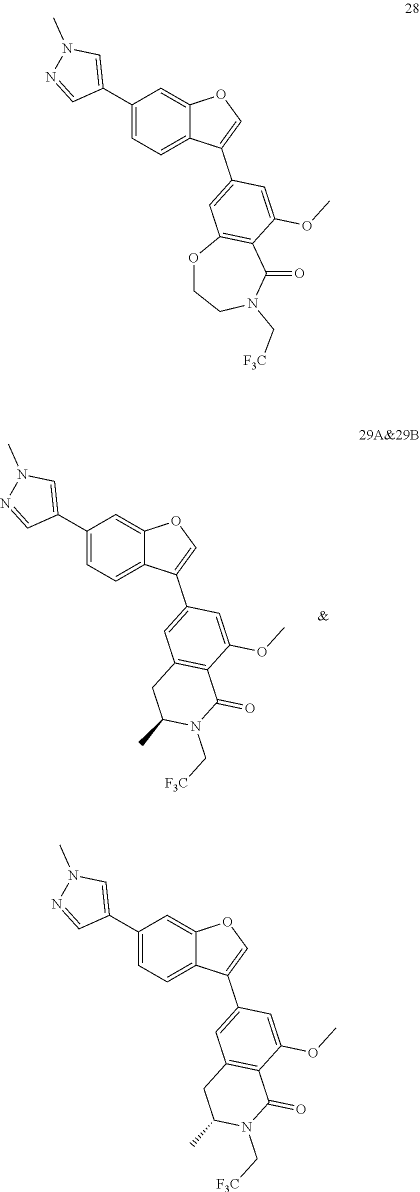 [SMILES: COc1cc(-c2coc3cc(-c4cnn(C)c4)ccc23)cc2c1C(=O)N(CC(F)(F)F)CCO2 ; COc1cc(-c2coc3cc(-c4cnn(C)c4)ccc23)cc2c1C(=O)N(CC(F)(F)F)[C@@H](C)C2 ; COc1cc(-c2coc3cc(-c4cnn(C)c4)ccc23)cc2c1C(=O)N(CC(F)(F)F)[C@H](C)C2]

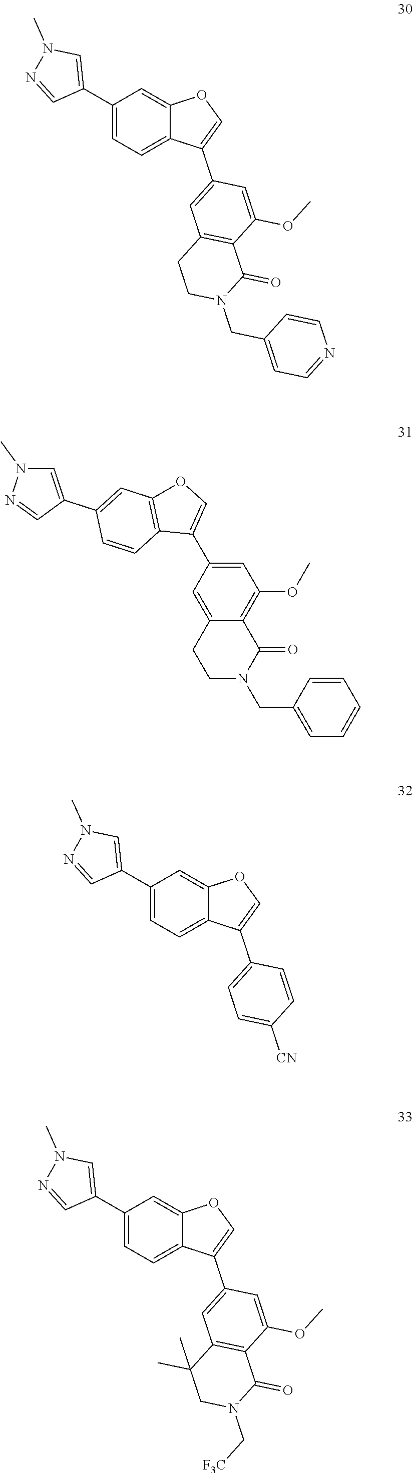 [SMILES: COc1cc(-c2coc3cc(-c4cnn(C)c4)ccc23)cc2c1C(=O)N(Cc1ccncc1)CC2 ; COc1cc(-c2coc3cc(-c4cnn(C)c4)ccc23)cc2c1C(=O)N(Cc1ccccc1)CC2 ; Cn1cc(-c2ccc3c(-c4ccc(C#N)cc4)coc3c2)cn1 ; COc1cc(-c2coc3cc(-c4cnn(C)c4)ccc23)cc2c1C(=O)N(CC(F)(F)F)CC2(C)C]

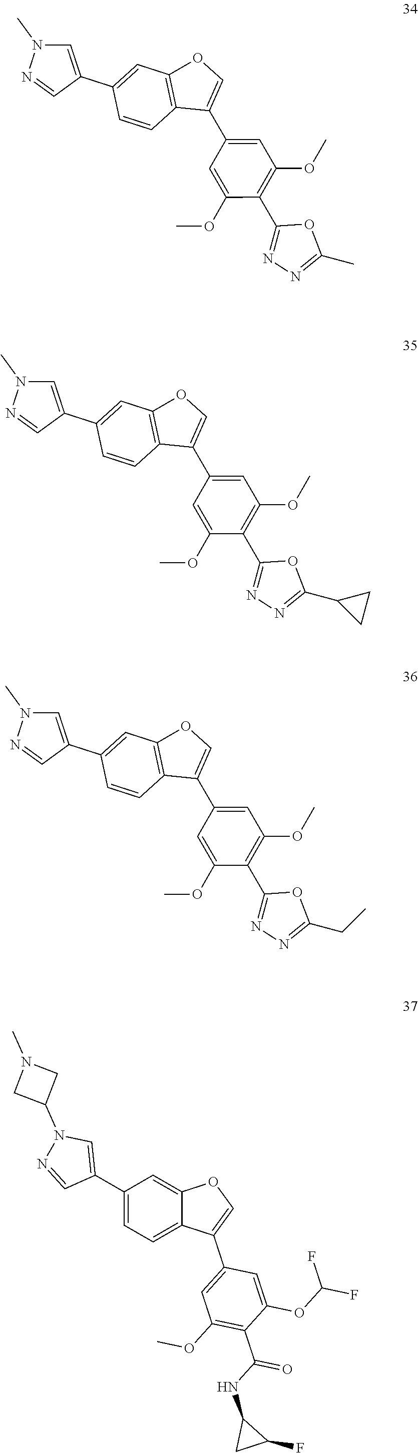

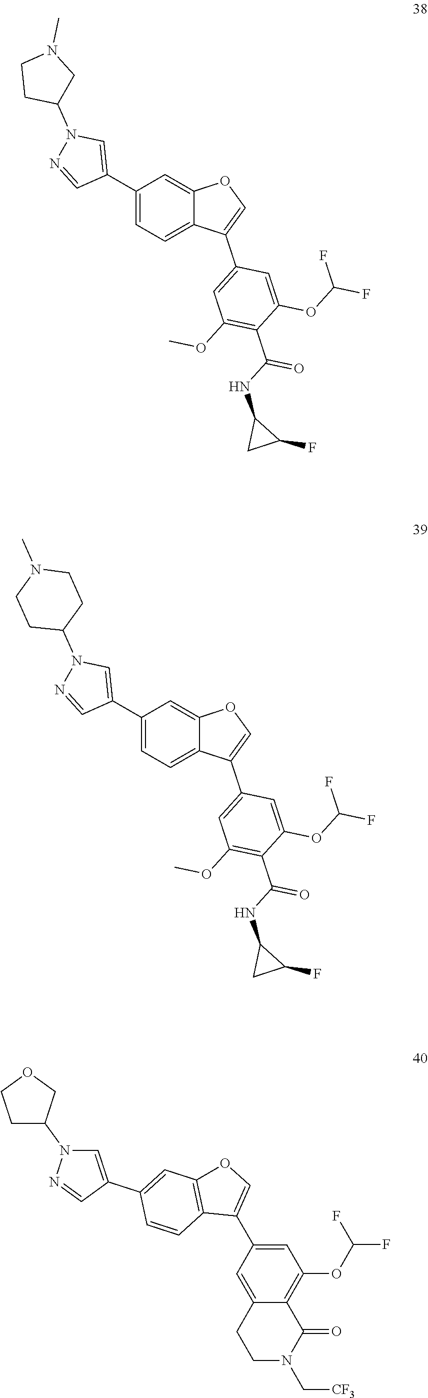 [SMILES: COc1cc(-c2coc3cc(-c4cnn(C5CCN(C)C5)c4)ccc23)cc(OC(F)F)c1C(=O)N[C@@H]1C[C@@H]1F ; COc1cc(-c2coc3cc(-c4cnn(C5CCN(C)CC5)c4)ccc23)cc(OC(F)F)c1C(=O)N[C@@H]1C[C@@H]1F ; O=C1c2c(cc(-c3coc4cc(-c5cnn(C6CCOC6)c5)ccc34)cc2OC(F)F)CCN1CC(F)(F)F]

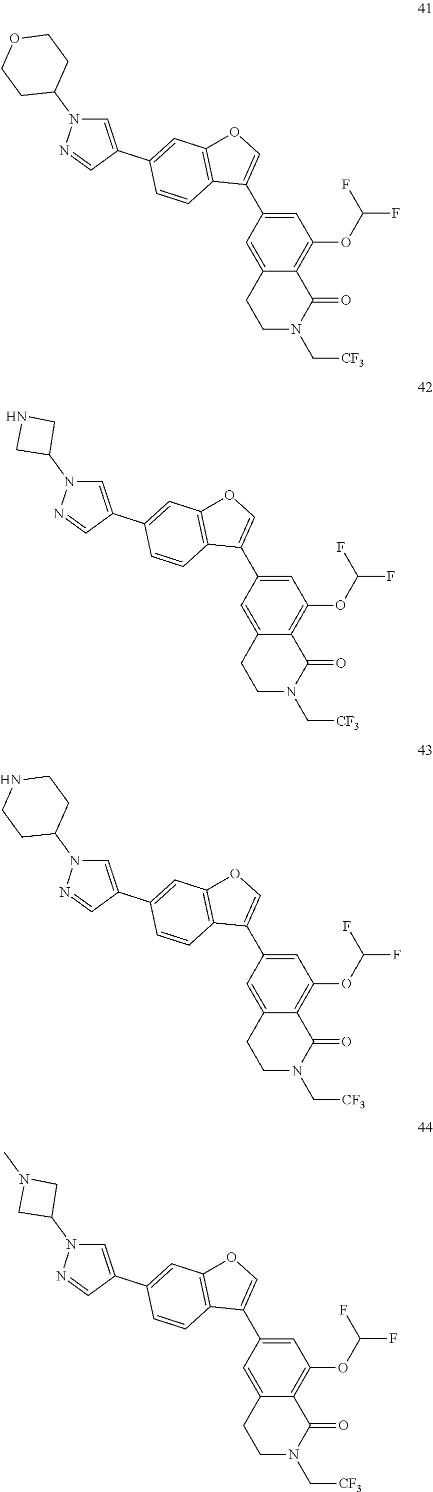 [SMILES: O=C1c2c(cc(-c3coc4cc(-c5cnn(C6CCOCC6)c5)ccc34)cc2OC(F)F)CCN1CC(F)(F)F ; O=C1c2c(cc(-c3coc4cc(-c5cnn(C6CNC6)c5)ccc34)cc2OC(F)F)CCN1CC(F)(F)F ; O=C1c2c(cc(-c3coc4cc(-c5cnn(C6CCNCC6)c5)ccc34)cc2OC(F)F)CCN1CC(F)(F)F ; CN1CC(n2cc(-c3ccc4c(-c5cc6c(c(OC(F)F)c5)C(=O)N(CC(F)(F)F)CC6)coc4c3)cn2)C1]

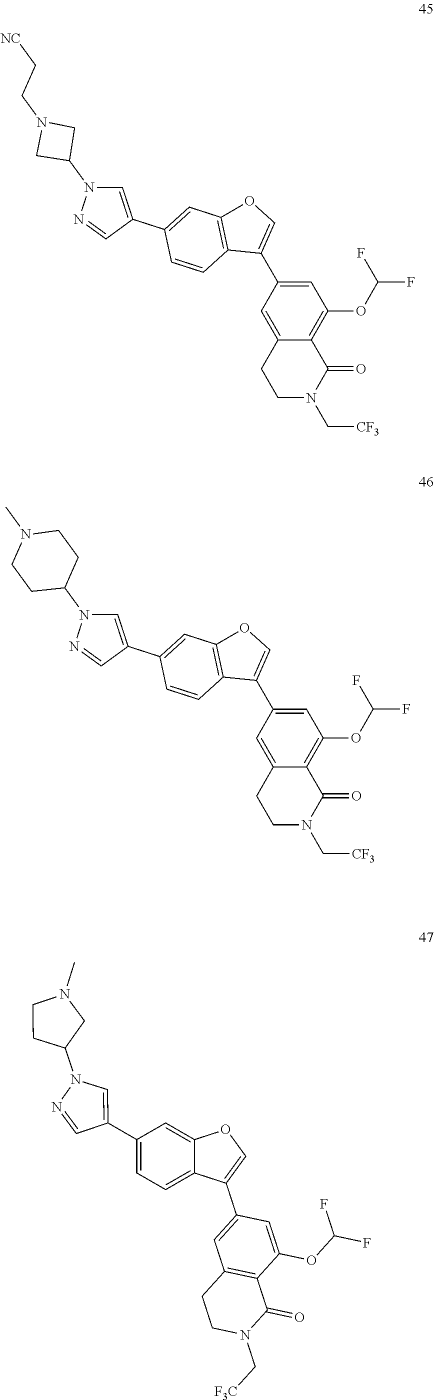 [SMILES: N#CCCN1CC(n2cc(-c3ccc4c(-c5cc6c(c(OC(F)F)c5)C(=O)N(CC(F)(F)F)CC6)coc4c3)cn2)C1 ; CN1CCC(n2cc(-c3ccc4c(-c5cc6c(c(OC(F)F)c5)C(=O)N(CC(F)(F)F)CC6)coc4c3)cn2)CC1 ; CN1CCC(n2cc(-c3ccc4c(-c5cc6c(c(OC(F)F)c5)C(=O)N(CC(F)(F)F)CC6)coc4c3)cn2)C1]

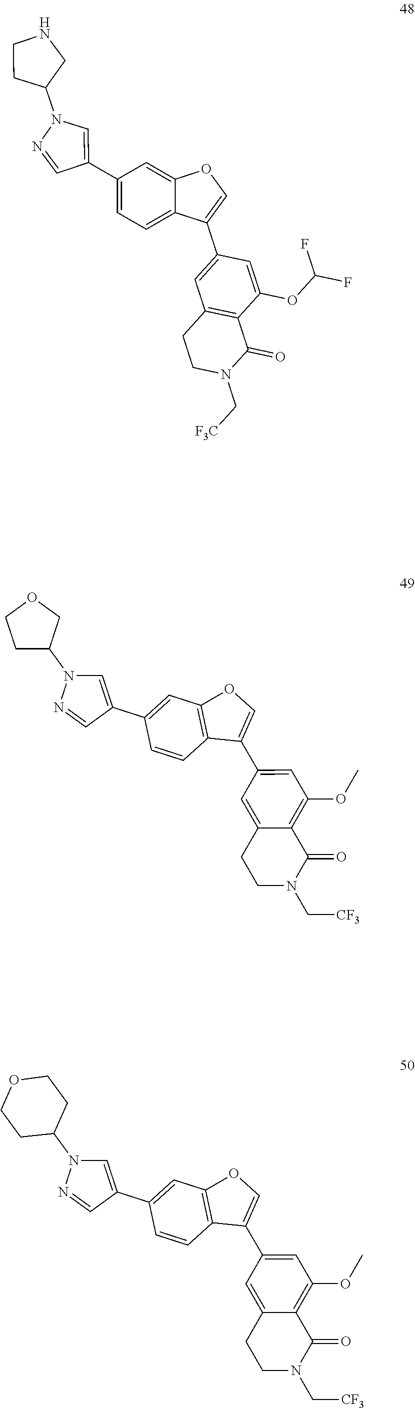 [SMILES: O=C1c2c(cc(-c3coc4cc(-c5cnn(C6CCNC6)c5)ccc34)cc2OC(F)F)CCN1CC(F)(F)F ; COc1cc(-c2coc3cc(-c4cnn(C5CCOC5)c4)ccc23)cc2c1C(=O)N(CC(F)(F)F)CC2 ; COc1cc(-c2coc3cc(-c4cnn(C5CCOCC5)c4)ccc23)cc2c1C(=O)N(CC(F)(F)F)CC2]

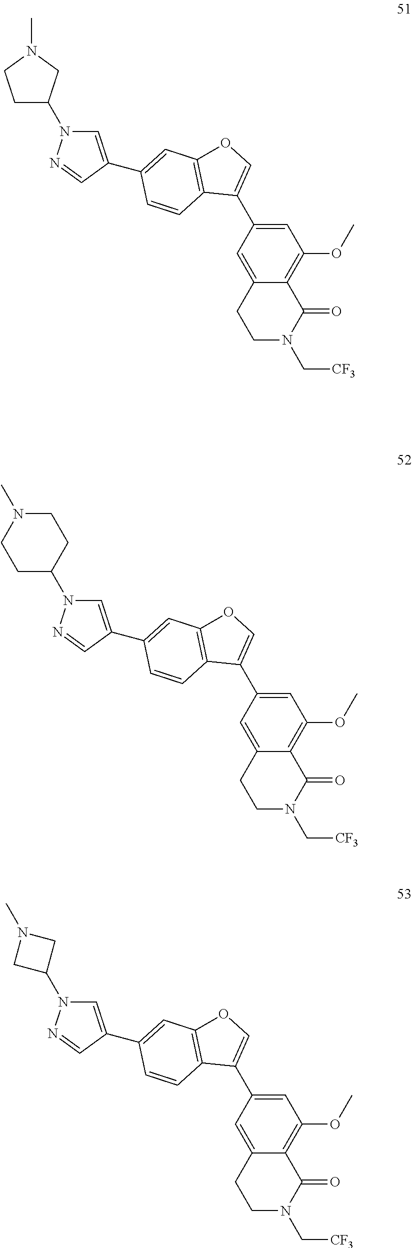

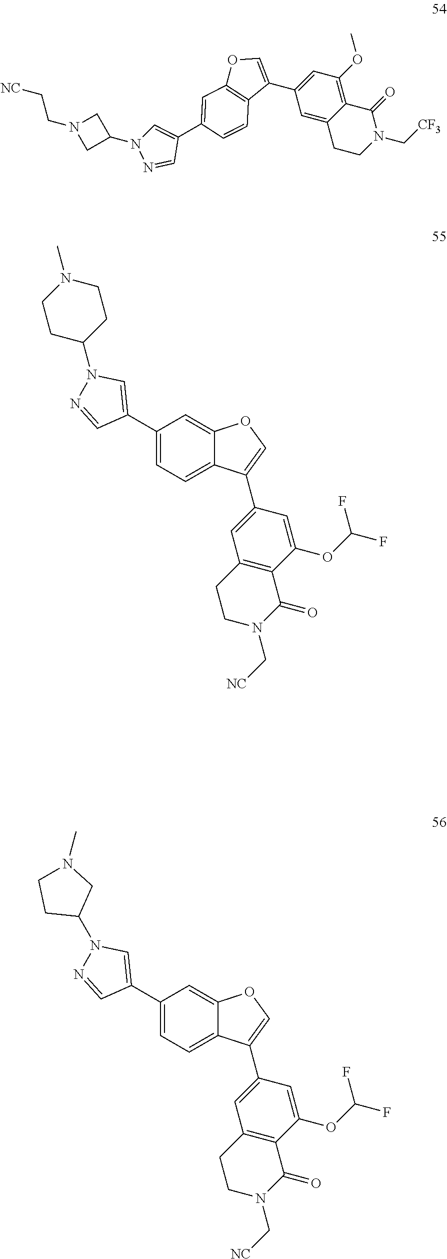 [SMILES: COc1cc(-c2coc3cc(-c4cnn(C5CN(CCC#N)C5)c4)ccc23)cc2c1C(=O)N(CC(F)(F)F)CC2 ; CN1CCC(n2cc(-c3ccc4c(-c5cc6c(c(OC(F)F)c5)C(=O)N(CC#N)CC6)coc4c3)cn2)CC1 ; CN1CCC(n2cc(-c3ccc4c(-c5cc6c(c(OC(F)F)c5)C(=O)N(CC#N)CC6)coc4c3)cn2)C1]

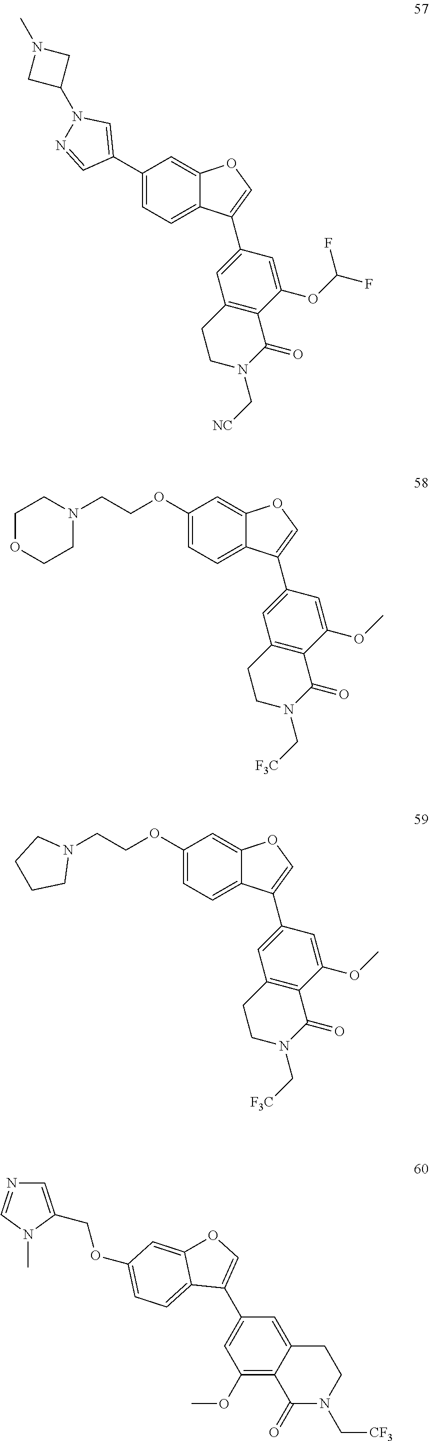 [SMILES: CN1CC(n2cc(-c3ccc4c(-c5cc6c(c(OC(F)F)c5)C(=O)N(CC#N)CC6)coc4c3)cn2)C1 ; COc1cc(-c2coc3cc(OCCN4CCOCC4)ccc23)cc2c1C(=O)N(CC(F)(F)F)CC2 ; COc1cc(-c2coc3cc(OCCN4CCCC4)ccc23)cc2c1C(=O)N(CC(F)(F)F)CC2 ; COc1cc(-c2coc3cc(OCc4cncn4C)ccc23)cc2c1C(=O)N(CC(F)(F)F)CC2]

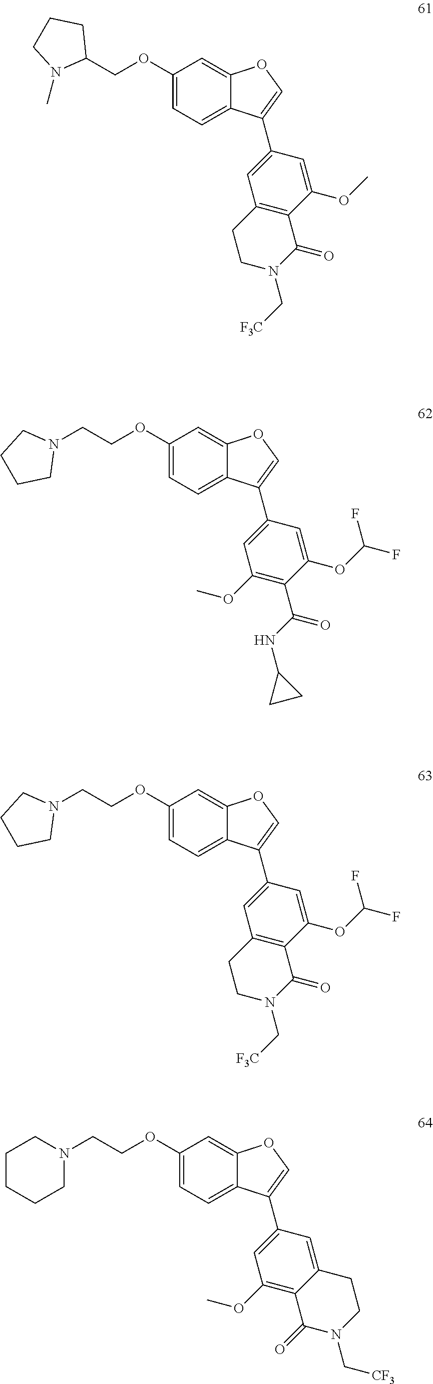 [SMILES: COc1cc(-c2coc3cc(OCC4CCCN4C)ccc23)cc2c1C(=O)N(CC(F)(F)F)CC2 ; COc1cc(-c2coc3cc(OCCN4CCCC4)ccc23)cc(OC(F)F)c1C(=O)NC1CC1 ; O=C1c2c(cc(-c3coc4cc(OCCN5CCCC5)ccc34)cc2OC(F)F)CCN1CC(F)(F)F ; COc1cc(-c2coc3cc(OCCN4CCCCC4)ccc23)cc2c1C(=O)N(CC(F)(F)F)CC2]

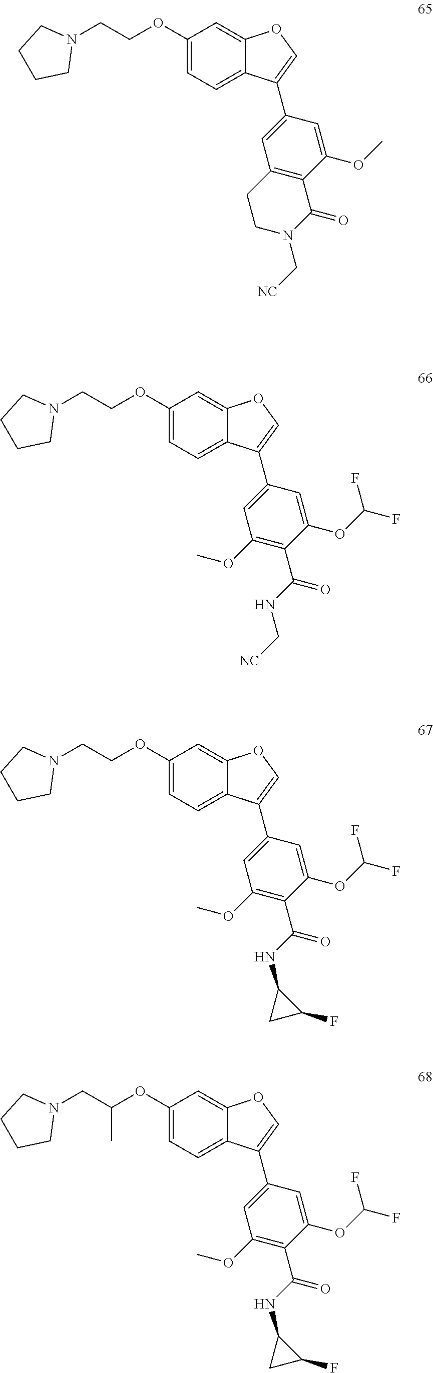 [SMILES: COc1cc(-c2coc3cc(OCCN4CCCC4)ccc23)cc2c1C(=O)N(CC#N)CC2 ; COc1cc(-c2coc3cc(OCCN4CCCC4)ccc23)cc(OC(F)F)c1C(=O)NCC#N ; COc1cc(-c2coc3cc(OCCN4CCCC4)ccc23)cc(OC(F)F)c1C(=O)N[C@@H]1C[C@@H]1F ; COc1cc(-c2coc3cc(OC(C)CN4CCCC4)ccc23)cc(OC(F)F)c1C(=O)N[C@@H]1C[C@@H]1F]

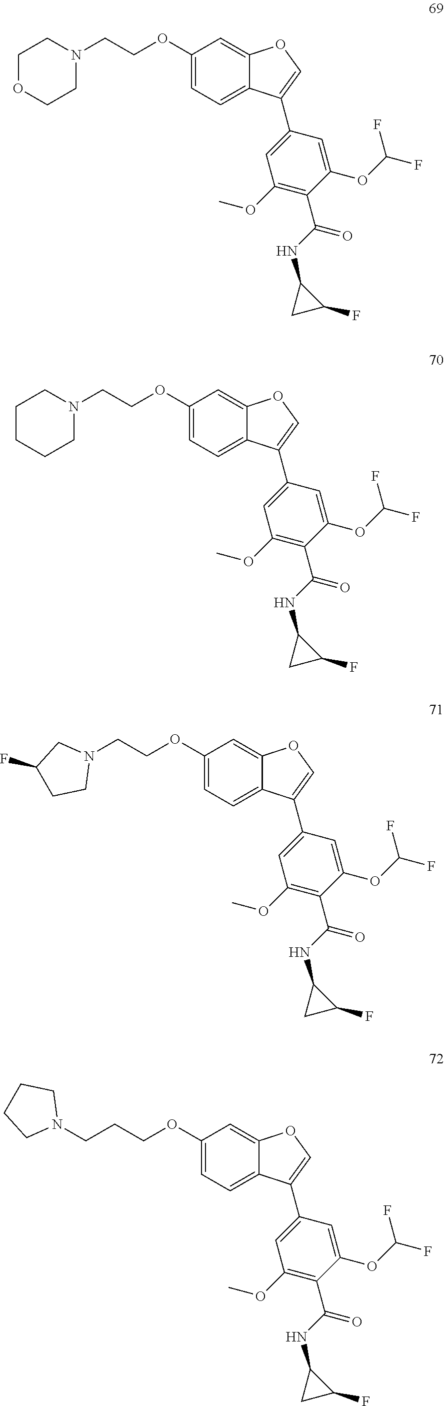 [SMILES: COc1cc(-c2coc3cc(OCCN4CCOCC4)ccc23)cc(OC(F)F)c1C(=O)N[C@@H]1C[C@@H]1F ; COc1cc(-c2coc3cc(OCCN4CCCCC4)ccc23)cc(OC(F)F)c1C(=O)N[C@@H]1C[C@@H]1F ; COc1cc(-c2coc3cc(OCCN4CC[C@@H](F)C4)ccc23)cc(OC(F)F)c1C(=O)N[C@@H]1C[C@@H]1F ; COc1cc(-c2coc3cc(OCCCN4CCCC4)ccc23)cc(OC(F)F)c1C(=O)N[C@@H]1C[C@@H]1F]

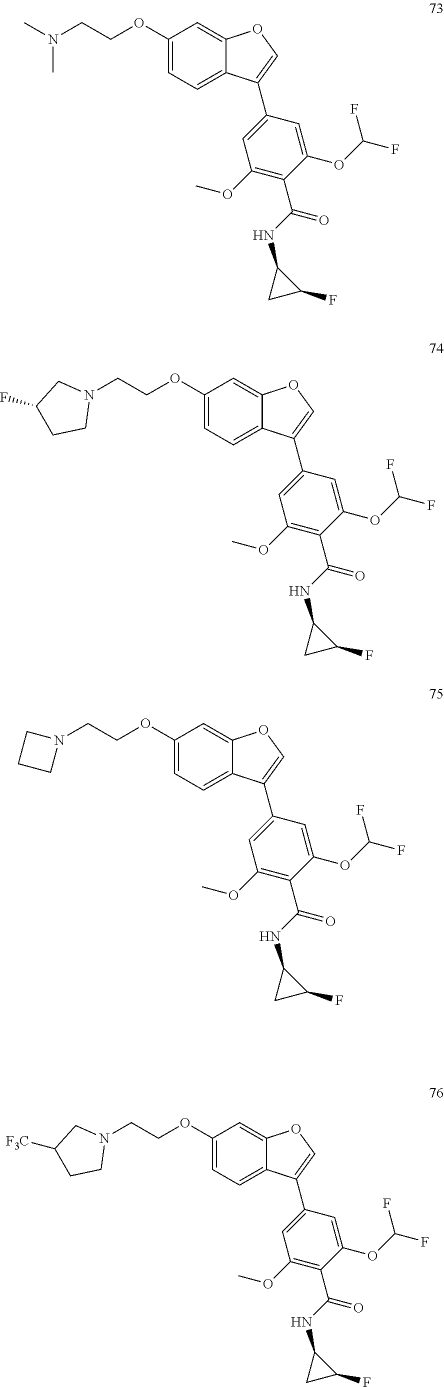 [SMILES: COc1cc(-c2coc3cc(OCCN(C)C)ccc23)cc(OC(F)F)c1C(=O)N[C@@H]1C[C@@H]1F ; COc1cc(-c2coc3cc(OCCN4CC[C@H](F)C4)ccc23)cc(OC(F)F)c1C(=O)N[C@@H]1C[C@@H]1F ; COc1cc(-c2coc3cc(OCCN4CCC4)ccc23)cc(OC(F)F)c1C(=O)N[C@@H]1C[C@@H]1F ; COc1cc(-c2coc3cc(OCCN4CCC(C(F)(F)F)C4)ccc23)cc(OC(F)F)c1C(=O)N[C@@H]1C[C@@H]1F]

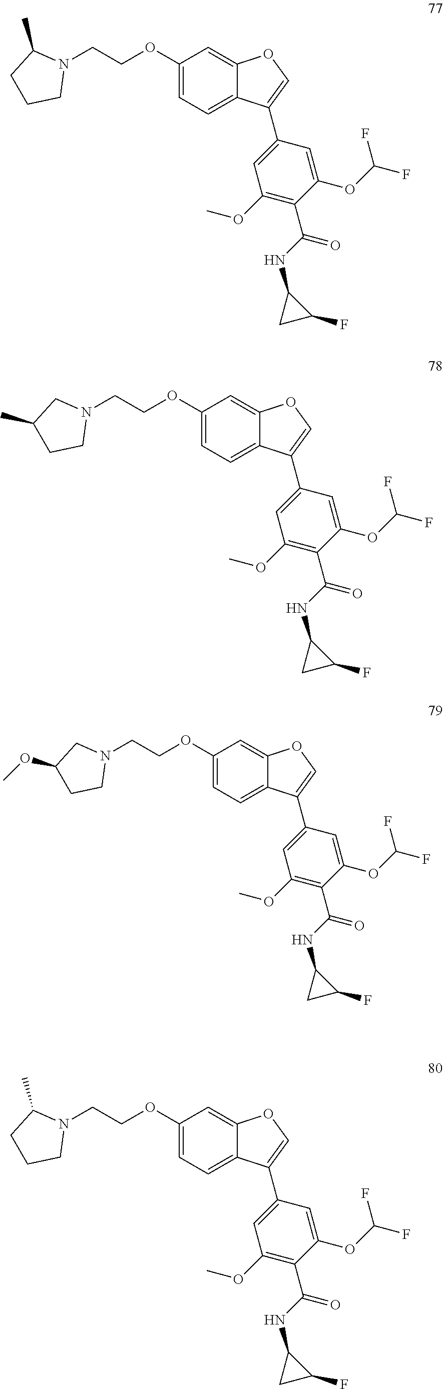 [SMILES: COc1cc(-c2coc3cc(OCCN4CCC[C@H]4C)ccc23)cc(OC(F)F)c1C(=O)N[C@@H]1C[C@@H]1F ; COc1cc(-c2coc3cc(OCCN4CC[C@@H](C)C4)ccc23)cc(OC(F)F)c1C(=O)N[C@@H]1C[C@@H]1F ; COc1cc(-c2coc3cc(OCCN4CC[C@@H](OC)C4)ccc23)cc(OC(F)F)c1C(=O)N[C@@H]1C[C@@H]1F ; COc1cc(-c2coc3cc(OCCN4CCC[C@@H]4C)ccc23)cc(OC(F)F)c1C(=O)N[C@@H]1C[C@@H]1F]

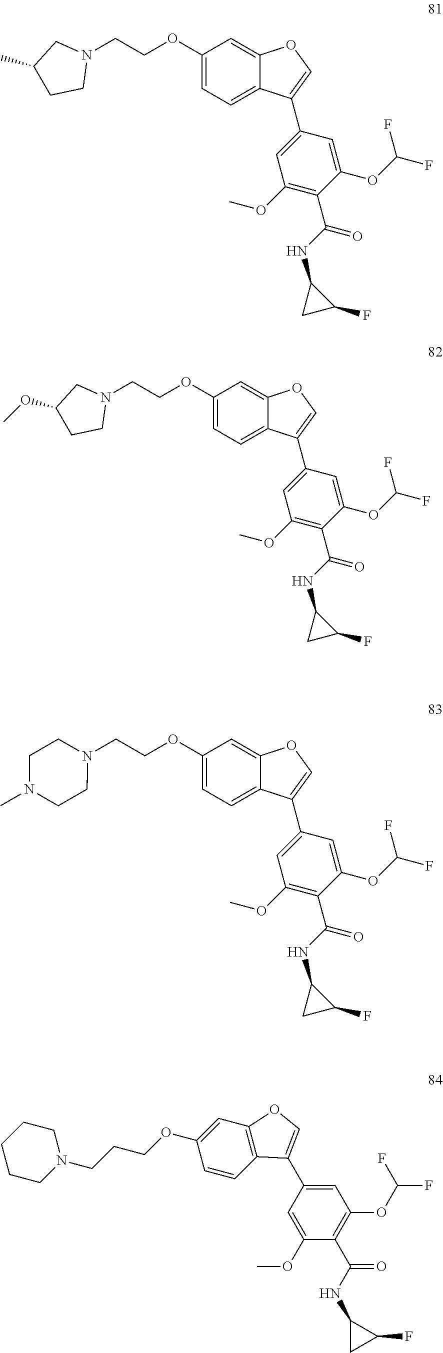 [SMILES: COc1cc(-c2coc3cc(OCCN4CC[C@H](C)C4)ccc23)cc(OC(F)F)c1C(=O)N[C@@H]1C[C@@H]1F ; COc1cc(-c2coc3cc(OCCN4CC[C@H](OC)C4)ccc23)cc(OC(F)F)c1C(=O)N[C@@H]1C[C@@H]1F ; COc1cc(-c2coc3cc(OCCN4CCN(C)CC4)ccc23)cc(OC(F)F)c1C(=O)N[C@@H]1C[C@@H]1F ; COc1cc(-c2coc3cc(OCCCN4CCCCC4)ccc23)cc(OC(F)F)c1C(=O)N[C@@H]1C[C@@H]1F]

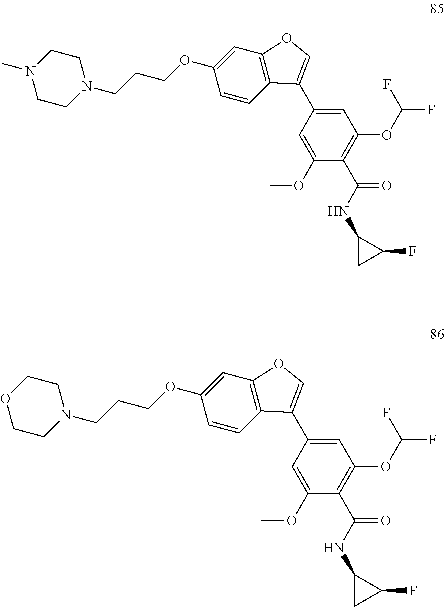 [SMILES: COc1cc(-c2coc3cc(OCCCN4CCN(C)CC4)ccc23)cc(OC(F)F)c1C(=O)N[C@@H]1C[C@@H]1F ; COc1cc(-c2coc3cc(OCCCN4CCOCC4)ccc23)cc(OC(F)F)c1C(=O)N[C@@H]1C[C@@H]1F]

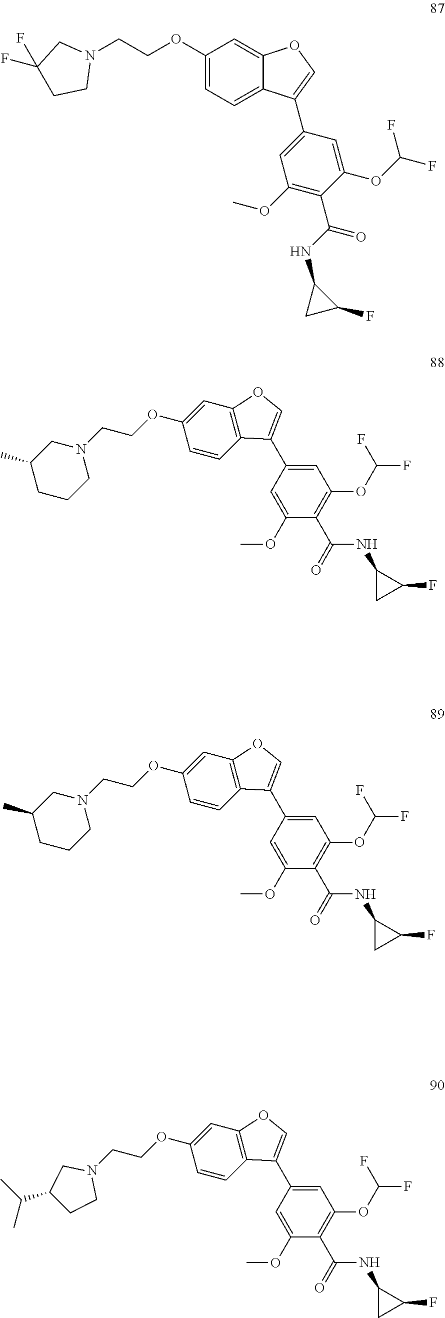 [SMILES: COc1cc(-c2coc3cc(OCCN4CCC(F)(F)C4)ccc23)cc(OC(F)F)c1C(=O)N[C@@H]1C[C@@H]1F ; COc1cc(-c2coc3cc(OCCN4CCC[C@H](C)C4)ccc23)cc(OC(F)F)c1C(=O)N[C@@H]1C[C@@H]1F ; COc1cc(-c2coc3cc(OCCN4CCC[C@@H](C)C4)ccc23)cc(OC(F)F)c1C(=O)N[C@@H]1C[C@@H]1F ; COc1cc(-c2coc3cc(OCCN4CC[C@H](C(C)C)C4)ccc23)cc(OC(F)F)c1C(=O)N[C@@H]1C[C@@H]1F]

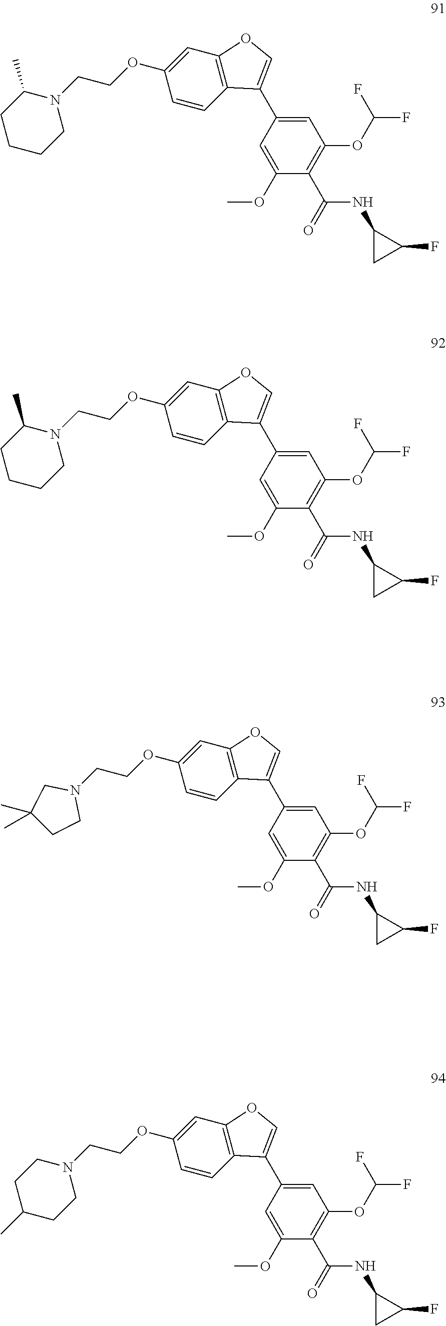 [SMILES: COc1cc(-c2coc3cc(OCCN4CCCC[C@@H]4C)ccc23)cc(OC(F)F)c1C(=O)N[C@@H]1C[C@@H]1F ; COc1cc(-c2coc3cc(OCCN4CCCC[C@H]4C)ccc23)cc(OC(F)F)c1C(=O)N[C@@H]1C[C@@H]1F ; COc1cc(-c2coc3cc(OCCN4CCC(C)(C)C4)ccc23)cc(OC(F)F)c1C(=O)N[C@@H]1C[C@@H]1F ; COc1cc(-c2coc3cc(OCCN4CCC(C)CC4)ccc23)cc(OC(F)F)c1C(=O)N[C@@H]1C[C@@H]1F]

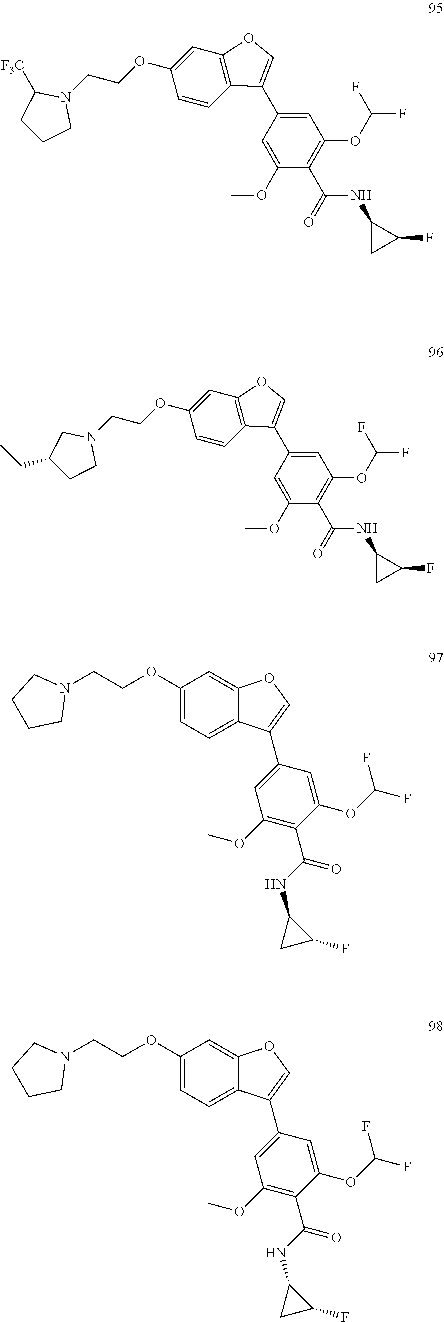 [SMILES: COc1cc(-c2coc3cc(OCCN4CCCC4C(F)(F)F)ccc23)cc(OC(F)F)c1C(=O)N[C@@H]1C[C@@H]1F ; CC[C@H]1CCN(CCOc2ccc3c(-c4cc(OC)c(C(=O)N[C@@H]5C[C@@H]5F)c(OC(F)F)c4)coc3c2)C1 ; COc1cc(-c2coc3cc(OCCN4CCCC4)ccc23)cc(OC(F)F)c1C(=O)N[C@@H]1C[C@H]1F ; COc1cc(-c2coc3cc(OCCN4CCCC4)ccc23)cc(OC(F)F)c1C(=O)N[C@H]1C[C@H]1F]

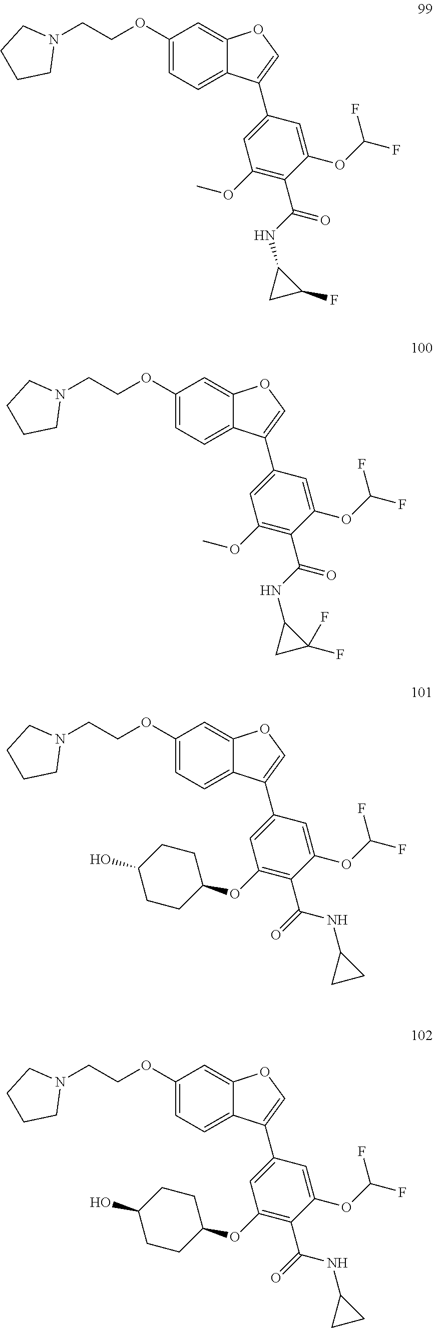 [SMILES: COc1cc(-c2coc3cc(OCCN4CCCC4)ccc23)cc(OC(F)F)c1C(=O)N[C@H]1C[C@@H]1F ; COc1cc(-c2coc3cc(OCCN4CCCC4)ccc23)cc(OC(F)F)c1C(=O)NC1CC1(F)F ; O=C(NC1CC1)c1c(OC(F)F)cc(-c2coc3cc(OCCN4CCCC4)ccc23)cc1O[C@H]1CC[C@H](O)CC1 ; O=C(NC1CC1)c1c(OC(F)F)cc(-c2coc3cc(OCCN4CCCC4)ccc23)cc1O[C@H]1CC[C@@H](O)CC1]

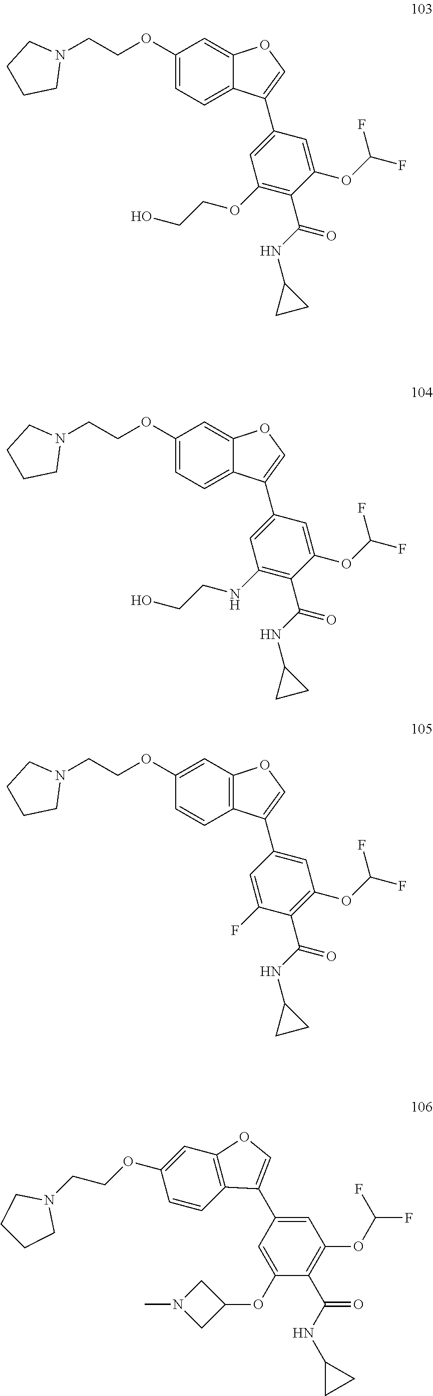 [SMILES: O=C(NC1CC1)c1c(OCCO)cc(-c2coc3cc(OCCN4CCCC4)ccc23)cc1OC(F)F ; O=C(NC1CC1)c1c(NCCO)cc(-c2coc3cc(OCCN4CCCC4)ccc23)cc1OC(F)F ; O=C(NC1CC1)c1c(F)cc(-c2coc3cc(OCCN4CCCC4)ccc23)cc1OC(F)F ; CN1CC(Oc2cc(-c3coc4cc(OCCN5CCCC5)ccc34)cc(OC(F)F)c2C(=O)NC2CC2)C1]

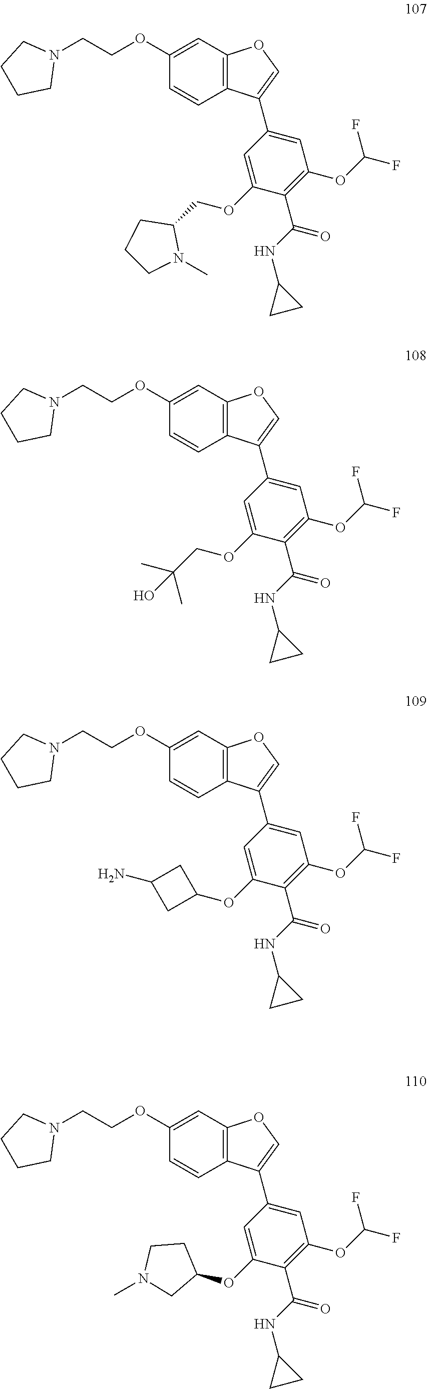 [SMILES: CN1CCC[C@@H]1COc1cc(-c2coc3cc(OCCN4CCCC4)ccc23)cc(OC(F)F)c1C(=O)NC1CC1 ; CC(C)(O)COc1cc(-c2coc3cc(OCCN4CCCC4)ccc23)cc(OC(F)F)c1C(=O)NC1CC1 ; NC1CC(Oc2cc(-c3coc4cc(OCCN5CCCC5)ccc34)cc(OC(F)F)c2C(=O)NC2CC2)C1 ; CN1CC[C@@H](Oc2cc(-c3coc4cc(OCCN5CCCC5)ccc34)cc(OC(F)F)c2C(=O)NC2CC2)C1]

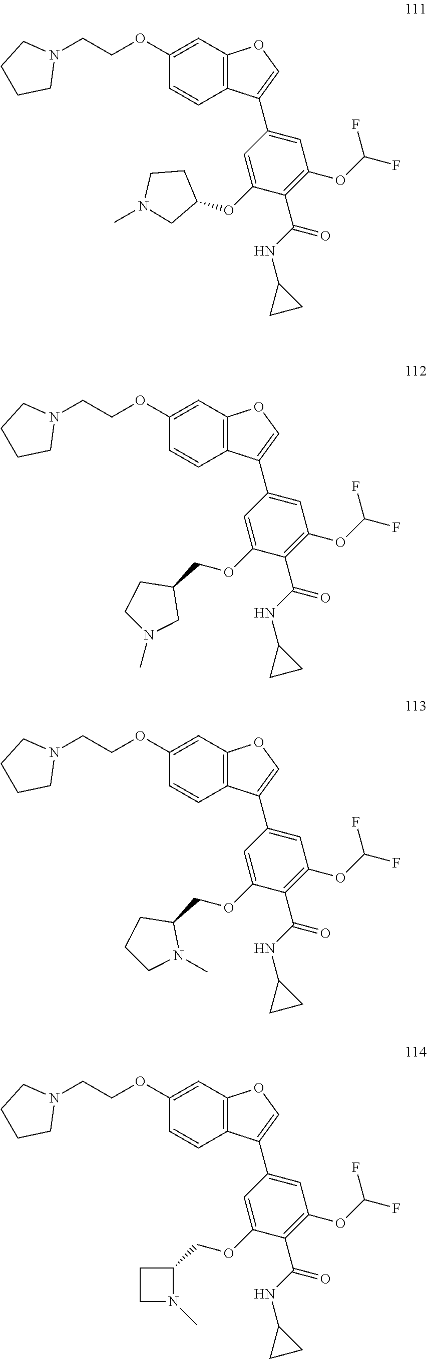 [SMILES: CN1CC[C@H](Oc2cc(-c3coc4cc(OCCN5CCCC5)ccc34)cc(OC(F)F)c2C(=O)NC2CC2)C1 ; CN1CC[C@@H](COc2cc(-c3coc4cc(OCCN5CCCC5)ccc34)cc(OC(F)F)c2C(=O)NC2CC2)C1 ; CN1CCC[C@H]1COc1cc(-c2coc3cc(OCCN4CCCC4)ccc23)cc(OC(F)F)c1C(=O)NC1CC1 ; CN1CC[C@@H]1COc1cc(-c2coc3cc(OCCN4CCCC4)ccc23)cc(OC(F)F)c1C(=O)NC1CC1]

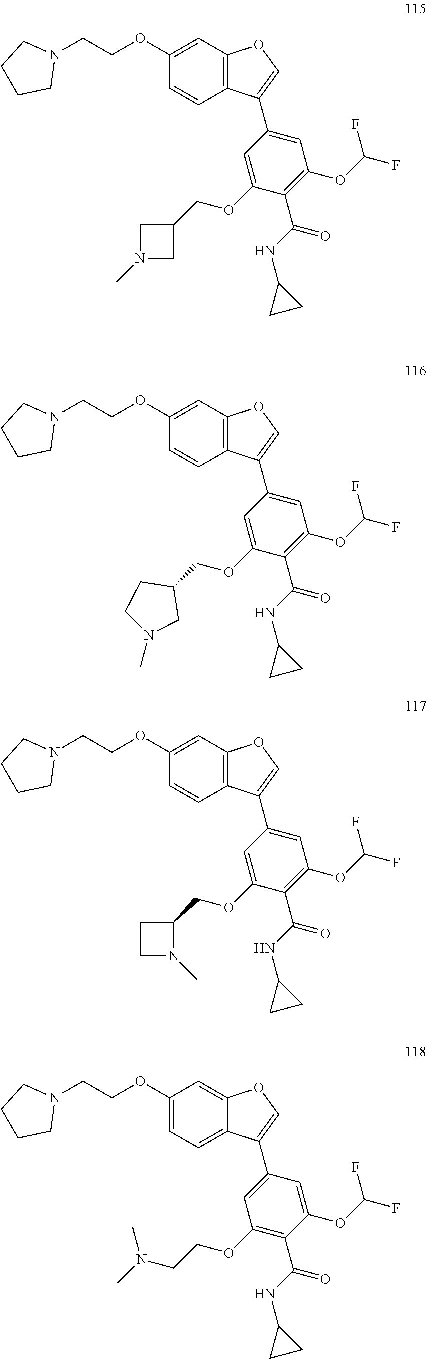 [SMILES: CN1CC(COc2cc(-c3coc4cc(OCCN5CCCC5)ccc34)cc(OC(F)F)c2C(=O)NC2CC2)C1 ; CN1CC[C@H](COc2cc(-c3coc4cc(OCCN5CCCC5)ccc34)cc(OC(F)F)c2C(=O)NC2CC2)C1 ; CN1CC[C@H]1COc1cc(-c2coc3cc(OCCN4CCCC4)ccc23)cc(OC(F)F)c1C(=O)NC1CC1 ; CN(C)CCOc1cc(-c2coc3cc(OCCN4CCCC4)ccc23)cc(OC(F)F)c1C(=O)NC1CC1]

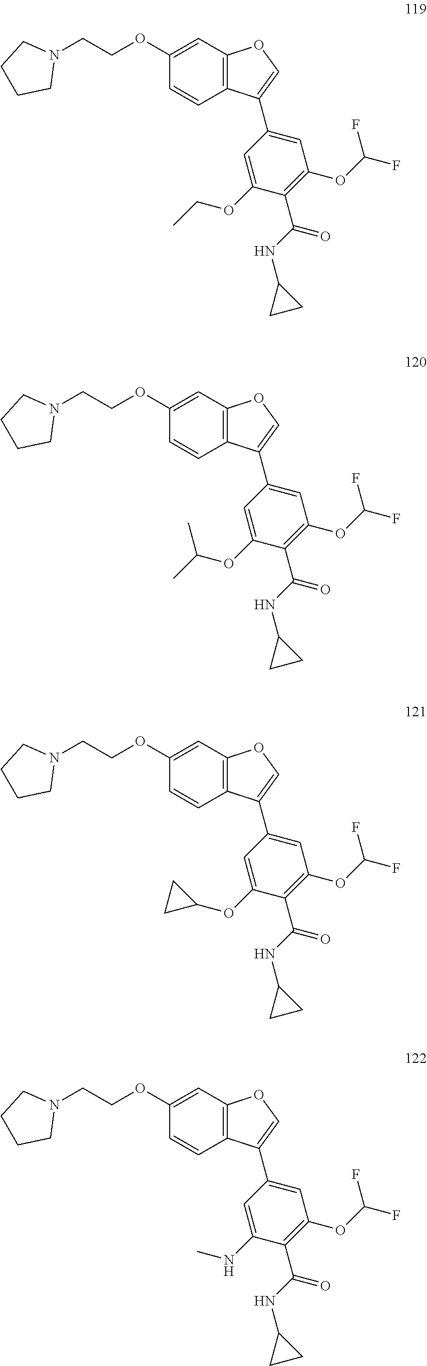 [SMILES: CCOc1cc(-c2coc3cc(OCCN4CCCC4)ccc23)cc(OC(F)F)c1C(=O)NC1CC1 ; CC(C)Oc1cc(-c2coc3cc(OCCN4CCCC4)ccc23)cc(OC(F)F)c1C(=O)NC1CC1 ; O=C(NC1CC1)c1c(OC(F)F)cc(-c2coc3cc(OCCN4CCCC4)ccc23)cc1OC1CC1 ; CNc1cc(-c2coc3cc(OCCN4CCCC4)ccc23)cc(OC(F)F)c1C(=O)NC1CC1]

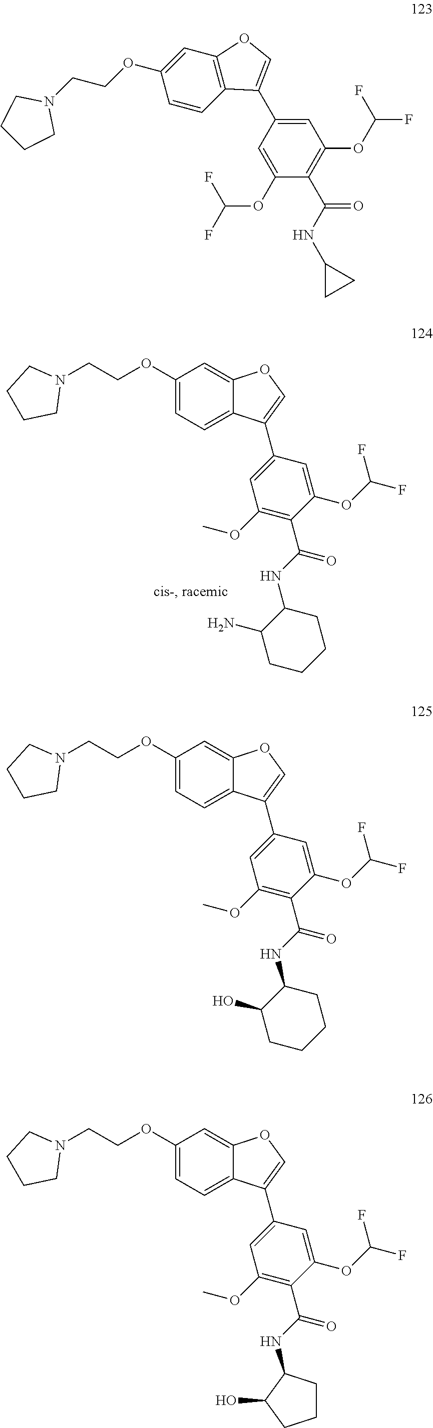 [SMILES: O=C(NC1CC1)c1c(OC(F)F)cc(-c2coc3cc(OCCN4CCCC4)ccc23)cc1OC(F)F ; COc1cc(-c2coc3cc(OCCN4CCCC4)ccc23)cc(OC(F)F)c1C(=O)NC1CCCCC1N ; COc1cc(-c2coc3cc(OCCN4CCCC4)ccc23)cc(OC(F)F)c1C(=O)N[C@H]1CCCC[C@H]1O ; COc1cc(-c2coc3cc(OCCN4CCCC4)ccc23)cc(OC(F)F)c1C(=O)N[C@H]1CCC[C@H]1O]

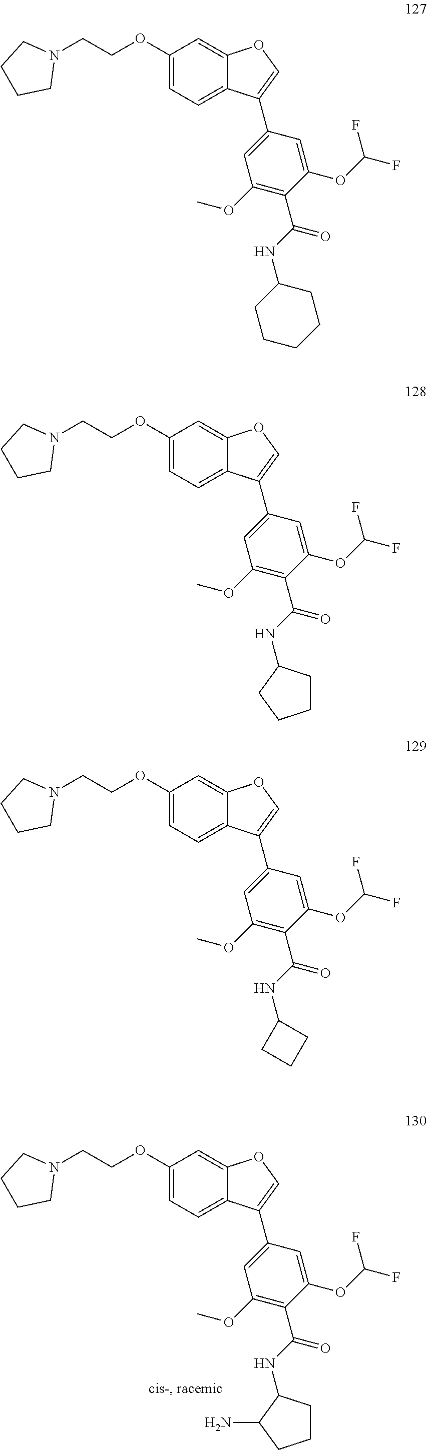 [SMILES: COc1cc(-c2coc3cc(OCCN4CCCC4)ccc23)cc(OC(F)F)c1C(=O)NC1CCCCC1 ; COc1cc(-c2coc3cc(OCCN4CCCC4)ccc23)cc(OC(F)F)c1C(=O)NC1CCCC1 ; COc1cc(-c2coc3cc(OCCN4CCCC4)ccc23)cc(OC(F)F)c1C(=O)NC1CCC1 ; COc1cc(-c2coc3cc(OCCN4CCCC4)ccc23)cc(OC(F)F)c1C(=O)NC1CCCC1N]

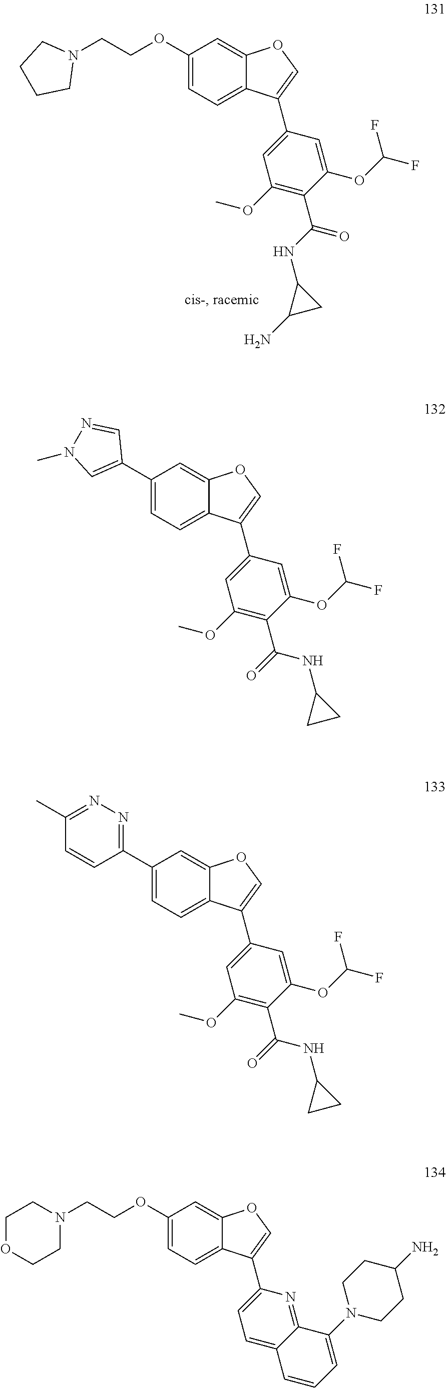 [SMILES: COc1cc(-c2coc3cc(OCCN4CCCC4)ccc23)cc(OC(F)F)c1C(=O)NC1CC1N ; COc1cc(-c2coc3cc(-c4cnn(C)c4)ccc23)cc(OC(F)F)c1C(=O)NC1CC1 ; COc1cc(-c2coc3cc(-c4ccc(C)nn4)ccc23)cc(OC(F)F)c1C(=O)NC1CC1 ; NC1CCN(c2cccc3ccc(-c4coc5cc(OCCN6CCOCC6)ccc45)nc23)CC1]

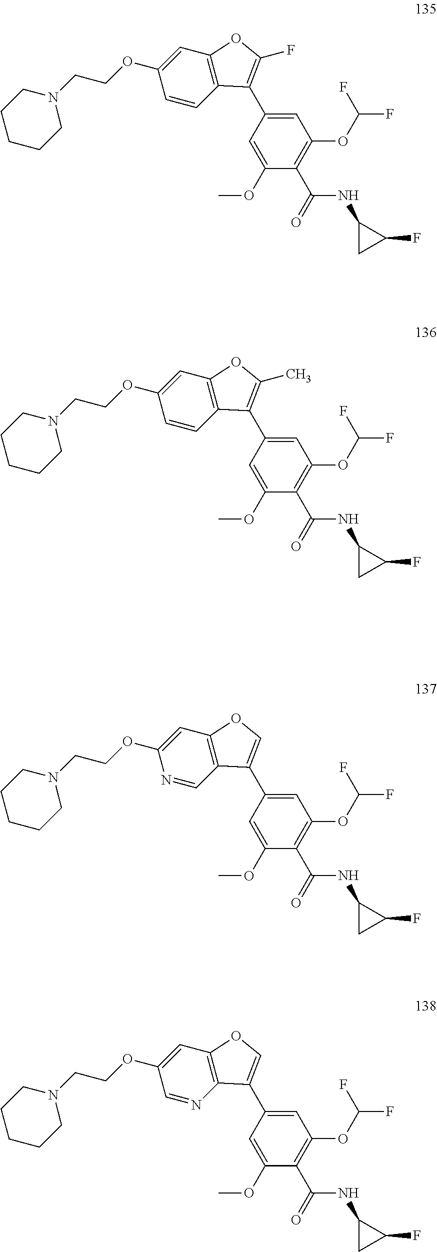 [SMILES: COc1cc(-c2c(F)oc3cc(OCCN4CCCCC4)ccc23)cc(OC(F)F)c1C(=O)N[C@@H]1C[C@@H]1F ; COc1cc(-c2c(C)oc3cc(OCCN4CCCCC4)ccc23)cc(OC(F)F)c1C(=O)N[C@@H]1C[C@@H]1F ; COc1cc(-c2coc3cc(OCCN4CCCCC4)ncc23)cc(OC(F)F)c1C(=O)N[C@@H]1C[C@@H]1F ; COc1cc(-c2coc3cc(OCCN4CCCCC4)cnc23)cc(OC(F)F)c1C(=O)N[C@@H]1C[C@@H]1F]

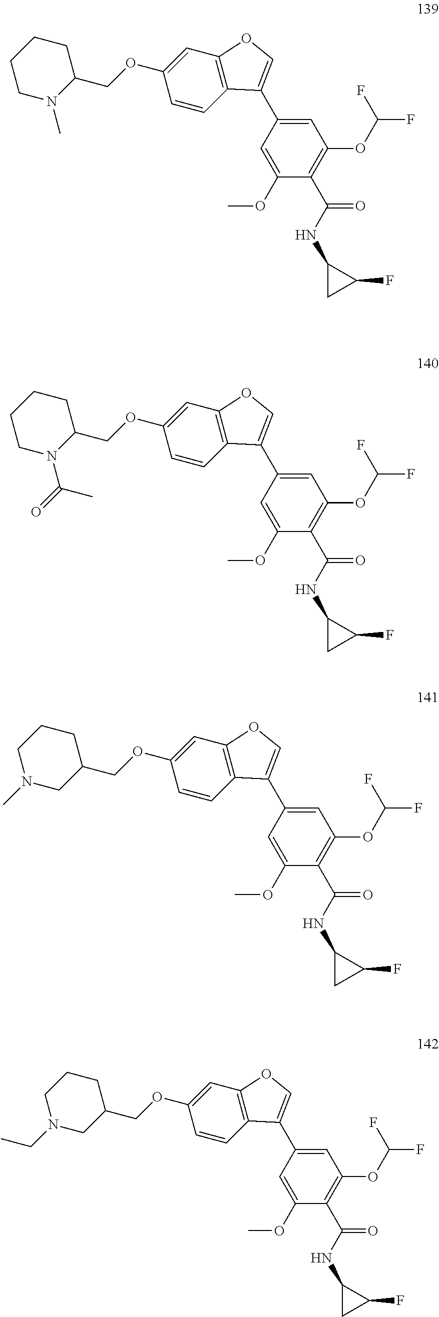 [SMILES: COc1cc(-c2coc3cc(OCC4CCCCN4C)ccc23)cc(OC(F)F)c1C(=O)N[C@@H]1C[C@@H]1F ; COc1cc(-c2coc3cc(OCC4CCCCN4C(C)=O)ccc23)cc(OC(F)F)c1C(=O)N[C@@H]1C[C@@H]1F ; COc1cc(-c2coc3cc(OCC4CCCN(C)C4)ccc23)cc(OC(F)F)c1C(=O)N[C@@H]1C[C@@H]1F ; CCN1CCCC(COc2ccc3c(-c4cc(OC)c(C(=O)N[C@@H]5C[C@@H]5F)c(OC(F)F)c4)coc3c2)C1]

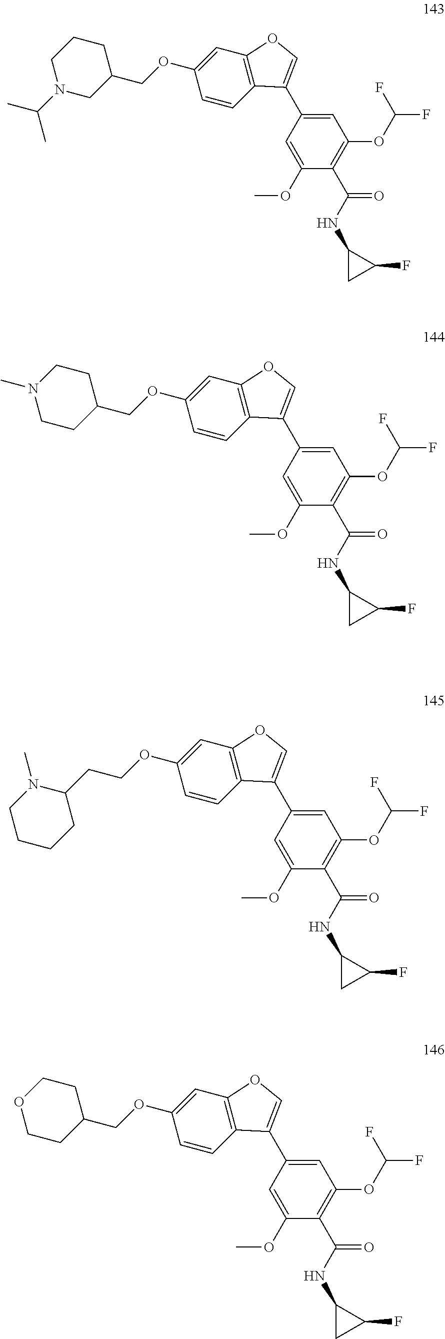 [SMILES: COc1cc(-c2coc3cc(OCC4CCCN(C(C)C)C4)ccc23)cc(OC(F)F)c1C(=O)N[C@@H]1C[C@@H]1F ; COc1cc(-c2coc3cc(OCC4CCN(C)CC4)ccc23)cc(OC(F)F)c1C(=O)N[C@@H]1C[C@@H]1F ; COc1cc(-c2coc3cc(OCCC4CCCCN4C)ccc23)cc(OC(F)F)c1C(=O)N[C@@H]1C[C@@H]1F ; COc1cc(-c2coc3cc(OCC4CCOCC4)ccc23)cc(OC(F)F)c1C(=O)N[C@@H]1C[C@@H]1F]

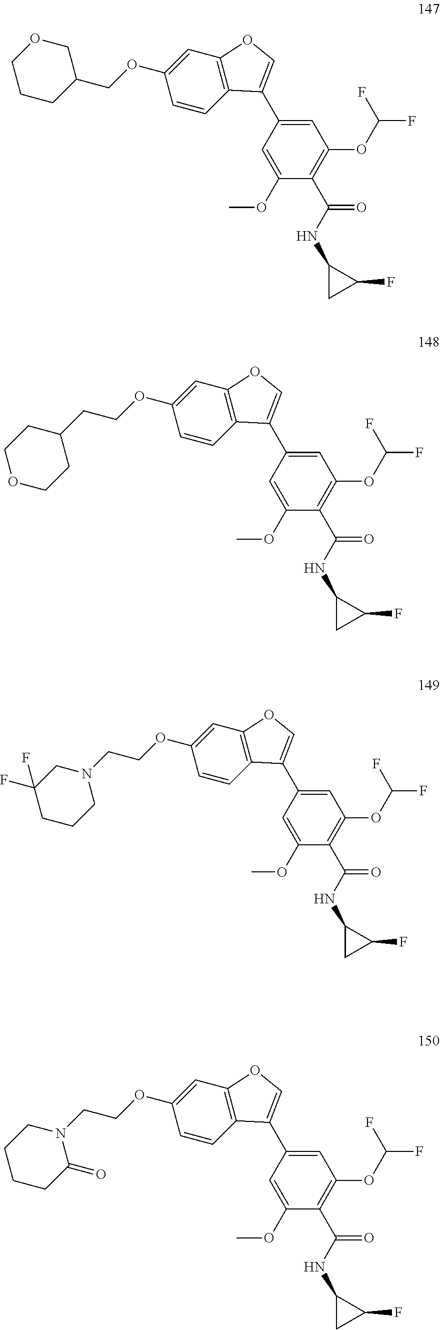 [SMILES: COc1cc(-c2coc3cc(OCC4CCCOC4)ccc23)cc(OC(F)F)c1C(=O)N[C@@H]1C[C@@H]1F ; COc1cc(-c2coc3cc(OCCC4CCOCC4)ccc23)cc(OC(F)F)c1C(=O)N[C@@H]1C[C@@H]1F ; COc1cc(-c2coc3cc(OCCN4CCCC(F)(F)C4)ccc23)cc(OC(F)F)c1C(=O)N[C@@H]1C[C@@H]1F ; COc1cc(-c2coc3cc(OCCN4CCCCC4=O)ccc23)cc(OC(F)F)c1C(=O)N[C@@H]1C[C@@H]1F]

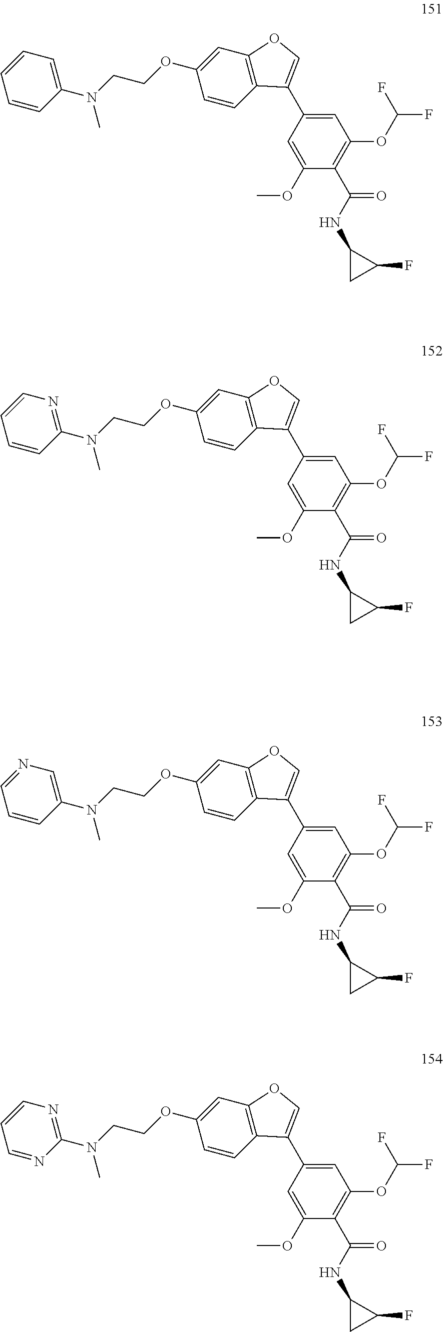 [SMILES: COc1cc(-c2coc3cc(OCCN(C)c4ccccc4)ccc23)cc(OC(F)F)c1C(=O)N[C@@H]1C[C@@H]1F ; COc1cc(-c2coc3cc(OCCN(C)c4ccccn4)ccc23)cc(OC(F)F)c1C(=O)N[C@@H]1C[C@@H]1F ; COc1cc(-c2coc3cc(OCCN(C)c4cccnc4)ccc23)cc(OC(F)F)c1C(=O)N[C@@H]1C[C@@H]1F ; COc1cc(-c2coc3cc(OCCN(C)c4ncccn4)ccc23)cc(OC(F)F)c1C(=O)N[C@@H]1C[C@@H]1F]

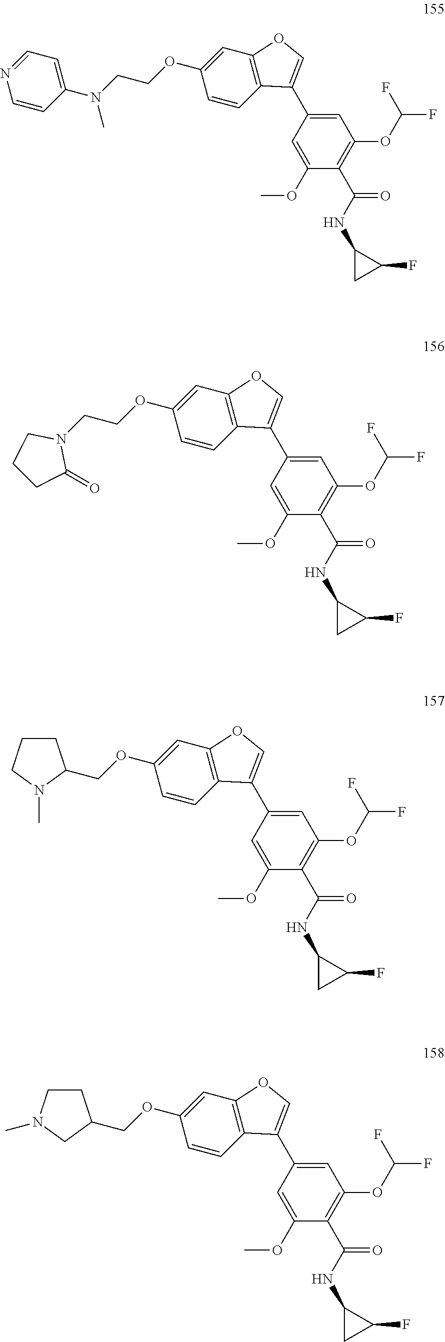 [SMILES: COc1cc(-c2coc3cc(OCCN(C)c4ccncc4)ccc23)cc(OC(F)F)c1C(=O)N[C@@H]1C[C@@H]1F ; COc1cc(-c2coc3cc(OCCN4CCCC4=O)ccc23)cc(OC(F)F)c1C(=O)N[C@@H]1C[C@@H]1F ; COc1cc(-c2coc3cc(OCC4CCCN4C)ccc23)cc(OC(F)F)c1C(=O)N[C@@H]1C[C@@H]1F ; COc1cc(-c2coc3cc(OCC4CCN(C)C4)ccc23)cc(OC(F)F)c1C(=O)N[C@@H]1C[C@@H]1F]

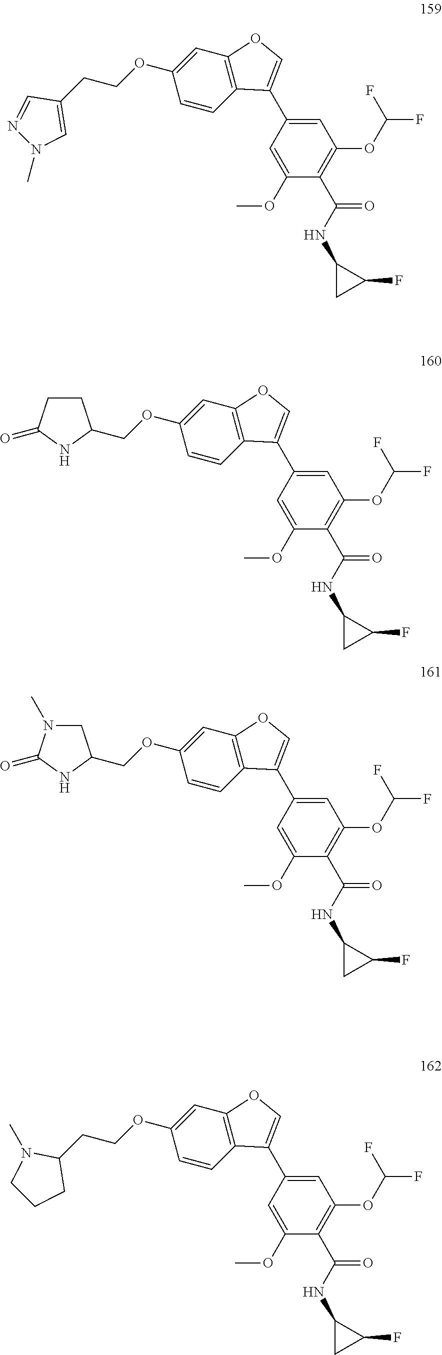 [SMILES: COc1cc(-c2coc3cc(OCCc4cnn(C)c4)ccc23)cc(OC(F)F)c1C(=O)N[C@@H]1C[C@@H]1F ; COc1cc(-c2coc3cc(OCC4CCC(=O)N4)ccc23)cc(OC(F)F)c1C(=O)N[C@@H]1C[C@@H]1F ; COc1cc(-c2coc3cc(OCC4CN(C)C(=O)N4)ccc23)cc(OC(F)F)c1C(=O)N[C@@H]1C[C@@H]1F ; COc1cc(-c2coc3cc(OCCC4CCCN4C)ccc23)cc(OC(F)F)c1C(=O)N[C@@H]1C[C@@H]1F]

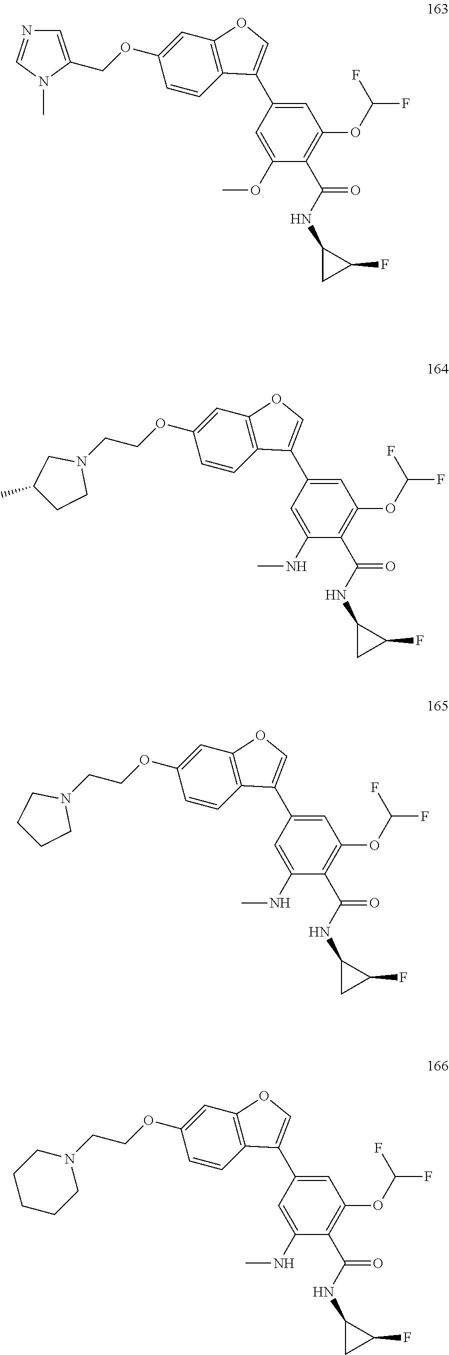 [SMILES: COc1cc(-c2coc3cc(OCc4cncn4C)ccc23)cc(OC(F)F)c1C(=O)N[C@@H]1C[C@@H]1F ; CNc1cc(-c2coc3cc(OCCN4CC[C@H](C)C4)ccc23)cc(OC(F)F)c1C(=O)N[C@@H]1C[C@@H]1F ; CNc1cc(-c2coc3cc(OCCN4CCCC4)ccc23)cc(OC(F)F)c1C(=O)N[C@@H]1C[C@@H]1F ; CNc1cc(-c2coc3cc(OCCN4CCCCC4)ccc23)cc(OC(F)F)c1C(=O)N[C@@H]1C[C@@H]1F]

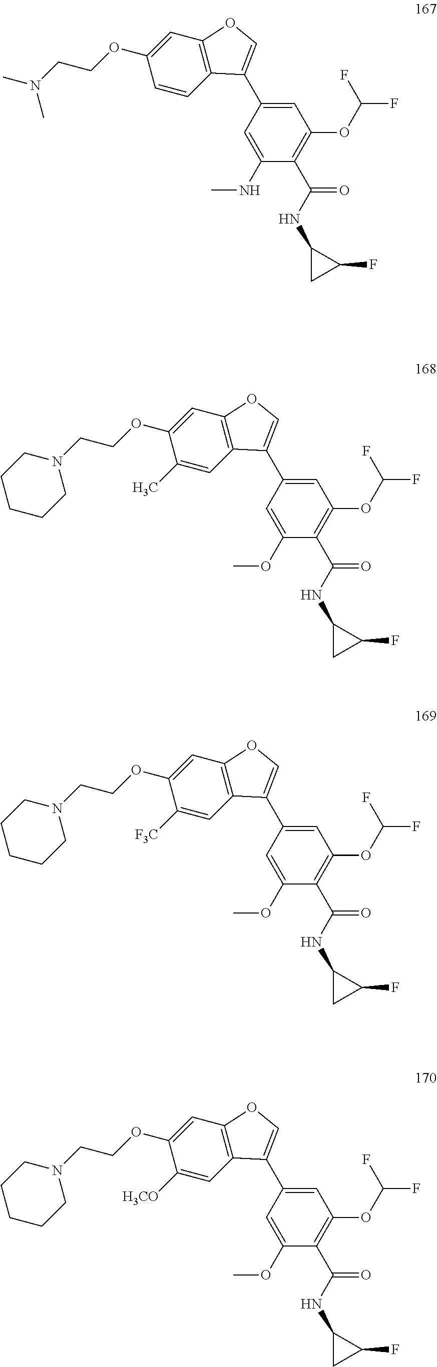 [SMILES: CNc1cc(-c2coc3cc(OCCN(C)C)ccc23)cc(OC(F)F)c1C(=O)N[C@@H]1C[C@@H]1F ; COc1cc(-c2coc3cc(OCCN4CCCCC4)c(C)cc23)cc(OC(F)F)c1C(=O)N[C@@H]1C[C@@H]1F ; COc1cc(-c2coc3cc(OCCN4CCCCC4)c(C(F)(F)F)cc23)cc(OC(F)F)c1C(=O)N[C@@H]1C[C@@H]1F ; COc1cc2c(-c3cc(OC)c(C(=O)N[C@@H]4C[C@@H]4F)c(OC(F)F)c3)coc2cc1OCCN1CCCCC1]

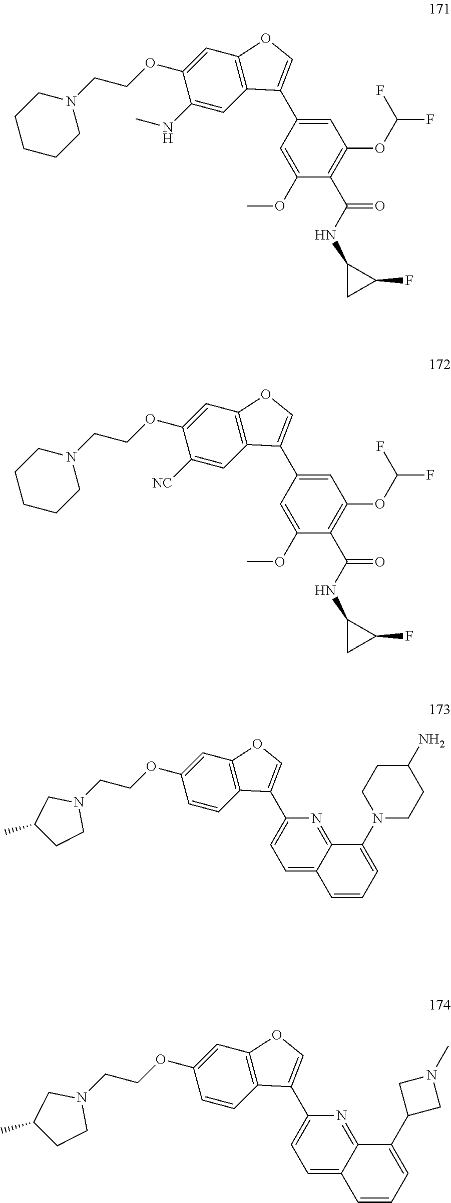

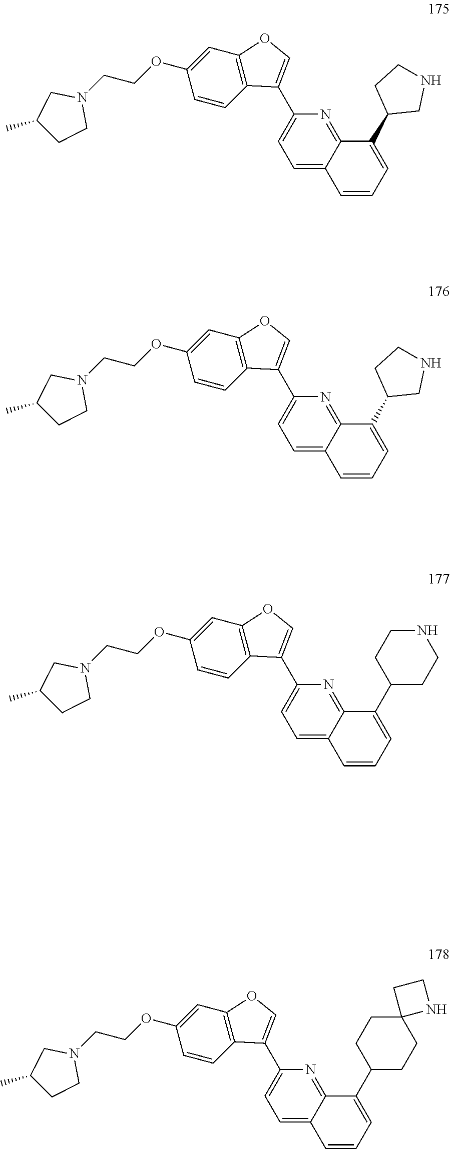 [SMILES: C[C@H]1CCN(CCOc2ccc3c(-c4ccc5cccc([C@H]6CCNC6)c5n4)coc3c2)C1 ; C[C@H]1CCN(CCOc2ccc3c(-c4ccc5cccc([C@@H]6CCNC6)c5n4)coc3c2)C1 ; C[C@H]1CCN(CCOc2ccc3c(-c4ccc5cccc(C6CCNCC6)c5n4)coc3c2)C1 ; C[C@H]1CCN(CCOc2ccc3c(-c4ccc5cccc(C6CCC7(CCN7)CC6)c5n4)coc3c2)C1]

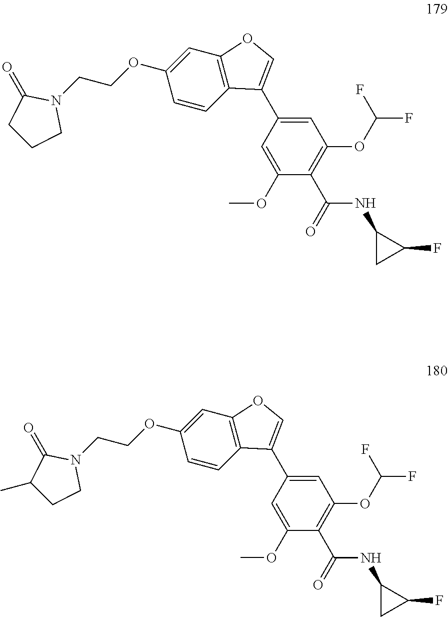 [SMILES: COc1cc(-c2coc3cc(OCCN4CCCC4=O)ccc23)cc(OC(F)F)c1C(=O)N[C@@H]1C[C@@H]1F ; COc1cc(-c2coc3cc(OCCN4CCC(C)C4=O)ccc23)cc(OC(F)F)c1C(=O)N[C@@H]1C[C@@H]1F]

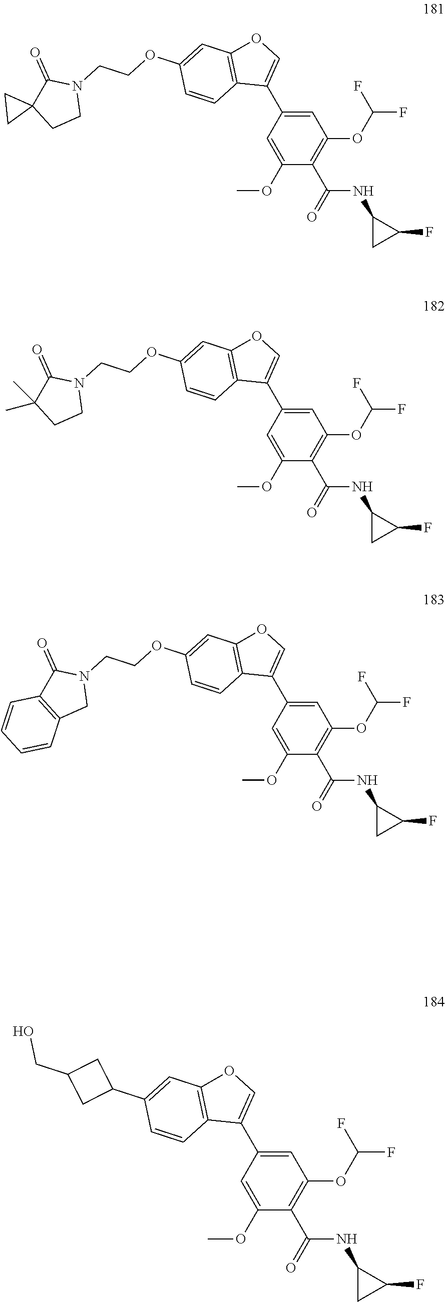 [SMILES: COc1cc(-c2coc3cc(OCCN4CCC5(CC5)C4=O)ccc23)cc(OC(F)F)c1C(=O)N[C@@H]1C[C@@H]1F ; COc1cc(-c2coc3cc(OCCN4CCC(C)(C)C4=O)ccc23)cc(OC(F)F)c1C(=O)N[C@@H]1C[C@@H]1F ; COc1cc(-c2coc3cc(OCCN4Cc5ccccc5C4=O)ccc23)cc(OC(F)F)c1C(=O)N[C@@H]1C[C@@H]1F ; COc1cc(-c2coc3cc(C4CC(CO)C4)ccc23)cc(OC(F)F)c1C(=O)N[C@@H]1C[C@@H]1F]

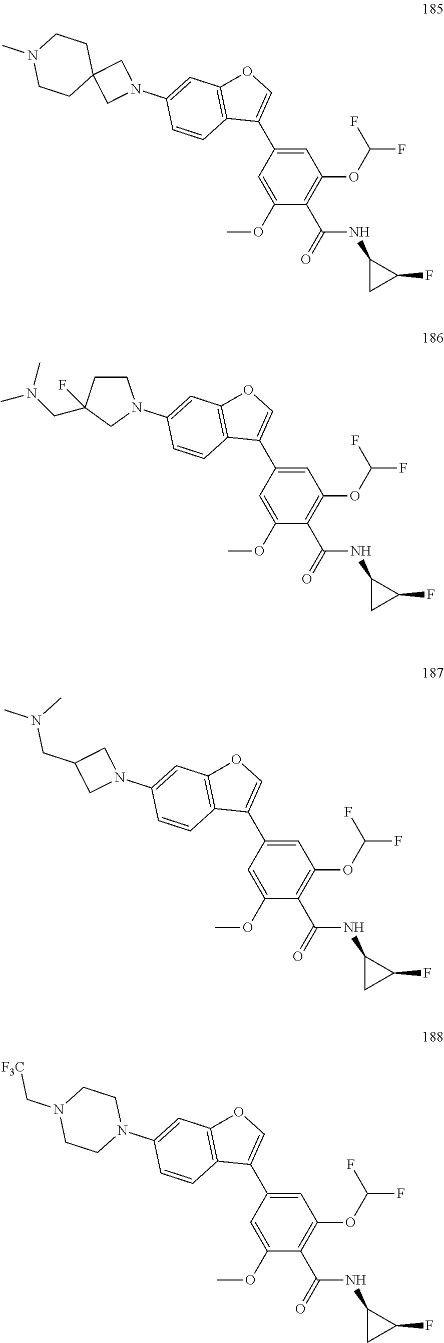 [SMILES: COc1cc(-c2coc3cc(N4CC5(CCN(C)CC5)C4)ccc23)cc(OC(F)F)c1C(=O)N[C@@H]1C[C@@H]1F ; COc1cc(-c2coc3cc(N4CCC(F)(CN(C)C)C4)ccc23)cc(OC(F)F)c1C(=O)N[C@@H]1C[C@@H]1F ; COc1cc(-c2coc3cc(N4CC(CN(C)C)C4)ccc23)cc(OC(F)F)c1C(=O)N[C@@H]1C[C@@H]1F ; COc1cc(-c2coc3cc(N4CCN(CC(F)(F)F)CC4)ccc23)cc(OC(F)F)c1C(=O)N[C@@H]1C[C@@H]1F]

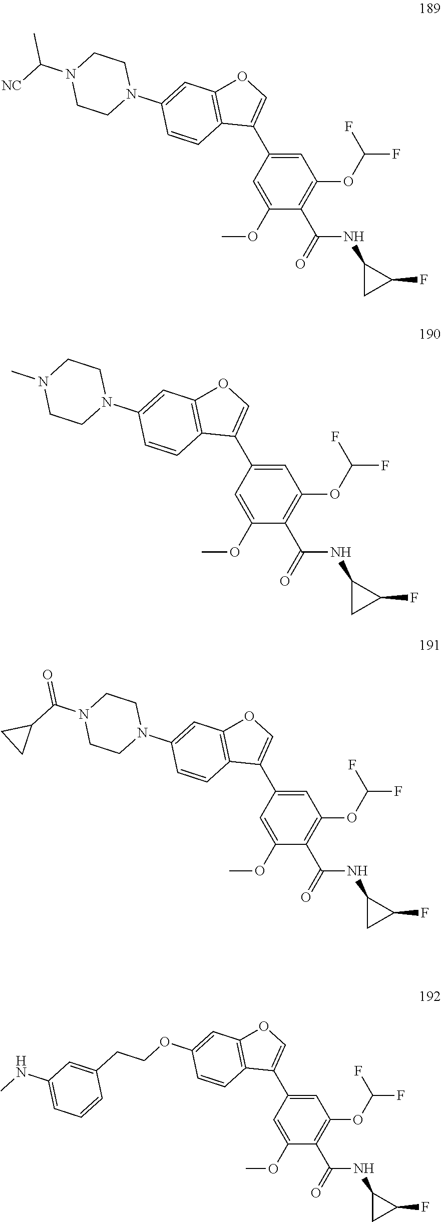 [SMILES: COc1cc(-c2coc3cc(N4CCN(C(C)C#N)CC4)ccc23)cc(OC(F)F)c1C(=O)N[C@@H]1C[C@@H]1F ; COc1cc(-c2coc3cc(N4CCN(C)CC4)ccc23)cc(OC(F)F)c1C(=O)N[C@@H]1C[C@@H]1F ; COc1cc(-c2coc3cc(N4CCN(C(=O)C5CC5)CC4)ccc23)cc(OC(F)F)c1C(=O)N[C@@H]1C[C@@H]1F ; CNc1cccc(CCOc2ccc3c(-c4cc(OC)c(C(=O)N[C@@H]5C[C@@H]5F)c(OC(F)F)c4)coc3c2)c1]

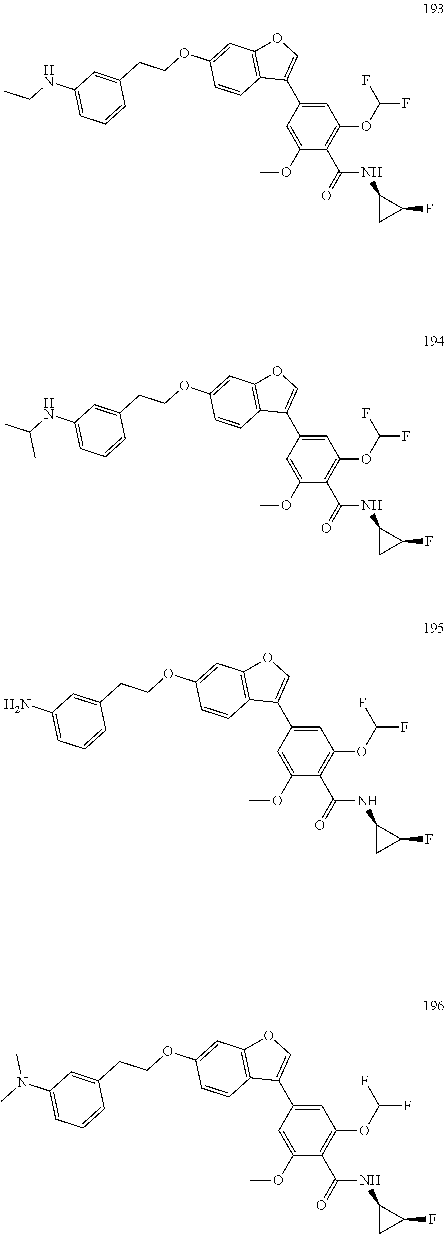 [SMILES: CCNc1cccc(CCOc2ccc3c(-c4cc(OC)c(C(=O)N[C@@H]5C[C@@H]5F)c(OC(F)F)c4)coc3c2)c1 ; COc1cc(-c2coc3cc(OCCc4cccc(NC(C)C)c4)ccc23)cc(OC(F)F)c1C(=O)N[C@@H]1C[C@@H]1F ; COc1cc(-c2coc3cc(OCCc4cccc(N)c4)ccc23)cc(OC(F)F)c1C(=O)N[C@@H]1C[C@@H]1F ; COc1cc(-c2coc3cc(OCCc4cccc(N(C)C)c4)ccc23)cc(OC(F)F)c1C(=O)N[C@@H]1C[C@@H]1F]

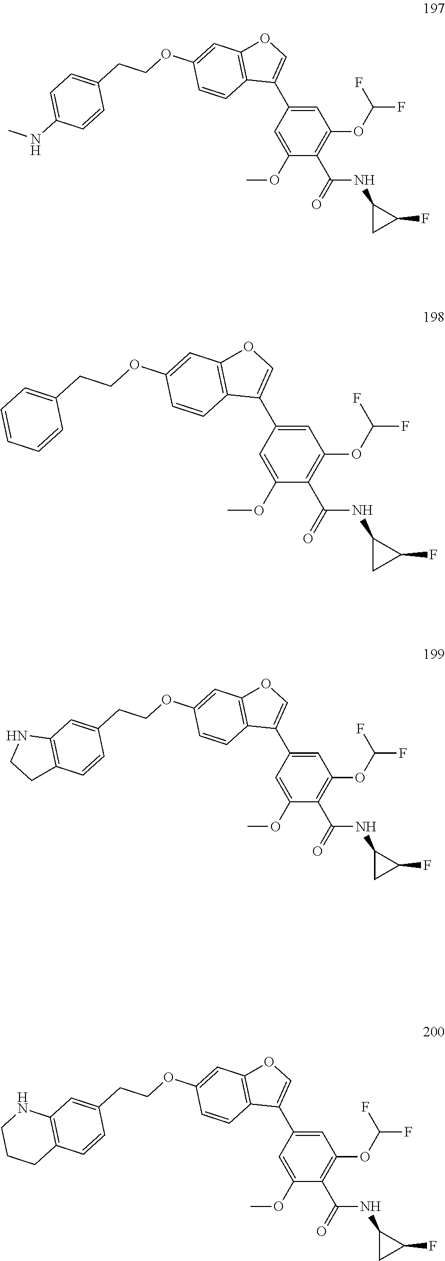 [SMILES: CNc1ccc(CCOc2ccc3c(-c4cc(OC)c(C(=O)N[C@@H]5C[C@@H]5F)c(OC(F)F)c4)coc3c2)cc1 ; COc1cc(-c2coc3cc(OCCc4ccccc4)ccc23)cc(OC(F)F)c1C(=O)N[C@@H]1C[C@@H]1F ; COc1cc(-c2coc3cc(OCCc4ccc5c(c4)NCC5)ccc23)cc(OC(F)F)c1C(=O)N[C@@H]1C[C@@H]1F ; COc1cc(-c2coc3cc(OCCc4ccc5c(c4)NCCC5)ccc23)cc(OC(F)F)c1C(=O)N[C@@H]1C[C@@H]1F]

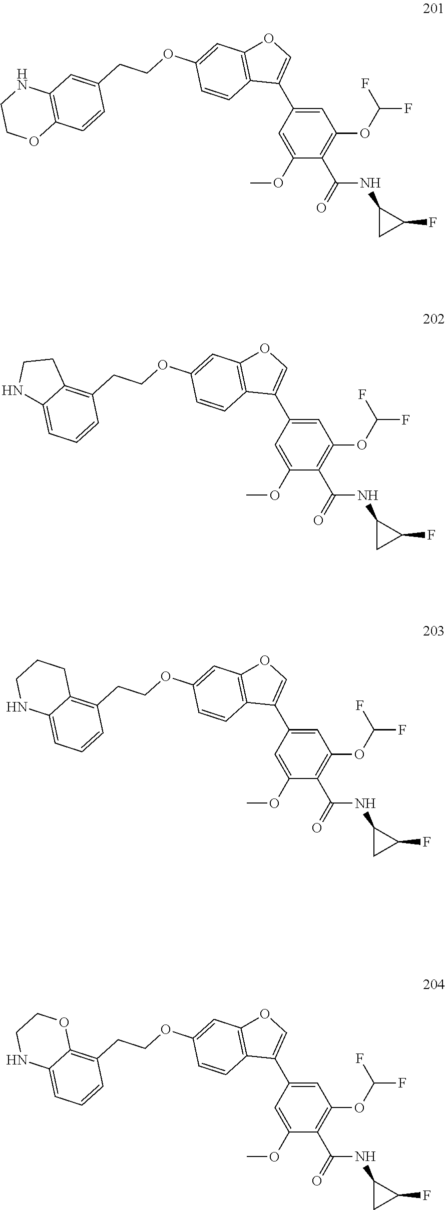 [SMILES: COc1cc(-c2coc3cc(OCCc4ccc5c(c4)NCCO5)ccc23)cc(OC(F)F)c1C(=O)N[C@@H]1C[C@@H]1F ; COc1cc(-c2coc3cc(OCCc4cccc5c4CCN5)ccc23)cc(OC(F)F)c1C(=O)N[C@@H]1C[C@@H]1F ; COc1cc(-c2coc3cc(OCCc4cccc5c4CCCN5)ccc23)cc(OC(F)F)c1C(=O)N[C@@H]1C[C@@H]1F ; COc1cc(-c2coc3cc(OCCc4cccc5c4OCCN5)ccc23)cc(OC(F)F)c1C(=O)N[C@@H]1C[C@@H]1F]

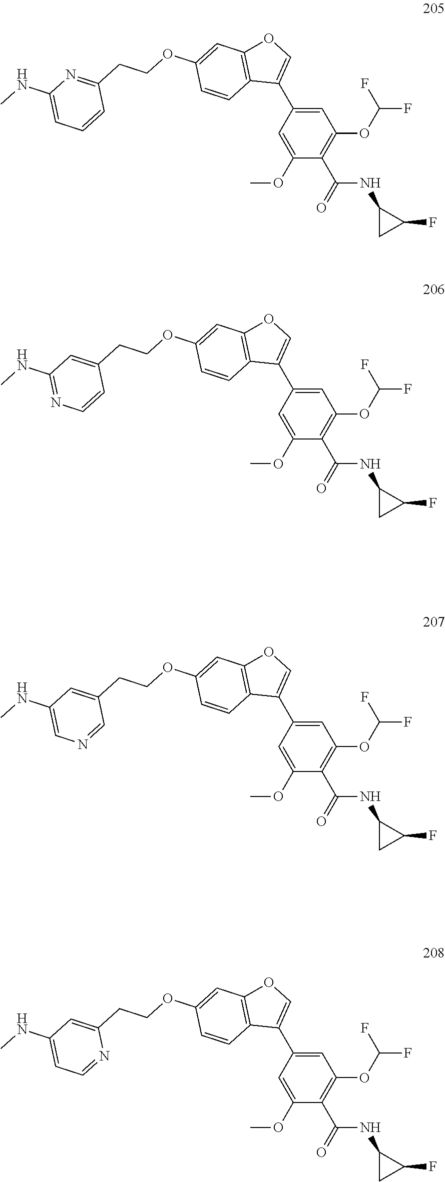 [SMILES: CNc1cccc(CCOc2ccc3c(-c4cc(OC)c(C(=O)N[C@@H]5C[C@@H]5F)c(OC(F)F)c4)coc3c2)n1 ; CNc1cc(CCOc2ccc3c(-c4cc(OC)c(C(=O)N[C@@H]5C[C@@H]5F)c(OC(F)F)c4)coc3c2)ccn1 ; CNc1cncc(CCOc2ccc3c(-c4cc(OC)c(C(=O)N[C@@H]5C[C@@H]5F)c(OC(F)F)c4)coc3c2)c1 ; CNc1ccnc(CCOc2ccc3c(-c4cc(OC)c(C(=O)N[C@@H]5C[C@@H]5F)c(OC(F)F)c4)coc3c2)c1]

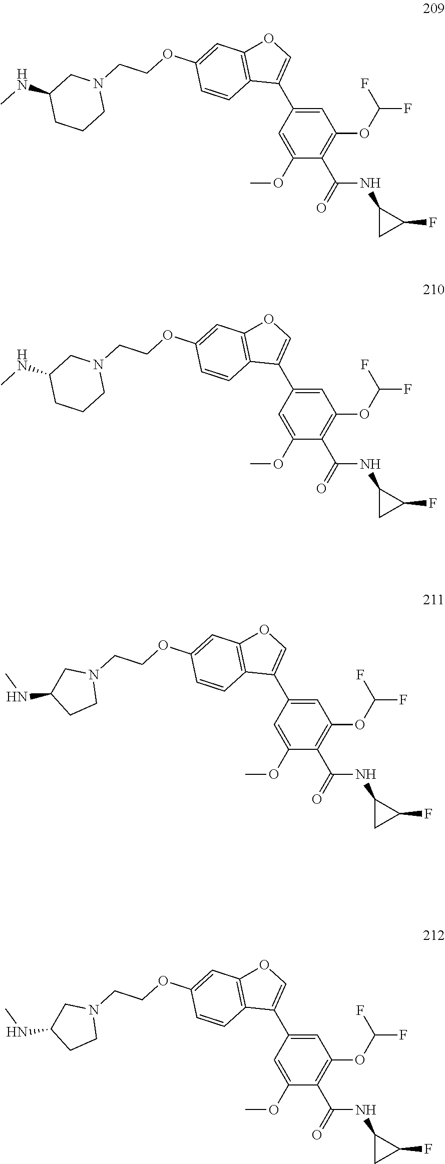 [SMILES: CN[C@@H]1CCCN(CCOc2ccc3c(-c4cc(OC)c(C(=O)N[C@@H]5C[C@@H]5F)c(OC(F)F)c4)coc3c2)C1 ; CN[C@H]1CCCN(CCOc2ccc3c(-c4cc(OC)c(C(=O)N[C@@H]5C[C@@H]5F)c(OC(F)F)c4)coc3c2)C1 ; CN[C@@H]1CCN(CCOc2ccc3c(-c4cc(OC)c(C(=O)N[C@@H]5C[C@@H]5F)c(OC(F)F)c4)coc3c2)C1 ; CN[C@H]1CCN(CCOc2ccc3c(-c4cc(OC)c(C(=O)N[C@@H]5C[C@@H]5F)c(OC(F)F)c4)coc3c2)C1]

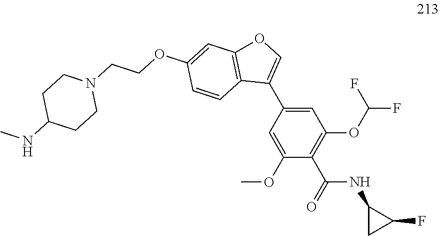 [SMILES: CNC1CCN(CCOc2ccc3c(-c4cc(OC)c(C(=O)N[C@@H]5C[C@@H]5F)c(OC(F)F)c4)coc3c2)CC1]

24. A pharmaceutical composition comprising the compound of any of claims 1-23, or a pharmaceutically acceptable salt, stereoisomer, or tautomer thereof, and a pharmaceutically acceptable excipient.

25. A method of treating a disease or disorder, comprising administering to a subject in need thereof a therapeutically effective amount of the compound of any of claims 1-23, or a pharmaceutically acceptable salt, stereoisomer, or tautomer thereof.

26. The method of claim 25, wherein the disease or disorder is selected from inflammatory diseases, autoinflammatory diseases, and autoimmune diseases.
